(19) United States
(12) Patent Application Publication
(10) Pub. No.: US 2025/0011289 A1
(43) Pub. Date: Jan. 9, 2025

(54) 1H-BENZO[B]AZEPIN-2(3H)-ONE COMPOUNDS, COMPOSITIONS AND METHODS OF TREATING CANCER

(71) Applicant: Chengdu Anticancer Bioscience, Ltd., Chengdu (CN)

(72) Inventors: Dun YANG, Chengdu (CN); Jian HUANG, Chengdu (CN); Jing ZHANG, Chengdu (CN); Shenqiu ZHANG, St. Andrews (GB); Thaddeus ALLEN, Daly City, CA (US); Qiong SHI, Chengdu (CN); Naganna NIMISHETTI, Chengdu (CN); Chengliang LI, Chenglu (CN); Linsheng ZHONG, Chengdu (CN); Hongmei LI, Chengdu (CN); Chenglu YANG, Chengdu (CN)

(21) Appl. No.: 18/667,441

(22) Filed: May 17, 2024

Related U.S. Application Data
(63) Continuation of application No. PCT/CN2022/132831, filed on Nov. 18, 2022.

(30) Foreign Application Priority Data
Nov. 19, 2021 (WO) PCT/CN2021/131734
Jul. 14, 2022 (WO) PCT/CN2022/105673

(51) Int. Cl. C07D 223/16; A61K 31/55; C07D 403/04; C07D 403/06; C07D 405/12; C07D 413/04; C07D 417/04

(52) U.S. Cl. CPC C07D 223/16; A61K 31/55; C07D 403/04; C07D 403/06; C07D 405/12; C07D 413/04; C07D 417/04

(57) ABSTRACT

The present disclose includes, among other things, compounds that treat or lessen the severity of cancer, pharmaceutical compositions and methods of making and using the same.

### RELATED APPLICATIONS

This application is a continuation of International Patent Application No. PCT/CN2022/132831, filed Nov. 18, 2022, which claims priority to Patent Application No. PCT/CN2022/105673, filed Jul. 14, 2022, and Patent Application No. PCT/CN2021/131734, filed Nov. 19, 2021, each of which are incorporated herein by reference in their entireties.

### BACKGROUND

Cancer is a term used for diseases in which abnormal cells divide without control and may invade other tissues. Cancer cells may also spread to other parts of the body through the blood and lymph systems.

There are more than 100 different types of cancer, with most cancers named for the organ or type of cell in which they start. For example, cancer that begins in the colon may be referred to as colon cancer; cancer that begins in basal cells of the skin may be referred to as basal cell carcinoma. Common types of cancer include breast cancer and lung cancer.

Cancer types can also be grouped into broader categories. The main categories of cancer include: carcinoma—cancer that begins in the skin or in tissues that line or cover internal organs; sarcoma—cancer that begins in bone, cartilage, fat, muscle, blood vessels, or other connective or supportive tissue; leukemia—cancer that starts in blood-forming tissue such as the bone marrow and causes large numbers of abnormal blood cells to be produced and enter the blood; lymphoma and myeloma-cancers that begin in the cells of the immune system; central nervous system cancers-cancers that begin in the tissues of the brain and spinal cord.

Several techniques for treating cancer are known in the art. Such techniques include chemotherapy, radiation therapy, surgery, and transplantation. Each of these techniques, however, have undesirable side effects and varying success rates. Therefore, a need exists to develop new methods for treating cancer and/or diseases associated with cellular proliferation.

### SUMMARY

The present disclosure provides for compounds of formula (I):

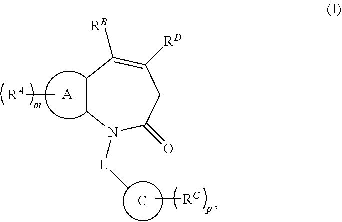

or a pharmaceutically acceptable salt thereof. Additionally, the present disclosure includes, among other things, pharmaceutical compositions, methods of using and methods of making a compound of formula (I).

### DETAILED DESCRIPTION

In some embodiments, the present disclosure includes a compound of Formula (I):

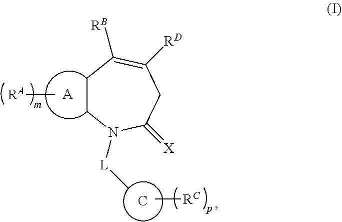

or a pharmaceutically acceptable salt thereof
wherein
X is O═ or S═;
Ring A is selected from the group consisting of optionally substituted phenyl, optionally substituted 5-membered heteroaryl, and optionally substituted 6-membered heteroaryl;
L is selected from the group consisting of a bond, optionally substituted C₁-C₃ alkylene, —CH(D)-, —C(D)₂-, —C(O)—, —C(O)O—, —C(O)NH—, and —S(O)₂—;
Group C is selected from the group consisting of C₁-C₆ aliphatic, —C≡CR₃, optionally substituted phenyl, optionally substituted 5-membered heteroaryl, optionally substituted 6-membered heteroaryl, optionally substituted 6-membered carbocyclyl, and optionally substituted 6-membered heterocyclyl;
each R^{A} is independently selected from the group consisting of halogen, —CN, —C≡CR³, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂; or
R^{B} is independently selected from the group consisting of halogen, —CFCR³, optionally substituted 5-6-membered heteroaryl, optionally substituted C₁-C₃ aliphatic, and optionally substituted C₁-C₃ alkoxy, and optionally substituted 3-6-membered carbocyclyl;
each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —N(R³)₂, —C(O)OR³, —C(O)N(R³)₂, —N(H)C(O)R³, and —N(H)C(O)N(R³)₂;
   wherein optionally two instances of R^{C} are taken together with the atoms on which they are attached to form an optionally substituted 6-membered aryl or optionally substituted 6-membered heteroaryl;
R^{D} is selected from the group consisting of optionally substituted 5-membered heteroaryl, —C(O)OR³, C(O)N(R³)₂, —(CH₂)₁₋₃N(R²)₂, —(CH₂)₁₋₃OR², —(CH₂)₁₋₃O(CH₂)₁₋₃R², and —CHO;
each R¹ is independently selected from the group consisting of hydrogen, —C(O)R³, —(CH₂)₁₋₃OR³, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
   wherein optionally two instances of R² are taken together to form an optionally substituted 3-7 membered heterocyclyl ring;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
m is 0, 1, 2, 3, or 4;
p is 0, 1, 2, 3, 4, or 5.

In some embodiments, present disclosure includes a compound of Formula (I-a), (I-b), or (I-c):

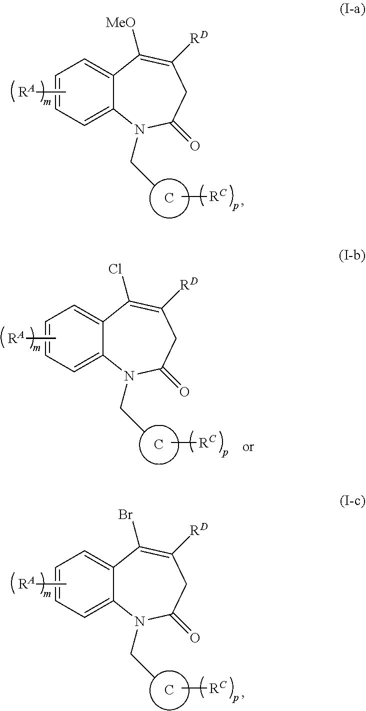

or a pharmaceutically acceptable salt thereof, wherein Group C, R^{A}, R^{C}, R^{D}, in, and p are defined above and described in classes and subclasses herein.

In some embodiments, present disclosure includes a compound of Formula (I-a1), (I-b1) or (1-c1):

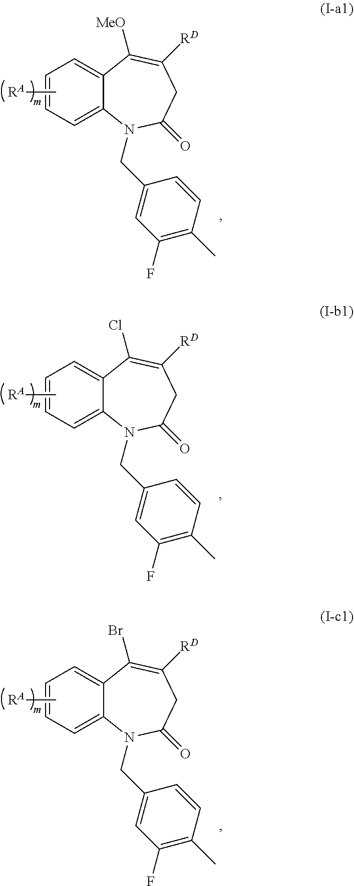

or a pharmaceutically acceptable salt thereof, wherein R^{A}, R^{D}, and m are defined above and described in classes and subclasses herein.

In some embodiments, present disclosure includes a compound of formula (I-b2):

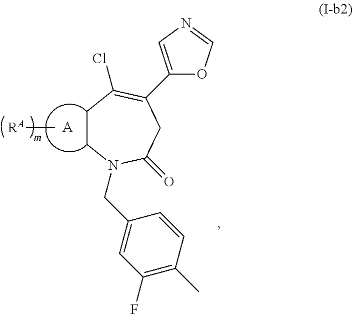

or a pharmaceutically acceptable salt thereof, wherein R^{A} and m are defined above and described in classes and subclasses herein.

In some embodiments, present disclosure includes a compound of formula (I-c2). (I-c3) or (I-c4)

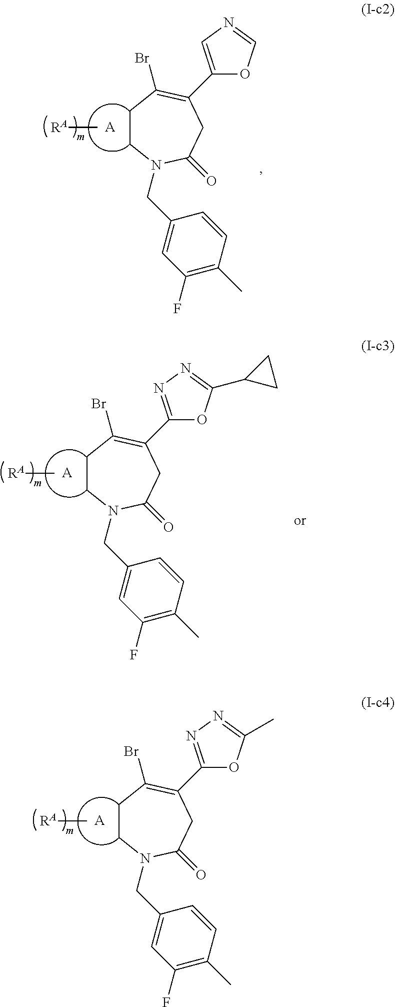

or a pharmaceutically acceptable salt thereof, wherein R^{A} and m are defined above and described in classes and subclasses herein.

### X

In some embodiments, X is O═ or S═. In some embodiments, X is O═. In some embodiments, X is S═.

### Ring A

In some embodiments, Ring A is selected from the group consisting of optionally substituted phenyl, optionally substituted 5-membered heteroaryl and optionally substituted 6-membered heteroaryl. In some embodiments, Ring A is optionally substituted phenyl. In some embodiments. Ring A is optionally substituted 6-membered heteroaryl. In some embodiments, Ring A is an optionally substituted 6-membered heteroaryl selected from pyridine, pyrazine, pyridazine, and pyrimidine. In some embodiments, Ring A is optionally substituted pyridine.

In some embodiments, Ring A is selected from the group consisting of

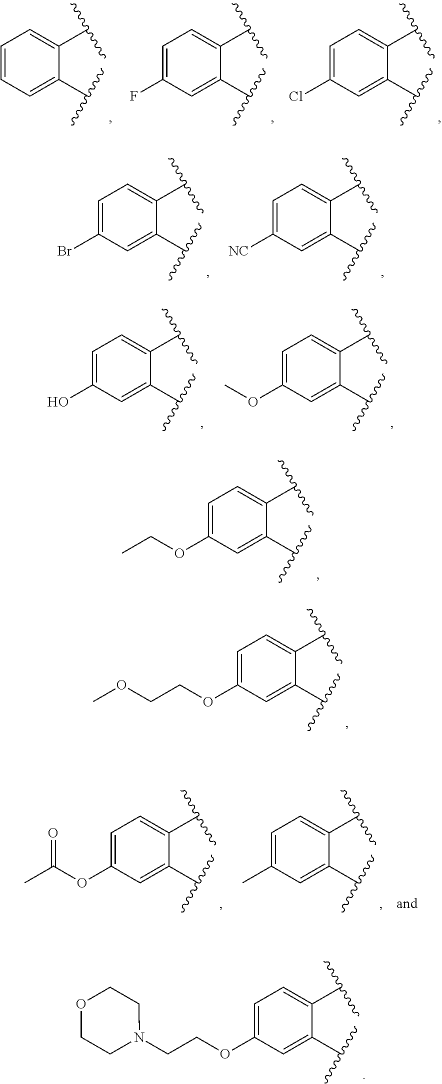

In some embodiments. Ring A is

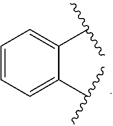 [SMILES: *c1ccccc1*]

### Group C

In some embodiments, Group C is selected from the group consisting of C₁-C₆ aliphatic, optionally substituted phenyl, optionally substituted 5-membered heteroaryl, optionally substituted 6-membered heteroaryl, optionally substituted 6-membered carbocyclyl, and optionally substituted 6-membered heterocyclyl. In some embodiments. Group C is selected from the group consisting of optionally substituted phenyl and optionally substituted 6-membered heteroaryl. In some embodiments, Group C is optionally substituted phenyl. In some embodiments, Group C is optionally substituted heteroaryl. In some embodiments, Group C is an optionally substituted 6-membered heteroaryl selected from pyridine, pyrazine, pyridazine, and pyrimidine. In some embodiments, Group C is optionally substituted pyridine. In some embodiments, Group C is optionally substituted phenyl or optionally substituted pyridine. In some embodiments, Group C is optionally substituted phenyl.

### L

In some embodiments. L is selected from the group consisting of a bond, optionally substituted C₁-C₃ alkylene, —C(O)—, —C(O)O—, —C(O)NH—, and —S(O)₂—. In some embodiments. L is selected from the group consisting of a bond, optionally substituted C₁-C₃ alkylene, and —C(O)—. In some embodiments, L is a bond. In some embodiments, L is an optionally substituted C₁-C₃ alkylene chain. In some embodiments, L is an optionally substituted C₁ alkylene chain. In some embodiments. L is an optionally substituted C₂ alkylene chain. In some embodiments. L is an optionally substituted C₃ alkylene chain. In some embodiments, L is an unsubstituted C₁-C₃ alkylene chain. In some embodiments, L is —CH₂—. In some embodiments, L is —C(H)(Me)-. In some embodiments, L is —CH₂CH₂—. In some embodiments. L is —CH₂CH₂CH₂—. In some embodiments, L is —C(O)—. In some embodiments, L is —S(O)₂—.

In some embodiments, L and Group C are taken together to form

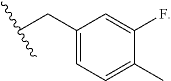 [SMILES: *Cc1ccc(C)c(F)c1]

### R^{A}

In some embodiments each R^{A} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂. In some embodiments each R^{A} is independently selected from the group consisting of halogen, —CN, optionally substituted C₁-C₆ aliphatic. —OR¹, —SR¹, —N(R¹)₂, —C(O)OR¹, C(O)N(R¹)₂, —N(H)C(O)R¹, and —N(H)C(O)N(R¹)₂. In some embodiments, R^{A} is selected from the group consisting of halogen, —CN, and —OR¹. In some embodiments R^{A} is selected from the group consisting of F, Cl, Br, CN, —OH, —OMe, OEt, —OCH₂CH₂OMe, —OAc, and —OCH₂CH₂(N-morpholine).

### R^{B}

In some embodiments, R^{B} is selected from the group consisting of halogen, optionally substituted 5-6-membered heteroaryl, optionally substituted C₁-C₃ aliphatic, and optionally substituted C₁-C₃ alkoxy. In some embodiments, R^{B} is halogen. In some embodiments, R^{B} is fluoro. In some embodiments, R^{B} is chloro. In some embodiments. R^{B} is bromo. In some embodiments. R^{B} is C₁-C₃ alkoxy. In some embodiments, R^{D} is methoxy.

### R^{C}

In some embodiments, each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —S(O)₂R³, —N(R³)₂, —C(O)OR³, C(O)N(R¹)₂, —N(H)C(O)R³, and —N(H)C(O)N(R³)₂. In some embodiments, each R^{C} is independently selected from the group consisting of halogen, —CN, optionally substituted C₁-C₆ aliphatic, optionally substituted phenyl, —OR³. —S(O)₂R³, and —C(O)OR³. In some embodiments, each R^{C} is independently selected from the group consisting of halogen and optionally substituted C₁-C₆ aliphatic.

In some embodiments, one instance of R^{C} is halogen and one instance is optionally substituted C₁-C₆ aliphatic.

In some embodiments, R^{C} is halogen. In some embodiments, R^{C} is fluorine. In some embodiments, R^{C} is chlorine. In some embodiments, R^{C} is bromine. In some embodiments, R^{C} is iodine.

In some embodiments. R^{C} is —CN. In some embodiments, R^{C} is —S(O)₂Me.

In some embodiments, each R^{C} is independently optionally substituted C₁-C₆ aliphatic. In some embodiments, each R^{C} is independently optionally substituted C₁-C₄ aliphatic. In some embodiments, each R^{C} is independently selected from the group consisting of optionally substituted methyl, optionally substituted ethyl, optionally substituted iso-propyl, optionally substituted n-propyl, optionally substituted n-butyl and optionally substituted tert-butyl. In some embodiments, each R^{C} is independently selected from the group consisting of —CF₃ and —CHF₂.

In some embodiments. R^{C} is —OR³, In some embodiments each R^{C} is independently selected from the group consisting of —OMe, —OCF₃ and —OCHF₂.

In some embodiments, two instances of R^{C} are taken together with the atoms on which they are attached to form an optionally substituted 6-membered aryl or optionally substituted 6-membered heteroaryl. In some embodiments two instances of R¹ are taken together with Group C to form an optionally substituted naphthyl or optionally substituted 10-membered heteroaryl. In some embodiments two instances of R^{C} are taken together with Group C to form an optionally substituted naphthyl, optionally substituted quinolinyl, or isoquinolinyl. In some embodiments two instances of R^{C} are taken together with Group C to form an optionally substituted naphthyl or optionally substituted quinolinyl. In some embodiments two instances of R^{C} are taken together with Group C to form an optionally substituted naphthyl. In some embodiments two instances of R^{C} are taken together with Group C to form an optionally substituted quinolinyl.

In some embodiments, one instance of R^{C} is C₁₋₃ alkyl and one instance of R^{C} is halogen. In some embodiments, one instance of R^{C} is methyl and one instance of R^{C} is fluoro.

### R^{D}

In some embodiments, R^{D} is selected from the group consisting of optionally substituted 5-membered heteroaryl, —C(O)OR³. C(O)N(R³)₂, —(CH₂)₁₋₃N(R²)₂, —(CH₂)₁₋₃OR², —(CH₂)₁₋₃O(CH₂)₁₋₃R², and —CHO. In some embodiments, R^{D} is optionally substituted 5-membered heteroaryl. In some embodiments, R^{D} is —(CH₂)₁₋₃OR². In some embodiments. R^{D} is —CHO.

In some embodiments, R^{D} is selected from the group consisting of

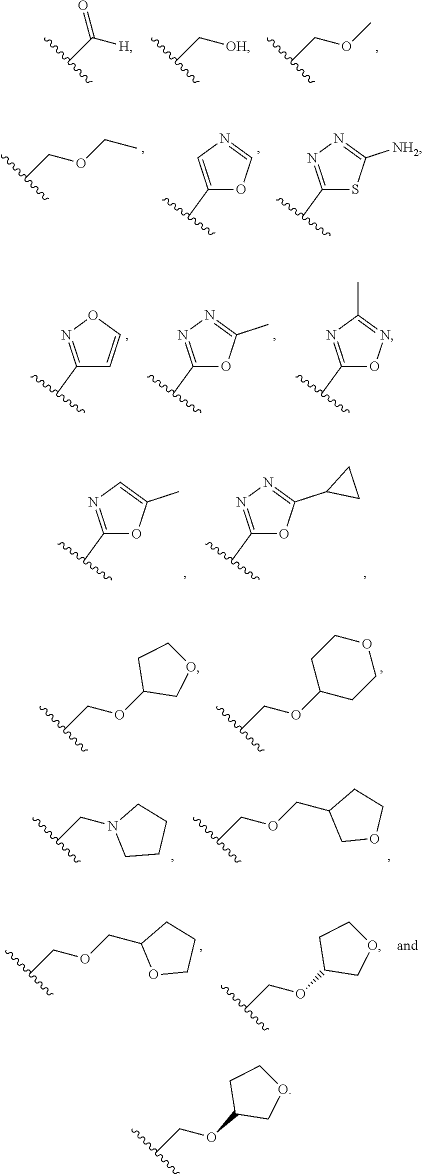

### R¹

In some embodiments, each R¹ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R¹ is independently optionally substituted C₁—C(aliphatic. In some embodiments, R¹ is optionally substituted methyl.

### R²

In some embodiments, each R² is independently selected from the group consisting of hydrogen, optionally substituted C₁—C(aliphatic, optionally substituted phenyl, optionally substituted 5-6-membered heteroaryl, optionally substituted 3-7 membered carbocyclyl, and optionally substituted 3-7 membered heterocyclyl. In some embodiments, each R² is independently optionally substituted C₁-C₆ aliphatic. In some embodiments. R² is optionally substituted methyl.

### R³

In some embodiments, each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R³ is independently optionally substituted C₁-C₆ aliphatic. In some embodiments, each R³ is independently selected from the group consisting of hydrogen, optionally substituted methyl, —CF₃, and —CHF₂.

In some embodiments, the present disclosure includes compounds described in Table 1

TABLE 1

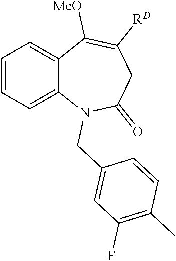

| Compound No. | R^{D} |
|---|---|
| 26-1 | 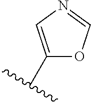 [SMILES: *c1cnco1] |
| 27-1 | 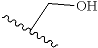 [SMILES: *CO] |
| 28-1 | 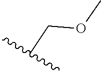 [SMILES: *COC] |
| 10-1 | 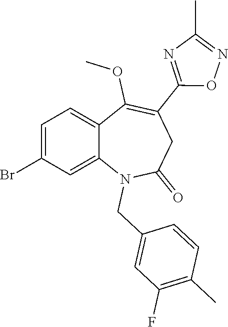 |

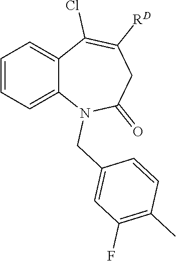

| Compound No. | R^{D} |
|---|---|
| 7-1 | 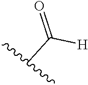 [SMILES: *C=O] |
| 23-1 | 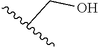 [SMILES: *CO] |
| 24-1 | 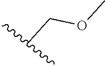 [SMILES: *COC] |
| 24-2 | 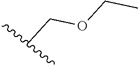 [SMILES: *COCC] |
| 8-1 | 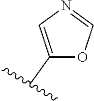 [SMILES: *c1cnco1] |
| 15-1 | 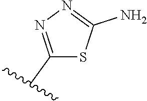 [SMILES: *c1nnc(N)s1] |
| 13-1 | 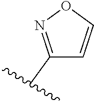 [SMILES: *c1ccon1] |
| 20-1 | 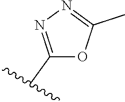 [SMILES: *c1nnc(C)o1] |
| 20-5 | 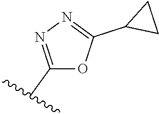 [SMILES: *c1nnc(C2CC2)o1] |

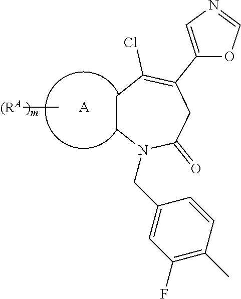

| Compound No. | Ring A |
|---|---|
| 8-1 | 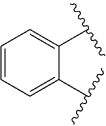 [SMILES: *c1ccccc1*] |
| 8-5 | 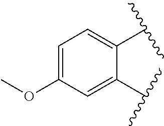 [SMILES: *c1ccc(OC)cc1*] |
| 8-7 | 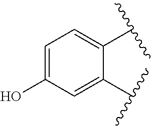 [SMILES: *c1ccc(O)cc1*] |
| 8-4 | 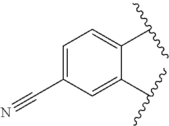 [SMILES: *c1ccc(C#N)cc1*] |
| 8-8 | 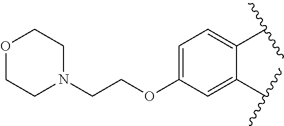 [SMILES: *c1ccc(OCCN2CCOCC2)cc1*] |
| 8-9 | 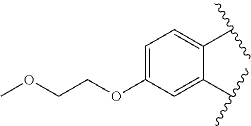 [SMILES: *c1ccc(OCCOC)cc1*] |

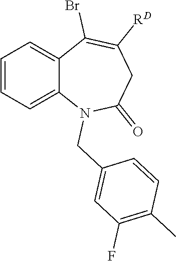

| Compound No. | R^{D} |
|---|---|
| 8-2 | 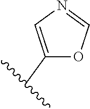 [SMILES: *c1cnco1] |
| 13-2 | 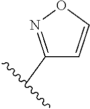 [SMILES: *c1ccon1] |
| 18-1 | 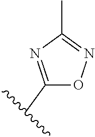 [SMILES: *c1nc(C)no1] |
| 20-2 | 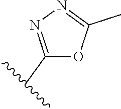 [SMILES: *c1nnc(C)o1] |
| 20-3 | 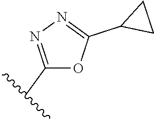 [SMILES: *c1nnc(C2CC2)o1] |
| 22-1 | 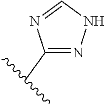 [SMILES: *c1nc[nH]n1] |
| 15-2 | 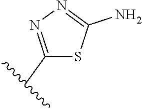 [SMILES: *c1nnc(N)s1] |
| 16-1 | 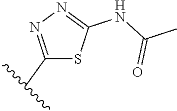 [SMILES: *c1nnc(NC(C)=O)s1] |
| 23-1 | 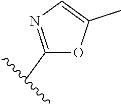 [SMILES: *c1ncc(C)o1] |

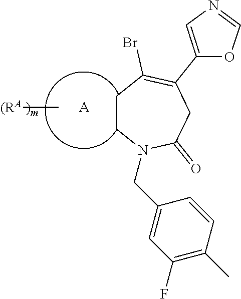

| Compound No. | Ring A |
|---|---|
| 8-2 | 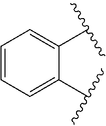 [SMILES: *c1ccccc1*] |
| 8-6 | 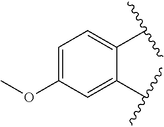 [SMILES: *c1ccc(OC)cc1*] |
| 8-11 | 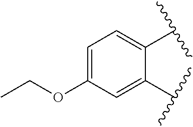 [SMILES: *c1ccc(OCC)cc1*] |
| 8-3 | 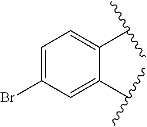 [SMILES: *c1ccc(Br)cc1*] |
| 8-13 | 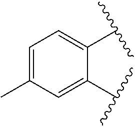 [SMILES: *c1ccc(C)cc1*] |
| 8-10 | 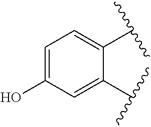 [SMILES: *c1ccc(O)cc1*] |
| 8-12 | 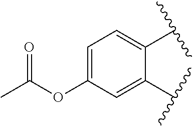 [SMILES: *c1ccc(OC(C)=O)cc1*] |

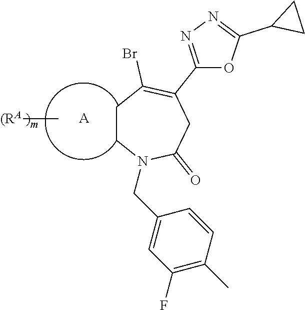

| Compound No. | Ring A |
|---|---|
| 20-3 | 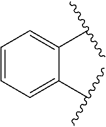 [SMILES: *c1ccccc1*] |
| 20-4 | 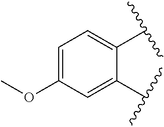 [SMILES: *c1ccc(OC)cc1*] |
| 20-6 | 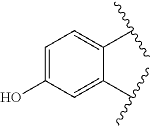 [SMILES: *c1ccc(O)cc1*] |
| 20-7 | 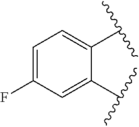 [SMILES: *c1ccc(F)cc1*] |
| 20-9 | 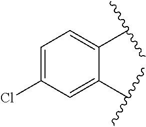 [SMILES: *c1ccc(Cl)cc1*] |
| 20-11 | 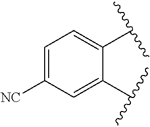 [SMILES: *c1ccc(C#N)cc1*] |
| 20-12 | 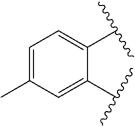 [SMILES: *c1ccc(C)cc1*] |

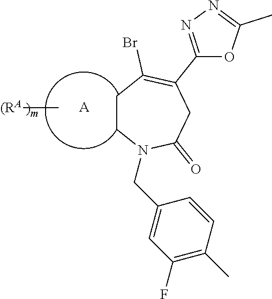

| Compound No. | Ring A |
|---|---|
| 20-2 | 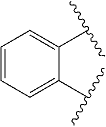 [SMILES: *c1ccccc1*] |
| 20-8 | 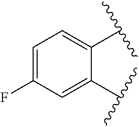 [SMILES: *c1ccc(F)cc1*] |
| 20-10 | 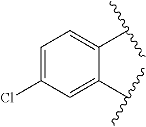 [SMILES: *c1ccc(Cl)cc1*] |
| 20-13 | 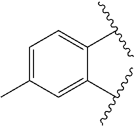 [SMILES: *c1ccc(C)cc1*] |
| Compound No. | Ring A |
| 20-14 | 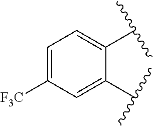 [SMILES: *c1ccc(C(F)(F)F)cc1*] |
| 20-15 | 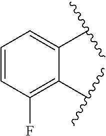 [SMILES: *c1cccc(F)c1*] |
| 20-16 | 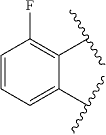 [SMILES: *c1cccc(F)c1*] |
| 20-17 | 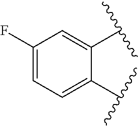 [SMILES: *c1ccc(F)cc1*] |
| 20-22 | 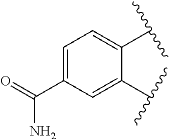 [SMILES: *c1ccc(C(N)=O)cc1*] |
| 20-23 | 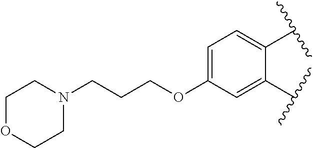 [SMILES: *c1ccc(OCCCN2CCOCC2)cc1*] |
| 20-20 | 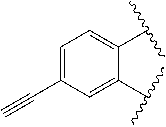 [SMILES: *c1ccc(C#C)cc1*] |
| 20-18 | 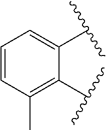 [SMILES: *c1cccc(C)c1*] |

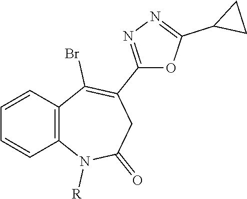

| Compound No. | R = |
|---|---|
| 20-21 | 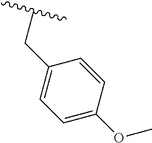 [SMILES: *Cc1ccc(OC)cc1] |
| 20-26 | 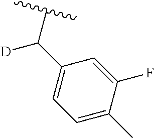 [SMILES: *C([2H])c1ccc(C)c(F)c1] |
| 20-27 | 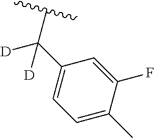 [SMILES: *C([2H])([2H])c1ccc(C)c(F)c1] |
| 20-25 | 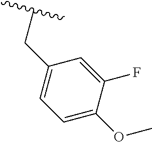 [SMILES: *Cc1ccc(OC)c(F)c1] |
| 20-28 | 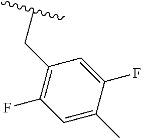 [SMILES: *Cc1cc(F)c(C)cc1F] |

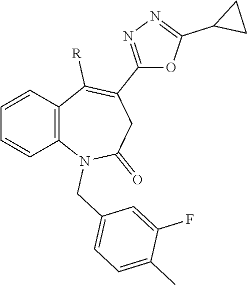

| Compound No. | R = |
|---|---|
| 20-30 | 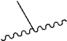 |
| 20-29 | 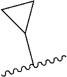 [SMILES: *C1CC1] |
| 20-32 | 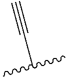 [SMILES: *C#C] |
| Compound No. | Compound |
| 24-1 | 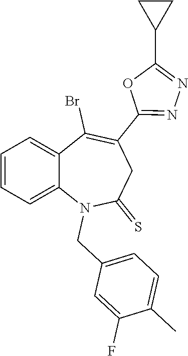 |
| 42 | 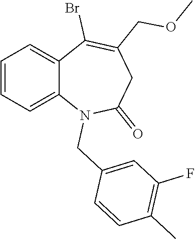 |
| 44-1 | 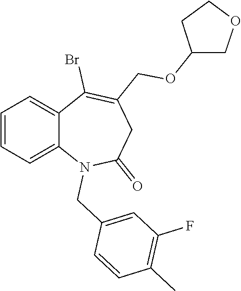 |
| 45 | 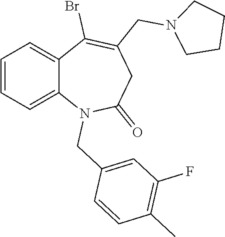 |
| 44-2 | 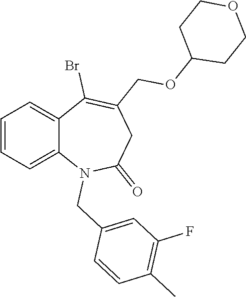 |
| 44-3 | 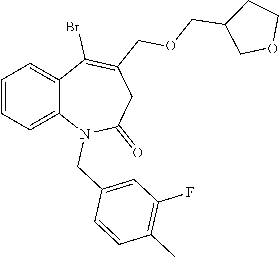 |
| 44-4 | 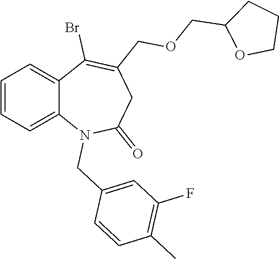 |
| 44-5 | 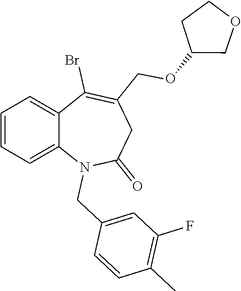 |
| 44-6 | 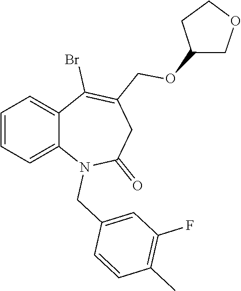 |
| 40 | 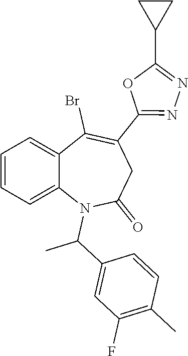 |
| 34-1 | 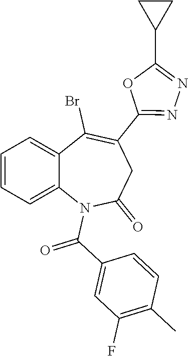 |
| 34-2 | 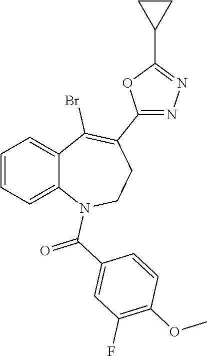 |
| 34-3 | 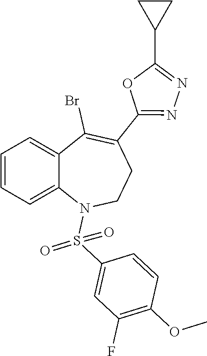 |
| 34-4 | 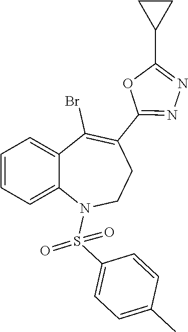 |

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl. (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure. “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms, having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The present disclosure includes tautomers of structures of compounds as drawn herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R*)₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}; —N(R^{∘})N(R^{∘})C(U)OR^{∘}; —(CH₂)₀₋₄C(O)R^{∘}; —C(S)R^{∘}; —(CH₂)₀₋₄C(O)OR^{∘}, —(CH₂)₀₋₄C((O)SR^{∘}; —(CH₂)₀₋₄C(O)OSiR^{∘}₃; (CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂R^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}2; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₂₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen. C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{∘}, -(haloR^{●}), —(CH₂)₀₋₂OH, —(CH₂)₀₋₂OR^{●}, —(CH₂)₀₋₂CH(OR)₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂Si, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include; —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition.

### I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²—H (D or deuterium) or ³H (T or tritium); carbon may be, for example, ¹³C or ¹⁴—C; oxygen may be, for example, ¹¹O; nitrogen may be, for example, ¹⁵N, and the like. In other embodiments, a particular isotope (e.g., ³H, ¹³C, ¹⁴C. ¹⁸, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water. Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is cancer.

In some embodiments, administration of a compound of the present disclosure results in arrest of mitosis or change in DNA content.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, administration of a compound of the present disclosure results in change in DNA content. In some embodiments, change of DNA content is induction of polyploidy.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is selected from the group consisting of lung cancer and breast cancer. In some embodiments, cancer is lung cancer. In some embodiments, lung cancer is non-small cell lung cancer. In some embodiments, non-small cell lung cancer is lung adenocarcinoma. In some embodiments, cancer is breast cancer. In some embodiments, breast cancer is mammary cancer. In some embodiments, breast cancer is breast adenocarcinoma.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### EXEMPLIFICATION

### 1. Chemistry

### 1.1 General Methods

Unless stated otherwise, all the chemicals required for synthesis were purchased from commercially available suppliers and used without further purification. ¹H NMR spectrum was determined with a Bruker Avance 111-400 at 400 MHz. LC-MS analysis was performed on a platform equipped with Agilent LC-MS 1260-6110 or Agilent LC-MS 1260-6120, using a Waters X Bridge C18: 50 mm×4.6 mm×3.5 um column. Flash column chromatography was conducted with silica gel (200-300 mesh. Qingdao Haiyang Chemical Co. Ltd. China). Analytical and preparative TLC analysis were performed on GF254 silica gel plates (Yantai Jiangyou Inc., China). Unless otherwise noted, reagents and all solvents are analytically pure grade and were obtained commercially from vendors such as Chron Chemical or Energy-Chemical.

Abbreviations: TLC: Thin Layer Chromatography. EA: Ethyl Acetate, PE: Petroleum Ether, DMF: N,N-dimethylformamide, THF: Eetrahydrofuran, DCM: Dichloromethane, DIPEA: N,N-diisopropylethylamine, DMAP: 4-dimethylaminopyridine, NaH: Sodium hydride 2. Synthesis and analytical data of compounds

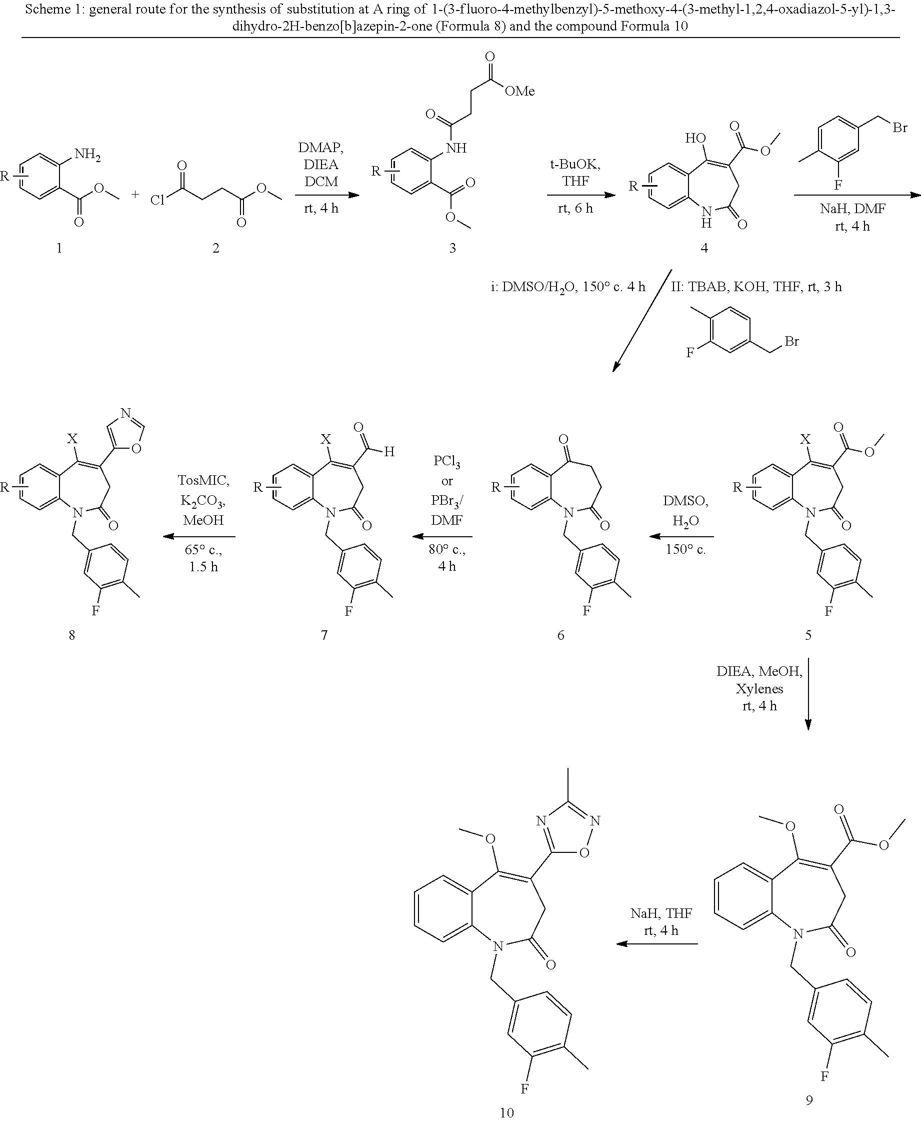

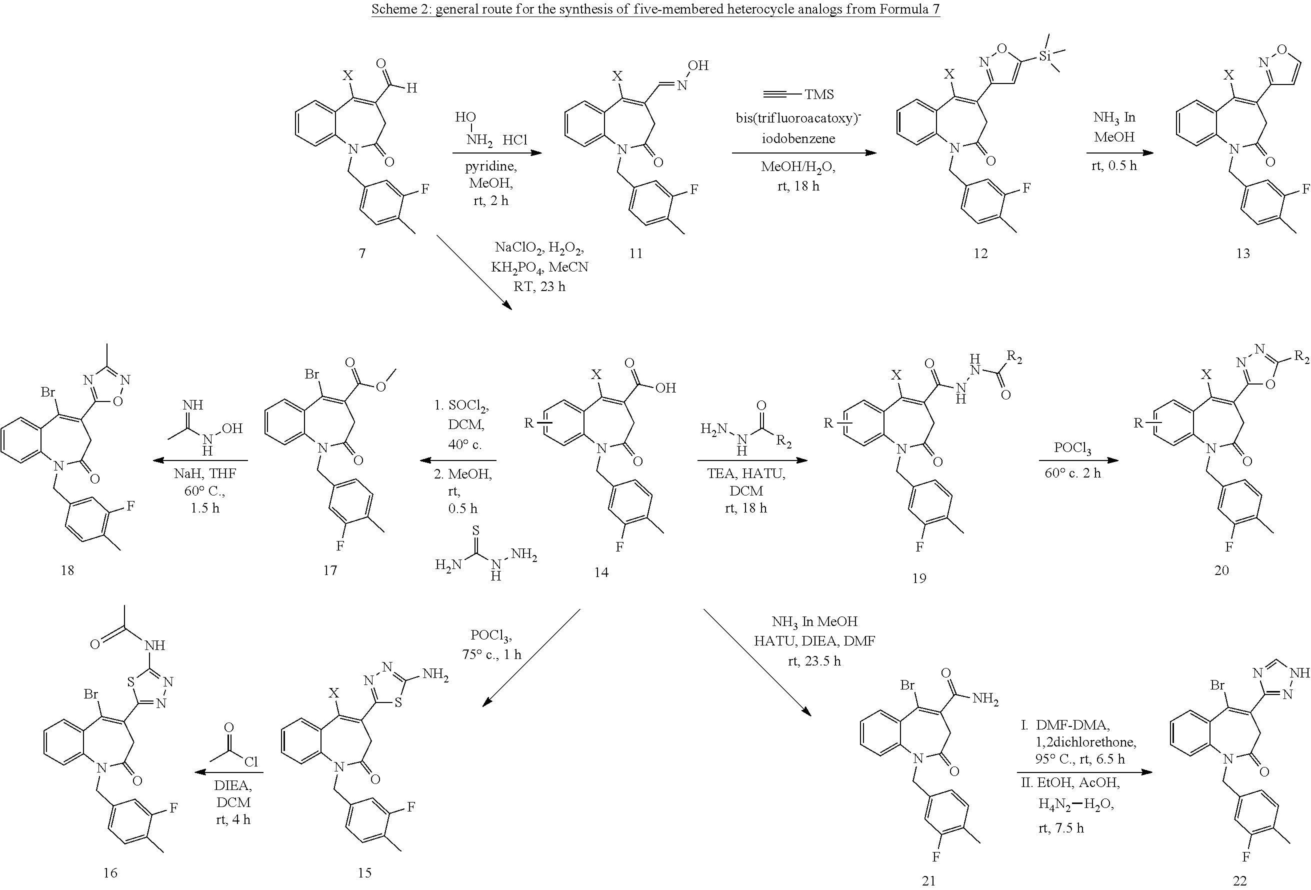

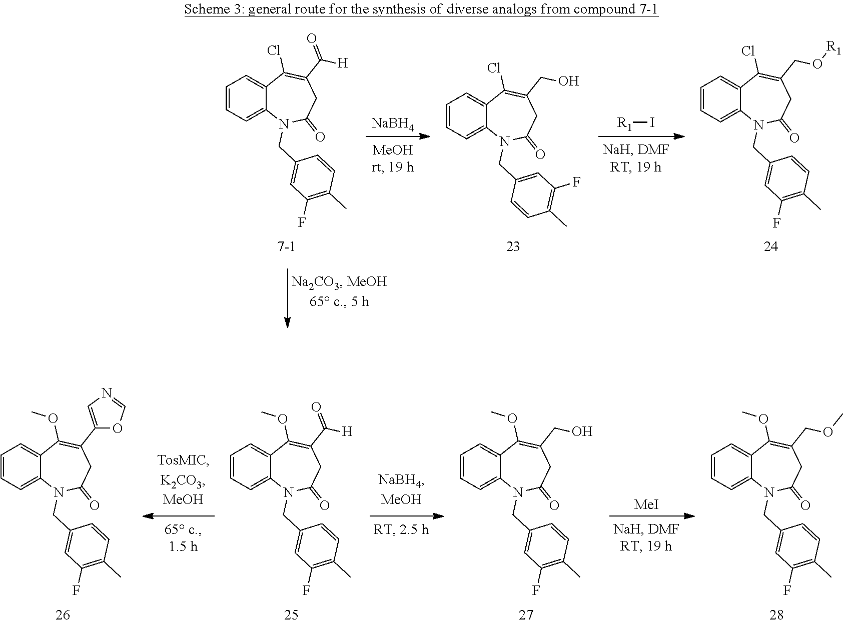

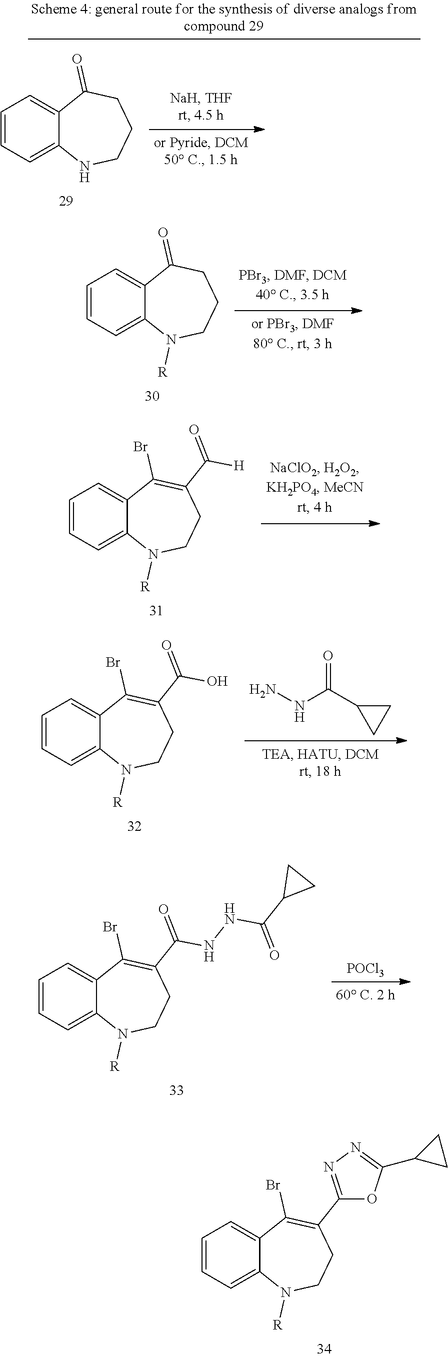

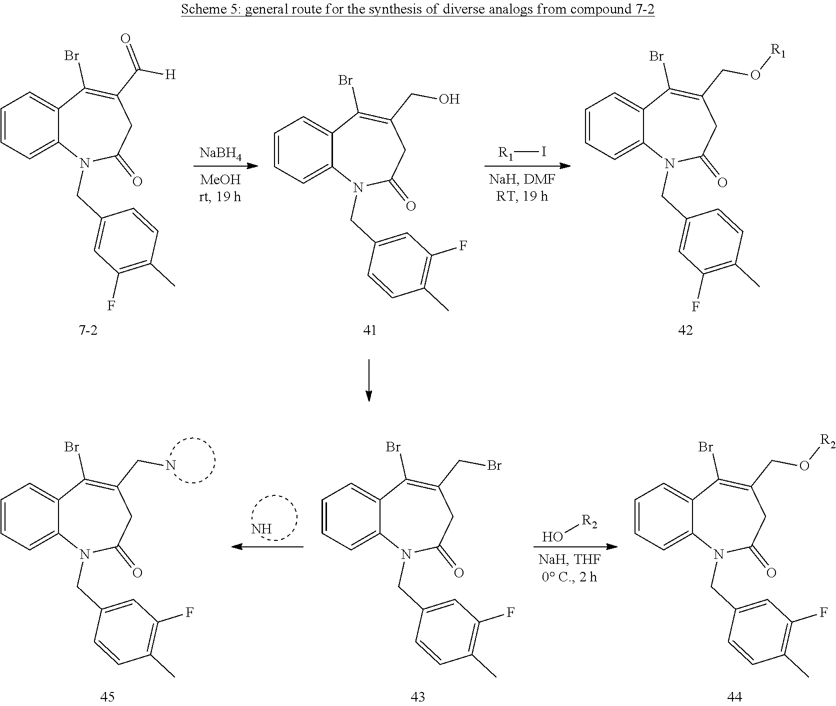

### Example 1. Representative Synthesis of Compounds of Formula 3

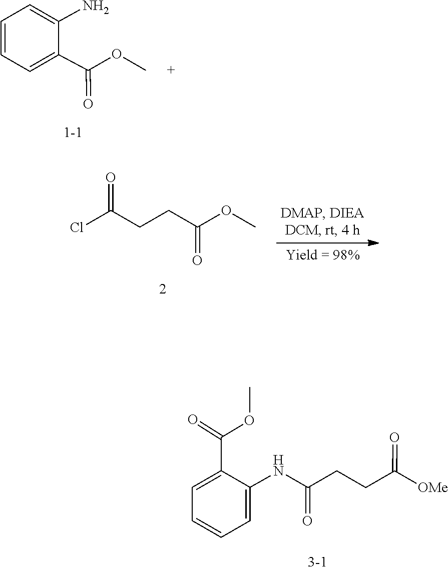

### Methyl 2-(3-methoxy-3-oxopropanamido) benzoate (3-1)

To a solution of methyl 2-aminobenzoate 1-1 (1.95 mL, 15 mmol, 1 equiv) in DCM (45 mL), was added DIEA (2.73 mL, 16.5 mmol, 1.3 equiv) and DMAP (30 mg, 0.075 mmol, 0.5% equiv) under nitrogen, to this solution was added 3-(carbomethoxy) propionyl chloride 2 (2.5 mL, 19.5 mmol, 1.1 equiv) dropwise over a period of 10 min, and the reaction mixture was allowed to stir at rt for 4 h. The reaction progress was monitored by TLC until the complete disappearance of compound 1-1. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 mL) and brine (1×100 mL) dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/8, flow rate: 20 mL/min, collections at 11-14 min elution time contained the target compound) to get 4.8 g (purity=98% s of the light-yellow oil compound with a yield of 98%.

TLC R_{f}=0.5 (PE:EA=2:1);

LCMS (m/z)=266.2 (M+H);

¹H NMR (400 MHz, DMSO-d₆): δ 10.65 (s, 1H), 8.24 (dd, J=8.4, 1.2 Hz, 1H), 7.89 (dd, J=7.9, 1.6 Hz, 1H), 7.58 (ddd, J=8.6, 7.3, 1.7 Hz, 1H), 7.16 (ddd, J=8.3, 7.4, 1.2 Hz, 1H), 3.85 (s, 3H), 3.60 (s, 3H), 2.69 (ddd, J=6.7, 5.4, 1.6 Hz, 2H), 2.62 (ddd, J=7.8, 5.5, 1.7 Hz, 2H). ¹³C NMR (100 MHz, DMSO-d₆): δ 172.65, 170.04, 167.62, 139.65, 133.92, 130.48, 123.05, 120.96, 117.47, 52.37, 51.41, 31.61, 28.44.

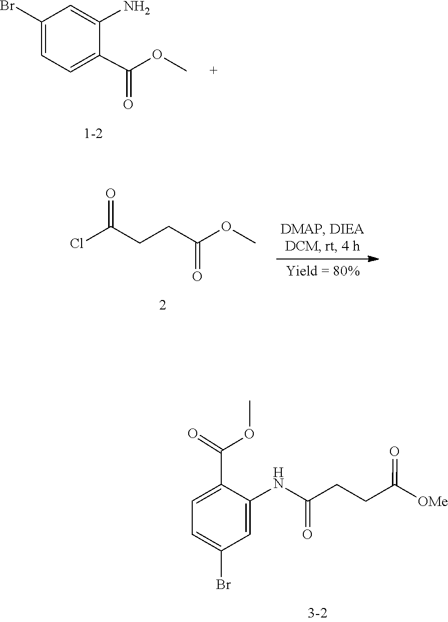

### Methyl 4-bromo-2-(4-methoxy-4-oxobutanamido) benzoate (3-2)

To a solution of methyl 2-amino-4-bromobenzoate 1-2 (10 g, 43.5 mmol, 1 equiv) in DCM (130 mL), was added DIEA (11.34 mL, 65.2 mmol, 1.5 equiv) and DMAP (265 mg, 2.17 mmol, 0.05 equiv) under nitrogen, to this solution was added 3-(carbomethoxy) propionyl chloride 2 (6.89 mL, 56.52 mmol, 1.3 equiv) dropwise over a period of 10 min, and the reaction mixture was allowed to stir at rt for 4 h. The reaction progress was monitored by TLC until the complete disappearance of compound 1-2. To the reaction mixture was then added H2O (40 mL) and the mixture was allowed to stir for 30 min. The organic solution was separated, and the aqueous solution was extracted with DCM (3×50 mL). The organic solutions were combined, washed with H2O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 11-14 min elution time contained the target compound) to get 12 g (purity=95%) of the light yellow oil compound with a yield of 80%.

TLC R_{f}=0.3 (PE:EA=2:1);

LCMS (m/z)=346.20 (M+H)

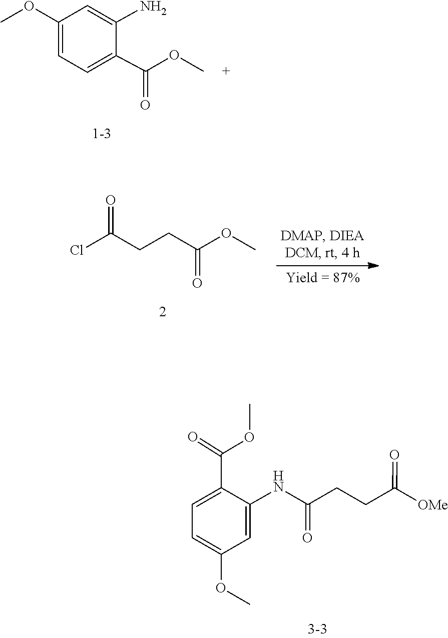

### Methyl 4-methoxy-2-(4-methoxy-4-oxobutanamido) benzoate (3-3)

To a solution of methyl 2-amino-4-methoxybenzoate 1-3 (5000 mg, 27 mmol, 1 equiv) in DCM (80 mL), was added DIEA (5100 mg, 30 mmol, 1.3 equiv) and DMAP (50 mg, 0.135 mmol, 0.05 equiv) under nitrogen, to this solution was added 3-(carbomethoxy) propionyl chloride 2 (4280 μL, 39 mmol, 1.3 equiv) dropwise over a period of 10 min, and the reaction mixture was allowed to stir at rt for 4 h. The reaction progress was monitored by TLC until the complete disappearance of compound 1-3. To the reaction mixture was then added 1-120 (40 mL) and the mixture was allowed to stir for 30 min. The organic solution was separated, and the aqueous solution was extracted with DCM (3×50 mL). The organic solutions were combined, washed with H2O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 7 g (purity=98%) of the light-yellow oil compound with a yield of 87%.

TLC R_{f}=0.6 (PE:EA=2:1);

LCMS (m/z)=296.50 (M+H)

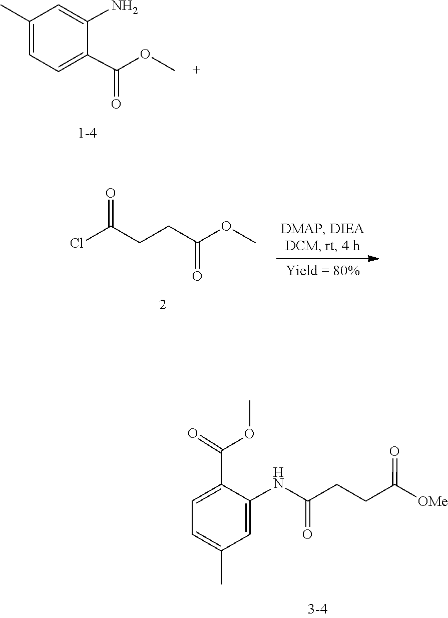

### Methyl 2-(4-methoxy-4-oxobutanamido)-4 methylbenzoate (3-4)

To a solution of methyl 2-amino-4-methylbenzoate 1-4 (5000 mg, 30 mmol, 1 equiv) in DCM (90 mL), was added DIEA (5460 mg, 39 mmol, 1.3 equiv) and DMAP (60 mg, 0.15 mmol, 0.05 equiv) under nitrogen, to this solution was added 3-(carbomethoxy) propionyl chloride 2 (5000 μL, 39 mmol, 1.3 equiv) dropwise over a period of 10 min, and the reaction mixture was allowed to stir at rt for 4 h. The reaction progress was monitored by TLC until the complete disappearance of compound 1-4. To the reaction mixture was then added H2O (40 mL) and the mixture was allowed to stir for 30 min. The organic solution was separated, and the aqueous solution was extracted with DCM (3×50 mL). The organic solutions were combined, washed with H2O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 6.7 g (purity=97%) of the light-yellow oil compound with a yield of 80%.

TLC R_{f}=0.6 (PE:EA=2:1);

LCMS (m/z)=280.0 (M+H)

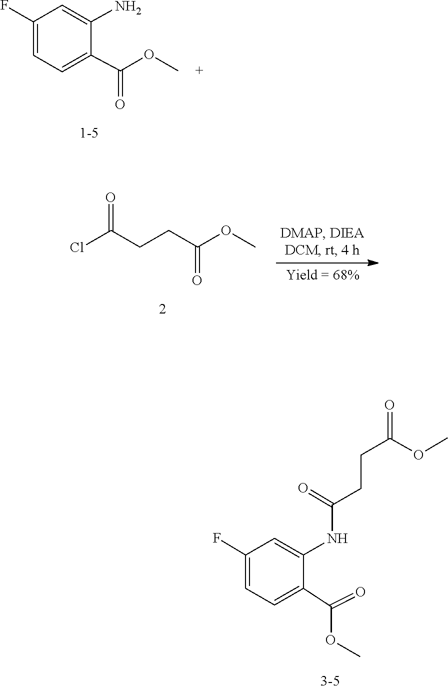

### Methyl 4-fluoro-2-(4-methoxy-4-oxobutanamido) benzoate (3-5)

To a solution of methyl 2-amino-4-fluorobenzoate (10.14 g, 60 mmol, 1 equiv) in DCM (180 mL), was added DMA (15 mL, 90 mmol, 1.5 equiv) and DMAP (366 mg, 3 mmol, 0.0:5 equiv) under nitrogen. To this solution was added methyl 4-chloro-4-oxobutanoate (10.51 mL, 78 mmol, 1.3 equiv) dropwise over a period of 10 min, and the reaction mixture was allowed to stir at it for 4 h, The reaction progress was monitored by TLC until the complete disappearance of compound 1-5. To the reaction mixture was then added 1-1 2 0 (40 mL) and the mixture was allowed to stir for 30 min, The organic solution was separated and the aqueous solution was extracted with DCM (3×50 mL), The organic solutions were combined, washed with H₂O(2×50 mL) and brine (1×100 mL), dried over anhydrous Na₂SO₄, filtered and concentrated by rotary evaporation to get the crude compound. Then, the resulting concentrate was purified on a silica column to get 11.5 g (purity=99%) yellow solid compound with a yield of 68%.

TLC R_{f}=0.5 (PE:EA=4:1);

LCMS(m/z)=284.30 (M+H)

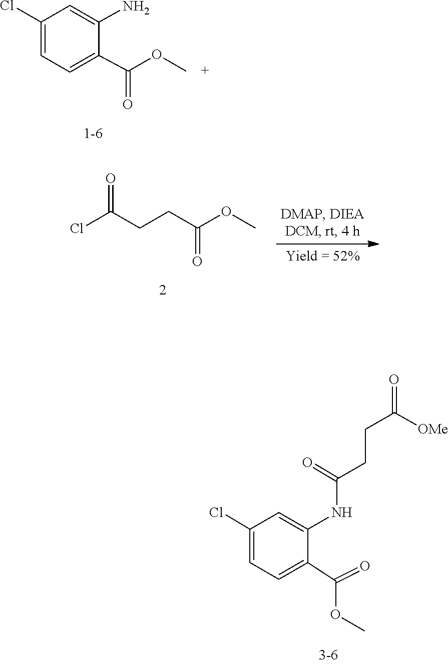

### Methyl 4-chloro-2-(4-methoxy-4-oxobutanamido)benzoate (3-6)

To a solution of methyl 2-amino-4-chlorobenzoate 1-6 (11.1 g, 60 mmol, 1 equiv) in DCM (180 mL), was added DIEA (15.7 mL, 90 mmol, 1.5 equiv) and DMAP (367 mg, 3 mmol, 0.05 equiv) under N₂. To this solution was added 3-(carbomethoxy)propionyl chloride 2 (3.6 mL, 29.8 mmol, 1.3 equiv) dropwise over a period of 10 min, and the reaction mixture was allowed to stir at rt for 4 h. The reaction progress was monitored by TLC until the complete disappearance of compound 1-6. To the reaction mixture was then added H₂O (40 mL) and the mixture was allowed to stir for 30 min. The organic solution was separated and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 9.3 g (purity=95%) of the light yellow oil compound with a yield of 52%.

TLC R_{f}=0.4 (PE:EA=4:1);

LCMS (m/z)=300.10 (M+H)

### Example 2. Representative Synthesis of Compounds of Formula 3

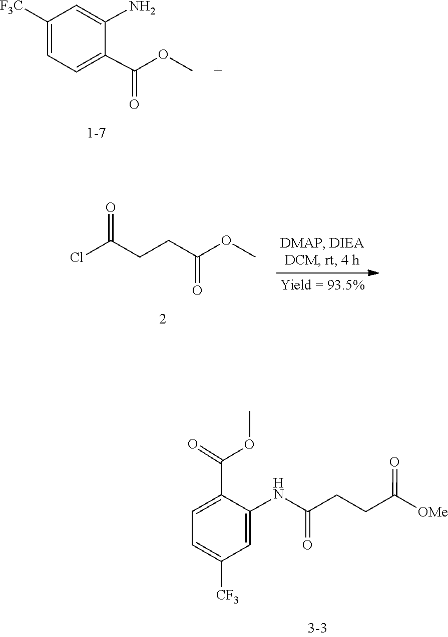

### Methyl 2-(4-methoxy-4-oxobutanamido)-4-(trifluoromethyl)benzoate (3-7)

To a solution of methyl 2-amino-4-(trifluoromethyl)benzoate (10 g, 45.6 mmol, 1 equiv) in DCM (137 mL), was added DIEA (11.4 mL, 68.5 mmol, 1.5 equiv) and DMAP (281 mg, 2.3 mmol, 5% equiv) under nitrogen. To this solution was added 3-(carbomethoxy) propionyl chloride (9.23 mL, 68.4 mmol, 1.5 equiv) dropwise over a period of 10 min, and the reaction mixture was allowed to stir at rt for 4 h. To the reaction mixture was then added H₂O (40 mL) and the mixture was allowed to stir for 30 min. The organic solution was separated, and the aqueous solution was extracted with EA (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 14.2 g (purity=99%) of the light-yellow oil compound with a yield of 93.5%.

TLC R_{f}=0.6 (PE:EA=2:1);

LCMS (m/z)=334.30 (M+H);

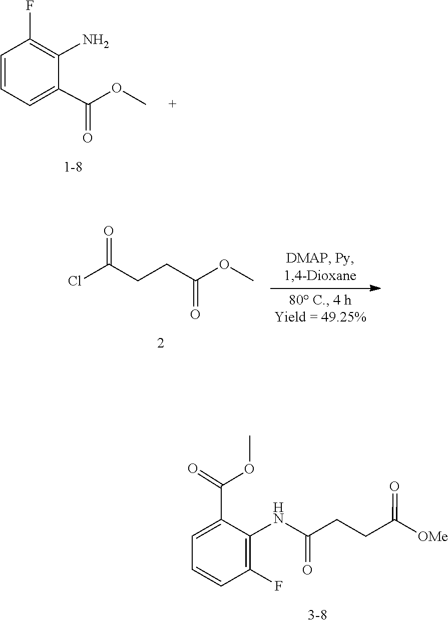

### methyl 3-fluoro-2-(4-methoxy-4-oxobutanamido)benzoate (3-8)

To a solution of methyl 2-amino-3-fluorobenzoate (4.8 g, 28.37 mmol, 1 equiv) in 1,4-Dioxane (27.89 mL), was added pyridine (2.8 mL, 34 mmol, 1.2 equiv) and DMAP (173 mg, 1.42 mmol, 0.05 equiv) under nitrogen. To this solution was added 3-(carbomethoxy)propionyl chloride (4.51 mL, 36.88 mmol, 1.3 equiv) dropwise over a period of 10 min, and the reaction mixture was allowed to stir at 80° C. for 4 h. To the reaction mixture was then added H₂O (40 mL) and the mixture was allowed to stir for 30 min. The organic solution was separated and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 3.958 g (purity=91.46%) of the light yellow solid compound with a yield of 49.25%.

TLC R_{f}=0.4 (PE:EA=2:1)

LCMS (m/z)=284.00 (M+H)

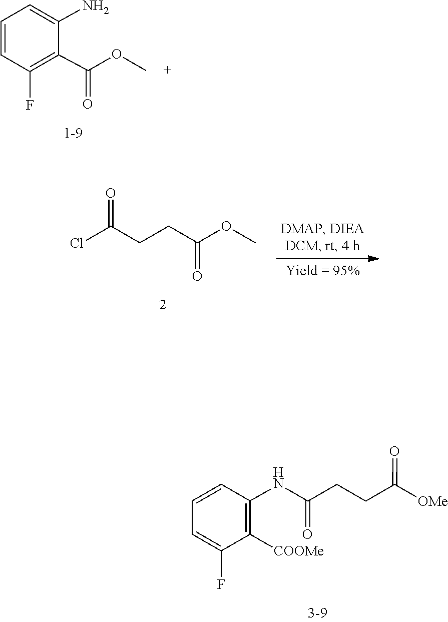

### Methyl 2-fluoro-6-(4-methoxy-4-oxobutanamido)benzoate (3-9)

To a solution of methyl 2-amino-6-fluorobenzoate (5 g, 29.56 mmol, 1 equiv) in DCM (89 mL), was added DIEA (7.5 mL, 44.34 mmol, 1.5 equiv) and DMAP (181 mg, 1.48 mmol, 0.05 equiv) under nitrogen. To this solution was added 3-(carbomethoxy)propionyl chloride 2 (4.7 mL, 38.42 mmol, 1.3 equiv) dropwise over a period of 10 min, and the reaction mixture was allowed to stir at rt for 4 h. To the reaction mixture was then added H₂O (40 mL) and the mixture was allowed to stir for 30 min. The organic solution was separated and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 8 g of the light-yellow oil compound with a yield of 95%. Purity=98%

TLC R_{f}=0.4 (EA:PE=1:2)

LCMS (m/z)=284.00 (M+H⁺)

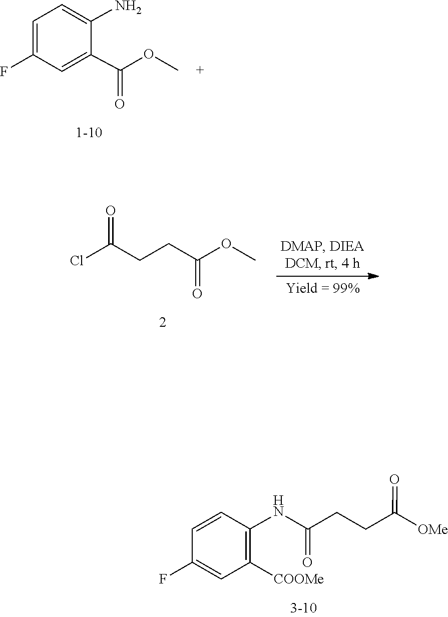

### Methyl 5-fluoro-2-(4-methoxy-4-oxobutanamido)benzoate (3-10)

To a solution of methyl 2-amino-5-fluorobenzoate (5 g, 29.56 mmol, 1 equiv) in DCM (89 mL), was added DIEA (7.5 mL, 44.3 mmol, 1.5 equiv) and DMAP (181 mg, 1.48 mmol, 0.05 equiv) under nitrogen. To this solution was added 3-(carbomethoxy) propionyl chloride 2 (4.7 mL, 38.4 mmol, 1.3 equiv) dropwise over a period of 10 min, and the reaction mixture was allowed to stir at rt for 4 h. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 8.4 g of the light-yellow oil compound with a yield of 99%. Purity=99%

TLC R_{f}=0.45 (EA:PE=1:4)

LCMS (m/z)=284.10 (M+H⁺)

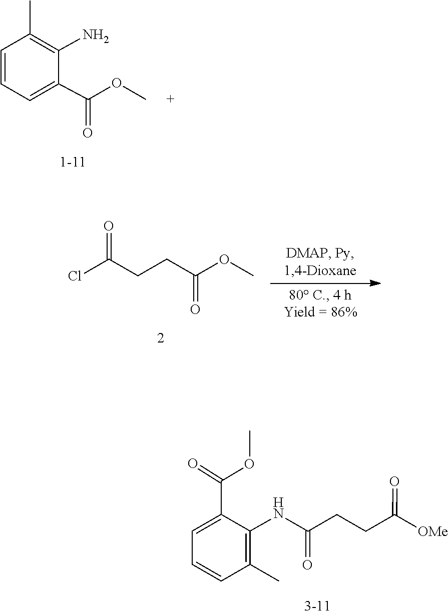

### Methyl 2-(4-methoxy-4-oxobutanamido)-3-methylbenzoate (3-11)

To a solution of methyl 2-amino-3-methylbenzoate (5 g, 30.3 mmol, 1 equiv) in 1,4-Dioxane (30 mL), was added pyridine (2.94 mL, 36.4 mmol, 1.2 equiv) and DMAP (184.8 mg, 1.5 mmol, 0.05 equiv) under nitrogen. To this solution was added 3-(carbomethoxy)propionyl chloride (4.82 mL, 39.4 mmol, 1.3 equiv) dropwise over a period of 10 min, and the reaction mixture was allowed to stir at 80° C. for 4 h. To the reaction mixture was then added H₂O (40 mL) and the mixture was allowed to stir for 30 min. The organic solution was separated and the aqueous solution was extracted with EA (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 6.94 g (purity=99%) of the light yellow oil compound with a yield of 82%.

TLC R_{f}=0.40 (EA:PE=1:4)

LCMS (m/z)=280.0 (M+H)

### Example 3. Representative Synthesis of Compounds of Formula 4

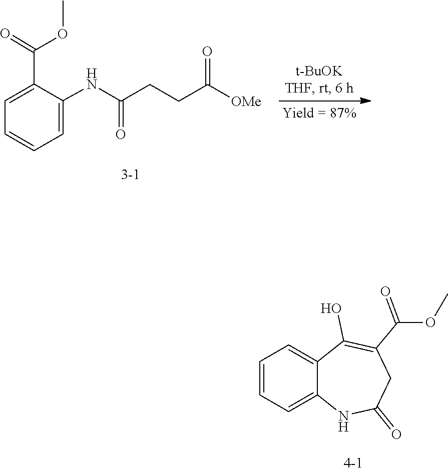 [SMILES: COC(=O)CCC(=O)Nc1ccccc1C(=O)OC ; COC(=O)C1=C(O)c2ccccc2NC(=O)C1]

### Methyl 5-hydroxy-2-oxo-2,3-dihydro-1H-benzo[b]azepine-4-carboxylate (4-1)

To a solution of methyl 2-(3-methoxy-3-oxopropanamido)benzoate 3-1 (4.8 g, 18.7 mmol, 1 equiv) in THF (74 mL) at 5° C. was added a 1 M solution of t-BuOK in THF (55 mL, 56 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 8-9 min elution time contained the target compound) to get 2.8 g of the white solid compound with a yield of 87%. Purity=95%

TLC R_{f}=0.3 (PE:EA=2:1);

LCMS (m/z)=234.3 (M+H)

¹H NMR (400 MHz, DMSO) δ 12.46 (s, 1H), 10.35 (s, 1H), 7.79 (dd, J=8.0, 1.5 Hz, 1H), 7.53 (ddd, J=8.3, 7.4, 1.6 Hz, 1H), 7.32-7.21 (m, 1H), 7.18 (dd, J=8.2, 0.8 Hz, 1H), 3.83 (s, 3H), 2.92 (s, 2H). ¹³C NMR (100 MHz, DMSO) δ 171.99, 170.80, 166.22, 137.92, 131.91, 128.15, 124.75, 123.47, 121.61, 95.82, 52.49, 30.70.

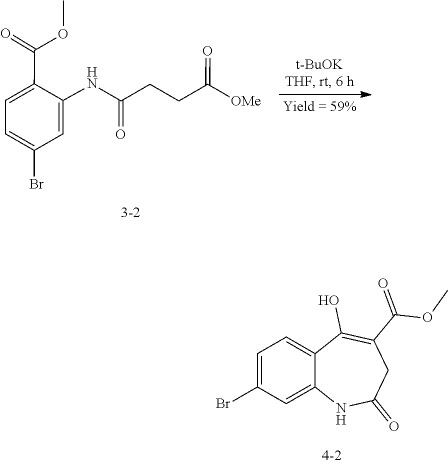 [SMILES: COC(=O)CCC(=O)Nc1cc(Br)ccc1C(=O)OC ; COC(=O)C1=C(O)c2ccc(Br)cc2NC(=O)C1]

### Methyl 9-bromo-5-hydroxy-2-oxo-2,3-dihydro-1H-benzo[b]azepine-4-carboxylate (4-2)

To a solution of methyl methyl 4-bromo-2-(4-methoxy-4-oxobutanamido) benzoate 3-2 (12 g, 34.88 mmol, 1 equiv) in THF (138 mL) at 5° C. was added a 1 M solution of t-BuOK in THF (103 mL, 105 mmol, 3 equiv) dropwise over 10 min while maintaining the temperature at 5° C. After 6 h, 20 mL of H₂O followed by 120 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 8-9 min elution time contained the target compound) to get 6.4 g of the white solid compound with a yield of 59%. Purity=99%

TLC R_{f}=0.3 (PE:EA=2:1);

LCMS (m/z)=313.83 (M+H)

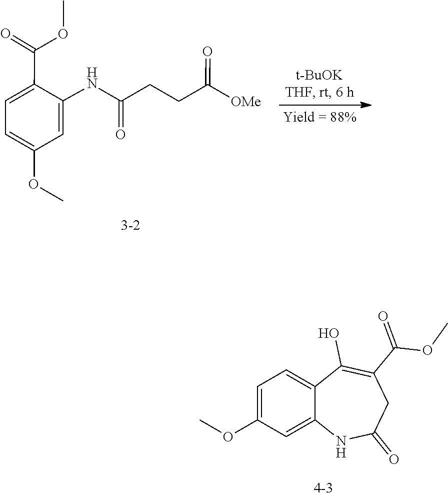

### Methyl 5-hydroxy-8-methoxy-2-oxo-2,3-dihydro-1H-benzo[b]azepine-4-carboxylate (4-3)

To a solution of 3-3 (7000 mg, 23.77 mmol, 1 equiv) in THF (90 mL) at 5° C. was added a 1 M solution of t-BuOK in THF (71 mL, 71 mmol, 3 equiv) dropwise over 10 min while maintaining the temperature at 5° C. After 6 h, 114 mL of H₂O followed by 114 mL of 1N HCl were added to bring the solution to pH=4. The resulting mixture was allowed to stir at rt for 40 min. The organic solution was separated and the aqueous solution was extracted with EtOAc (3×50 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 1 h elution time contained the target compound) to get 5500 mg of the white solid with a yield of 88%. Purity=91%

TLC R_{f}=0.3 (PE:EA=2:1);

LCMS (m/z)=296.30 (M+H)

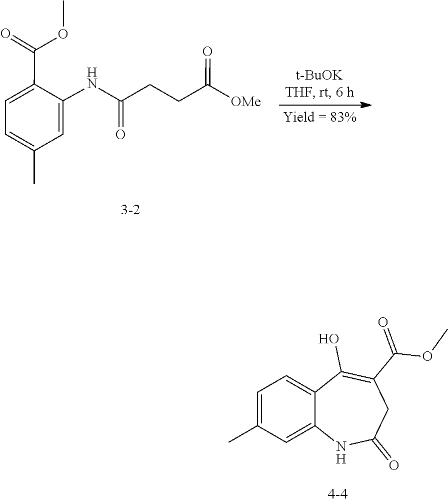

### Methyl 5-hydroxy-8-methyl-2-oxo-2,3-dihydro-1H-benzo[b]azepine-4-carboxylate (4-4)

To a solution of 3-4 (6700 mg, 24 mmol, 1 equiv) in THF (96 mL) at 5° C. was added a 1 M solution of t-BuOK in THF (72 mL, 72 mmol, 3 equiv) dropwise over 10 min while maintaining the temperature at 5° C. After 6 h, 114 mL of H₂O followed by 114 mL of 1N HCl were added to bring the solution to pH=4. The resulting mixture was allowed to stir at rt for 40 min. The organic solution was separated and the aqueous solution was extracted with EtOAc (3×50 ml). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 1 h elution time contained the target compound) to get 4900 mg of the white solid with a yield of 83%. Purity=93%

TLC R_{f}=0.3 (PE:EA=2:1);

LCMS (m/z)=248.1 (M+H)

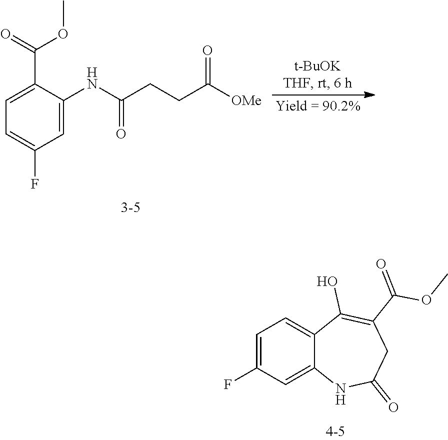 [SMILES: COC(=O)CCC(=O)Nc1cc(F)ccc1C(=O)OC ; COC(=O)C1=C(O)c2ccc(F)cc2NC(=O)C1]

### methyl 8-fluoro-5-hydroxy-2-oxo-2,3-dihydro-1H-benzo[b]azepine-4-carboxylate 4-5

To a solution of methyl 4-fluoro-2-(4-methoxy-4-oxobutanamido)benzoate 3-5 (11.5 g, 40.6 mmol, 1 equiv) in THF (160 mL) at 5° C. was added a 1 M solution of t-BuOK in THF (120 mL, 122 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 9.2 g (purity=99%) white solid compound with a yield of 90.2%.

TLC R_{f}=0.5 (PE:EA=2:1);

LCMS (m/z)=252.0 (M+H)

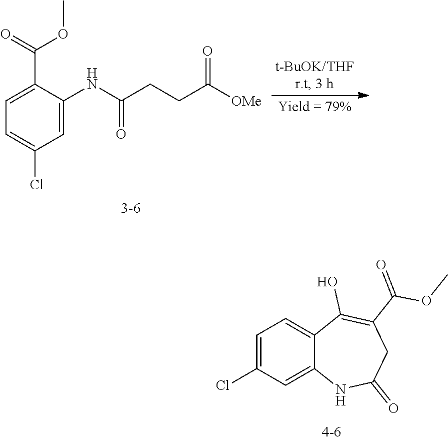 [SMILES: COC(=O)CCC(=O)Nc1cc(Cl)ccc1C(=O)OC ; COC(=O)C1=C(O)c2ccc(Cl)cc2NC(=O)C1]

### Methyl 8-chloro-5-hydroxy-2-oxo-2,3-dihydro-1H-benzo[b]azepine-4-carboxylate (4-6)

To a solution of methyl 4-chloro-2-(4-methoxy-4-oxobutanamido)benzoate (3-6) (9.3 g, 31.0 mol, 1 equiv) in THF (119 mL) at 5° C. was added 1 M solution of t-BuOK in THF (89 mL, 93.1 mmol, 3 equiv) dropwise over 10 min while maintaining the temperature at 5° C. After 3 h, 20 mL of H₂O followed by 70 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column to get 6.5 g of the white solid compound with a yield of 79%. Purity=99%

TLC R_{f}=0.5 (PE:EA=2:1);

LCMS (m/z)=267.93 (M+H).

### Example 4. Representative Synthesis of Compounds of Formula 4

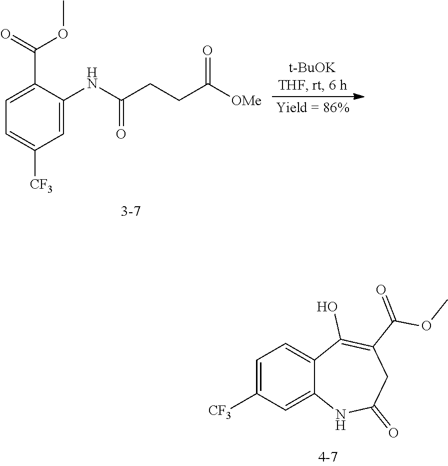 [SMILES: COC(=O)CCC(=O)Nc1cc(C(F)(F)F)ccc1C(=O)OC ; COC(=O)C1=C(O)c2ccc(C(F)(F)F)cc2NC(=O)C1]

### Methyl 5-hydroxy-2-oxo-2,3-dihydro-1H-benzo[b]azepine-4-carboxylate (4-7)

To a solution of methyl 2-(4-methoxy-4-oxobutanamido)-4-(trifluoromethyl)benzoate (14.2 g, 43 mmol, 1 equiv) in THF (170 mL) at 5° C. was added a 1 M solution of t-BuOK in THF (157 mL 128 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 11.6 g of the white solid compound with a yield of 86%. Purity=99%

TLC R_{f}=0.7 (PE:EA=2:1);

LCMS (m/z)=270.20 (M+H)

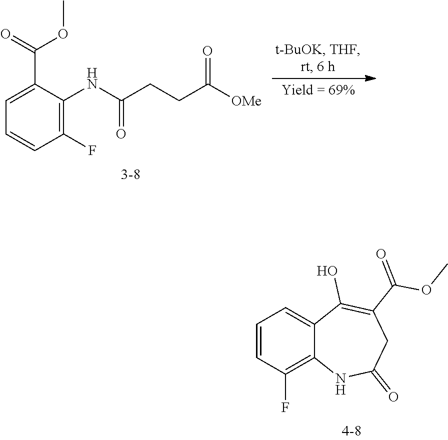 [SMILES: COC(=O)CCC(=O)Nc1c(F)cccc1C(=O)OC ; COC(=O)C1=C(O)c2cccc(F)c2NC(=O)C1]

### methyl 9-fluoro-5-hydroxy-2-oxo-2,3-dihydro-1H-benzo[b]azepine-4-carboxylate (4-8)

To a solution of methyl 3-fluoro-2-(4-methoxy-4-oxobutanamido)benzoate (3.958 g, 13.97 mmol, 1 equiv) in THF (55 mL) at 5° C. was added a 1 M solution of t-BuOK in THF (51 mL, 41.92 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at it for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 2.422 g of the white solid compound with a yield of 69%. Purity=86.46%

TLC R_{f}=0.6 (PE:EA=2:1)

LCMS (m/z)=220.00 (M+H)

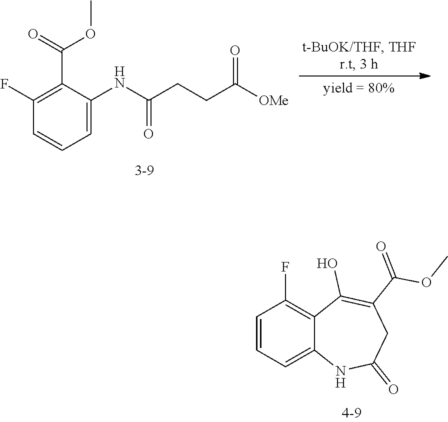 [SMILES: COC(=O)CCC(=O)Nc1cccc(F)c1C(=O)OC ; COC(=O)C1=C(O)c2c(F)cccc2NC(=O)C1]

### Methyl 8-fluoro-5-hydroxy-2-oxo-2,3-dihydro-1H-benzo[b]azepine-4-carboxylate (4-9)

To a solution of methyl Methyl 2-fluoro-6-(4-methoxy-4-oxobutanamido)benzoate (8 g, 28.27 mol, 1 equiv) in THF (113 mL) at 5° C. was added a 1 M solution of t-BuOK in THF (85 mL, 84.81 mmol, 3 equiv) dropwise over 10 min while maintaining the temperature at 5° C. After 3 h, 20 mL of 1120 followed by 70 mL of 1N HC were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column to get 6.5 g of the white solid compound with a yield of 80%. Purity=97%

TLC R_{f}=0.2 (EA:PE=1:2)

LCMS (m/z)=251.90 (M+H⁺)

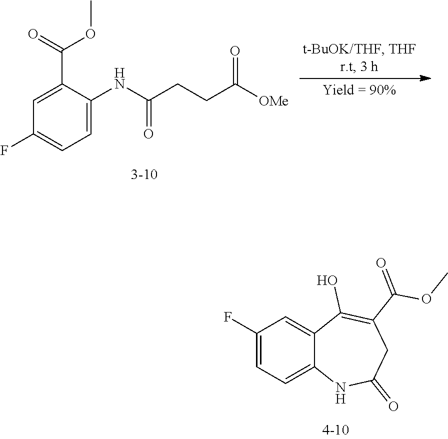 [SMILES: COC(=O)CCC(=O)Nc1ccc(F)cc1C(=O)OC ; COC(=O)C1=C(O)c2cc(F)ccc2NC(=O)C1]

### Methyl-7-fluoro-5-hydroxy-2-oxo-2,3-dihydro-1H-benzo[b]azepine-4-carboxylate (4-10)

To a solution of methyl 5-fluoro-2-(4-methoxy-4-oxobutanamido)benzoate (8.4 g, 29.6 mol, 1 equiv) in THF (I 18 mL) at 5° C. was added a 1 M solution of t-BuOK in THF (88.7 mL, 84.8 mmol, 3 equiv) dropwise over 10 min while maintaining the temperature at 5° C. After 3 h, 20 mL of H₂O followed by 70 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column to get 6.732 g of the white solid compound with a yield of 90%. Purity=96%

TLC R_{f}=0.2 (EA:PE=1:2)

LCMS (m/z)=251.93 (M+H⁺)

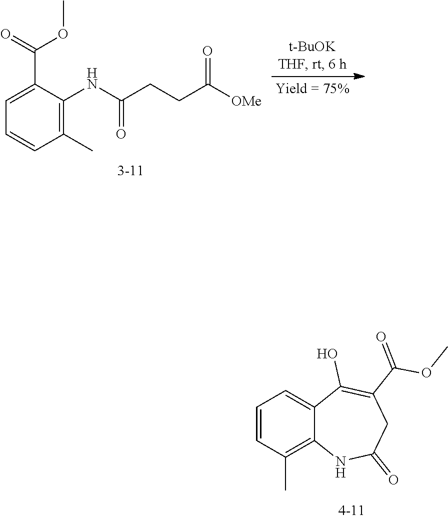 [SMILES: COC(=O)CCC(=O)Nc1c(C)cccc1C(=O)OC ; COC(=O)C1=C(O)c2cccc(C)c2NC(=O)C1]

### Methyl-5-hydroxy-9-methyl-2-oxo-2,3-dihydro-1H-benzo[b]azepine-4-carboxylate (4-11)

To a solution of methyl 2-(4-methoxy-4-oxobutanamido)-3-methylbenzoate (7.63 g, 27.3 mmol, 1 equiv) in THF (108 mL) at 5° C. was added a 1 M solution of t-BuOK in THF (101 mL, 82 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 5.088 g of the white solid compound with a yield of 75%. Purity=90%.

TLC R_{f}=0.4 (PE:EA=2:1);

LCMS (m/z)=216.00 (M+H)

### Example 5. Method A and B for the Synthesis of Compounds of Formula 6

### Method A:

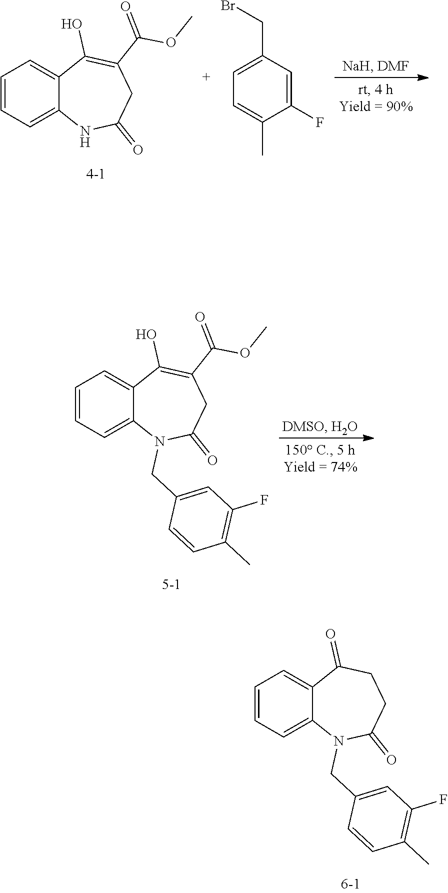

### 1-(3-fluoro-4-methylbenzyl)-3,4-dihydro-1H-benzo[b]azepine-2,5-dione (6-1)

To a solution of methyl 5-hydroxy-2-oxo-2,3-dihydro-1H-benzol[b]azepine-4-carboxylate 4-1 (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-2-fluoro-1-methylbenzene (0.12 mmol, 1.2 equiv), and the reaction mixture was allowed to stir at rt for 4 h. The reaction progress was monitored by TLC until the complete disappearance of 4-1. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 39 mg of the white solid compound with a yield of 90%, purity=90%.

TLC R_{f}=0.5 (PE:EA=2:1)

LCMS (m/z)=356.2 (M+H).

¹H NMR (400 MHz, DMSO) δ 12.28 (s, 1H), 7.74 (dd, J=7.9, 1.4 Hz, 1H), 7.60-7.43 (m, 2H), 7.36-7.24 (m, 1H), 7.13 (t, J=8.0 Hz, 1H), 6.84-6.61 (m, 2H), 5.20 (d, J=16.1 Hz, 1H), 4.94 (d, J=16.1 Hz, 1H), 3.85 (s, 3H), 3.49 (d, J=13.9 Hz, 1H), 2.66 (d, J=13.9 Hz, 1H), 2.13 (s, 3H). ¹³C NMR (100 MHz, DMSO) δ 170.45, 170.22, 165.59, 161.71, 159.29, 140.55, 137.59, 137.52, 131.84, 131.57, 131.52, 128.14, 127.76, 125.14, 123.09, 122.71, 122.54, 122.31, 122.28, 113.22, 112.99, 97.72, 52.54, 49.30, 31.06, 13.71, 13.68.

To the solution of resulting compound of methyl 1-(3-fluoro-4-methylbenzyl)-5-hydroxy-2-oxo-2,3-dihydro-1H-benzo[b]azepine-4-carboxylate 5-1 (800 mg, 2.254 mmol, 1 equiv) in DMSO (6.33 mL), was added H₂O (0.18 mL) under Nitrogen. and the reaction mixture was allowed to stir at 150° C. for 5 h. The reaction mixture was allowed to cool to it, ice (11.30 mL) was added, and the mixture was allowed to stir 12 h. To the flask was added 1N HCl (11.30 mL) at 0° C. and the mixture allowed to stir for 3 h. The resulting precipitate was filtered. Then, the crude product was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 580 mg (purity=80%) light yellow oil compound with a yield of 74%.

TLC R_{f}=0.4 (PE:EA=1:1);

LCMS (m/z)=298.60 (M+H)

¹H NMR (400 MHz, DMSO) δ 7.54 (ddd, J=8.2, 7.3, 1.7 Hz, 1H), 7.40 (dd, J=7.8, 1.7 Hz, 2H), 7.27 (td, J=7.6, 1.0 Hz, 1H), 7.13 (t, J=7.8 Hz, 1H), 6.84 (dd, J=13.8, 6.1 Hz, 2H), 5.03 (s, 2H), 2.99 (dd, J=7.1, 5.7 Hz, 2H), 2.81-2.73 (m, 2H), 2.13 (d, J=1.2 Hz, 3H). ¹³C NMR (100 MHz, DMSO) δ 203.00, 171.72, 161.68, 159.26, 140.37, 137.51, 137.44, 133.86, 132.99, 131.58, 131.53, 128.95, 126.09, 123.29, 122.81, 122.78, 122.66, 113.69, 113.46, 48.93, 41.04, 30.65, 13.75, 13.72.

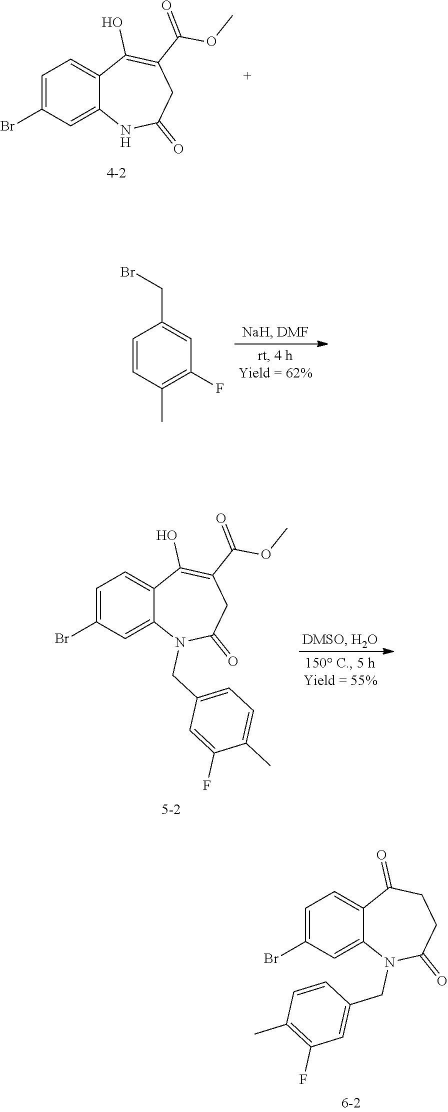

### 8-bromo-1-(3-fluoro-4-methylbenzyl)-3,4-dihydro-1H-benzo[b]azepine-2,5-dione (6-2)

To a solution of methyl 9-bromo-5-hydroxy-2-oxo-2,3-dihydro-1H-benzo[b]azepine-4-carboxylate (4-2) (6.4 g, 20.51 mmol, 1 equiv) in DMF (120 mL) was added NaH 60% dispersion in mineral oil (2462 mg, 30.77 mmol, 3 equiv). To this solution was added 4-(bromomethyl)-2-fluoro-1-methylbenzene (3.72 mL, 26.66 mmol, 1.3 equiv), and the reaction mixture was allowed to stir at rt for 4 h. The resulting solution was poured into water (120 mL) and extracted with Ethyl acetate (3×120 mL). The combined organic layers were washed with brine (1×120 mL), dried over anhydrous Na₂SO₄, and concentrated by rotary evaporation. Then, the resulting concentrate was purified on a silica column to get 5.5 g of the white solid compound with a yield of 62%, purity=99%.

TLC R_{f}=0.75 (PE:EA=2:1)

LCMS (m/z)=436.20 (M+H).

To the solution of resulting compound 5-2 (5.5 g, 12.67 mmol, 1 equiv) in DMSO (29 mL), was added H₂O (1 mL) under Nitrogen, and the reaction mixture was allowed to stir at 150° C. for 5 h. The reaction mixture was allowed to cool to rt, ice (45 mL) was added, and the mixture was allowed to stir 12 h. To the flask was added 1N HCl (45 mL) at 0° C. and the mixture allowed to stir for 3 h. The resulting precipitate was filtered. Then, the crude product was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 2.6 g (purity=95%) light yellow oil compound with a yield of 55%.

TLC R_{f}=0.4 (PE:EA=1:1)

LCMS (m/z)=377.90 (M+H)

### Method B:

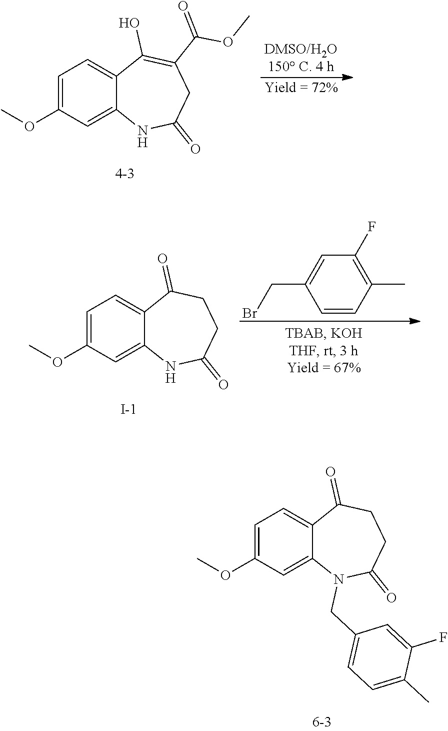

1-(3-fluoro-4-methylbenzyl)-8-methoxy-3,4-dihydro-1H-benzo[b]azepine-2,5-dione (6-3)

A mixture of methyl 5-hydroxy-8-methoxy-2-oxo-2,3-dihydro-11-benzo[b]azepine-4-carboxylate 4-3 (5.5 g, 0.02 mol) in DMSO (58 mL) and H2O (2 mL) was heated at 150° C. for 4 h. The reaction mixture was allowed to cool to it, ice (0.5 L) was added, and the mixture was allowed to stir 12 h. To the flask was added 1N HCl (0.5 L) at 0° C. and the mixture allowed to stir for 3 h. The resulting precipitate was filtered and dried under reduced pressure to afford product 3.1 g (purity=99%) light yellow solid compound with a yield of 72%.

TLC R_{f}=0.3 (PE:EA=1:1)

LCMS (m/z)=205.70 (M+H)

To the solution of resulting compound i-1 (205 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was 4-(bromomethyl)-2-fluoro-1-methylbenzene (244 mg, 1.2 mmol, 1.2 equiv) at ambient temperature. The reaction mixture was then stirred at RT for 3 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 219 mg (purity=99%) light yellow solid compound with a yield of 67%.

TLC R_{f}=0.5 (EA:PE=1:1)

LCMS (m/z)=328.50 (M+H)

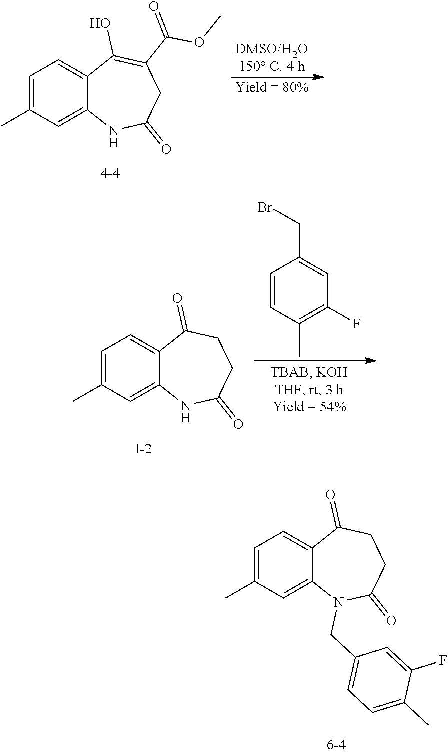

### 1-(3-fluoro-4-methylbenzyl)-8-methyl-3,4-dihydro-1H-benzo[b]azepine-2,5-dione (6-4)

A mixture of methyl 5-hydroxy-8-methyl-2-oxo-2,3-dihydro-1H-benzo[b]azepine-4-carboxylate 4-4 (4.9 g, 0.019 mol) in DMSO (55 mL) and H2O (2 mL) was heated at 150° C. for 4 h. The reaction mixture was allowed to cool to rt, ice (0.5 L) was added, and the mixture was allowed to stir 12 h. To the flask was added 1N HCl (0.5 L) at 0° C. and the mixture allowed to stir for 3 h. The resulting precipitate was filtered and dried under reduced pressure to afford product 2 g (purity=97%) light yellow solid compound with a yield of 80%.

TLC R_{f}=0.3 (PE:EA=1:1)

LCMS (m/z)=189.80 (M+H)

To the solution of resulting compound i-2 (189 mg, 1 mmol, 1 equiv), Tetrabutylammonium bromide (32 mg, 0.1 mmol, 0.1 equiv) and KOH (67 mg, 1.4 mmol, 1.4 equiv) in THF (4 mL) was added 4-(bromomethyl)-2-fluoro-1-methylbenzene (153 mg, 1.1 mmol, 1.2 equiv) at ambient temperature. The reaction mixture was then stirred at RT for 3 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 170 mg (purity=99%) light yellow solid compound with a yield of 54%.

TLC R_{f}=0.5 (PE:EA=1:1)

LCMS (m/z)=312.40 (M+H)

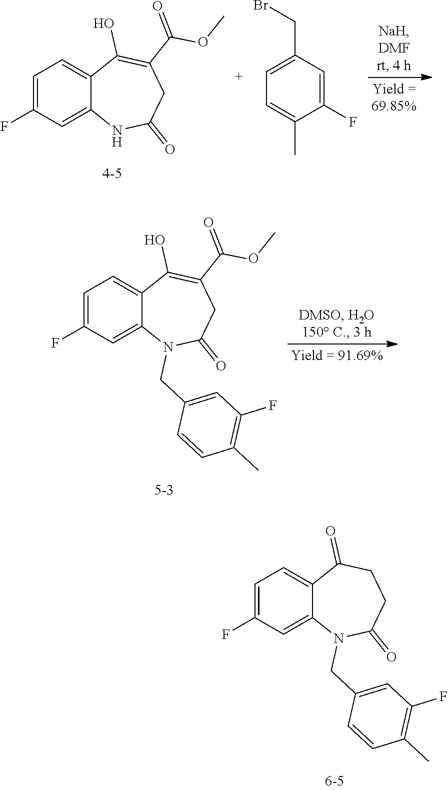

### 8-fluoro-1-(3-fluoro-4-methylbenzyl)-3,4-dihydro-1H-benzo[b]azepine-2,5-dione (6-5)

To a solution of methyl 8-fluoro-5-hydroxy-2-oxo-2,3-dihydro-1H-benzo[b]azepine-4-carboxylate 4-5 (23 mg, 0.1 mmol, 1 equiv) in DMF (0.7 mL) was added NaH 60% dispersion in mineral oil (20 mg, 0.3 mmol, 3.6 equiv) and stir for 1 h. To this solution was added 4-(bromomethyl)-2-fluoro-1-methylbenzene (0.12 mmol, 1.2 equiv), and the reaction mixture was allowed to stir at rt for 4 h. The resulting solution was poured into water (30 mL) and extracted with EA (3×30 mL). The combined organic layers were washed with brine (1×40 mL), dried over anhydrous Na₂SO₄, and concentrated by rotary evaporation. Then, the resulting concentrate was purified on a silica column to get 39 ng of the straw-colored solid compound (3.573 g) with a yield of 69.85%.

TLC R_{f}=0.7 (PE:EA=2:1)

LCMS (m/z)=374.10 (M+H)

To a solution of 5-3 (3.573 g, 9.57 mmol, 1 equiv) in DMSO (26.91 mL), was added H₂O (2 mL) under Nitrogen, and the reaction mixture was allowed to stir at 150° C. for 5 h. The reaction progress was monitored by TLC until the complete disappearance of compound 5-3. The reaction mixture was allowed to cool to rt, ice (11.30 mL) was added, and the mixture was allowed to stir 12 h. To the flask was added 1N HCl (11.30 mL) at 0° C. and the mixture allowed to stir for 3 h. The resulting precipitate was filtered. Then, the crude product was purified on a silica column to get 2.767 g (purity=96.52%) tinge pink solid compound with a yield of 91.69%.

TLC R_{f}=0.4 (PE:EA=2:1)

LCMS (m/z)=316.10 (M+H)

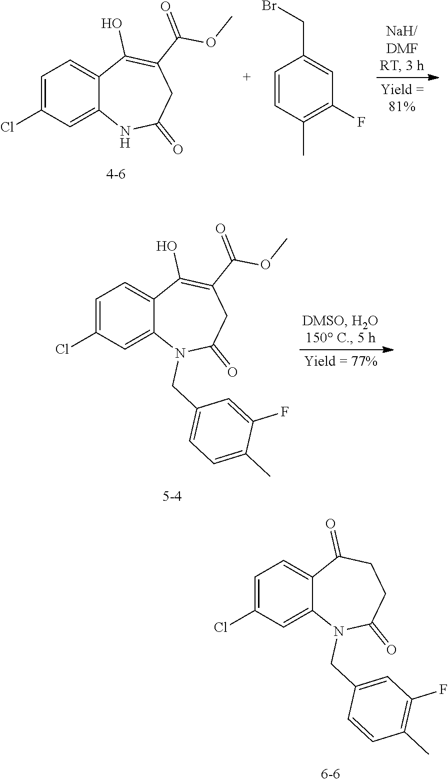

### 8-chloro-1-(3-fluoro-4-methylbenzyl)-3,4-dihydro-1H-benzo[b]azepine-2,5-dione (6-6)

To a stirred solution of 4-6 (4.54 g, 17 mmol, 1 equiv) in DMF (500 mL), was added NaH 60% dispersion in mineral oil (2.45 g, 61.2 mmol, 3.6 equiv), after stirring for 1 h, the mixture was added 4-(bromomethyl)-2-fluoro-1-methylbenzene (2.85 mL, 20.4 mmol, 1.2 equiv), and stir at rt for 3 h. The solution was quenched with H₂O (10 mL), acid by 1 M H₂SO₄ solution to PH=5 and extracted with EA (3×10 mL). The organic solutions were combined, brine (1×20 mL), dried over anhydrous Na₂SO₄, filtered and concentrated by rotary evaporation to get the crude compound. Then, the resulting concentrate was purified on a silica column to get 5.39 g (purity=96%) white solid compound with a yield of 81%.

TLC R_{f}=0.75 (EA:PE=1:2)

LCMS (FA) m/z=390.10 (M+H)

To the solution of resulting compound of methyl 8-chloro-1-(3-fluoro-4-methylbenzyl)-5-hydroxy-2-oxo-2,3-dihydro-1H-benzo[b]azepine-4-carboxylate 5-4 (5.39 g, 13.83 mmol, 1 equiv) in DMSO (21 mL), was added H₂O (I mL) under Nitrogen, and the reaction mixture was allowed to stir at 150° C. for 5 h. The reaction mixture was allowed to cool to rt, ice (70 mL) was added, and the mixture was allowed to stir 12 h. To the flask was added 1N H₂SO₄ solution (35 mL) at 0° C. and the mixture allowed to stir for 3 h. The resulting precipitate was filtered. Then, the crude product was purified on a silica column to get 3.51 g (purity=95%) light yellow oil compound with a yield of 77%.

TLC R_{f}=0.7 (PE:EA=2:1);

LCMS (m/z)=332.40 (M+H)

### Example 6. Method A and B for the Synthesis of Compounds of Formula 6

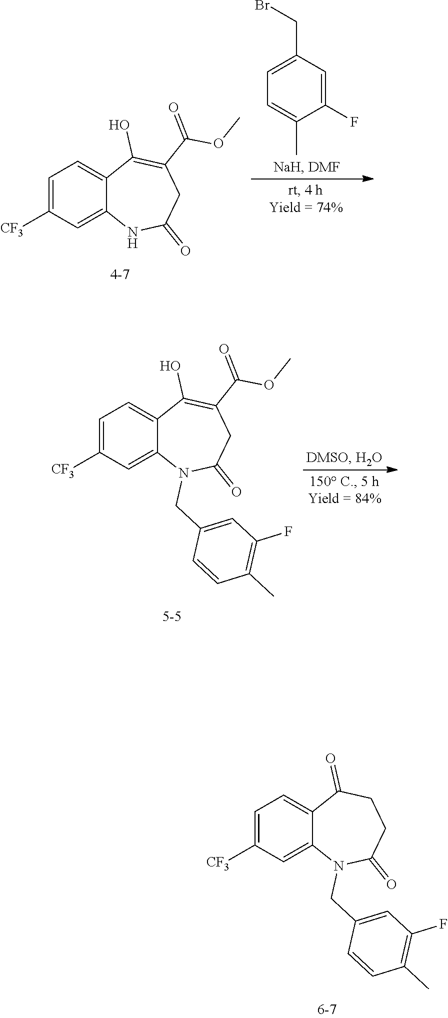

### 1-(3-fluoro-4-methylbenzyl)-8-(trifluoromethyl)-3,4-dihydro-1H-benzo[b]azepine-2,5-dione (6-7)

To a solution of methyl 5-hydroxy-2-oxo-2,3-dihydro-1H-benzo[b]azepine-4-carboxylate (5.12 g, 17 mmol, 1 equiv) in DMF (120 mL) was added NaH 60% dispersion in mineral oil (2.45 g, 61.2 mmol, 3.6 equiv). To this solution was added 4-(bromomethyl)-2-fluoro-1-methylbenzene (2.84 ml, 20.4 mmol, 1.2 equiv), and the reaction mixture was allowed to stir at rt for 4 h. The resulting solution was poured into water (30 mL) and extracted with Ethyl acetate (3×30 mL). The combined organic layers were washed with brine (40 mL), dried over anhydrous Na₂SO₄, and concentrated by rotary evaporation. Then, the resulting concentrate was purified on a silica column to get 5.35 g of the white solid compound with a yield of 74%, purity=97%.

TLC R_{f}=0.5 (PE:EA=4:1)

LCMS (m/z)=424.30 (M+H).

To the solution of resulting compound Methyl 1-(3-fluoro-4-methylbenzyl)-5-hydroxy-2-oxo-8-(trifluoromethyl)-2,3-dihydro-1H-benzo[b]azepine-4-carboxylate (5.35 g, 12.64 mmol, 1 equiv) in DMSO (36 mL), was added H₂O (1 mL) under nitrogen, and the reaction mixture was allowed to stir at 150° C. for 5 h. The reaction mixture was allowed to cool to rt, ice (60 mL) was added, and the mixture was allowed to stir 12 h. To the flask was added 1N H₂SO₄ (30 mL) at 0° C. and the mixture allowed to stir for 3 h. The resulting precipitate was filtered. Then, the crude product was purified on a silica column to get 3.89 g (purity=99%) light yellow oil compound with a yield of 84%.

TLC R_{f}=0.7 (PE:EA=2:1);

LCMS (m/z)=366.40 (M+H)

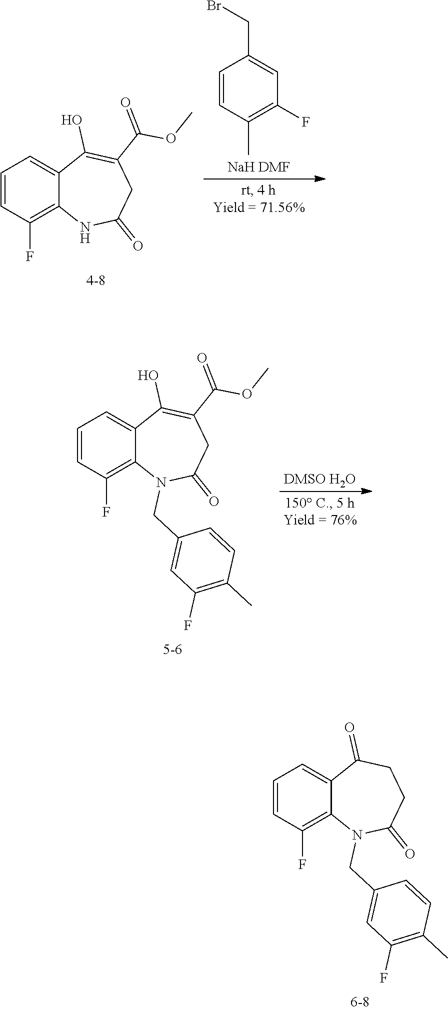

### 9-fluoro-1-(3-fluoro-4-methylbenzyl)-3,4-dihydro-1H-benzo[b]azepine-2,5-dione (6-8)

To a solution of methyl 9-fluoro-5-hydroxy-2-oxo-2,3-dihydro-1H-benzo[b]azepine-4-carboxylate (2.322 g, 9.24 mmol, 1 equiv) in DMF (75 mL) was added NaH—60% dispersion in mineral oil (1.331 g, 33.27 mmol, 3.6 equiv). To this solution was added 4-(bromomethyl)-2-fluoro-1-methylbenzene (11.09 mmol, 1.2 equiv), and the reaction mixture was allowed to stir at rt for 4 h. The resulting solution was poured into water (30 mL) and extracted with Ethyl acetate (3×30 mL). The combined organic layers were washed with brine (40 mL), dried over anhydrous Na₂SO₄, and concentrated by rotary evaporation. Then, the resulting concentrate was purified on a silica column to get 2.469 g of the yellow oil compound with a yield of 71.56%, purity=96.88%.

TLC R_{f}=0.7 (PE:EA=2:1)

LCMS (m/z)=374.70 (M+H)

To the solution of resulting compound 9-fluoro-1-(3-fluoro-4-methylbenzyl)-5-hydroxy-2-oxo-2,3-dihydro-1H-benzo[b]azepine-4-carboxylate (2.469 g, 6.613 mmol, 1 equiv) in DMSO (18.59 mL), was added H₂O (1.38 mL) under nitrogen, and the reaction mixture was allowed to stir at 150° C. for 5 h. The reaction mixture was allowed to cool to rt, ice (11.30 mL) was added, and the mixture was allowed to stir 12 h. To the flask was added 1N HCl (11.3 mL) at 0° C. and the mixture allowed to stir for 3 h. The resulting precipitate was filtered. Then, the crude product was purified on a silica column to get 1.602 g (purity=97.46%) pink oil compound with a yield of 76%.

TLC R_{f}=0.4 (PE:EA=2:1)

LCMS (m/z)=316.60 (M+H)

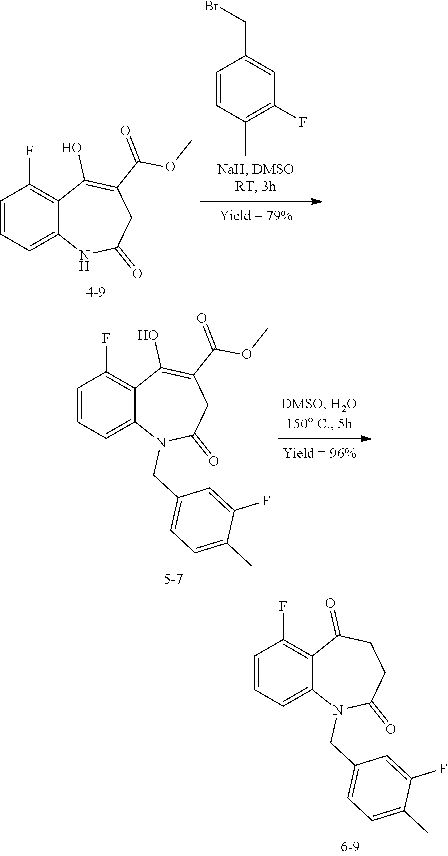

### 6-fluoro-1-(3-fluoro-4-methylbenzyl)-3,4-dihydro-1H-benzo[b]azepine-2,5-dione (6-9)

To a stirred solution of methyl 6-fluoro-5-hydroxy-2-oxo-2,3-dihydro-1H-benzo[b]azepine-4-carboxylate (5.924 g, 23.58 mmol, 1 equiv) in DMSO (94 mL), was added NaH 60% dispersion in mineral oil (3.396 g, 84.89 mmol, 3.6 equiv) at it. After 0.5 h, the mixture was added 4-(bromomethyl)-2-fluoro-1-methylbenzene (3.945 mL, 28.3 mmol, 1.2 equiv) at ambient temperature for 3 h. The solution was quenched with saturated ammonium chloride and extracted with EA (3×10 mL). The organic solutions were combined, washed with 1 M H₂SO₄ (2×10 mL) and brine (1×20 mL), dried over anhydrous Na₂SO₄, filtered and concentrated by rotary evaporation to get the crude compound. Then, the resulting concentrate was purified on a silica column to get 7 g white solid compound with a yield of 79%. Purity=90%

TLC R_{f}=0.4 (EA:PE=1:2)

LCMS (m/z)=374.10 (M+H)

To a solution of methyl 6-fluoro-1-(3-fluoro-4-methylbenzyl)-5-hydroxy-2-oxo-2,3-dihydro-1H-benzo[b]azepine-4-carboxylate (7 g, 18.75 mmol, 1 equiv) in DMSO (29 mL), was added H₂O (I mL) under nitrogen, and the reaction mixture was allowed to stir at 150° C. for 5 h. The reaction mixture was allowed to cool to rt, ice (45 mL) was added, and the mixture was allowed to stir 12 h. To the flask was added 1N HCl (45 mL) at 0° C. and the mixture allowed to stir for 3 h. The resulting precipitate was filtered. Then, the crude product was purified on a silica column to get 5.685 g light yellow oil compound with a yield of 96%. Purity=89%.

TLC R_{f}=0.3 (EA:PE=1:2)

LCMS (m/z)=316.00 (M+H)

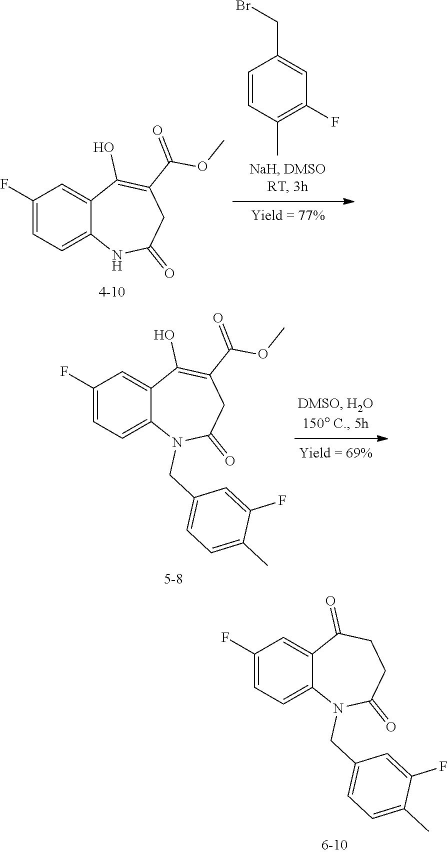

### 7-fluoro-1-(3-fluoro-4-methylbenzyl)-3,4-dihydro-1H-benzo[b]azepine-2,5-dione (6-10)

To a stirred solution of methyl 7-fluoro-5-hydroxy-2-oxo-2,3-dihydro-1H-benzo[b]azepine-4-carboxylate (6.732 g, 26.80 mmol, 1 equiv) in DMSO (107 mL), was added NaH 60% dispersion in mineral oil (3.859 g, 96.47 mmol, 3.6 equiv) at rt. After 0.5 h, the mixture was added 4-(bromomethyl)-2-fluoro-1-methylbenzene (4.48 mL, 32.16 mmol, 1.2 equiv) at ambient temperature for 3 h. The solution was quenched with saturated ammonium chloride and extracted with EA (3×10 mL). The organic solutions were combined, washed with 1 M H₂SO₄ (2×10 mL) and brine (1×20 mL), dried over anhydrous Na₂SO₄, filtered and concentrated by rotary evaporation to get the crude compound. Then, the resulting concentrate was purified on a silica column to get 7.747 g white solid compound with a yield of 77%. Purity=89%

TLC R_{f}=0.5 (EA:PE=1:2)

LCMS (FA) m/z=374.10 (M+H)

To a solution of Methyl 7-fluoro-1-(3-fluoro-4-methylbenzyl)-5-hydroxy-2-oxo-2,3-dihydro-1H-benzo[b]azepine-4-carboxylate (7.747 g, 20.75 mmol, 1 equiv) in DMSO (32 mL), was added H₂O (1 mL) under nitrogen, and the reaction mixture was allowed to stir at 150° C. for 5 h. The reaction mixture was allowed to cool to rt, ice (50 mL) was added, and the mixture was allowed to stir 12 h. To the flask was added 1N HCl (50 mL) at 0° C. and the mixture allowed to stir for 3 h. The resulting precipitate was filtered. Then, the crude product was purified on a silica column to get 4.538 g light yellow oil compound with a yield of 69%. Purity=97%.

TLC R_{f}=0.35 (EA:PE=1:2)

LCMS (FA) m/z=316.00 (M+H)

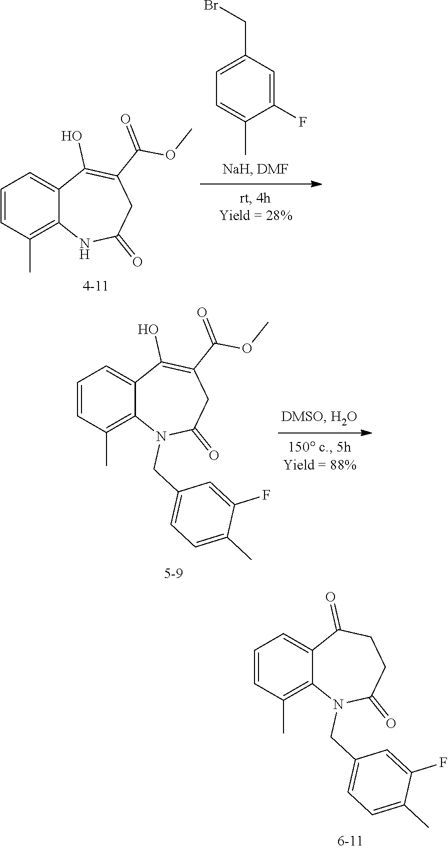

### 1-(3-fluoro-4-methylbenzyl)-9-methyl-3,4-dihydro-1H-benzo[b]azepine-2,5-dione (6-11)

To a solution of methyl 5-hydroxy-9-methyl-2-oxo-2,3-dihydro-1H-benzo[b]azepine-4-carboxylate (2.6 g, 10.5 mmol, 1 equiv) in DMF (74 mL) was added NaH 60% dispersion in mineral oil (907 mg, 37.8 mmol, 3 equiv). To this solution was added 4-(bromomethyl)-2-fluoro-1-methylbenzene (12.6 mmol, 1.2 equiv), and the reaction mixture was allowed to stir at rt for 4 h. The resulting solution was poured into water (30 mL) and extracted with Ethyl acetate (3×30 mL). The combined organic layers were washed with brine (40 mL), dried over anhydrous Na₂SO₄, and concentrated by rotary evaporation. Then, the resulting concentrate was purified on a silica column to get 1.08 g of the white solid compound with a yield of 28%, purity=97%.

TLC R_{f}=0.6 (PE:EA=2:1)

LCMS (m/z)=370.10 (M+H).

To the solution of resulting compound of Methyl 1-(3-fluoro-4-methylbenzyl)-5-hydroxy-9-methyl-2-oxo-2,3-dihydro-1H-benzo[b]azepine-4-carboxylate (1.08 g, 2.92 mmol, 1 equiv) in DMSO (8.2 mL), was added H₂O (233 uL) under nitrogen, and the reaction mixture was allowed to stir at 150′C for 5 h. The reaction mixture was allowed to cool to rt, ice (11.3 mL) was added, and the mixture was allowed to stir 12 h. To the flask was added 1 N HCl (11.3 mL) at 0° C. and the mixture allowed to stir for 3 h. The resulting precipitate was filtered. Then, the crude product was purified on a silica column to get 800 mg (purity=98%) light yellow oil compound with a yield of 88%.

TLC R_{f}=0.4 (PE:EA=2:1);

LCMS (m/z)=312.10 (M+H)

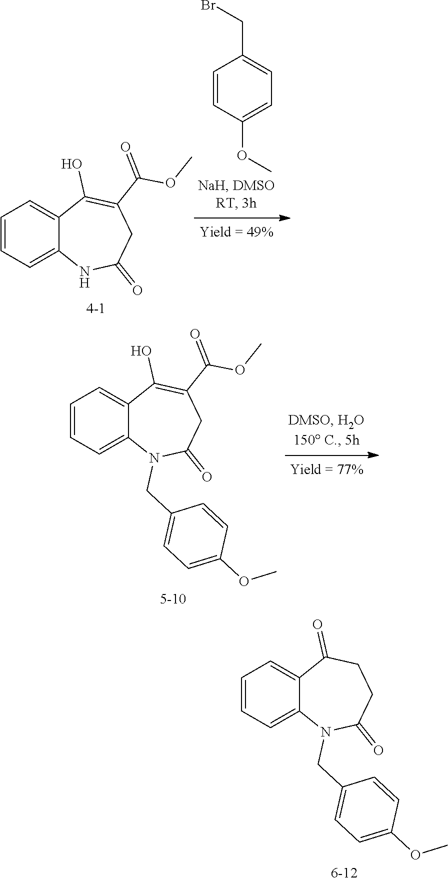

### 1-(4-methoxybenzyl)-3,4-dihydro-1H-benzo[b]azepine-2,5-dione (6-12)

To a stirred solution of methyl 5-hydroxy-2-oxo-2,3-dihydro-1l-1-benzo[b]azepine-4-carboxylate (19 g, 81.47 mmol, 1 equiv) in DMSO (244 mL), was added NaH 60% dispersion in mineral oil (11.7 g, 293.28 mmol, 3.6 equiv) at rt. After 0.5 h, the mixture was added 1-(bromomethyl)-4-methoxybenzene (11.9 mL, 97.76 mmol, 1.2 equiv) at ambient temperature for 3 h. The solution was quenched with saturated ammonium chloride and extracted with EA (3×100 mL). The organic solutions were combined, washed with 1 M H₂SO₄ (2×100 mL) and brine (200 mL), dried over anhydrous Na₂SO₄, filtered and concentrated by rotary evaporation to get the crude compound. Then, the resulting concentrate was purified on a silica column to get 14 g white solid compound with a yield of 49%. Purity=73%

TLC R_{f}=0.6 (EA:PE=1:2)

LCMS (m/z)=354.20 (M+H)

To a solution of methyl 5-hydroxy-1-(4-methoxybenzyl)-2-oxo-2,3-dihydro-1H-benzo[b]azepine-4-carboxylate (20 g, 56.6 mmol, 1 equiv) in DMSO (88 mL), was added H₂O (3 mL) under nitrogen, and the reaction mixture was allowed to stir at 150° C. for 5 h. The reaction mixture was allowed to cool to rt, ice (150 mL) was added, and the mixture was allowed to stir 12 h. To the flask was added 1N HCl (150 mL) at 0° C. and the mixture allowed to stir for 3 h. The resulting precipitate was filtered. Then, the crude product was purified on a silica column to get 12.9 g light yellow oil compound with a yield of 77%. Purity=91%

TLC R_{f}=0.5 (EA:PE=1:1)

LCMS (m/z)=296.100 (M+H)

### Example 7. Representative Synthesis of Compounds of Formula 7

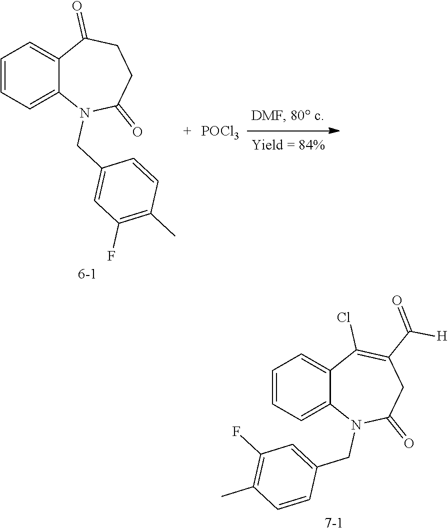

### 5-chloro-1-(3-fluoro-4-methylbenzyl)-2-oxo-2,3-dihydro-1H-benzo[b]azepine-4-carbaldehyde (7-1)

Phosphorus oxychloride (61 μL, 0.65 mmol) was added to a flask containing N, N-dimethylformamide (0.5 mL) in an ice bath and stirred for 10 min. The ice bath was replaced with an ambient temperature water bath and stirred for an additional 10-15 min. 1-(3-fluoro-4-methylbenzyl)-3,4-dihydro-1H-benzo[b]azepine-2,5-dione 6-1 (148 mg, 0.5 mmol) was added and stirred for 15 min. The reaction mixture was heated to 80° C. and stirred for an additional 3 h. The orange solution was diluted with ice water and neutralised with 20% sodium acetate solution and extracted with EA (3×10-20 mL). The combined organic extracts were washed with saturated aqueous sodium bicarbonate solution 50 mL and brine 50 mL, dried over anhydrous Na₂SO₄, filtered and concentrated by rotary evaporation to get the crude compound. Then, the resulting concentrate was purified on a silica column to get 144 mg (purity=99%) yellow solid compound with a yield of 84%.

TLC R_{f}=0.5 (PE:EA=2:1)

LCMS (m/z)=344.20 (M+H)

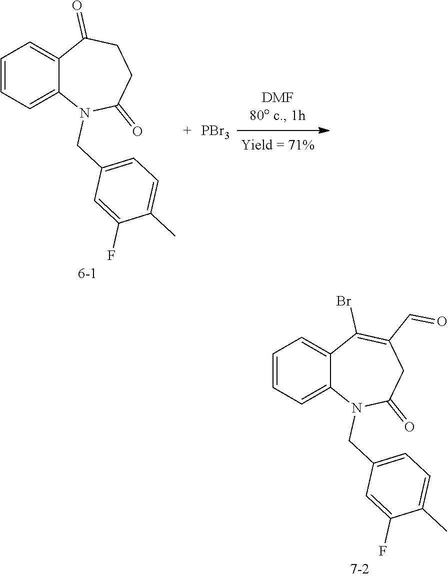

### 5-bromo-1-(3-fluoro-4-methylbenzyl)-2-oxo-2,3-dihydro-1H-benzo[b]azepine-4-carbaldehyde (7-2)

Phosphorus tribromide (271.6 μL, 2.86 mmol, 1.3 equiv) was added to a flask containing N, N-dimethylformamide (22 mL) in an ice bath and stirred for 10 min. The ice bath was replaced with an ambient temperature water bath and stirred for an additional 10-15 min. 1-(3-fluoro-4-methylbenzyl)-3,4-dihydro-1H-benzo[b]azepine-2,5-dione 6-1 (653.4 mg, 2.2 mmol, 1 equiv) was added and stirred for 15 min. The reaction mixture was heated to 80° C. and stirred for an additional 3 h. The orange solution was diluted with ice water and neutralised with 20% sodium acetate solution and extracted with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 608 mg (purity=99%) yellow solid compound with a yield of 71%.

TLC R_{f}=0.6 (PE:EA=2:1)

LCMS (m/z)=390.04 (M+2H)

¹H NMR (400 MHz, DMSO) δ 10.03 (s, 1H), 7.89-7.78 (m, 1H), 7.65-7.50 (m, 2H), 7.36 (ddd, J=8.2, 6.2, 2.3 Hz, 1H), 7.12 (t, J=7.8 Hz, 1H), 6.74 (d, J=9.3 Hz, 2H), 5.33 (d, J=16.0 Hz, 1H), 4.89 (d, J=16.0 Hz, 1H), 3.73 (d, J=12.9 Hz, 1H), 2.63 (d, J=12.9 Hz, 1H), 2.12 (s, 3H). ¹³C NMR (100 MHz, DMSO) δ 190.39, 169.23, 162.22, 159.80, 140.38, 137.70, 137.63, 136.09, 134.20, 132.78, 132.52, 132.03, 131.98, 130.70, 126.15, 123.93, 123.30, 123.13, 123.01, 122.98, 113.83, 113.60, 49.69, 33.66, 14.24, 14.21.

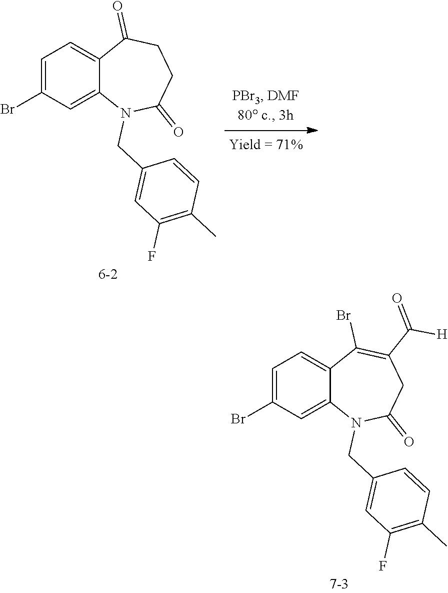 [SMILES: Cc1ccc(CN2C(=O)CCC(=O)c3ccc(Br)cc32)cc1F ; Cc1ccc(CN2C(=O)CC(C=O)=C(Br)c3ccc(Br)cc32)cc1F]

### 5,8-dibromo-1-(3-fluoro-4-methylbenzyl)-2-oxo-2,3-dihydro-1H-benzo[b]azepine-4-carbaldehyde (7-3)

Phosphorus tribromide (185 μL, 1.95 mmol, 1.3 equiv) was added to a flask containing N, N-dimethylformamide (15 mL) in an ice bath and stirred for 10 min. The ice bath was replaced with an ambient temperature water bath and stirred for an additional 10-15 min. 8-bromo-1-(3-fluoro-4-methylbenzyl)-3,4-dihydro-1H-benzo[b]azepine-2,5-dione 6-2 (564 mg, 1.5 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 700.5 ng (purity=98%) yellow solid compound with a yield of 71%.

TLC R_{f}=0.6 (PE:EA=4:1)

LCMS (m/z)=467.90 (M+H)

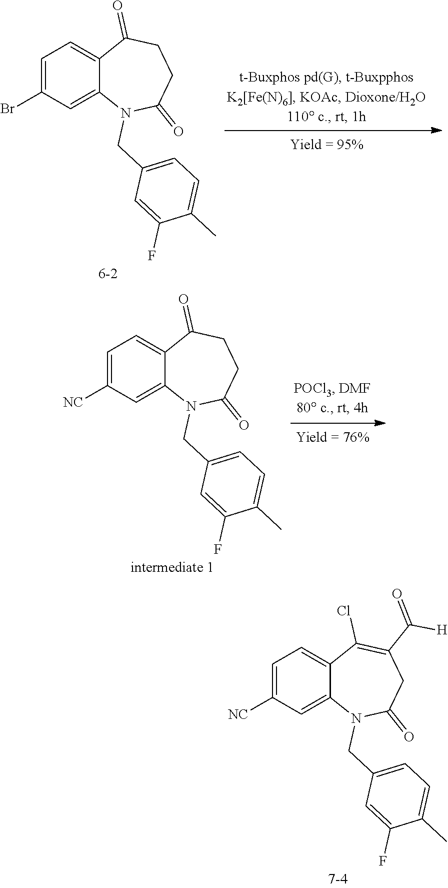 [SMILES: Cc1ccc(CN2C(=O)CCC(=O)c3ccc(Br)cc32)cc1F ; Cc1ccc(CN2C(=O)CCC(=O)c3ccc(C#N)cc32)cc1F ; Cc1ccc(CN2C(=O)CC(C=O)=C(Cl)c3ccc(C#N)cc32)cc1F]

### 5-chloro-1-(3-fluoro-4-methylbenzyl)-4-formyl-2-oxo-2,3-dihydro-1H-benzo[b]azepine-8-carbonitrile (7-4)

To a solu-tion of 8-bromo-1-(3-fluoro-4-methylbenzyl)-3,4-dihydro-1H-benzo[b]azepine-2,5-dione 6-2 (1 g, 2.66 mmol, 1 equiv) in 1,4-dioxane (9.4 mL) were added potassium acetate (32.3 mg, 0.33 mmol, 0.25 equiv), potassium hexacyanoferrate (II) trihydrate (437.6 mg, 1.33 mmol, 0.5 equiv), t-BuXPhos palladium (I) phe-nethylamine chloride (89 mg, 0.13 mmol, 0.05 equiv), 2-di-tert-butylpho-sphino-2′,4′,6′-trisopropylbiphenyl (55 mg, 0.13 mmol, 0.05 equiv), and water(9.4 ml). After being heated with stirring at 110° C. for 1 h, the reaction mixture was diluted with EA. After removal of the catalyst by filtration, the filtrate was washed with water and brine. The organic layer was dried over Na₂SO₄ and concentrated in vacuo. Then, the resulting concentrate was purified on a silica column to get 815 mg (purity=99%) yellow solid compound with a yield of 95%.

TLC R_{f}=0.3 (PE:EA=2:1)

LCMS (m/z)=323.40 (M+H)

Phosphorus oxychloride (61 μL, 0.65 mmol, 1.3 equiv) was added to a flask containing N, N-dimethylformamide (0.5 mL) in an ice bath and stirred for 10 min. Ice bath was replaced with an ambient temperature water bath and stirred for an additional 10-15 min. 1-(3-fluoro-4-methylbenzyl)-2,5-dioxo-2,3,4,5-tetrahydro-1Hbenzo[b]azepine-8-carbonitrile (intermediate 1) (161 mg, 0.5 mmol, 1 equiv) was added and stirred for 15 miii. The reaction mixture was heated to 80° C. and stirred for an additional 4 h. The orange solution was diluted with ice water and neutralised with 20% sodium acetate solution and extracted with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 140 mg (purity=99%) yellow solid compound with a yield of 76%.

TLC R_{f}=0.6 (PE:EA=2:1)

LCMS (m/z)=369.50 (M+H)

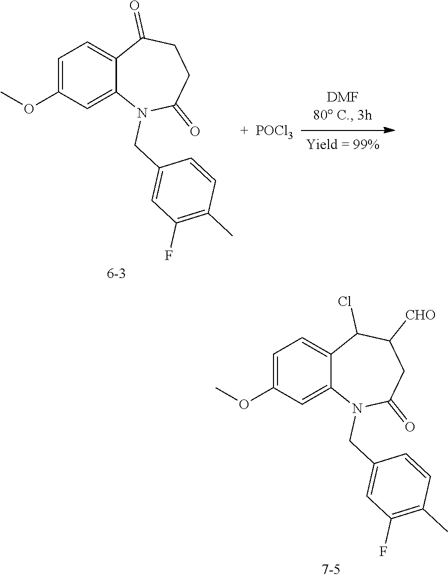

### 5-chloro-1-(3-fluoro-4-methylbenzyl)-8-methoxy-2-oxo-2,3-dihydro-1H-benzo[b]azepine-4-carbaldehyde (7-5)

Phosphorus oxychloride (0.12 mL, 1.19 mmol, 1.3 equiv) was added to a flask containing N, N-dimethylformamide (1 mL) in an ice bath and stirred for 10 min. The ice bath was replaced with an ambient temperature water bath and stirred for an additional 10-15 min. 1-(3-fluoro-4-methylbenzyl)-8-methoxy-3,4-dihydro-1H-benzo[b]azepine-2,5-dione 6-3 (300 mg, 0.917 mmol, 1 equiv) was added and stirred for 15 min. The reaction mixture was heated to 80° C. and stirred for an additional 3 h. The orange solution was diluted with ice water and neutralised with 20% sodium acetate solution and extracted with EA (3×10-20 mL). The combined organic extracts were washed with saturated aqueous sodium bicarbonate solution 50 mL, brine 50 mL, and water (3×50 mL), dried over anhydrous Na₂SO₄, filtered and concentrated by rotary evaporation to get the crude compound. Then, the resulting concentrate was purified on a silica column to get 343 mg (purity=99%) yellow solid compound with a yield of 99%.

TLC R_{f}=0.6 (EA:PE=1:2)

LCMS (m/z)=374.60 (M+H)

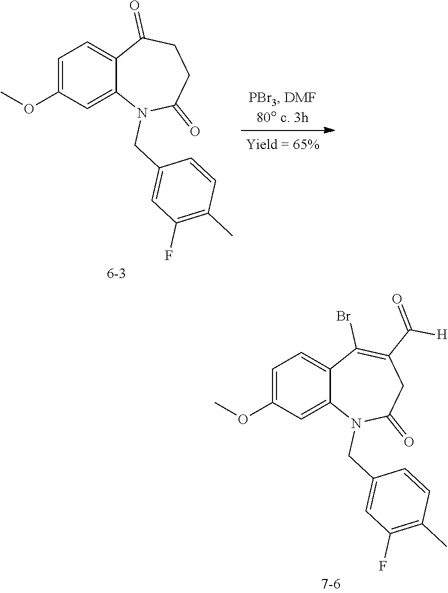

### 5-bromo-1-(3-fluoro-4-methylbenzyl)-8-methoxy-2-oxo-2,3-dihydro-1H-benzo[b]azepine-4-carbaldehyde (7-6)

Phosphorus tribromide (112 μL, 1.19 mmol, 1.3 equiv) was added to a flask containing N, N-dimethylformamide (20 mL) in an ice bath and stirred for 10 min. The ice bath was replaced with an ambient temperature water bath and stirred for an additional 10-15 min. 1-(3-fluoro-4-methylbenzyl)-3,4-dihydro-1H-benzo[b]azepine-2,5-dione 6-3 (300 mg, 0.917 mmol, 1 equiv) was added and stirred for 15 min. The reaction mixture was heated to 80° C. and stirred for an additional 3 h. The solution was diluted with ice water and neutralised with 20% sodium acetate solution and with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 248 mg (purity=98%) yellow solid compound with a yield of 65%.

TLC R_{f}=0.6 (PE:EA=2:1)

LCMS (m/z)=418.40 (M+H)

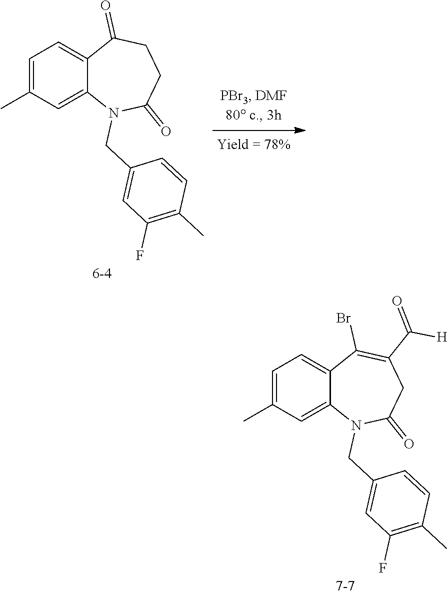

### 5-bromo-1-(3-fluoro-4-methylbenzyl)-8-methyl-2-oxo-2,3-dihydro-1H-benzo[b]azepine-4-carbaldehyde (7-7)

Phosphorus tribromide (126 μL, 1.326 mmol, 1.3 equiv) was added to a flask containing N, N-dimethylformamide (1 mL) in an ice bath and stirred for 10 min. The ice bath was replaced with an ambient temperature water bath and stirred for an additional 10-15 min. 1-(3-fluoro-4-methylbenzyl)-8-methyl-3,4-dihydro-1H-benzo[b]azepine-2,5-dione 6-4 (316.8 mg, 1.02 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 320 mg (purity=98%) yellow solid compound with a yield of 78%.

TLC R_{f}=0.8 (PE:EA=2:1)

LCMS (m/z)=402.30 (M+H)

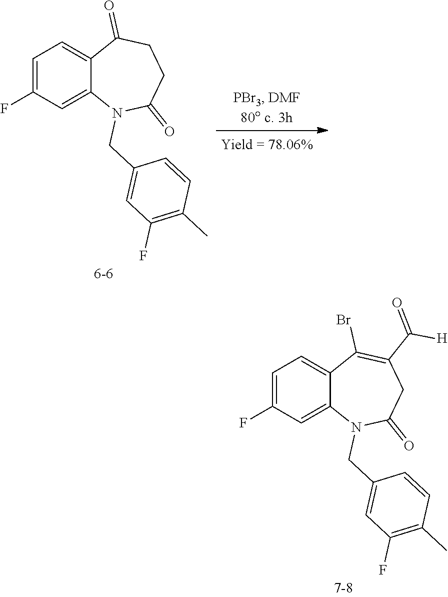 [SMILES: Cc1ccc(CN2C(=O)CCC(=O)c3ccc(F)cc32)cc1F ; Cc1ccc(CN2C(=O)CC(C=O)=C(Br)c3ccc(F)cc32)cc1F]

### 5-bromo-8-fluoro-1-(3-fluoro-4-methylbenzyl)-2-oxo-2,3-dihydro-1H-benzo[b]azepine-4-carbaldehyde (7-8)

Phosphorus tribromide (274.1 uL, 2.88 mmol, 1.3 equiv) was added to a flask containing N, N-dimethylformamide (22.33 ml) in an ice bath and stirred for 10 min. The ice bath was replaced with an ambient temperature water bath and stirred for an additional 10-15 min. 8-fluoro-1-(3-fluoro-4-methylbenzyl)-3,4-dihydro-1H-benzo[b]azepine-2,5-dione 6-6 (700 mg, 2.22 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 704 mg (purity=99%) orange solid compound with a yield of 78.06%.

TLC R_{f}=0.6 (PE:EA=2:1)

LCMS (m/z)=406.0 (M+H)

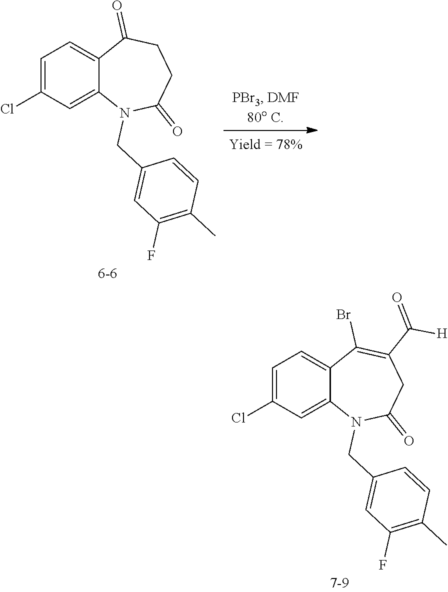 [SMILES: Cc1ccc(CN2C(=O)CCC(=O)c3ccc(Cl)cc32)cc1F ; Cc1ccc(CN2C(=O)CC(C=O)=C(Br)c3ccc(Cl)cc32)cc1F]

### 5-bromo-8-chloro-1-(3-fluoro-4-methylbenzyl)-2-oxo-2,3-dihydro-1H-benzo[b]azepine-4-carbaldehyde(7-9)

Phosphorus tribromide (0.75 ml, 7.84 mmol, 1.3 equiv) was added to a flask containing N, N-dimethylformamide (66 mL) in an ice bath and stirred for 10 min. The ice bath was replaced with an ambient temperature water bath and stirred for an additional 10-15 min. 8-chloro-1-(3-fluoro-4-methylbenzyl)-3,4-dihydro-1H-benzo[b]azepine-2,5-dione 6-6 (2 g, 6 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 1.97 g (purity=97%) yellow solid compound with a yield of 78%.

TLC R_{f}=0.6 (PE:EA=2:1);

LCMS (m/z)=423.70 (M+H)

### Example 8. Representative Synthesis of Compounds of Formula 7

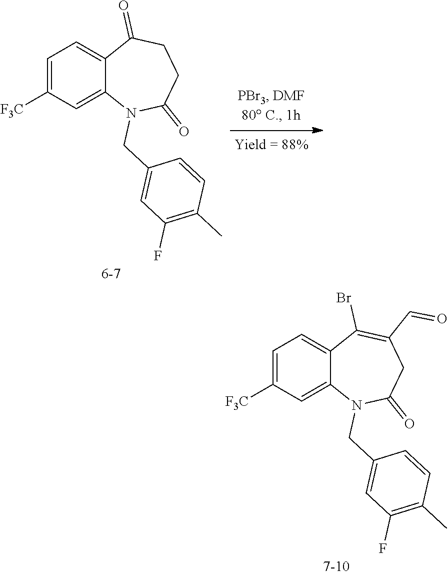 [SMILES: Cc1ccc(CN2C(=O)CCC(=O)c3ccc(C(F)(F)F)cc32)cc1F ; Cc1ccc(CN2C(=O)CC(C=O)=C(Br)c3ccc(C(F)(F)F)cc32)cc1F]

### 5-bromo-1-(3-fluoro-4-methylbenzyl)-2-oxo-8-(trifluoromethyl)-2,3-dihydro-1H-benzo[b]azepine-4-carbaldehyde (7-10)

Phosphorus tribromide (889 μL, 9.36 mmol, 1.3 equiv) was added to a flask containing N,N-dimethylformamide (72 mL) in an ice bath and stirred for 10 min. The ice bath was replaced with an ambient temperature water bath and stirred for an additional 10-15 min. I-(3-fluoro-4-methylbenzyl)-8-(trifluoromethyl)-3,4-dihydro-1H-benzo[b]azepine-2,5-dione (2.6 g, 7.2 mmol, 1.0 equiv) was added and stirred for 15 min. The reaction mixture was heated to 80° C. and stirred for an additional 1 h. The orange solution was diluted with ice water and neutralised with 20% sodium acetate solution and with diethyl ether (3×10-20 mL). The combined organic extracts were washed with saturated aqueous sodium bicarbonate solution (50 mL), brine (50 mL), and water (3×50 mL), dried over anhydrous Na₂SO₄, filtered and concentrated by rotary evaporation to get the crude compound. Then, the resulting concentrate was purified on a silica column to get 2.889 g (purity=91%) yellow oil compound with a yield of 88%.

TLC R_{f}=0.85 (PE:EA=1:1);

LCMS (m/z)=459.00 (M+H)

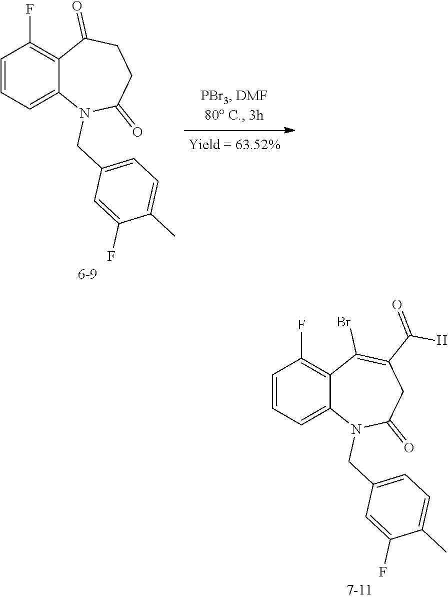 [SMILES: Cc1ccc(CN2C(=O)CCC(=O)c3c(F)cccc32)cc1F ; Cc1ccc(CN2C(=O)CC(C=O)=C(Br)c3c(F)cccc32)cc1F]

### 5-bromo-9-fluoro-1-(3-fluoro-4-methylbenzyl)-2-oxo-2,3-dihydro-1H-benzo[b]azepine-4-carbaldehyde (7-11)

Tribromophosphane (627.3 μL, 6.60 mmol, 1.3 equiv) was added to a flask containing N, N-dimethylformamide (51.09 mL) in an ice bath and stirred for 10 min. The ice bath was replaced with an ambient temperature water bath and stirred for an additional 10-15 min. 9-fluoro-1-(3-fluoro-4-methylbenzyl)-3,4-dihydro-1H-benzo[b]azepine-2,5-dione (1.602 g, 5.08 mmol, 1.0 equiv) was added and stirred for 15 min. The reaction mixture was heated to 80° C. and stirred for an additional 3 h. The orange solution was diluted with ice water and neutralised with 20% sodium acetate solution and extracted with EA (3×10-20 mL). The combined organic extracts were washed with saturated aqueous sodium bicarbonate solution 50 mL and brine 50 mL, dried over anhydrous Na₂SO₄, filtered and concentrated by rotary evaporation to get the crude compound. Then, the resulting concentrate was purified on a silica column to get 1.3 g (purity=99%) orange solid compound with a yield of 63.52%.

TLC R_{f}=0.6 (PE:EA=2:1)

LCMS (m/z)=406.70 (M+H)

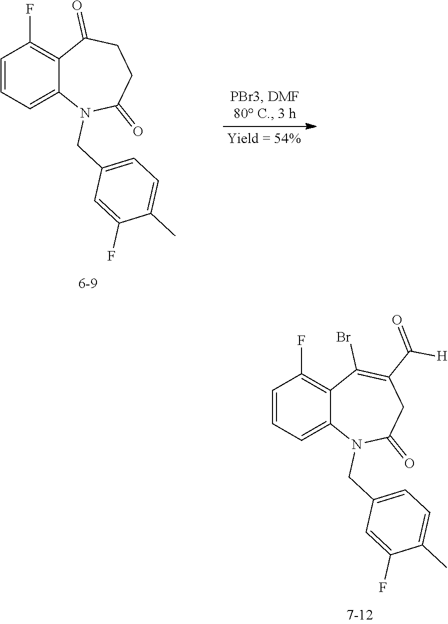 [SMILES: Cc1ccc(CN2C(=O)CCC(=O)c3c(F)cccc32)cc1F ; Cc1ccc(CN2C(=O)CC(C=O)=C(Br)c3c(F)cccc32)cc1F]

### 5-bromo-6-fluoro-1-(3-fluoro-4-methylbenzyl)-2-oxo-2,3-dihydro-1H-benzo[b]azepine-4-carbaldehyde (7-12)

Phosphorus tribromide (2.23 mL, 23.46 mmol, 1.3 equiv) was added to a flask containing N, N-dimethylformamide (180 mL) in an ice bath and stirred for 10 min. The ice bath was replaced with an ambient temperature water bath and stirred for an additional 10-15 min. 6-fluoro-1-(3-fluoro-4-methylbenzyl)-3,4-dihydro-1H-benzo[b]azepine-2,5-dione (5.685 g, 18.02 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×10-20 mL). The combined organic extracts were washed with saturated aqueous sodium bicarbonate solution (50 mL), brine (0.50 mL), and water (3×50 mL), dried over anhydrous Na₂SO₄, filtered and concentrated by rotary evaporation to get the crude compound. Then, the resulting concentrate was purified on a silica column to get 3.95 g yellow solid compound with a yield of 54%. Purity=87%

TLC R_{f}=0.5 (EA:PE=1:2)

LCMS (m/z)=407.60 (M+H)

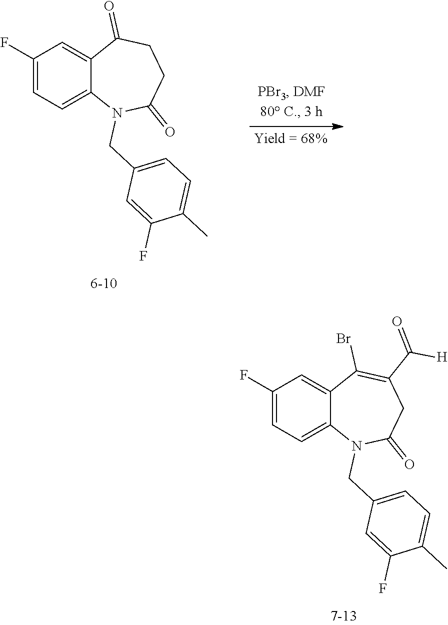 [SMILES: Cc1ccc(CN2C(=O)CCC(=O)c3cc(F)ccc32)cc1F ; Cc1ccc(CN2C(=O)CC(C=O)=C(Br)c3cc(F)ccc32)cc1F]

### 5-bromo-7-fluoro-1-(3-fluoro-4-methylbenzyl)-2-oxo-2,3-dihydro-1H-benzo[b]azepine-4-carbaldehyde (7-13)

Phosphorus tribromide (1.76 mL, 18.73 mmol, 1.3 equiv) was added to a flask containing N, N-dimethylfomamide (144 mL) in an ice bath and stirred for 10 min. The ice bath was replaced with an ambient temperature water bath and stirred for an additional 10-15 min. 7-fluoro-1-(3-fluoro-4-methylbenzyl)-3,4-dihydro-1H-benzo[b]azepine-2,5-dione (4.538 g, 14.4 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×10-20 mL). The combined organic extracts were washed with saturated aqueous sodium bicarbonate solution (50 mL), brine (50 mL), and water (3×50 mL), dried over anhydrous Na₂SO₄, filtered and concentrated by rotary evaporation to get the crude compound. Then, the resulting concentrate was purified on a silica column to get 4 g yellow solid compound with a yield of 68%. Purity=98%

TLC R_{f}=0.6 (EA:PE=1:2)

LCMS (m/z)=407.70 (M+H)

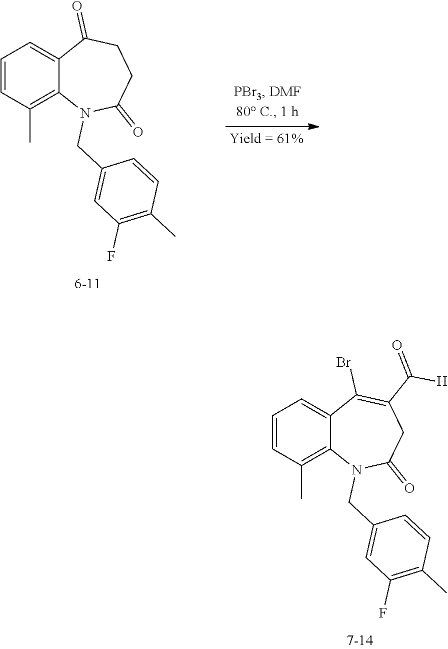 [SMILES: Cc1ccc(CN2C(=O)CCC(=O)c3cccc(C)c32)cc1F ; Cc1ccc(CN2C(=O)CC(C=O)=C(Br)c3cccc(C)c32)cc1F]

### bromo-1-(3-fluoro-4-methylbenzyl)-9-methyl-2-oxo-2,3-dihydro-1H-benzo[b]azepine-4-carbaldehyde (7-14)

Phosphorus tribromide (306 ul, 3.21 mmol, 1.3 equiv) was added to a flask containing N, N-dimethylformamide (24.7 mL) in an ice bath and stirred for 10 min. The ice bath was replaced with an ambient temperature water bath and stirred for an additional 10-15 min. 1-(3-fluoro-4-methylbenzyl)-9-methyl-3,4-dihydro-1H-benzo[b]azepine-2,5-dione (770 mg, 2.47 mmol, 1 equiv) was added and stirred for 15 min. The reaction mixture was heated to 80° C. and stirred for an additional 1 h. The orange solution was diluted with ice water and neutralised with 20% sodium acetate solution and with EA (3×10-20 mL). The combined organic extracts were washed with saturated aqueous sodium bicarbonate solution (50 mL), brine (50 mL), and water (3×50 mL), dried over anhydrous Na₂SO₄, filtered and concentrated by rotary evaporation to get the crude compound. Then, the resulting concentrate was purified on a silica column to get 610 mg (purity=91%) yellow solid compound with a yield of 61%.

TLC R_{f}=0.6 (PE:EA=2:1);

LCMS (m/z)=404.00 (M+H)

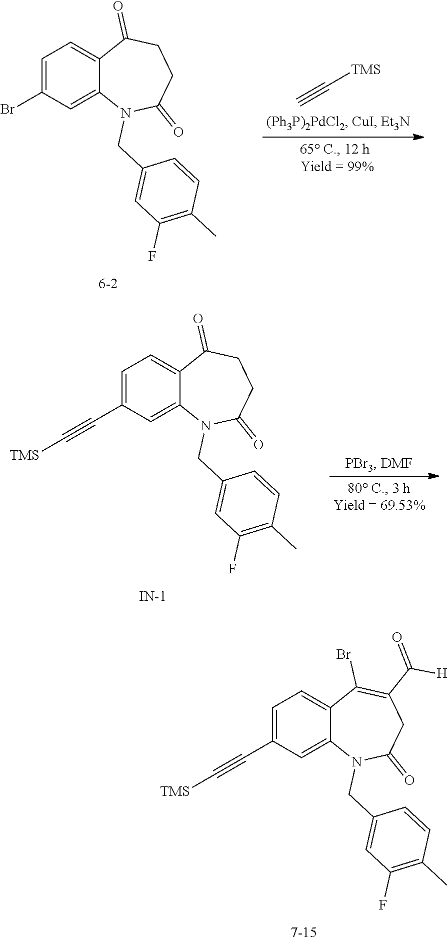

### 5-bromo-1-(3-fluoro-4-methylbenzyl)-2-oxo-8-((trimethylsilyl)ethynyl)-2,3-dihydro-1H-benzo[b]azepine-4-carbaldehyde (7-15)

A mixture of 8-bromo-1-(3-fluoro-4-methylbenzyl)-3,4-dihydro-1H-benzo[b]azepine-2,5-dione (1 g, 2.658 mmol, 1.0 equiv). (Ph₃P)PdCl₂ (23.369 mg, 0.05315 mmol, 0.02 equiv) and CuI (2.453 mg, 0.02658 mmol, 0.01 equiv) in a flask was degassed for 10 min, then Et₃N (0.2 M for substrate) was added under argon atmosphere. To this solution ethynyl trimethylsilane (1.878 mL, 13.289 mmol, 5 equiv) was added by syringe. The mixture was warmed to 65° C. for 12 hours. After cooling to room temperature, the reaction mixture was filtrated through celite. The filtrate was concentrated and purified by chromatography to get the product 1.045 g (purity=89.40%) as a brown solid. Yield: 99%.

TLC R_{f}=0.7 (PE:EA=2:1)

LCMS (m/z)=394.10 (M+H)

Tribromophosphane (271 uL, 2.854 mmol, 1.3 equiv) was added to a flask containing N, N-dimethylformamide (21.95 mL) in an ice bath and stirred for 10 min. The ice bath was replaced with an ambient temperature water bath and stirred for an additional 10-15 min. 1-(3-fluoro-4-methylbenzyl)-8-((trimethylsilyl)ethynyl)-3,4-dihydro-1H-benzo[b]azepine-2,5-dione (865 mg, 2.195 mmol, 1 equiv) was added and stirred for 15 min. The reaction mixture was heated to 80° C. and stirred for an additional 3 h. The orange solution was diluted with ice water and neutralised with 20% sodium acetate solution and with EA (3×10-20 mL). The combined organic extracts were washed with saturated aqueous sodium bicarbonate solution (50 mL), brine (50 mL), and water (3×50 mL), dried over anhydrous Na₂SO₄, filtered and concentrated by rotary evaporation to get the crude compound. Then, the resulting concentrate was purified on a silica column to get 968 mg (purity=99%) brown solid compound with a yield of 69.53%.

TLC R_{f}=0.6 (PE:EA=4:1)

LCMS (m/z)=485.80 (M+H)

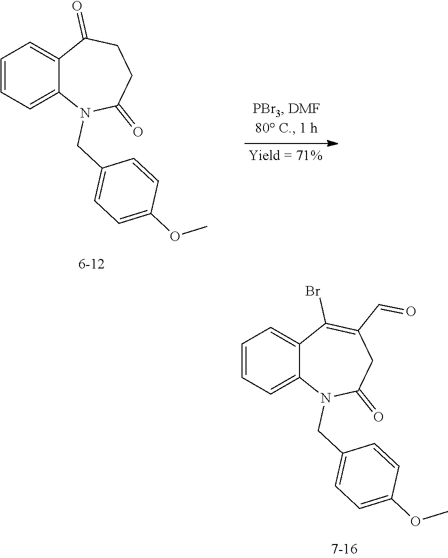 [SMILES: COc1ccc(CN2C(=O)CCC(=O)c3ccccc32)cc1 ; COc1ccc(CN2C(=O)CC(C=O)=C(Br)c3ccccc32)cc1]

### 5-bromo-1-(4-methoxybenzyl)-2-oxo-2,3-dihydro-1H-benzo[b]azepine-4-carbaldehyde (7-15)

Phosphorus tribromide (986 μL, 10.4 mmol, 1.3 equiv) was added to a flask containing N,N-dimethylformamide (80 mL) in an ice bath and stirred for 10 min. The ice bath was replaced with an ambient temperature water bath and stirred for an additional 10-15 min. 1-(4-methoxybenzyl)-3,4-dihydro-1H-benzo[b]azepine-2,5-dione (2.3 g, 8 mmol, 1.0 equiv) was added and stirred for 15 min. The reaction mixture was heated to 80° C. and stirred for an additional 1 h. The orange solution was diluted with ice water and neutralised with 20% sodium acetate solution and extracted with EA (3×10-20 mL). The combined organic extracts were washed with saturated aqueous sodium bicarbonate solution (50 mL), brine (50 mL), and water (3×50 ml), dried over anhydrous Na₂SO₄, filtered and concentrated by rotary evaporation to get the crude compound. Then, the resulting concentrate was purified on a silica column to get 2.14 g (purity=82%) yellow oil compound with a yield of 71%.

TLC R_{f}=0.65 (PE:EA=1:1);

LCMS (m/z)=385.90 (M+H)

### Example 9. Representative Synthesis of Compounds of Formula 8

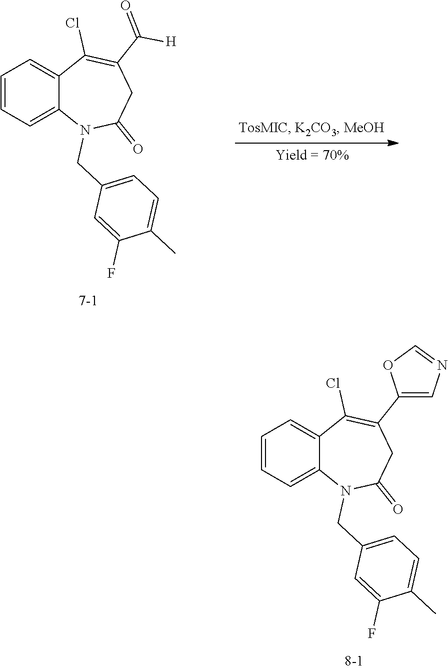 [SMILES: Cc1ccc(CN2C(=O)CC(C=O)=C(Cl)c3ccccc32)cc1F ; Cc1ccc(CN2C(=O)CC(c3cnco3)=C(Cl)c3ccccc32)cc1F]

### 5-chloro-1-(3-fluoro-4-methylbenzyl)-4-(oxazol-5-yl)-1,3-dihydro-2H-benzo[b]azepin-2-one (8-1)

The toluenesulfonylmethyl isocyanide (TosMIC, 234 mg, 0.35 mmol) was placed in a dry round-bottom flask and dry MeOH (9 mL) added under an argon atmosphere. At rt, solid K₂CO₃ (92.2 g, 0.87 mmol) and the 5-chloro-1-(3-fluoro-4-methylbenzyl)-2-oxo-2,3-dihydro-1H-benzo[b]azepine-4-carbaldehyde 7-1 (100 mg, 0.29 mmol) were added to the mixture, and the mixture was heated to reflux for 1.5 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue purified by flash chromatography (silica gel) to give the pure white solid (78 mg, yield=70%, purity=99%)

TLC R_{f}=0.3 (PE:EA=2:1)

LCMS (m/z)=383.60 (M+H)

¹H NMR (400 MHz, DMSO) δ 8.62 (s, 1H), 7.94 (s, 1H), 7.77 (dd, J=8.0, 1.4 Hz, 1H), 7.60-7.52 (m, 1H), 7.52-7.43 (m, 1H), 7.37-7.28 (m, 1H), 7.12 (t, J=8.0 Hz, 1H), 6.78-6.66 (m, 2H), 5.32 (d, J=16.1 Hz, 1H), 4.93 (d, J=16.1 Hz, 1H), 3.75 (d, J=13.2 Hz, 1H), 3.03 (d, J=13.2 Hz, 1H), 2.12 (s, 3H).

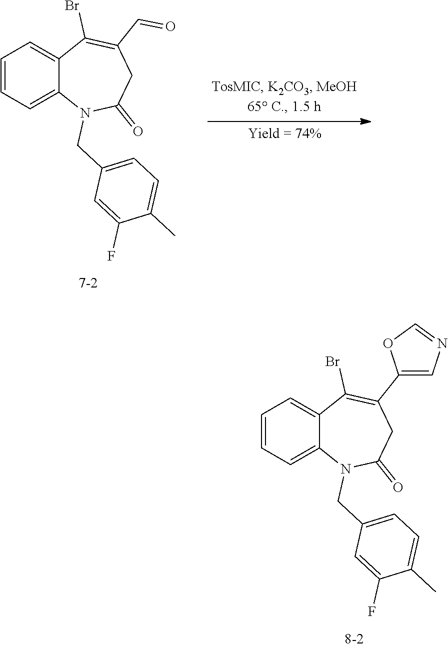 [SMILES: Cc1ccc(CN2C(=O)CC(C=O)=C(Br)c3ccccc32)cc1F ; Cc1ccc(CN2C(=O)CC(c3cnco3)=C(Br)c3ccccc32)cc1F]

### 5-bromo-1-(3-fluoro-4-methylbenzyl)-4-(oxazol-5-yl)-1,3-dihydro-2H-benzo[b]azepin-2-one (8-2)

The toluenesulfonylmethyl isocyanide (TosMIC, 25.35 mg, 0.13 mmol) was placed in a dry round-bottom flask and dry MeOH (3.5 mL) added under an argon atmosphere. At rt, solid K₂CO₃ (35 mg, 0.33 mmol) and the 5-bromo-1-(3-fluoro-4-methylbenzyl)-2-oxo-2,3-dihydro-1-benzo[b]azepine-4-carbaldehyde 7-2 (41 mg, 0.11 mmol) were added to the mixture, and the mixture was heated to reflux for 1.5 h. The mixture was quenched with cold water. After the mixture was quenched, all of the solvent was removed under reduced pressure and the product extracted with ethyl acetate (3×30 mL). The combined organic layers were washed with water, brine and dried by Na₂SO₄. The solvent was then removed under reduced presume and the residue purified by flash chromatography (silica gel) to give the pure yellow solid (purity=99%, 35 mg, yield=74%).

TLC R_{f}=0.6 (EA:PE=1:2)

LCMS (m/z)=429.20 (M+H)

¹H NMR (400 MHz, DMSO) δ 8.63 (s, 1H), 8.01 (s, 1H), 7.76 (dd, J=8.0, 1.5 Hz, 1H), 7.54 (dd, J=8.3, 0.9 Hz, 1H), 7.48-7.40 (m, 1H), 7.34-7.25 (m, 1H), 7.11 (t, J=8.0 Hz, 1H), 6.70 (t, J=8.6 Hz, 2H), 5.36 (d, J=16.1 Hz, 1H), 4.91 (d, J=16.1 Hz, 1H), 3.70 (d, J=13.1 Hz, 1H), 3.01 (d, J=13.1 Hz, 1H), 2.12 (s, 3H). ¹³C NMR (100 MHz, DMSO) δ 168.98, 162.23, 159.82, 152.82, 148.48, 139.71, 137.79, 137.71, 133.64, 132.01, 131.96, 131.11, 130.69, 128.67, 125.92, 125.48, 123.40, 123.25, 123.08, 122.77, 122.73, 116.35, 113.57, 113.34, 49.36, 38.71, 14.22, 14.18.

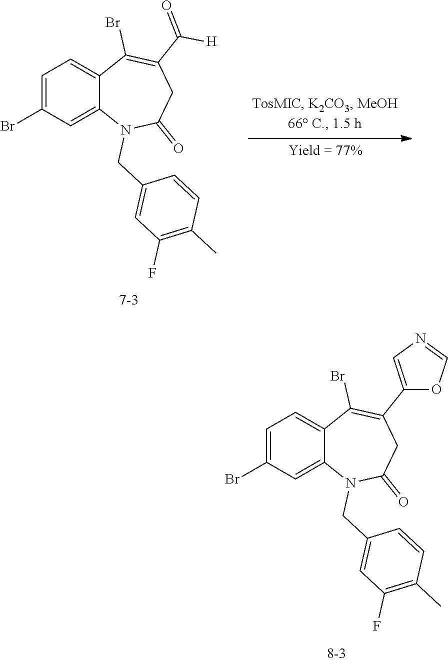 [SMILES: Cc1ccc(CN2C(=O)CC(C=O)=C(Br)c3ccc(Br)cc32)cc1F ; Cc1ccc(CN2C(=O)CC(c3cnco3)=C(Br)c3ccc(Br)cc32)cc1F]

### 5,8-dibromo-1-(3-fluoro-4-methylbenzyl)-4-(oxazol-5-yl)-1,3-dihydro-2H-benzo[b]azepin-2-one (8-3)

The toluenesulfonylmethyl isocyanide (TosMIC, 251 mg, 1.28 mmol) was placed in a dry two-neck round-bottom flask and dry MeOH (40 mL) added under an argon atmosphere. At it, solid K₂CO₃ (443 mg, 3.21 mmol) and the 5,8-dibromo-1-(3-fluoro-4-methylbenzyl)-2-oxo-2,3-dihydro-1H-benzo[b]azepine-4-carbaldehyde 7-3 (500 mg, 1.07 mmol) were added to the mixture, and the mixture was heated to reflux for 1.5 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue was purified by flash chromatography (silica gel) to give the pure yellow solid (purity=95%, 420 mg, yield=77%).

TLC R_{f}=0.6 (EA:PE=1:2)

LCMS (m/z)=506.90 (M+H)

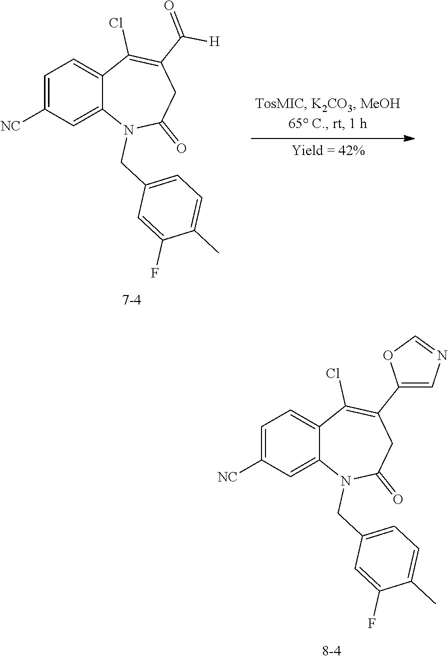 [SMILES: Cc1ccc(CN2C(=O)CC(C=O)=C(Cl)c3ccc(C#N)cc32)cc1F ; Cc1ccc(CN2C(=O)CC(c3cnco3)=C(Cl)c3ccc(C#N)cc32)cc1F]

### 5-chloro-1-(3-fluoro-4-methylbenzyl)-4-(oxazol-5-yl)-2-oxo-2,3-dihydro-1H-benzo[b]azepine-8-carbonitrile (8-4)

The toluenesulfonylmethyl isocyanide (TosMIC, 89 mg, 0.456 mmol, 1.2 equiv) was placed in a dry two-neck round-bottom flask and dry MeOH (11.8 mL) added under an nitrogen atmosphere. At rt, solid K₂CO₃ (121 mg, 1.14 mmol, 3 equiv) and the 5-chloro-1-(3-fluoro-4-methylbenzyl)-4-formyl-2-oxo-2,3-dihydro-1H-benzo[b]azepine-8-carbonitrile (7-4) (140 mg, 0.38 mmol, 1 equiv) were added to the mixture, and the mixture was heated to reflux for 1 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue purified by flash chromatography (silica gel) to give the pure yellow solid (purity=95%, 65 mg, yield=42%).

TLC R_{f}=0.25 (PE:EA=2:1)

LCMS (m/z)=408.50 (M+H)

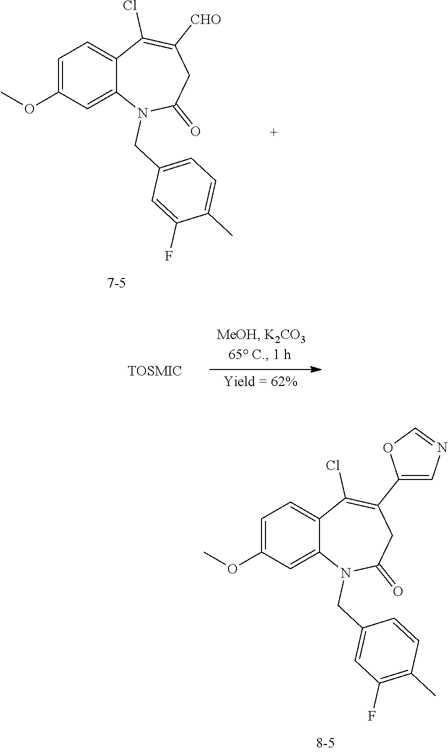

### 5-chloro-1-(3-fluoro-4-methylbenzyl)-8-methoxy-4-(oxazol-5-yl)-1,3-dihydro-2H-benzo[b]azepin-2-one (8-5)

The toluenesulfonylmethyl isocyanide (215 mg, 1.10 mmol, 1.2 equiv) was placed in a dry round-bottom flask and dry MeOH (30 mL) added under an argon atmosphere. At rt, solid K₂CO₃ (292 mg, 2.75 mmol, 3 equiv) and the 5-chloro-1-(3-fluoro-4-methylbenzyl)-8-methoxy-2-oxo-2,3-dihydro-1H-benzo[b]azepine-4-carbaldehyde (7-5) (343 mg, 0.918 mmol, 1 equiv) were added to the mixture, and the mixture was heated to reflux for 1.5 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue purified by flash chromatography (silica gel) to give the pure white solid (purity=99%, 235 mg, yield=62%)

TLC R_{f}=0.3 (EA:PE=1:2)

LCMS (m/z)=413.60 (M+H)

¹H NMR (400 MHz, DMSO) δ 8.59 (s, 1H), 7.88 (s, 1H), 7.68 (d, J=8.9 Hz, 1H), 7.11 (t, J=7.9 Hz, 1H), 7.06 (d, J=2.5 Hz, 1H), 6.93 (dd, J=8.9, 2.5 Hz, 1H), 6.72 (t, J=8.9 Hz, 2H), 5.32 (d, J=16.1 Hz, 1H), 4.97 (d, J=16.1 Hz, 1H), 3.79 (s, 3H), 3.73 (d, J=13.1 Hz, 1H), 3.02 (d, J=13.1 Hz, 1H), 2.12 (s, 3H).

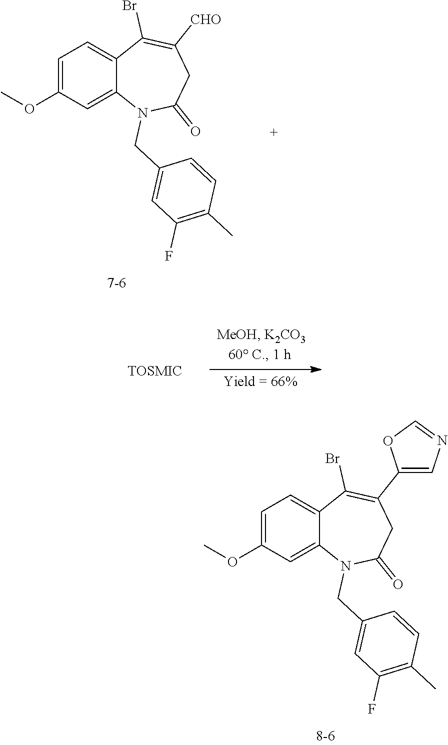

### 5-bromo-1-(3-fluoro-4-methylbenzyl)-8-methoxy-4-(oxazol-5-yl)-1,3-dihydro-2H-benzo[b]azepin-2-one (8-6)

The toluenesulfonylmethyl isocyanide (TosMIC, 139 mg, 0.71 mmol, 1.2 equiv) was placed in a dry two-neck round-bottom flask and dry MeOH (19 mL) added under an nitrogen atmosphere. At rt, solid K₂CO₃ (189 mg, 1.78 mmol, 3 equiv) and 7-6 (248 mg, 0.593 mmol, 1 equiv) were added to the mixture, and the mixture was heated to reflux for 3 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue purified by flash chromatography (silica gel) to to get 180 mg (purity=97%) light yellow solid compound with a yield of 66%.

TLC R_{f}=0.3 (EA:PE=1:2)

LCMS (m/z)=457.40 (M+H)

¹H NMR (400 MHz, DMSO) δ 8.60 (s, 1H), 7.94 (s, 1H), 7.68 (d, J=8.9 Hz, 1H), 7.12 (t, J=8.0 Hz, 1H), 7.05 (d, J=2.5 Hz, 1H), 6.92 (dd, J=9.0, 2.5 Hz, 1H), 6.71 (dd, J=12.6, 9.4 Hz, 2H), 5.36 (d, J=16.1 Hz, 1H), 4.94 (d, J=16.1 Hz, 1H), 3.79 (s, 3H), 3.67 (d, J=13.0 Hz, 1H), 3.01 (d, J=13.0 Hz, 1H), 2.13 (s, 3H).

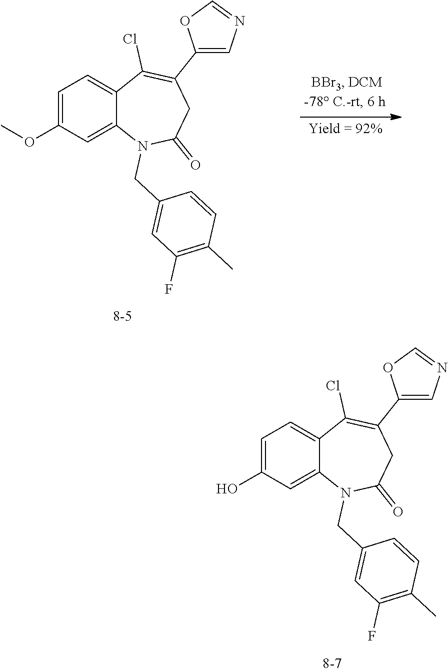 [SMILES: COc1ccc2c(c1)N(Cc1ccc(C)c(F)c1)C(=O)CC(c1cnco1)=C2Cl ; Cc1ccc(CN2C(=O)CC(c3cnco3)=C(Cl)c3ccc(O)cc32)cc1F]

### 5-chloro-1-(3-fluoro-4-methylbenzyl)-8-hydroxy-4-(oxazol-5-yl)-1,3-dihydro-2H-benzo[b]azepin-2-one(8-7)

To a stirred solution of 5-chloro-1-(3-fluoro-4-methylbenzyl)-8-methoxy-4-(oxazol-5-yl)-1,3-dihydro-2H-benzo[b]azepin-2-one (8-5) (203 mg, 0.492 mmol, 1 equiv), in dry DCM (3 mL), was cooled to −78° C. then BBr₃ (240 mg, 2.5 mmol, 5 equiv) was added. The resulting mixture was slowly warmed to ambient temperature and then stirred for 6 h. The solution was quenched with saturated NaHCO₃ solution and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 180 mg (purity=96%) light yellow solid compound with a yield of 92%.

TLC R_{f}=0.15 (EA:PE=1:2)

LCMS (m/z)=399.60 (M+H)

¹H NMR (400 MHz, DMSO) δ 10.24 (s, 1H), 8.57 (s, 1H), 7.84 (s, 1H), 7.60 (d, J=8.7 Hz, 1H), 7.16 (t, J=7.8 Hz, 1H), 7.06-6.54 (m, 4H), 5.15 (d, J=16.2 Hz, 1H), 4.90 (d, J=16.3 Hz, 1H), 3.70 (d, J=13.1 Hz, 1H), 3.05 (d, J=13.0 Hz, 1H), 2.15 (s, 3H).

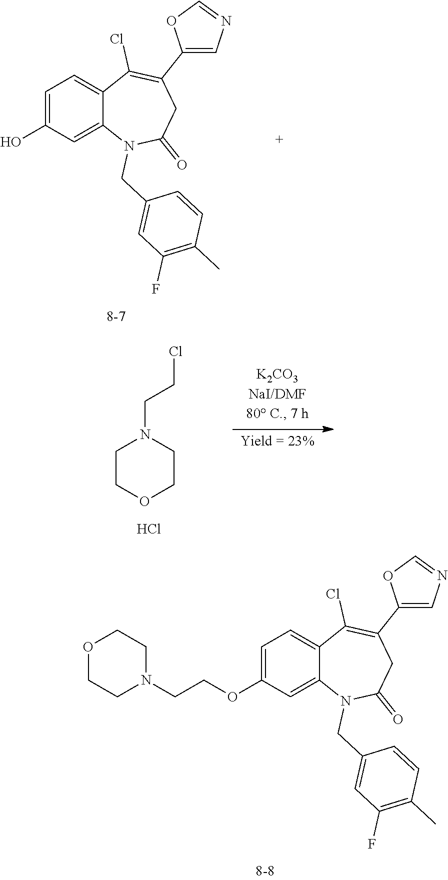 [SMILES: Cc1ccc(CN2C(=O)CC(c3cnco3)=C(Cl)c3ccc(O)cc32)cc1F ; ClCCN1CCOCC1 ; Cl ; Cc1ccc(CN2C(=O)CC(c3cnco3)=C(Cl)c3ccc(OCCN4CCOCC4)cc32)cc1F]

### chloro-1-(3-fluoro-4-methylbenzyl)-8-(2-morpholinoethoxy)-4-(oxazol-5-yl)-1,3-dihydro-2H-benzo[b]azepin-2-one(8-8)

To a solution of 5-chloro-1-(3-fluoro-4-methylbenzyl)-8-hydroxy-4-(oxazol-5-yl)-1,3-dihydro-2H-benzo[b]azepin-2-one 8-7 (50 mg, 0.125 mmol, 1 equiv) in 1 mL DMF are added (86 mg, 0.625 mmol, 5 equiv) K₂CO₃ and 4-(2-Chloroethyl)morpholine hydrochloride (46 mg, 0.25 mmol, 2 equiv) and NaH (19 mg, 0.125 mmol, 1 equiv). The mixture is stirred for 7 hi at 80° C. Then, the mixture is poured into 150 mL water and extracted with 3×200 mL dichloromethane. The organic layer is washed with 2×150 mL NaOH (20%) dried over Na₂SO₄ and evaporated in vacuum and purified by silica gel chromatography to get 15 mg (purity=94%) with a yield of 23%

TLC R_{f}=0.5 (DCM:MeOH=20:1)

LCMS (m/z)=512.30 (M+H)

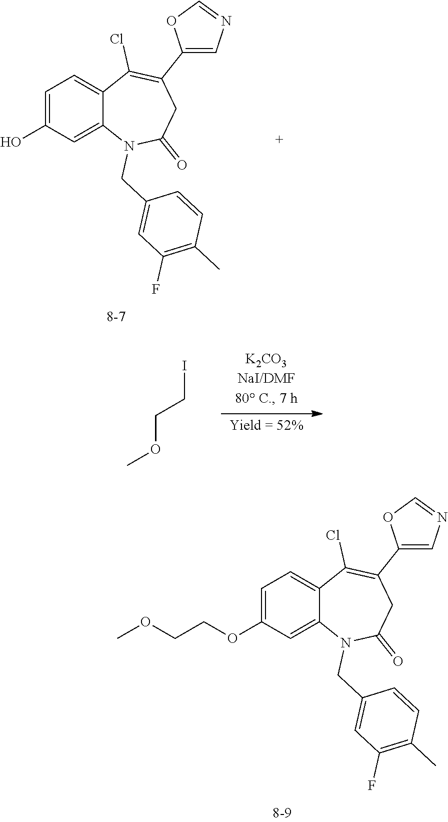

### chloro-1-(3-fluoro-4-methylbenzyl)-8-(2-methoxyethoxy)-4-(oxazol-5-yl)-1,3-dihydro-2H-benzo[b]azepin-2-one (8-9)

To a solution of 5-chloro-1-(3-fluoro-4-methylbenzyl)-8-hydroxy-4-(oxazol-5-yl)-1,3-dihydro-2H-benzo[b]azepin-2-one 8-7 (79 mg, 0.2 mmol, 1 equiv) in 1.5 mL DMF are added (110 mg, 0.8 mmol, 4 equiv) K₂CO₃ and 1-iodo-2-methoxyethane (31 μL, 0.3 mmol, 1.5 equiv). The mixture is stirred for 7 h at 80° C. Then, the mixture is poured into 150 mL water and extracted with 3×200 mL dichloromethane. The organic layer is washed with 2×150 mL NaOH (20%) dried over MgSO4 and evaporated in vacuum and purified by silica gel chromatography to get 48 mg (purity=93%) with a yield of 52%.

TLC R_{f}=0.6 (DCM:MeOH=20:1)

LCMS (m/z)=457.70 (M+H)

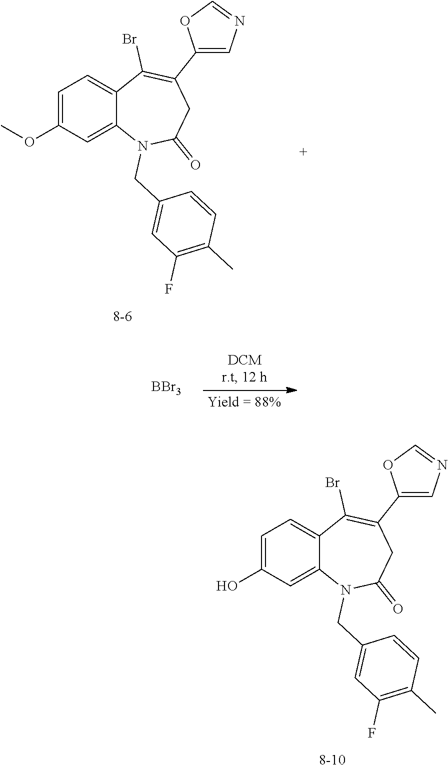 [SMILES: COc1ccc2c(c1)N(Cc1ccc(C)c(F)c1)C(=O)CC(c1cnco1)=C2Br ; "BBr3" ; Cc1ccc(CN2C(=O)CC(c3cnco3)=C(Br)c3ccc(O)cc32)cc1F]

### 5-bromo-1-(3-fluoro-4-methylbenzyl)-8-hydroxy-4-(oxazol-5-yl)-1,3-dihydro-2H-benzo[b]azepin-2-one (8-10)

To a stirred solution of 5-6 (135 mg, 0.295 mmol, 1 equiv) in DCM (2 mL), was added BBr₃ (0.141 mL, 1.48 mmol, 1.2 equiv) at −78° C. The resulting mixture was slowly warmed to ambient temperature and then stirred for 12 h. The solution was quenched with saturated ammonium chloride and extracted with EA (3×20 mL). The organic solutions were combined, washed with H₂O (2×20 mL) and brine (20 mL), dried over anhydrous Na₂SO₄, filtered and concentrated by rotary evaporation to get the crude compound. Then, the resulting concentrate was purified on a silica column to get 116 mg (purity=99%) light yellow solid compound with a yield of 88%.

TLC R_{f}=0.2 (EA:PE=1:2)

LCMS (m/z)=443.40 (M+H)

¹H NMR (400 MHz, DMSO) δ 10.24 (s, 1H), 8.58 (s, 1H), 7.92 (s, 1H), 7.61 (d, J=8.8 Hz, 1H), 7.16 (t, J=7.9 Hz, 1H), 6.87-6.66 (m, 4H), 5.20 (d, J=16.3 Hz, 1H), 4.87 (d, J=16.3 Hz, 1H), 3.65 (d, J=13.0 Hz, 1H), 3.04 (d, J=13.0 Hz, 1H), 2.15 (s, 3H).

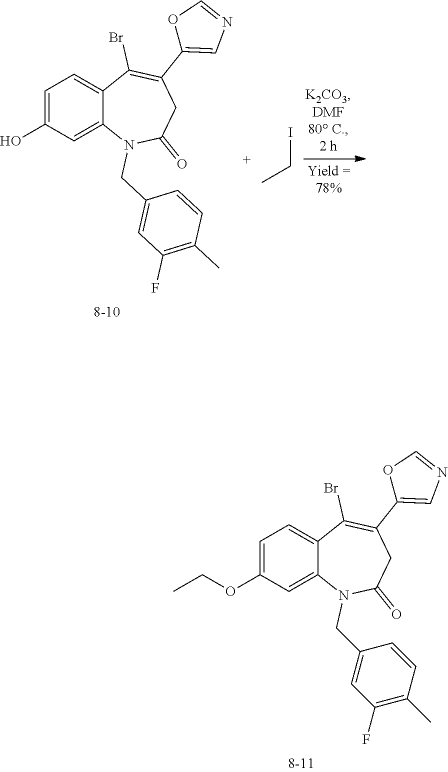

### 5-bromo-8-ethoxy-1-(3-fluoro-4-methylbenzyl)-4-(oxazol-5-yl)-1,3-dihydro-2H-benzo[b]azepin-2-one (8-11)

To a solution of 5-chloro-1-(3-fluoro-4-methylbenzyl)-8-hydroxy-4-(oxazol-5-yl)-1,3-dihydro-2H-benzo[b]azepin-2-one (8-10) (116 mg, 0.262 mmol, 1 equiv) in 2 mL DMF were added K₂CO₃ (145 mg, 1.05 mmol, 4 equiv) and 1-iodo-2-methoxyethane (31 μL, 0.393 mmol, 1.5 equiv). The mixture was stirred for 2 h at 80° C. Then, the mixture was poured into 150 mL water and extracted with 3×200 mL dichloromethane. The organic solutions were combined and washed with saturated NaCl solution, dried over MgSO4, filtered and concentrated by rotary evaporation. Then, the resulting concentrate was purified by silica gel chromatography to get 96 mg (purity=99%) with a yield of 78%.

TLC R_{f}=0.4 (EA:PE=1:2)

LCMS (m/z)=471.60 (M+H)

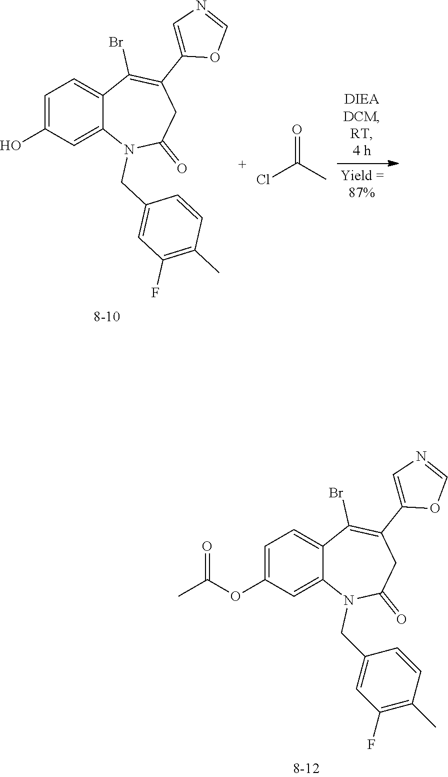

### 5-bromo-1-(3-fluoro-4-methylbenzyl)-4-(oxazol-5-yl)-2-oxo-2,3-dihydro-1H-benzo[b]azepin-8-yl acetate (8-12)

To a solution of 8-10 (100 mg, 0.226 mmol, 1 equiv) in DCM (2 mL), was added DIEA (39 uL, 0.0391 mmol, 1.3 equiv) under Nitrogen. To this solution was added acetyl chloride (19 uL, 0.0268 mmol, 1.2 equiv), and the reaction mixture was allowed to stir at rt for 4 h. To the reaction mixture was then added H2O (4 mL), the organic solution was separated and the aqueous solution was extracted with DCM (3×50 mL). The organic solutions were combined, washed with H2O (2×50 mL) and brine (1×100 mL), dried over anhydrous Na₂SO₄, filtered and concentrated by rotary evaporation to get the crude compound. Then, the resulting concentrate was purified on a silica column to get 95 mg (purity=99%) of the light yellow oil compound with a yield of 87%.

TLC R_{f}=0.6 (EA:PE=1:1)

LCMS (m/z)=485.30 (M+H)

¹H NMR (400 MHz, DMSO) δ 8.63 (s, 1H), 8.00 (s, 1H), 7.80 (d, J=8.8 Hz, 1H), 7.42 (d, J=2.0 Hz, 1H), 7.19-7.07 (m, 2H), 6.67 (t, J=9.4 Hz, 2H), 5.41 (d, J=16.1 Hz, 1H), 4.83 (d, J=16.1 Hz, 1H), 3.71 (d, J=13.2 Hz, 1H), 3.05 (d, J=13.2 Hz, 1H), 2.29 (s, 3H), 2.12 (s, 3H).

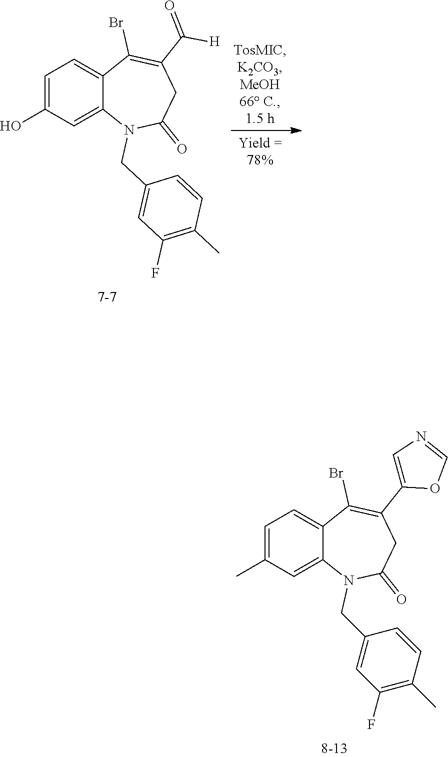 [SMILES: Cc1ccc(CN2C(=O)CC(C=O)=C(Br)c3ccc(O)cc32)cc1F ; Cc1ccc2c(c1)N(Cc1ccc(C)c(F)c1)C(=O)CC(c1cnco1)=C2Br]

### 5-bromo-1-(3-fluoro-4-methylbenzyl)-8-methyl-4-(oxazol-5-yl)-1,3-dihydro-2H-benzo[b]azepin-2-one (8-13)

The toluenesulfonylmethyl isocyanide (TosMIC, 58.5 mg, 0.3 mmol) was placed in a dry two-neck round-bottom flask and dry MeOH (8 mL) added under an argon atmosphere. At rt, solid K₂CO₃ (103.5 mg, 0.75 mmol) and the 5-bromo-1-(3-fluoro-4-methylbenzyl)-8-methyl-2-oxo-2,3-dihydro-1H-benzo[b]azepine-4-carbaldehyde 7-7 (100 mg, 0.25 mmol) were added to the mixture, and the mixture was heated to reflux for 1.5 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried by (Na₂SO₄). The solvent was then removed under reduced pressure and the residue was purified by flash chromatography (silica gel) to give the pure yellow solid (purity=95%, 83 mg, yield=75%).

TLC R_{f}=0.5 (EA:PE=1:2)

LCMS (m/z)=441.40 (M+H)

### Example 10. Representative Synthesis of Compounds of Formula 10

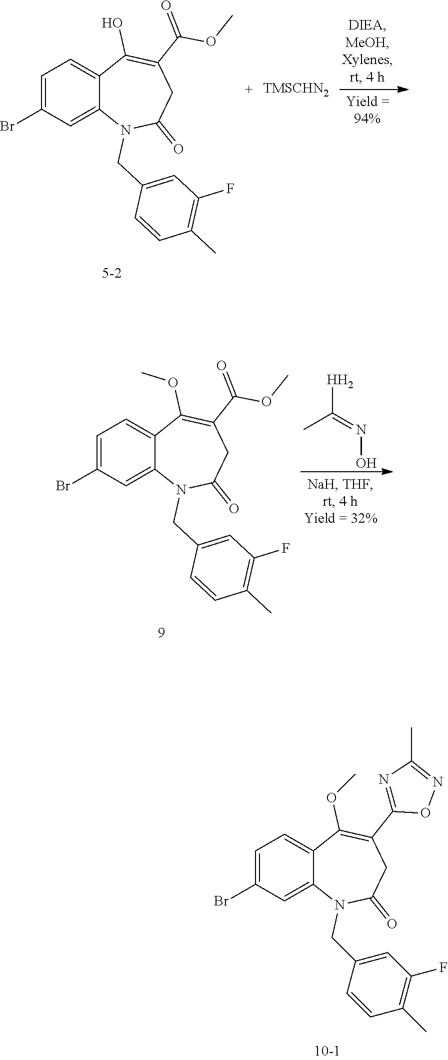

### 8-bromo-1-(3-fluoro-4-methylbenzyl)-5-methoxy-4-(3-methyl-1,2,4-oxadiazol-5-yl)-1,3-dihydro-2H-benzo[b]azepin-2-one (10-1)

To a stirred solution of methyl 8-bromo-1-(3-fluoro-4-methylbenzyl)-5-hydroxy-2-oxo-2,3-dihydro-1H-benzo[b]azepine-4-carboxylate 5-2 (868 mg, 2 mmol, 1 equiv) and DIEA (496 μL, 3 mmol, 1.5 equiv) in MeOH (6 mL) and Xylenes (6 mL), was added TMSCHN₂ (3 mL, 6 mmol, 3 equiv) at ambient temperature. The reaction mixture was then stirred at room temperature for 4 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 850 mg (purity=94%) white solid compound with a yield of 94%.

TLC R_{f}=0.6 (EA:PE=1:2)

LCMS (m/z)=417.90 (M-29)

¹H NMR (400 MHz, DMSO) δ 7.90 (s, 1H), 7.47 (s, 2H), 7.14 (t, J=7.9 Hz, 1H), 6.92-6.65 (m, 2H), 5.47 (d, J=15.9 Hz, 1H), 4.88 (d, J=15.8 Hz, 1H), 3.77 (s, 3H), 3.43 (d, J=13.5 Hz, 1H), 3.38 (s, 3H), 2.64 (d, J=13.5 Hz, 1H), 2.11 (s, 3H). ¹³C NMR (100 MHz, DMSO) δ 170.78, 165.32, 162.17, 159.76, 158.95, 142.61, 137.58, 137.51, 132.09, 132.03, 129.88, 128.69, 128.05, 127.16, 124.58, 123.53, 123.36, 123.33, 114.05, 113.83, 112.42, 60.55, 52.52, 48.70, 34.28, 14.20, 14.17.

To the resulting solution of 9 (methyl 8-bromo-1-(3-fluoro-4-methylbenzyl)-5-methoxy-2-oxo-2,3-dihydro-1H-benzo[b]azepine-4-carboxylate) (89 mg, 0.2 mmol, 1 equiv) and (E)-N′-hydroxyacetimidamide (36 mg, 0.48 mmol, 2.4 equiv) in THF (1.5 mL), was added NaH 60% dispersion in mineral oil (21 mg, 0.52 mmol, 2.6 equiv) at ambient temperature under N₂ atmosphere. The reaction mixture was then stirred at room temperature r.t for 4 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 30 mg (purity=99%) white solid compound with a yield of 32%.

TLC R_{f}=0.3 (PE:EA=4:1)

LCMS (m/z)=472.40 (M+H)

### Example 11. Representative Synthesis of Compounds of Formula 13

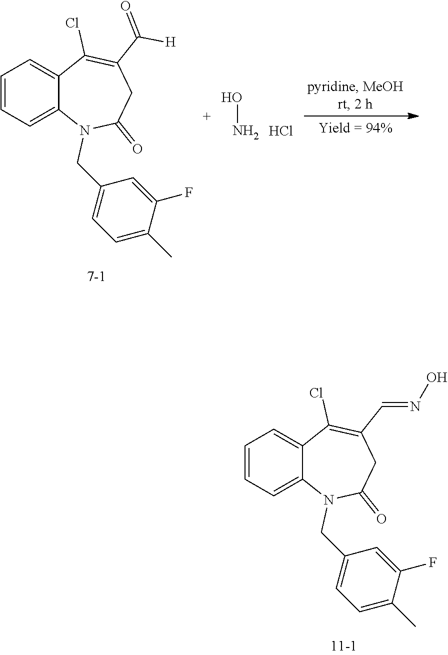

### (E)-5-chloro-1-(3-fluoro-4-methylbenzyl)-2-oxo-2,3-dihydro-1H-benzo[b]azepine-4-carbaldehyde oxime (11-1)

A solution of 5-chloro-1-(3-fluoro-4-methylbenzyl)-2-oxo-2,3-dihydro-1H-benzo[b]azepine-4-carbaldehyde 7-1 (343 mg, 1 mmol, 1 equiv) in methanol (25 mL) was treated with hydroxylamine hydrochloride (90 mg, 1.3 mmol, 1.3 equiv) and pyridine (0.1 mL, 1.2 mmol, 1.2 equiv). The reaction was stirred at room temperature for 2 hours and then concentrated. The residue was purified by silica gel chromatography to get 340 mg (purity=98%) white solid compound with a yield of 94%.

TLC R_{f}=0.15 (DCM:MeOH=10:1)

LCMS (m/z)=359.30 (M+H)

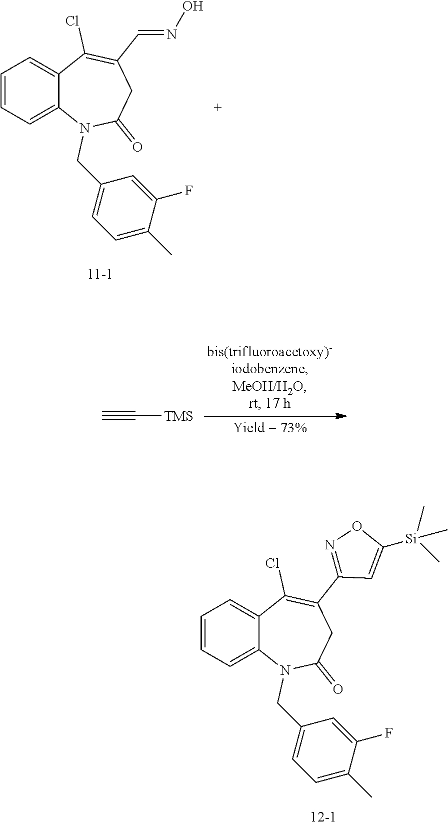

### 5-chloro-1-(3-fluoro-4-methylbenzyl)-4-(5-(trimethylsilyl)isoxazol-3-yl)-1,3-dihydro-2H-benzo[b]azepin-2-one (12-1)

To a solution of (E)-5-chloro-1-(3-fluoro-4-methylbenzyl)-2-oxo-2,3-dihydro-1H-benzo[b]azepine-4-carbaldehyde oxime 11-1 (179 mg, 0.11 mmol, 1 equiv) and trimethylsilyl acetylene (133 μL, 1 mmol, 2 equiv) in methanol (6.8 mL) and water (1.4 mL) was added bis(trifluoroacetoxy)-iodobenzene (237 mg, 0.55 mmol, 1.1 equiv) and the reaction was stirred overnight. The reaction mixture was poured into MTBE, and washed with water and satd. aq. NaHCO₃. The organic layer was concentrated, and the residue was purified by silica gel chromatography to get 166 mg (purity=95%) white solid compound with a yield of 73%.

TLC R_{f}=0.4 (DCM)

LCMS (m/z)=455.60 (M+H)

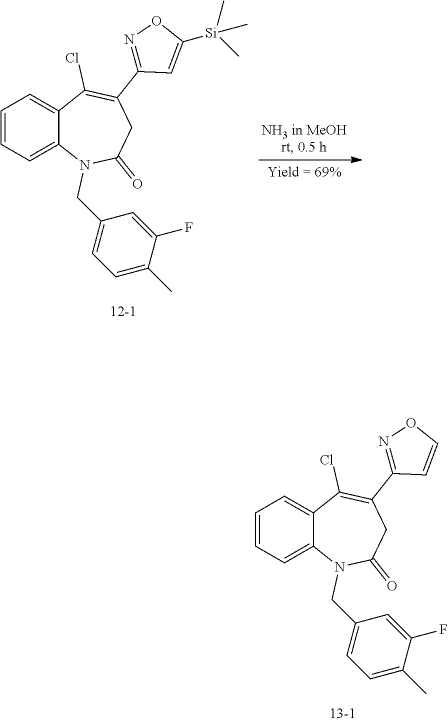 [SMILES: Cc1ccc(CN2C(=O)CC(c3cc([Si](C)(C)C)on3)=C(Cl)c3ccccc32)cc1F ; Cc1ccc(CN2C(=O)CC(c3ccon3)=C(Cl)c3ccccc32)cc1F]

### 5-chloro-1-(3-fluoro-4-methylbenzyl)-4-(isoxazol-3-yl)-1,3-dihydro-2H-benzo[b]azepin-2-one (13-1)

A solution of 5-chloro-1-(3-fluoro-4-methylbenzyl)-4-(5-(trimethylsilyl)isoxazol-3-yl)-1,3-dihydro-2H-benzo[b]azepin-2-one (166 mg, 0.36 mmol) in 7M ammonia in methanol (100 mL, 306 mmol) was stirred at room temperature for 0.5 hours. The reaction was concentrated under vacuum and purified by silica gel chromatography to get 95 mg (purity=99%) oil with a yield of 69%.

TLC R_{f}=0.5 (PE:EA=2:1)

LCMS (m/z)=383.60 (M+H)

¹H NMR (400 MHz, DMSO) δ 9.13 (d, J=1.7 Hz, 1H), 7.79 (dd, J=8.0, 1.2 Hz, 1H), 7.58 (d, J=7.8 Hz, 1H), 7.55-7.48 (m, 1H), 7.42-7.29 (m, 1H), 7.18 (d, J=1.7 Hz, 1H), 7.14 (t, J=8.0 Hz, 1H), 6.75 (dd, J=8.9, 4.6 Hz, 2H), 5.35 (d, J=16.1 Hz, 1H), 4.94 (d, J=16.1 Hz, 1H), 3.81 (d, J=13.0 Hz, 1H), 3.09 (d, J=13.0 Hz, 1H), 2.13 (s, 3H).

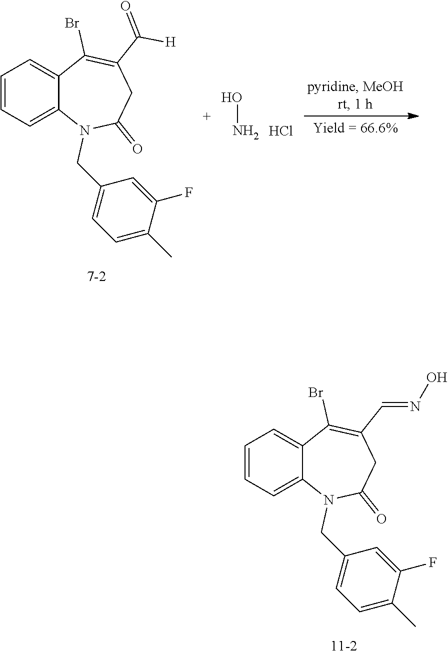

### (E)-5-bromo-1-(3-fluoro-4-methylbenzyl)-2-oxo-2,3-dihydro-1H-benzo[b]azepine-4-carbaldehyde oxime (11-2)

A solution of 5-bromo-1-(3-fluoro-4-methylbenzyl)-2-oxo-2,3-dihydro-1-benzo[b]azepine-4-carbaldehyde 7-2 (194.12 ng, 0.5 mmol, 1 equiv) in methanol (12.5 mL) was treated with hydroxylamine hydrochloride (45.16 mg, 0.65 mmol, 1.3 equiv) and pyridine (49.6 uL, 0.6 mmol, 1.2 equiv). The reaction was stirred at room temperature for 1 hours and then concentrated. The residue was purified by silica gel chromatography to get 134.4 mg (purity=99%) white solid compound with a yield of 66.6%.

TLC R_{f}=0.5 (PE:EA=4:1)

LCMS (m/z)=403.30 (M+H)

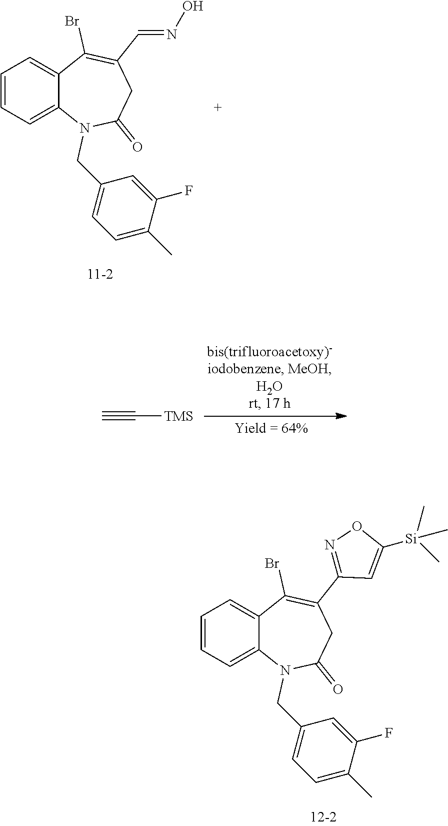

### bromo-1-(3-fluoro-4-methylbenzyl)-4-(5-(trimethylsilyl)isoxazol-3-yl)-1,3-dihydro-2H-benzo[b]azepin-2-one(12-2)

To a solution of (E)-5-bromo-1-(3-fluoro-4-methylbenzyl)-2-oxo-2,3-dihydro-1H-benzo[b]azepine-4-carbaldehyde oxime 11-2 (134.4 mg, 0.33 mmol, 1 equiv) and trimethylsilyl acetylene (94 μL, 0.67 mmol, 2 equiv) in methanol (20.4 mL) and water (4.2 mL) was added bis(trifluoroacetoxy)-iodobenzene (157.66 mg, 0.37 mmol, 1.1 equiv) and the reaction was stirred overnight. The reaction mixture was poured into MTBE, and washed with water and satd. aq. NaHCO₃. The organic layer was concentrated, and the residue was purified by silica gel chromatography to get 105.5 mg (purity=99%) white solid compound with a yield of 64%.

TLC R_{f}=0.5 (PE:EA=4:1)

LCMS (m/z)=499.10 (M+H)

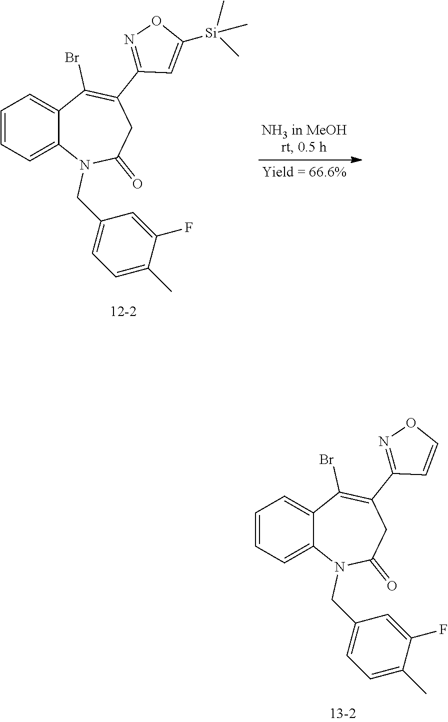 [SMILES: Cc1ccc(CN2C(=O)CC(c3cc([Si](C)(C)C)on3)=C(Br)c3ccccc32)cc1F ; Cc1ccc(CN2C(=O)CC(c3ccon3)=C(Br)c3ccccc32)cc1F]

### bromo-1-(3-fluoro-4-methylbenzyl)-4-(isoxazol-3-yl)-1,3-dihydro-2H-benzo[b]azepin-2-one (13-2)

A solution of 5-bromo-1-(3-fluoro-4-methylbenzyl)-4-(5-(trimethylsilyl)isoxazol-3-yl)-1,3-dihydro-2H-benzo[b]azepin-2-one 12-2 (105.5 mg, 0.21 mmol) in 7M ammonia in methanol (58.3 mL 178 mmol) was stirred at room temperature for 0.5 hours. The reaction was concentrated under vacuum and purified by silica gel chromatography to get 95 mg (purity=99%) yellow oil with a yield of 66.6%.

TLC R_{f}=0.6 (PE:EA=2:1)

LCMS (m/z)=426.90 (M+H)

¹H NMR (400 MHz, DMSO) δ 9.12 (d, J=1.6 Hz, 1H), 7.78 (dd, J=8.0, 1.1 Hz, 1H), 7.57 (d, J=8.1 Hz, 1H), 7.53-7.44 (m, 1H), 7.33 (t, J=7.6 Hz, 1H), 7.14 (t, J=8.0 Hz, 1H), 7.10 (d, J=1.6 Hz, 1H), 6.75 (t, J=8.4 Hz, 2H), 5.41 (d, J=16.0 Hz, 1H), 4.91 (d, J=16.1 Hz, 1H), 3.64 (d, J=12.9 Hz, 1H), 3.11 (d, J=12.9 Hz, 1H), 2.13 (s, 3H).

### Example 12. Representative Synthesis of Compounds of Formula 16

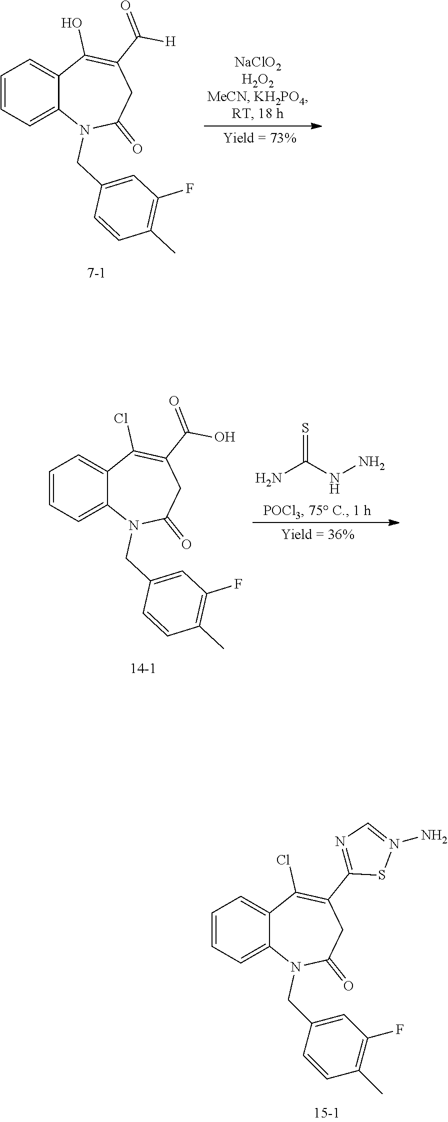

### 4-(5-amino-1,3,4-thiadiazol-2-yl)-5-chloro-1-(3-fluoro-4-methylbenzyl)-1,3-dihydro-2H-benzo[b]azepin-2-one(15-1)

Reactions were carried out by addition of aqueous NaClO₂ (461 mg, 4.61 mmol, 1.76 equiv) to a solution of 5-chloro-1-(3-fluoro-4-methylbenzyl)-2-oxo-2,3-dihydro-1H-benzo[b]azepine-4-carbaldehyde 7-1 (1 g, 2.92 mmol, 1 equiv) and (414 mg, 2.67 mmol, 1.02 equiv) of 30% H₂O₂ in aqueous acetonitrile, at 0° C., buffered with KH₂PO₄ at pH 4.3. The reaction mixture was stirred overnight. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 770 mg (purity=97%) white solid compound with a yield of 73%.

TLC R_{f}=0.2 (DCM:MeOH=10:1)

LCMS (m/z)=360.10 (M+H)

A stirring mixture of 5-chloro-1-(3-fluoro-4-methylbenzyl)-2-oxo-2,3-dihydro-1H-benzo[b]azepine-4-carboxylic acid 14-1 (71 mg, 0.2 mmol, 1 equiv), N-aminothiourea (37 mg, 0.4 mmol, 2 equiv) and POCl₃ (0.5 mL) was heated at 75° C. for 0.5 h. After cooling to room temperature, water (30 ml) was added. The reaction mixture was refluxed for 4 h. After cooling, the mixture was basified to PH=8 by drop wise addition of 50% NaOH solution under stirring, and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 30 mg (purity=96%) light yellow solid compound with a yield of 36%.

TLC R_{f}=0.5 (DCM:MeOH=10:1)

LCMS (m/z)=415.70 (M+H)

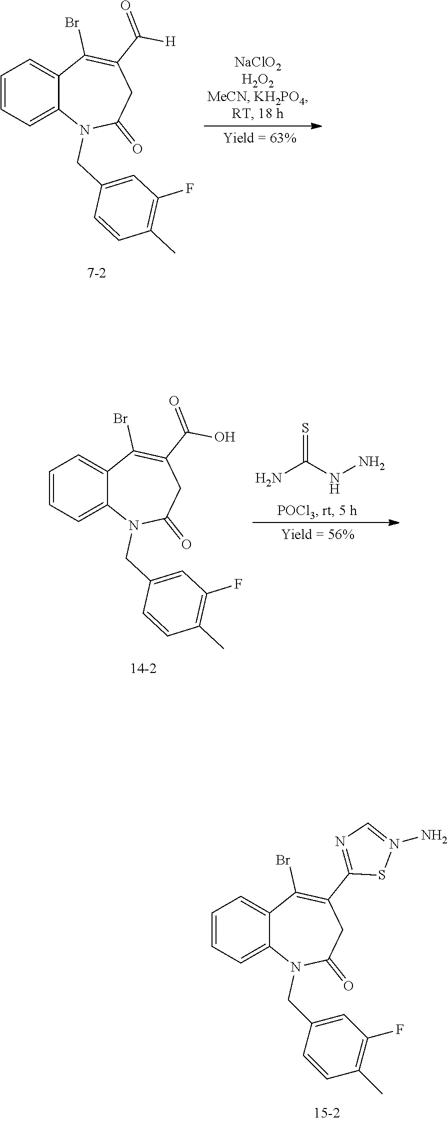

### 4-(5-amino-1,3,4-thiadiazol-2-yl)-5-bromo-1-(3-fluoro-4-methylbenzyl)-1,3-dihydro-2H-benzo[b]azepin-2-one (15-2)

Reactions were carried out by addition of aqueous NaClO₂ (324.7 mg, 3.61 mmol, 3.6 equiv) to a solution of 5-bromo-1-(3-fluoro-4-methylbenzyl)-2-oxo-2,3-dihydro-1H-benzo[b]azepine-4-carbaldehyde 7-2 (795 mg, 2.05 mmol, 1 equiv) and (237 mg, 2.09 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 528 mg (purity=96%) white solid compound with a yield of 63%.

TLC R_{f}=0.2 (DCM:MeOH=10:1)

LCMS (m/z)=405.90 (M+H)

¹H NMR (400 MHz, DMSO) δ 13.61 (s, 1H), 7.72 (d, J=8.0 Hz, 1H), 7.51 (d, J=8.1 Hz, 1H), 7.44 (t, J=7.7 Hz, 1H), 7.29 (t, J=7.5 Hz, 1H), 7.12 (t, J=8.0 Hz, 1H), 6.75 (dd, J=8.0, 6.1 Hz, 2H), 5.38 (d, J=16.1 Hz, 1H), 4.89 (d, J=16.1 Hz, 1H), 3.41 (d, J=13.2 Hz, 1H), 2.85 (d, J=13.2 Hz, 1H), 2.13 (s, 3H). ¹³C NMR (100 MHz, DMSO) δ 169.48, 167.13, 162.28, 159.86, 139.79, 137.82, 137.75, 132.75, 132.30, 131.98, 131.93, 131.11, 130.88, 125.90, 123.53, 123.23, 123.06, 122.83, 122.80, 120.43, 113.64, 113.42, 49.45, 38.42, 14.23, 14.20.

A stirring mixture of 5-bromo-1-(3-fluoro-4-methylbenzyl)-2-oxo-2,3-dihydro-1H-benzo[b]azepine-4-carboxylic acid 14-2 (194 mg, 0.5 mmol, 1 equiv), N-aminothiourea (91 mg, 1 mmol, 2 equiv) and POCl₃ (1.2 mL) was stirred at rt for 5 h. Then, water (30 ml) was added, the mixture was basified to pH=8 by drop wise addition of 50% NaOH solution under stirring, and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 30 mg (purity=91%) light yellow solid compound with a yield of 56%.

TLC R_{f}=0.5 (DCM:MeOH=10:1)

LCMS (m/z)=458.90 (M+H)

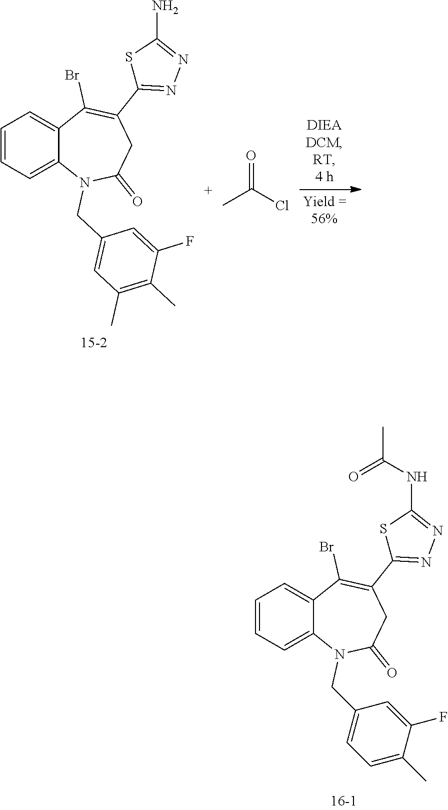

### N-(5-(5-bromo-1-(3-fluoro-4-methylbenzyl)-2-oxo-2,3-dihydro-1Hbenzo[b]azepin-4-yl)-1,3,4-thiadiazol-2-yl)acetamide (16-1)

To the solution of 15-2 (80 mg, 0.17 mmol, 1 equiv), DIEA (56 uL, 0.34 mmol, 2 equiv) in DCM (0.5 mL) were added acetyl chloride (13 uL, 0.17 mmol, 1 equiv), then the reaction mixture was allowed to stir at it for 4 h. The reaction mixture was added H2O (4 mL) and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 30 mg (purity=89%) light yellow solid compound with a yield of 56%.

TLC R_{f}=0.4 (DCM:MeOH=10:1)

LCMS (m/z)=503.0 (M+H)

### Example 13. Representative Synthesis of Compounds of Formula 18

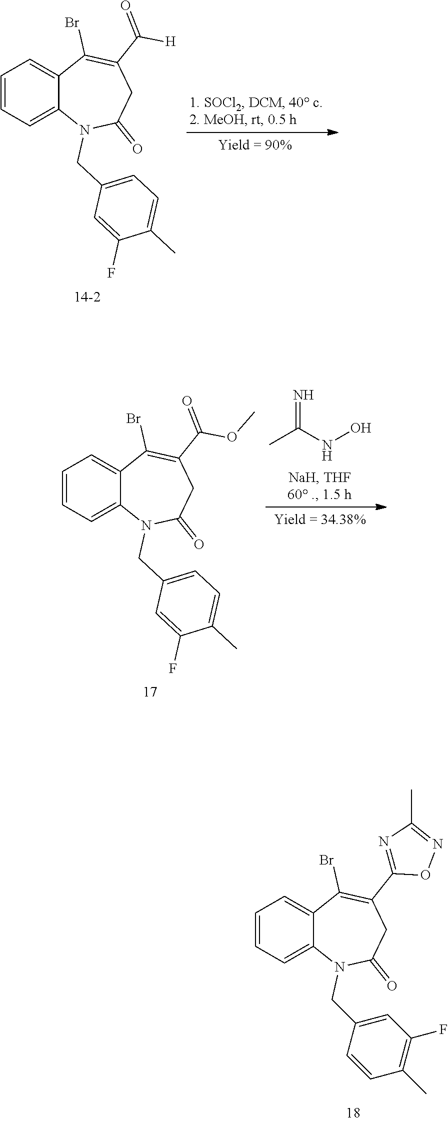

### 5-bromo-1-(3-fluoro-4-methylbenzyl)-4-(3-methyl-1,2,4-oxadiazol-5-yl)-1,3-dihydro-2H-benzo[b]azepin-2-one(18-1)

To a solution of 5-bromo-1-(3-fluoro-4-methylbenzyl)-2-oxo-2,3-dihydro-1H-benzo[b]azepine-4-carboxylic acid (14-2) (271 mg, 0.67 mmol, 1 equiv) in DCM (9.6 mL), SOCl₂ (243 μL, 3.35 mmol, 5 equiv) was added and the mixture was stirred at reflux conditions for 3 h. After formation of the corresponding acid chloride reaction as shown by the TLC, then added MeOH (3 mL) to the mixture and stirred for 0.5 h. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue purified by flash chromatography (silica gel) to give the pure yellow solid (purity=99%, 255 mg, yield=90%).

TLC R_{f}=0.9 (DCM:MeOH=10:1)

LCMS (m/z)=418.00 (M+H)

N-hydroxyacetimidamide (64.20 mg, 0.87 mmol, 2.4 eq) suspended in THF (2.7 mL) under N₂ was stirred with NaH 60% dispersion in oil (22.46 mg, 0.94 mmol, 2.6 eq) for 1 h, 1-(3-fluoro-4-methylbenzyl)-5-methoxy-2-oxo-2,3-dihydro-1H-benzo[b]azepine-4-carboxylate 17 (151.2 mg, 0.36 mmol, 1 eq) in THF (0.2 mL) was added and the reaction heated under reflux for 1.5 h. After cooling, the solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 54.5 mg (purity=99%) yellow oil compound with a yield of 34.38%

TLC R_{f}=0.5 (PE:EA=4:1)

LCMS (m/z)=442.20 (M+H)

¹H NMR (400 MHz, DMSO) δ 7.91-7.73 (m, 1H), 7.58 (d, J=7.7 Hz, 1H), 7.55-7.48 (m, 1H), 7.35 (dd, J=11.1, 4.0 Hz, 1H), 7.12 (t, J=7.8 Hz, 1H), 6.74 (d, J=9.1 Hz, 2H), 5.39 (d, J=16.0 Hz, 1H), 4.91 (d, J=16.0 Hz, 1H), 3.85 (d, J=13.4 Hz, 1H), 3.13 (d, J=13.4 Hz, 1H), 2.46 (s, 3H), 2.13 (s, 3H).

### Example 14. Representative Synthesis of Compounds of Formula 20

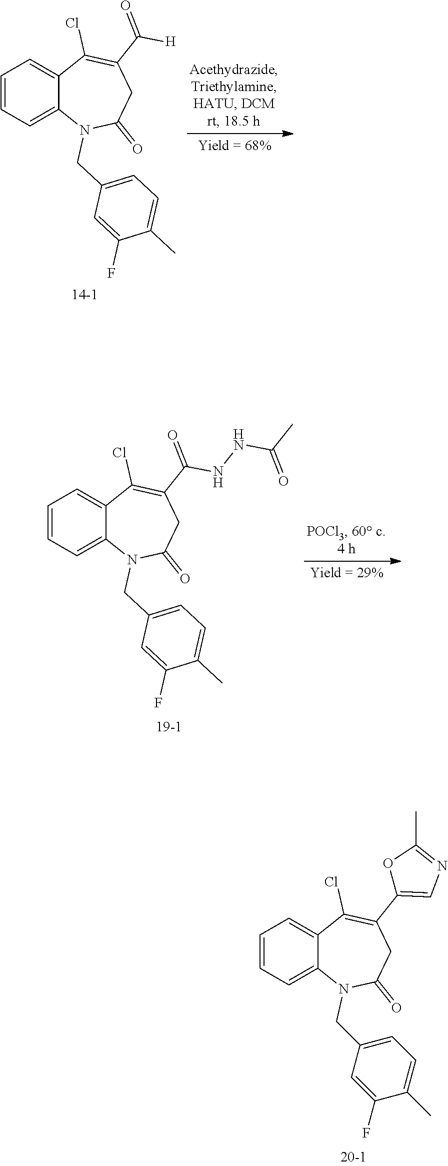 [SMILES: Cc1ccc(CN2C(=O)CC(C=O)=C(Cl)c3ccccc32)cc1F ; CC(=O)NNC(=O)C1=C(Cl)c2ccccc2N(Cc2ccc(C)c(F)c2)C(=O)C1 ; Cc1ncc(C2=C(Cl)c3ccccc3N(Cc3ccc(C)c(F)c3)C(=O)C2)o1]

### 5-chloro-1-(3-fluoro-4-methylbenzyl)-4-(5-methyl-1,3,4-oxadiazol-2-yl)-1,3-dihydro-2H-benzo[b]azepin-2-one (20-1)

To a solution of 5-chloro-1-(3-fluoro-4-methylbenzyl)-2-oxo-2,3-dihydro-1H-benzo[b]azepine-4-carboxylic acid (14-1) (200 mg, 0.56 mmol, 1 equiv) in dichloromethane (7.5 mL) was added acethydrazide (62.2 mg, 0.84 mmol, 1.5 equiv), triethylamine (116.7 μL, 0.84 mmol, 1.5 equiv), and HATU (425.6 mg, 1.12 mmol, 2 equiv). The mixture was stirred at room temperature for 18.5 h. Then it was concentrated to give a crude, which was purified by silica gel column chromatography (petroleum ether/EtOAc, 5:1 to 3:1) to get the product 160 mg (purity=96%) as a white solid. Yield: 68%.

TLC R_{f}=0.55 (DCM:MeOH=10:1)

LCMS (m/z)=416.70 (M+H)

N′-acetyl-5-chloro-1-(3-fluoro-4-methylbenzyl)-2-oxo-2,3-dihydro-1H benzo[b]azepine-4-carbohydrazide (19-1) (160 mg, 0.38 mmol, 1 equiv) was dissolved in phosphorus oxychloride (3.7 mL), and the mixture was stirred at 60° C. for 4 h. After the reaction mixture had cooled to room temperature it was poured into a mixture of ice and water. The resulting solution was extracted with portions of ethyl acetate (3×20 mL). The combined organic extracts were dried over anhydrous sodium sulfate and concentrated to give a crude, which was purified by silica gel column chromatography (PE/EA, 2:1) to get the product (44 mg) as a white solid. Yield: 29%. Purity: 99%.

TLC R_{f}=0.8 (DCM:MeOH=10:1)

LCMS (m/z)=398.30 (M+H)

¹H NMR (400 MHz, DMSO) δ 7.88-7.75 (m, 1H), 7.62-7.48 (m, 2H), 7.41-7.29 (m, 1H), 7.13 (t, J=8.0 Hz, 1H), 6.82-6.68 (m, 2H), 5.34 (d, J=16.1 Hz, 1H), 4.95 (d, J=16.1 Hz, 1H), 3.94 (d, J=13.3 Hz, 1H), 3.13 (d, J=13.3 Hz, 1H), 2.63 (s, 3H), 2.13 (s, 3H).

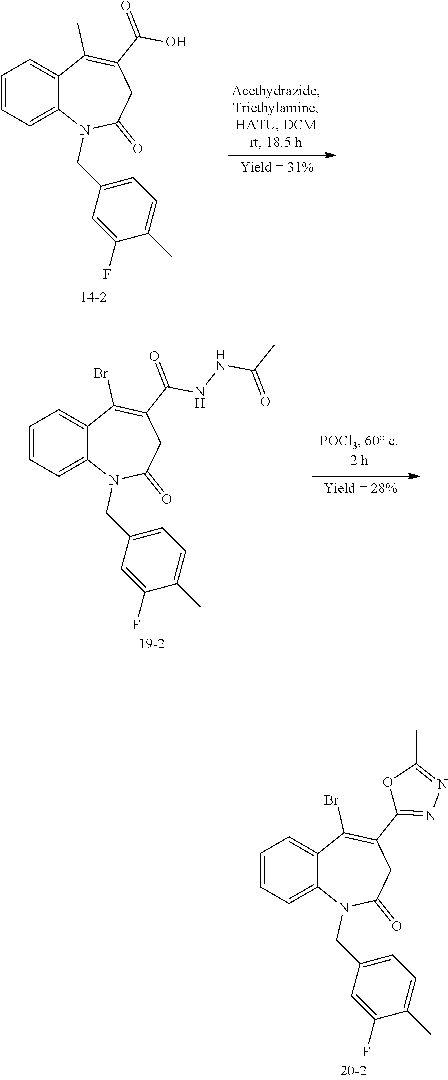 [SMILES: CC1=C(C(=O)O)CC(=O)N(Cc2ccc(C)c(F)c2)c2ccccc21 ; CC(=O)NNC(=O)C1=C(Br)c2ccccc2N(Cc2ccc(C)c(F)c2)C(=O)C1 ; Cc1nnc(C2=C(Br)c3ccccc3N(Cc3ccc(C)c(F)c3)C(=O)C2)o1]

### 5-bromo-1-(3-fluoro-4-methylbenzyl)-4-(5-methyl-1,3,4-oxadiazol-2-yl)-1,3-dihydro-2H-benzo[b]azepin-2-one (20-2)

5-bromo-1-(3-fluoro-4-methylbenzyl)-2-oxo-2,3-dihydro-1H-benzo[b]azepine-4-carboxylic acid (14-2) (200 mg, 0.56 mmol, 1 equiv) in dichloromethane (6.6 mL) was added acethydrazide (55 mg, 0.743 mmol, 1.5 equiv), triethylamine (103 μL, 0.743 mmol, 1.5 equiv), and HATU (376.2 mg, 0.99 mmol, 2 equiv). The mixture was stirred at room temperature for 18.5 h. Then it was concentrated to give a crude, which was purified by silica gel column chromatography to get the product 71 mg (purity=92%) as a white solid. Yield: 31%.

TLC R_{f}=0.3 (DCM:MeOH=10:1)

LCMS (m/z)=462.40 (M+H)

Compound 5-bromo-N′-(cyclopropanecarbonyl)-1-(3-fluoro-4-methylbenzyl)-2-oxo-2,3-dihydro-1H-benzo[b]azepine-4-carbohydrazide (19-2) (100 mg, 0.21 mmol, 1 equiv) was dissolved in phosphorus oxychloride (2.1 mL), and the mixture was stirred at 60° C. for 2 h. After the reaction mixture had cooled to room temperature it was poured into a mixture of ice and water. The resulting solution was extracted with portions of ethyl acetate (3×20 mL). The combined organic extracts were dried over anhydrous sodium sulfate and concentrated to give a crude, which was purified by silica gel column chromatography to get the product (22 mg) as a white solid. Yield: 28%. Purity: 93%.

TLC R_{f}=0.8 (PE:EA=1:1)

LCMS (m/z)=442.40 (M+H)

¹H NMR (400 MHz, DMSO) δ 7.80 (dd, J=8.0, 1.0 Hz, 1H), 7.56 (d, J=8.0 Hz, 1H), 7.53-7.46 (m, 1H), 7.33 (t, J=7.2 Hz, 1H), 7.12 (t, J=8.0 Hz, 1H), 6.84-6.65 (m, 2H), 5.38 (d, J=16.1 Hz, 1H), 4.92 (d, J=16.1 Hz, 1H), 3.87 (d, J=13.2 Hz, 1H), 3.09 (d, J=13.2 Hz, 1H), 2.62 (s, 3H), 2.12 (s, 3H). ¹³C NMR (100 MHz, DMSO) δ 169.09, 164.91, 162.94, 162.24, 159.82, 139.85, 137.69, 137.62, 133.37, 132.03, 131.97, 131.54, 131.24, 126.00, 123.54, 123.32, 123.15, 123.12, 122.91, 122.88, 122.45, 113.75, 113.53, 49.34, 38.99, 14.21, 14.18, 11.24.

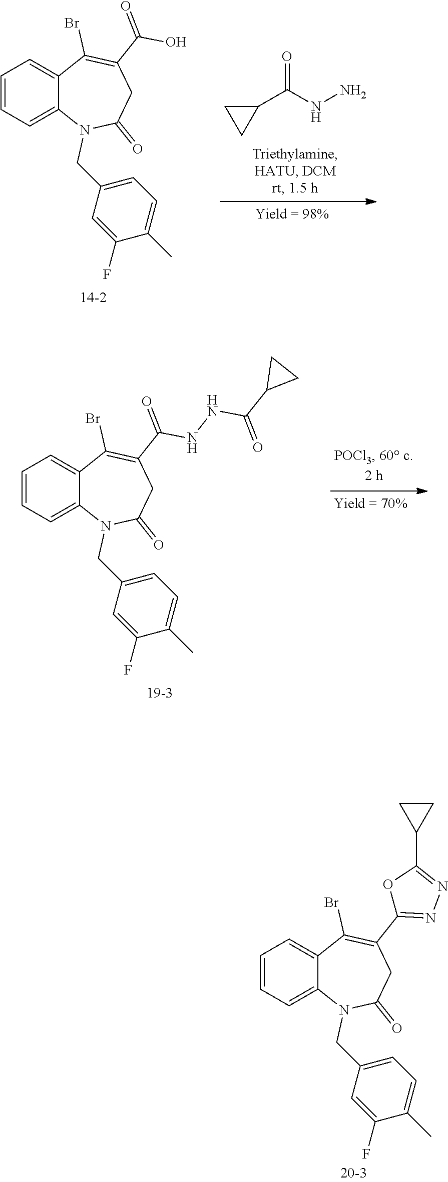 [SMILES: Cc1ccc(CN2C(=O)CC(C(=O)O)=C(Br)c3ccccc32)cc1F ; NNC(=O)C1CC1 ; Cc1ccc(CN2C(=O)CC(C(=O)NNC(=O)C3CC3)=C(Br)c3ccccc32)cc1F ; Cc1ccc(CN2C(=O)CC(c3nnc(C4CC4)o3)=C(Br)c3ccccc32)cc1F]

### 5-bromo-4-(5-cyclopropyl-1,3,4-oxadiazol-2-yl)-1-(3-fluoro-4-methylbenzyl)-1,3-dihydro-2H-benzo[b]azepin-2-one(20-3)

5-bromo-1-(3-fluoro-4-methylbenzyl)-2-oxo-2,3-dihydro-1H-benzo[b]azepine-4-carboxylic acid (14-2) (300 mg, 0.74 mmol, 1 equiv) in dichloromethane (6.6 mL) was added Cyclopropanecarboxylic acid hydrazide (111.1 mg, 1.11 mmol, 1.5 equiv), triethylamine (154 μL, 1.11 mmol, 1.5 equiv), and HATU (562.4 mg, 1.48 mmol, 2 equiv). The mixture was stirred at room temperature for 1.5 h. Then it was concentrated to give a crude, which was purified by silica gel column chromatography to get the product 342 mg (purity=93%) as a white solid. Yield: 98%.

TLC R_{f}=0.45 (DCM:MeOH=10:1)

LCMS (m/z)=488.20 (M+H)

Compound(5-bromo-N′-(cyclopropanecarbonyl)-1-(3-fluoro-4-methylbenzyl)-2-oxo-2,3-dihydro-1H-benzo[b]azepine-4-carbohydrazide) 19-3 (524 mg, 1.08 mmol, 1 equiv) was dissolved in phosphorus oxychloride (11 mL), and the mixture was stirred at 60° C. for 2 h. After the reaction mixture had cooled to room temperature, ethyl acetate was added and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography to get the product 356 mg as a yellow solid. Yield: 70%. Purity: 99%.

TLC R_{f}=0.7 (DCM:MeOH=10:1);

LCMS (m/z)=468.40 (M+H)

¹H NMR (400 MHz, DMSO) δ 7.79 (d, J=8.0 Hz, 1H), 7.55 (d, J=8.1 Hz, 1H), 7.49 (t, J=7.7 Hz, 1H), 7.33 (t, J=7.6 Hz, 1H), 7.11 (t, J=8.0 Hz, 1H), 6.73 (t, J=8.3 Hz, 2H), 5.37 (d, J=16.1 Hz, 1H), 4.91 (d, J=16.1 Hz, 1H), 3.88 (d, J=13.2 Hz, 1H), 3.06 (d, J=13.2 Hz, 1H), 2.42-2.29 (m, 1H), 2.12 (s, 3H), 1.30-1.19 (m, 2H), 1.12 (d, J=3.0 Hz, 2H). ¹³C NMR (100 MHz, DMSO) δ 169.21, 169.03, 162.25, 162.12, 159.83, 139.87, 137.69, 137.62, 133.39, 132.02, 131.96, 131.53, 131.23, 126.01, 123.59, 123.32, 123.15, 122.93, 122.90, 122.83, 122.29, 113.70, 113.48, 49.46, 38.93, 14.22, 14.19, 9.05, 8.91, 6.48.

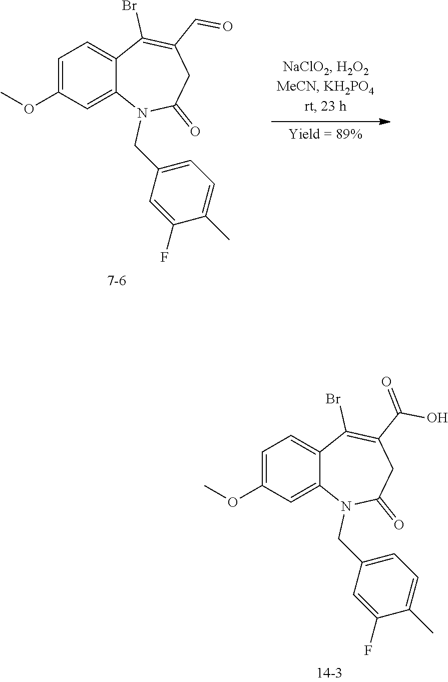

### 5-bromo-1-(3-fluoro-4-methylbenzyl)-7-methoxy-2-oxo-2,3-dihydro-1H-benzo[b]azepine-4-carboxylic acid (14-3)

Reactions were carried out by addition of aqueous NaClO₂ (572 mg, 6.32 mmol, 3.6 equiv) to a solution of 5-bromo-1-(3-fluoro-4-methylbenzyl)-7-methoxy-2-oxo-2,3-dihydro-1H-benzo[b]azepine-4-carbaldehyde 7-6 (734 mg, 1.76 mmol, 1 equiv) and (61 mg, 1.79 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 682 mg (purity=99%) white solid compound with a yield of 89%.

TLC R_{f}=0.05 (EA:PE=1:2)

LCMS (m/z)=434.60 (M+H)

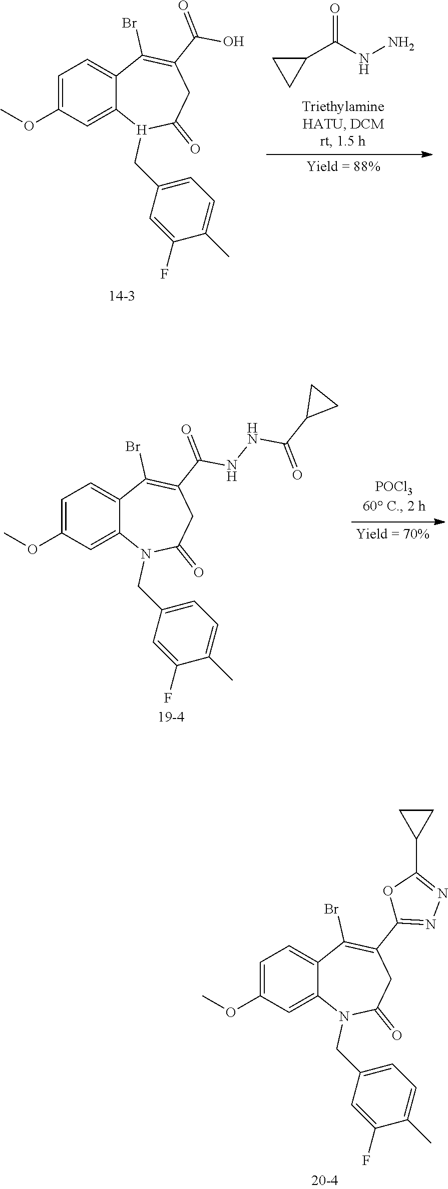

### 5-bromo-4-(5-cyclopropyl-1,3,4-oxadiazol-2-yl)-1-(3-fluoro-4-methylbenzyl)-8-methoxy-1,3-dihydro-2H-benzo[b]azepin-2-one (20-4)

5-bromo-1-(3-fluoro-4-methylbenzyl)-8-methoxy-2-oxo-2,3-dihydro-1H-benzo[b]azepine-4-carboxylic acid (14-3) (682 mg, 1.57 mmol, 1 equiv) in dichloromethane (14 mL) was added Cyclopropanecarboxylic acid hydrazide (236 mg, 2.36 mmol, 1.5 equiv), triethylamine (0.327 mL, 2.36 mmol, 1.5 equiv), and HATU (I 193 mg, 3.14 mmol, 2 equiv). The mixture was stirred at room temperature for 1.5 h. Then it was concentrated to give a crude, which was purified by silica gel column chromatography (DCM/MeOH, 10:1) to get the product 714 mg (purity=83%) as a white solid. Yield: 88%.

TLC R_{f}=0.5 (DCM:MeOH=10:1)

LCMS (m/z)=518.10 (M+H)

Compound (5-bromo-N′-(cyclopropanecarbonyl)-1-(3-fluoro-4-methylbenzyl)-7-methoxy-2-oxo-2,3-dihydro-1H-benzo[b]azepine-4-carbohydrazide) 19-4 (714 mg, 1.38 mmol, 1 equiv) was dissolved in phosphorus oxychloride (13 mL), and the mixture was stirred at 60° C. for 2 h. After the reaction mixture had cooled to room temperature, ethyl acetate was added to dilute it and the solution was slowly poured into room temperature water under a water bath. The resulting solution was extracted with portions of ethyl acetate (3×20 mL). The combined organic extracts were dried over anhydrous sodium sulfate and concentrated to give a crude compound 310 mg (purity=97%) of the light yellow oil compound with a yield of 45%

TLC R_{f}=0.85 (MeOH:DCM=1:10)

LCMS (m/z)=498.00 (M+H)

¹H NMR (400 MHz, DMSO) δ 7.72 (d, J=8.9 Hz, 1H), 7.13 (t, J=7.9 Hz, 1H), 7.06 (d, J=2.3 Hz, 1H), 6.95 (dd, J=9.0, 2.4 Hz, 1H), 6.74 (dd, J=12.8, 9.6 Hz, 2H), 5.36 (d, J=16.0 Hz, 1H), 4.94 (d, J=16.0 Hz, 1H), 3.85 (d, J=13.1 Hz, 1H), 3.80 (s, 3H), 3.07 (d, J=13.1 Hz, 1H), 2.39-2.30 (m, 1H), 2.13 (s, 3H), 1.28-1.19 (m, 2H), 1.12 (dt, J=7.5, 3.6 Hz, 2H).

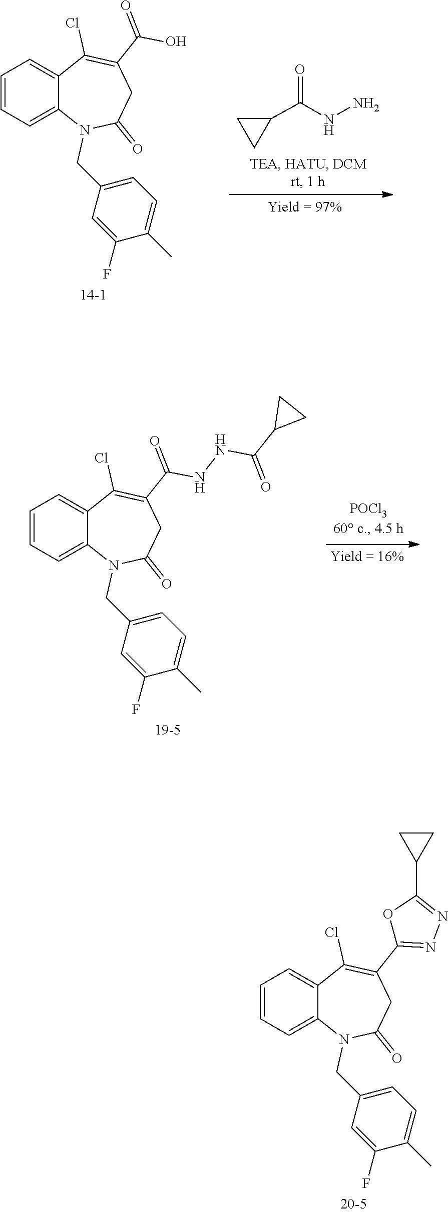 [SMILES: Cc1ccc(CN2C(=O)CC(C(=O)O)=C(Cl)c3ccccc32)cc1F ; NNC(=O)C1CC1 ; Cc1ccc(CN2C(=O)CC(C(=O)NNC(=O)C3CC3)=C(Cl)c3ccccc32)cc1F ; Cc1ccc(CN2C(=O)CC(c3nnc(C4CC4)o3)=C(Cl)c3ccccc32)cc1F]

### 5-chloro-4-(5-cyclopropyl-1,3,4-oxadiazol-2-yl)-1-(3-fluoro-4-methylbenzyl)-1,3-dihydro-2H-benzo[b]azepin-2-one (20-5)

5-chloro-1-(3-fluoro-4-methylbenzyl)-2-oxo-2,3-dihydro-1H-benzo[b]azepine-4-carboxylic acid (14-1) (829 mg, 2.31 mmol, 1 equiv) in dichloromethane (31 mL) was added Cyclopropanecarboxylic acid hydrazide (347 mg, 3.465 mmol, 1.5 equiv), triethylamine (481 μL, 3.465 mmol, 1.5 equiv), and HATU (1755.6 mg, 4.62 mmol, 2 equiv). The mixture was stirred at room temperature for 1.5 h. Then it was concentrated to give a crude, which was purified by silica gel column chromatography (DCM/MeOH, 10:1) to get the product 996 mg (purity=99%) as a white solid. Yield: 97%.

TLC R_{f}=0.45 (DCM:MeOH=10:1);

LCMS (m/z)=442.10 (M+H)

Compound (5-chloro-N′-(cyclopropanecarbonyl)-1-(3-fluoro-4-methylbenzyl)-2-oxo-2,3-dihydro-1H-benzo[b]azepine-4-carbohydrazide) 19-5 (952 mg, 2.15 mmol, 1 equiv) was dissolved in phosphorus oxychloride (21.5 mL), and the mixture was stirred at 60° C. for 4.5 h. After the reaction mixture had cooled to room temperature, ethyl acetate was added to dilute it and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography (PE/EA, 2:3) to get the product (150 mg) as a yellow solid. Yield: 16%. Purity: 92%.

TLC R_{f}=0.8 (DCM:MeOH=10:1);

LCMS (m/z)=424.40 (M+H)

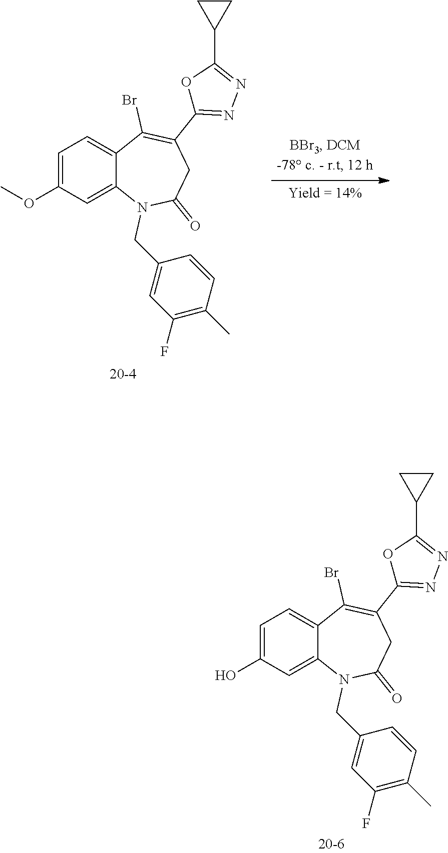 [SMILES: COc1ccc2c(c1)N(Cc1ccc(C)c(F)c1)C(=O)CC(c1nnc(C3CC3)o1)=C2Br ; Cc1ccc(CN2C(=O)CC(c3nnc(C4CC4)o3)=C(Br)c3ccc(O)cc32)cc1F]

### 5-bromo-4-(5-cyclopropyl-1,3,4-oxadiazol-2-yl)-1-(3-fluoro-4-methylbenzyl)-7-hydroxy-1,3-dihydro-2H-benzo[b]azepin-2-one (20-6)

To a stirred solution of 20-4 (240 mg, 0.482 mmol, 1 equiv) in DCM (6 mL), was added BBr₃ (0.185 mL, 2.41 mmol, 5 equiv) at −78° C. The reaction mixture was then stirred at R.T for 12 h. The reaction progress was monitored by TLC until the complete disappearance of compound LCL174. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 30 mg (purity=95%) light yellow solid compound with a yield of 14%.

TLC R_{f}=0.2 (EA:PE=1:1)

LCMS (m/z)=486.10 (M+H)

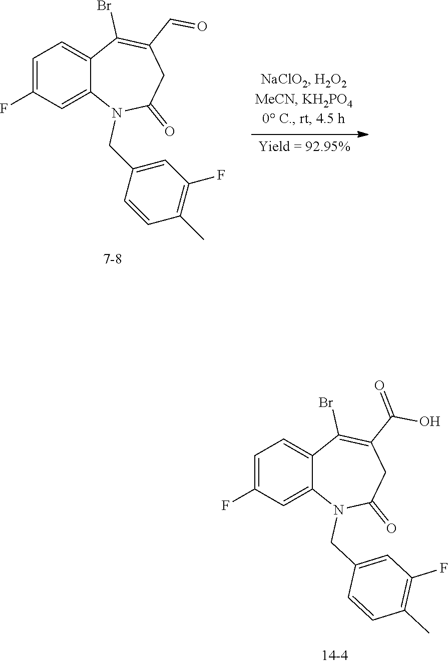 [SMILES: Cc1ccc(CN2C(=O)CC(C=O)=C(Br)c3ccc(F)cc32)cc1F ; Cc1ccc(CN2C(=O)CC(C(=O)O)=C(Br)c3ccc(F)cc32)cc1F]

### 5-bromo-7-fluoro-1-(3-fluoro-4-methylbenzyl)-2-oxo-2,3-dihydro-1H-benzo[b]azepine-4-carboxylic acid (14-4)

Reactions were carried out by addition of aqueous NaClO₂ (274.51 mg, 3.05 mmol, 1.76 eq) to a solution of 5-bromo-8-fluoro-1-(3-fluoro-4-methylbenzyl)-2-oxo-2,3-dihydro-1H-benzo[b]azepine-4-carbaldehyde 7-8 (704 mg, 1.73 mmol, 1 eq) and (60 mg, 1.76 mmol, 1.02 eq) 30% H₂O₂ in aqueous acetonitrile, at 0° C., buffered with KH₂PO₄ at pH 4.3. The reaction mixture was stirred for 4.5 h. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 679 mg (purity=99.27%) yellow solid compound with a yield of 92.95.%

TLC R_{f}=0.2 (DCM:MeOH=10:1)

LCMS (m/z)=422.0 (M+H)

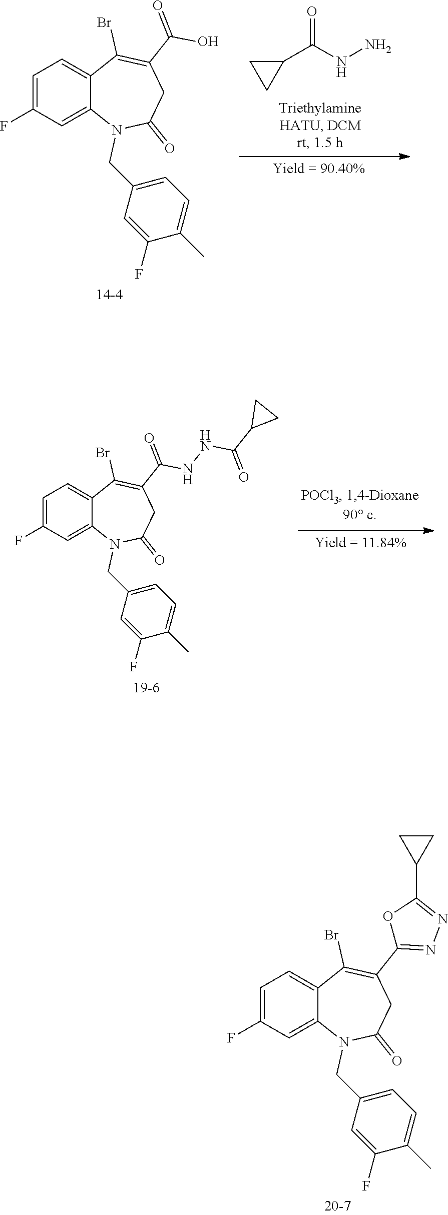 [SMILES: Cc1ccc(CN2C(=O)CC(C(=O)O)=C(Br)c3ccc(F)cc32)cc1F ; NNC(=O)C1CC1 ; Cc1ccc(CN2C(=O)CC(C(=O)NNC(=O)C3CC3)=C(Br)c3ccc(F)cc32)cc1F ; Cc1ccc(CN2C(=O)CC(c3nnc(C4CC4)o3)=C(Br)c3ccc(F)cc32)cc1F]

### 5-bromo-4-(5-cyclopropyl-1,3,4-oxadiazol-2-yl)-8-fluoro-1-(3-fluoro-4-methylbenzyl)-1,3-dihydro-2H-benzo[b]azepin-2-one (20-7)

5-bromo-8-fluoro-1-(3-fluoro-4-methylbenzyl)-2-oxo-2,3-dihydro1Hbenzo[b]azepine-4-carboxylic acid 14-4 (340 mg, 0.805 mmol, 1 equiv) in dichloromethane (7 mL) was added Cyclopropanecarboxylic acid hydrazide (120.93 mg, 1.208 mmol, 1.5 equiv), triethylamine (167 μL, 1.208 mmol, 1.5 equiv), and HATU (612.37 mg, 1.610 mmol, 2 equiv). The mixture was stirred at room temperature for 1.5 h. Then it was concentrated to give a crude, which was purified by silica gel column chromatography (DCM/MeOH, 10:1) to get the product 367 mg (purity=89.52%) as a white solid. Yield: 90.40%.

TLC R_{f}=0.6 (DCM:MeOH=10:1)

LCMS (m/z)=505.80 (M+H)

bromo-N′-(cyclopropanecarbonyl)-8-fluoro-1-(3-fluoro-4-methylbenzyl)-2-oxo-2,3-dihydro-1H-benzo[b]azepine-4-carbohydrazide 19-6 (167 mg, 0.33 mmol, 1 equiv) was dissolved in 1,4-dioxane (4.86 mL) and phosphoryl chloride (202 μL, 2017 mmol, 6.57 equiv) was added and the mixture was heated at 90° C. for 2 h, thein cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (19 mg) as a yellow solid. Yield: 11.84%; Purity: 91%.

TLC R_{f}=0.8 (DCM:MeOH=20:1)

LCMS (m/z)=487.80 (M+H)

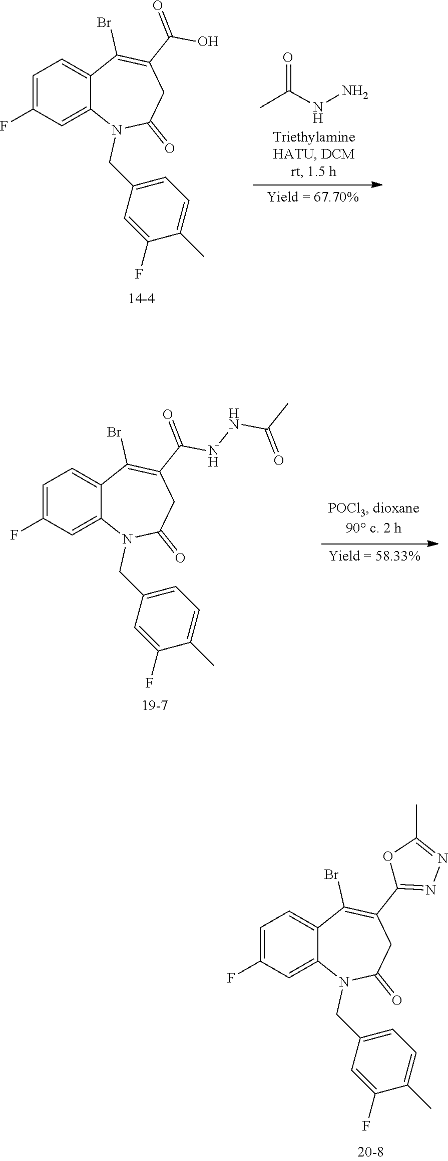

### 5-bromo-8-fluoro-1-(3-fluoro-4-methylbenzyl)-4-(5-methyl-1,3,4-oxadiazol-2-yl)-1,3-dihydro-2H-benzo[b]azepin-2-one (20-8)

5-bromo-7-fluoro-1-(3-fluoro-4-methylbenzyl)-2-oxo-2,3-dihydro-1H-benzo[b]azepine-4-carboxylic acid (339 mg, 0.803 mmol, 1 equiv) in dichloromethane (7 mL) was added acetohydrazide (89.216 mg, 1.204 mmol, 1.5 equiv), triethylamine (167.9 μL, 1.204 mmol, 1.5 equiv), and HATU (609.90 mg, 1.604 mmol, 2 equiv). The mixture was stirred at room temperature for 1.5 h. Then it was concentrated to give a crude, which was purified by silica gel column chromatography (DCM/MeOH, 10:1) to get the product 260 mg (purity=93.92%) as a white solid. Yield: 67.70%.

TLC R_{f}=0.8 (DCM:MeOH=20:1)

LCMS (m/z)=479.90 (M+H)

N′-acetyl-5-bromo-8-fluoro-1-(3-fluoro-4-methylbenzyl)-2-oxo-2,3-dihydro-1H-benzo[b]azepine-4-carbohydrazide 19-7 (260 mg, 0.54 mmol, 1 equiv) was dissolved in 1,4-dioxane (7.95 mL) and phosphoryl chloride (332.89 μL, 3.57 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (145 mg) as an orange solid. Yield: 58.33%; Purity: 98.91%.

TLC R_{f}=0.5 (DCM:MeOH=20:1)

LCMS (m/z)=461.90 (M+H)

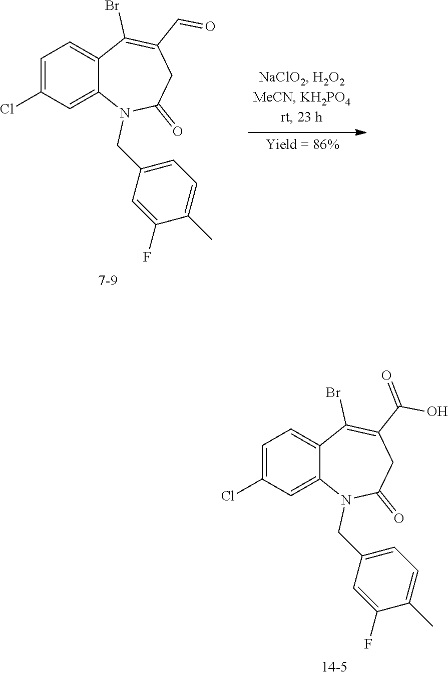 [SMILES: Cc1ccc(CN2C(=O)CC(C=O)=C(Br)c3ccc(Cl)cc32)cc1F ; Cc1ccc(CN2C(=O)CC(C(=O)O)=C(Br)c3ccc(Cl)cc32)cc1F]

### 5-bromo-8-chloro-1-(3-fluoro-4-methylbenzyl)-2-oxo-2,3-dihydro-1H-benzo[b]azepine-4-carboxylic acid (14-5)

Reactions were carried out by addition of aqueous NaClO₂ (766.5 mg, 8.52 mmol, 3.6 equiv) to a solution of 5-bromo-8-chloro-1-(3-fluoro-4-methylbenzyl)-2-oxo-2,3-dihydro-1H-benzo[b]azepine-4-carbaldehyde 7-9 (1 g, 2.37 mmol, 1 equiv) and (242 μL, 2.41 mmol, 1.02 equiv) of 30% H₂O₂ in aqueous acetonitrile, at 0° C. buffered with KH₂PO₄ at pH 4˜3. The reaction mixture was stirred overnight. After complete reaction, a small amount of the Na₂SO₃ (˜400 mg) was added to destroy the unreacted HOCl and H₂O₂. Acidification with NH₄Cl, the mixture was diluted with H₂O (20 mL). The compound was extracted with EA (3×10-20 mL). The organic layer was separated and extracted with water and brine and then dried over sodium sulphate and concentrated. Then, the resulting concentrate was purified on a silica column to get 890 mg (purity=90%) white solid compound with a yield of 86%.

TLC R_{f}=0.7 (PE:EA=4:1);

LCMS (m/z)=439.80 (M+H)

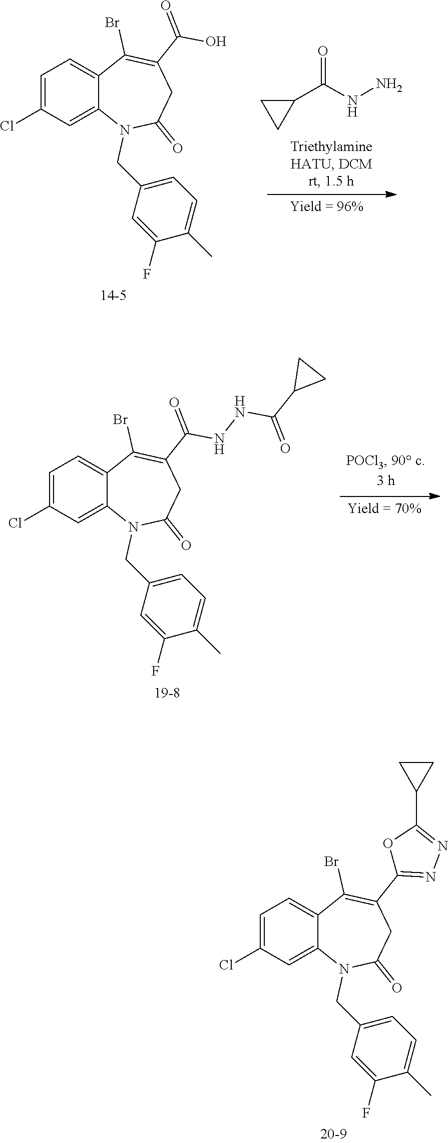 [SMILES: Cc1ccc(CN2C(=O)CC(C(=O)O)=C(Br)c3ccc(Cl)cc32)cc1F ; NNC(=O)C1CC1 ; Cc1ccc(CN2C(=O)CC(C(=O)NNC(=O)C3CC3)=C(Br)c3ccc(Cl)cc32)cc1F ; Cc1ccc(CN2C(=O)CC(c3nnc(C4CC4)o3)=C(Br)c3ccc(Cl)cc32)cc1F]

### 5-bromo-8-chloro-4-(5-cyclopropyl-1,3,4-oxadiazol-2-yl)-1-(3-fluoro-4-methylbenzyl)-1,3-dihydro-2H-benzo[b]azepin-2-one (20-9)

5-bromo-8-chloro-1-(3-fluoro-4-methylbenzyl)-2-oxo-2,3-dihydro-1H-benzo[b]azepine-4-carboxylic acid 14-5 (890 mg, 2.03 mmol, 1 equiv) in dichloromethane (20 mL) was added Cyclopropanecarboxylic acid hydrazide (304.3 mg, 3.32 mmol, 1.5 equiv), triethylamine (423 μL, 3.32 mmol, 1.5 equiv), and HATU (1.54 g, 4.42 mmol, 2 equiv). The mixture was stirred at room temperature for 1.5 h. Then it was concentrated to give a crude, which was purified by silica gel column chromatography to get the product 1.02 g (purity=99%) as a white solid. Yield: 96%.

TLC R_{f}=0.4 (DCM:MeOH=10:1)

LCMS (m/z)=521.90 (M+H)

5-bromo-8-chloro-N′-(cyclopropanecarbonyl)-1-(3-fluoro-4-methylbenzyl)-2-oxo-2,3-dihydro-1H-benzo[b]azepine-4-carbohydrazide 19-8 (1.02 g, 1.96 mmol, 1 equiv) in 1,4-Dioxane (20 mL) was added phosphorus oxychloride (1.12 ml, 12.87 mmol, 6.57 equiv), and the mixture was stirred at 90° C. for 3 h. After the reaction mixture had cooled to room temperature, ethyl acetate was added and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography to get the product 670 mg as a yellow solid. Yield: 70%. Purity: 99%.

TLC R_{f}=0.4 (PE:EA=2:1);

LCMS (m/z)=503.90 (M+H)

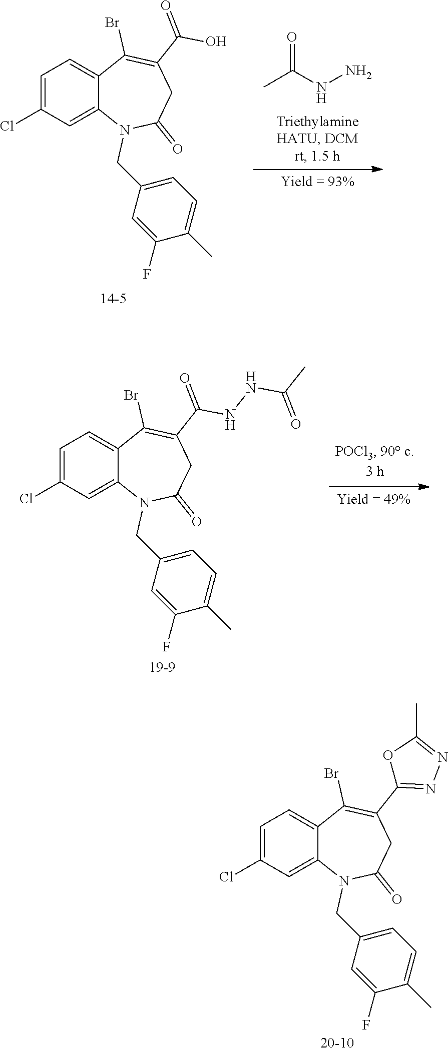

### 5-bromo-8-chloro-1-(3-fluoro-4-methylbenzyl)-4-(5-methyl-1,3,4-oxadiazol-2-yl)-1,3-dihydro-2H-benzo[b]azepin-2-one (20-10)

5-bromo-8-chloro-1-(3-fluoro-4-methylbenzyl)-2-oxo-2,3-dihydro-1H-benzo[b]azepine-4-carboxylic acid 14-5 (400 mg, 0.91 mmol, 1 equiv) in dichloromethane (10 mL) was added Cyclopropanecarboxylic acid hydrazide (101.5 mg, 1.37 mmol, 1.5 equiv), triethylamine (190 μL, 1.37 mmol, 1.5 equiv), and HATU (693.4 g, 1.82 mmol, 2 equiv). The mixture was stirred at mom temperature for 1.5 h. Then it was concentrated to give a crude, which was purified by silica gel column chromatography to get the product 470 mg (purity=89%) as a white solid. Yield: 93%.

TLC R_{f}=0.5 (DCM:MeOH=10:1)

LCMS (m/z)=495.80 (M+H)

N′-acetyl-5-bromo-8-chloro-1-(3-fluoro-4-methylbenzyl)-2-oxo-2,3-dihydro-1H-benzo[b]azepine-4-carbohydrazide 19-9 (470 mg, 0.95 mmol, 1 equiv) in 1,4-Dioxane (15 mL) was added phosphorus oxychloride (582 ul, 6.24 mmol, 6.57 equiv), and the mixture was stirred at 90° C. for 3 h. After the reaction mixture had cooled to room temperature, ethyl acetate was added and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography to get the product 220 mg as a yellow solid. Yield: 49%. Purity: 98%.

TLC R_{f}=0.6 (PE:EA=1:1);

LCMS (m/z)=478.3 (M+H)

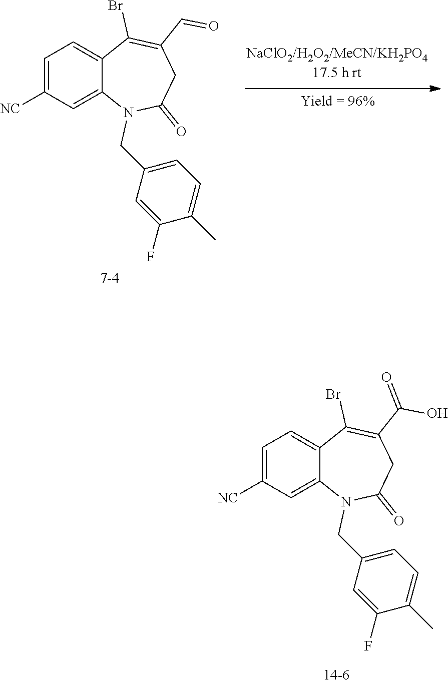 [SMILES: Cc1ccc(CN2C(=O)CC(C=O)=C(Br)c3ccc(C#N)cc32)cc1F ; Cc1ccc(CN2C(=O)CC(C(=O)O)=C(Br)c3ccc(C#N)cc32)cc1F]

### 5-bromo-8-cyano-1-(3-fluoro-4-methylbenzyl)-2-oxo-2,3-dihydro-1H-benzo[b]azepine-4-carboxylic acid (14-6)

Reactions were carried out by addition of aqueous NaClO₂ (144.62 mg, 1.607 mmol, 1.76 equiv) to a solution of 5-bromo-1-(3-fluoro-4-methylbenzyl)-4-formyl-2-oxo-2,3-dihydro-1H-benzo[b]azepine-8-carbonitrile (7-4) (377 mg, 0.913 mmol, 1 equiv) and (31.65 mg, 0.931 mmol, 1.02 equiv) of 30% H₂O₂ in aqueous acetonitrile, at 0° C. buffered with KH₂PO₄ at pH 4˜3. The reaction mixture was stirred overnight. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 380 mg (purity=96%) white solid compound with a yield of 96%.

TLC R_{f}=0.3 (DCM:MeOH=10:1);

LCMS (m/z)=430.70 (M+H)

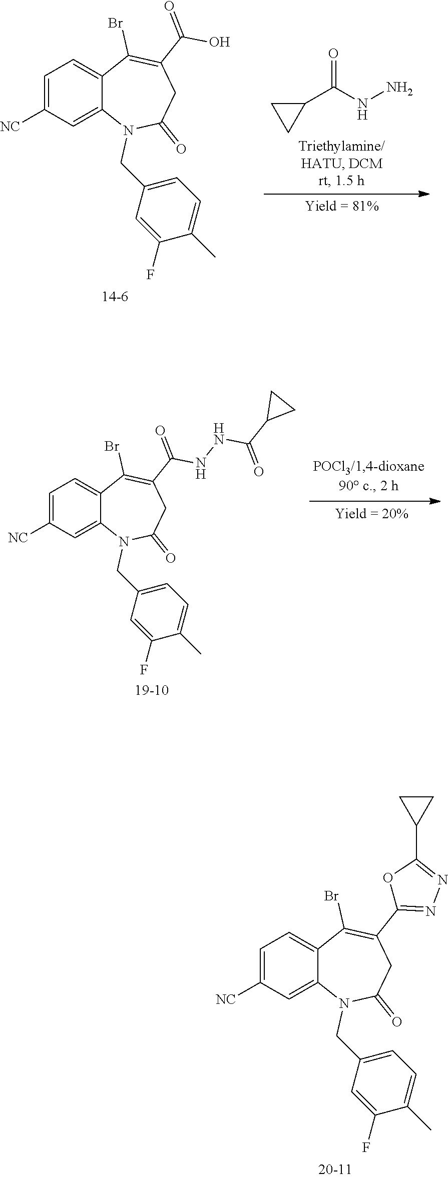 [SMILES: Cc1ccc(CN2C(=O)CC(C(=O)O)=C(Br)c3ccc(C#N)cc32)cc1F ; NNC(=O)C1CC1 ; Cc1ccc(CN2C(=O)CC(C(=O)NNC(=O)C3CC3)=C(Br)c3ccc(C#N)cc32)cc1F ; Cc1ccc(CN2C(=O)CC(c3nnc(C4CC4)o3)=C(Br)c3ccc(C#N)cc32)cc1F]

### 5-bromo-4-(5-cyclopropyl-1,3,4-oxadiazol-2-yl)-1-(3-fluoro-4-methylbenzyl)-2-oxo-2,3-dihydro-1H-benzo[b]azepine-8-carbonitrile (20-11)

5-bromo-8-cyano-1-(3-fluoro-4-methylbenzyl)-2-oxo-2,3-dihydro-11H-benzo[b]azepine-4-carboxylic acid (14-6) (380 mg, 0.885 mmol, 1 equiv) in dichloromethane (11.9 mL) was added Cyclopropanecarboxylic acid hydrazide (133 mg, 1.33 mmol, 1.5 equiv), triethylamine (184.6.9l L, 1.33 mmol, 1.5 equiv), and HATU (673 mg, 1.77 mmol, 2 equiv). The mixture was stirred at room temperature for 1.5 h. Then it was concentrated to give a crude, which was purified by silica gel column chromatography to get the product 370 mg (purity=92%) as a white solid. Yield: 81%.

TLC R_{f}=0.45 (DCM:MeOH=10:1);

LCMS (m/z)=513.00 (M+H)

5-bromo-8-cyano-N′-(cyclopropanecarbonyl)-1-(3-fluoro-4-methylbenzyl)-2-oxo-2,3-dihydro-1H-benzo[b]azepine-4-carbohydrazide (19-10) (370 mg, 0.724 mmol, 1 equiv) was dissolved in 1,4-dioxane (10.6 mL) and phosphoryl chloride (443 μL, 5.36 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (21 mg) as a yellow solid. Yield: 20%; Purity: 99%.

TLC R_{f}=0.8 (PE:EA=1:1);

LCMS (m/z)=494.80 (M+H)

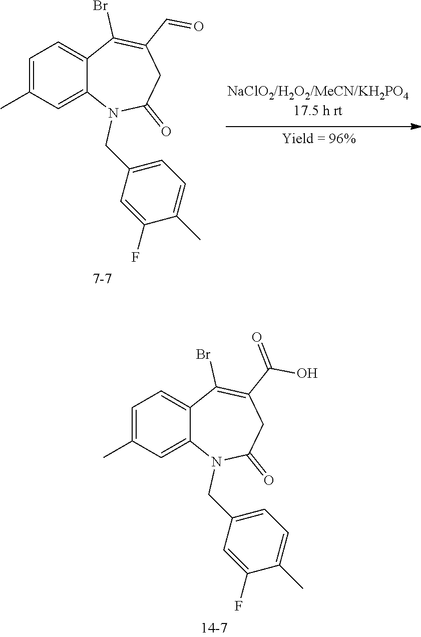

### 5-bromo-1-(3-fluoro-4-methylbenzyl)-8-methyl-2-oxo-2,3-dihydro-1H-benzo[b]azepine-4-carboxylic acid (14-7)

Reactions were carried out by addition of aqueous NaClO₂ (194.52 mg, 2.16 mmol, 1.76 eq) to a solution of 5-bromo-1-(3-fluoro-4-methylbenzyl)-8-methyl-2-oxo-2,3-dihydro-1-H-benzo[b]azepine-4-carbaldehyde 7-7 (494 mg, 1.23 mmol, 1 eq) and (42.59 mg, 127.89 uL, 1.76 mmol, 1.02 eq) 30% H₂O₂ in aqueous acetonitrile, at 0° C., buffered with KH₂PO₄ at pH 4.3. The reaction mixture was stirred 4.5 h. After complete reaction, a small amount of the Na₂SO₃ (˜0.5 g) was added to destroy the unreacted HOCl and H₂O₂. Acidification with 10% HCl, the mixture was diluted with 1-120 (10-20 mL). The compound was extracted with Et₂O (10 mL). The organic layer was separated and extracted with water and brine and then dried over sodium sulphate and concentrated. The product was purified by column chromatography using 60-120 mesh silica gel (hexane/EtOAc), dried over anhydrous Na₂SO₄, filtered and concentrated by rotary evaporation to get the crude compound. Then, the resulting concentrate was purified on a silica column to get 490 mg (purity=96.69%) yellowish solid compound with a yield of 95.24%.

TLC R_{f}=0.3 (DCM:MeOH=10:1)

LCMS (m/z)=420.20 (M+H)

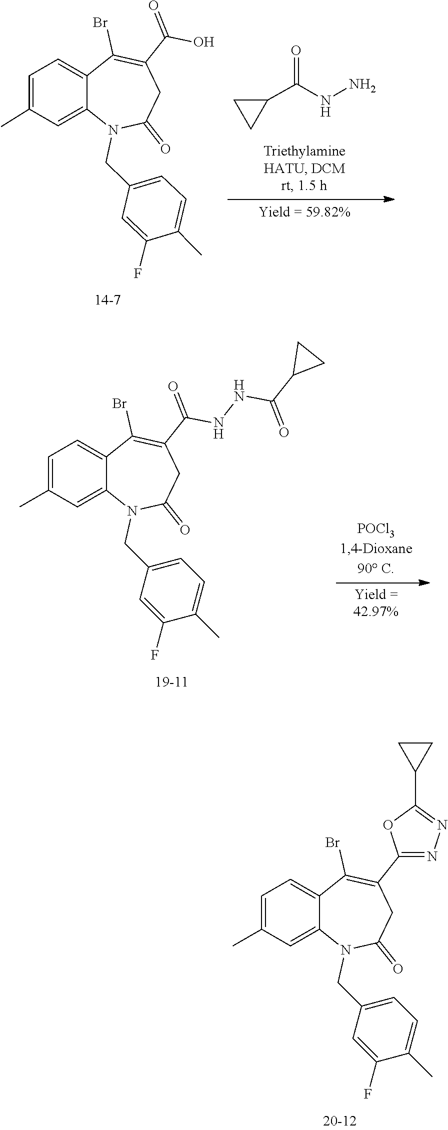

### 5-bromo-4-(5-cyclopropyl-1,3,4-oxadiazol-2-yl)-1-(3-fluoro-4-methylbenzyl)-8-methyl-1,3-dihydro-2H-benzo[b]azepin-2-one (20-12)

bromo-1-(3-fluoro-4-methylbenzyl)-8-methyl-2-oxo-2,3-dihydro-1H benzo[b]azepine-4-carboxylic acid 14-7 (250 mg, 0.598 mmol, 1 equiv) in dichloromethane (5.33 mL) was added acetohydrazide (89.76 mg, 0.896 mmol, 1.5 equiv), triethylamine (124.62 μL, 0.896 mmol, 1.5 equiv), and HATU (454.55 mg, 1.195 mmol, 2 equiv). The mixture was stirred at room temperature for 1.5 h. Then it was concentrated to give a crude, which was purified by silica gel column chromatography (DCM/MeOH, 10:1) to get the product 179 mg (purity=90.60%) as a white solid. Yield: 59.82%.

TLC R_{f}=0.5 (DCM:MeOH=10:1)

LCMS (m/z)=502.20 (M+H)

N′-acetyl-5-bromo-8-fluoro-1-(3-fluoro-4-methylbenzyl)-2-oxo-2,3-dihydro-1H-benzo[b]azepine-4-carbohydrazide 19-11 (179 mg, 0.357 mmol, 1 equiv) was dissolved in 1,4-dioxane (5.25 mL) and phosphoryl chloride (219 μL, 2.35 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (74 mg) as a yellow oil. Yield: 58.33%; Purity: 97.52%.

TLC R_{f}=0.5 (PE:EA=1:1)

LCMS (m/z)=482.40 (M+H)

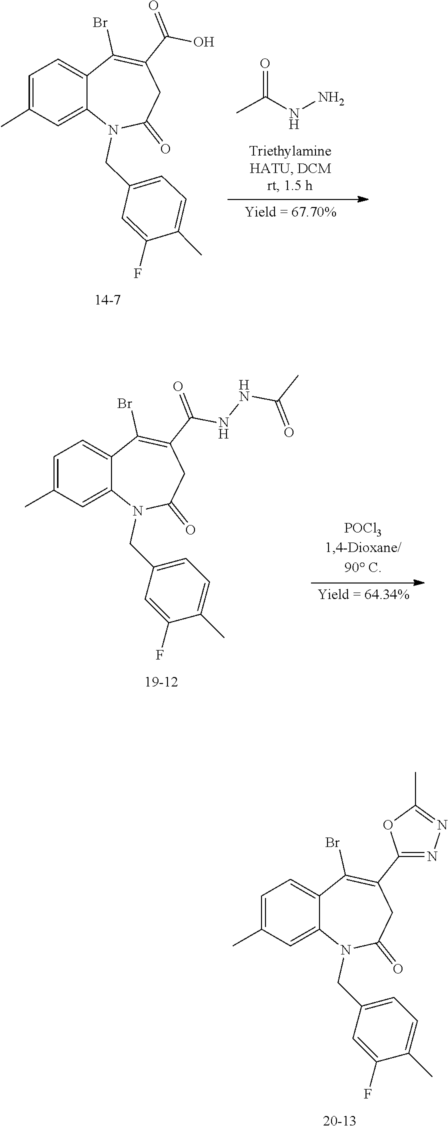

### 5-bromo-1-(3-fluoro-4-methylbenzyl)-8-methyl-4-(5-methyl-1,3,4-oxadiazol-2-yl)-1,3-dihydro-2H-benzo[b]azepin-2-one (20-13)

bromo-1-(3-fluoro-4-methylbenzyl)-8-methyl-2-oxo-2,3-dihydro-1H-benzo[b]azepine-4-carboxylic acid 14-7 (240 mg, 0.574 mmol, 1 equiv) in dichloromethane (5.12 mL) was added acetohydrazide (63.76 mg, 0.861 mmol, 1.5 equiv), triethylamine (119.63 μL, 0.861 mmol, 1.5 equiv), and HATU (436.36 mg, 1.147 mmol, 2 equiv). The mixture was stirred at room temperature for 1.5 h. Then it was concentrated to give a crude, which was purified by silica gel column chromatography (DCM/MeOH, 10:1) to get the product 223 mg (purity=93.51%) as a white solid. Yield: 81.90%.

TLC R_{f}=0.5 (DCM:MeOH=10:1)

LCMS (m/z)=474.30 (M+H)

N′-acetyl-5-bromo-8-fluoro-1-(3-fluoro-4-methylbenzyl)-2-oxo-2,3-dihydro-1H-benzo[b]azepine-4-carbohydrazide 19-12 (223 mg, 0.47 mmol, 1 equiv) was dissolved in 1,4-dioxane (6.9 mL) and phosphoryl chloride (287.90 μL, 33.08 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (138 mg) as a orange solid. Yield: 64.34%; Purity: 99%.

TLC R_{f}=0.4 (PE:EA=1:1)

LCMS (m/z)=456.40 (M+H)

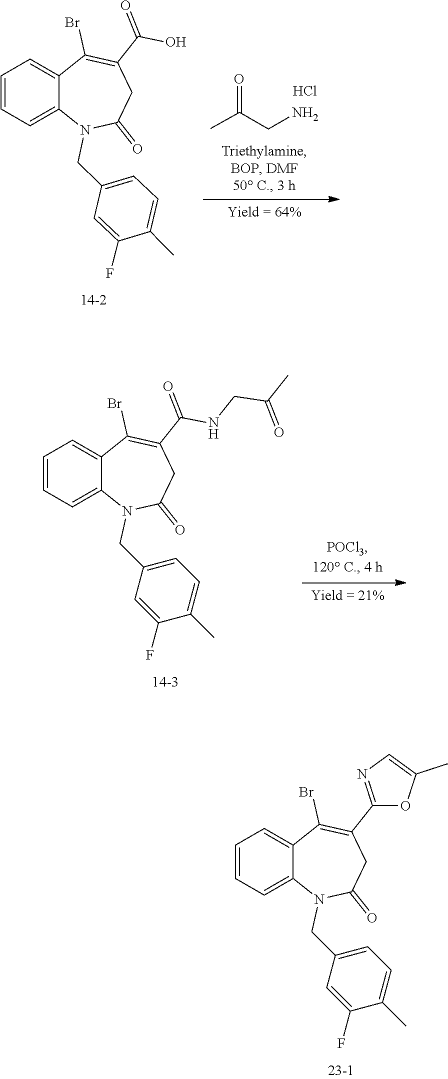

### 5-bromo-1-(3-fluoro-4-methylbenzyl)-4-(5-methyloxazol-2-yl)-1,3-dihydro-2H-benzo[b]azepin-2-one (23-1)

A mixture of 5-bromo-1-(3-fluoro-4-methylbenzyl)-2-oxo-2,3-dihydro-1H-benzo[b]azepine-4-carboxylic acid 14-2 (0.137 g, 0.34 mmol, 1 eq), 1-aminoacetone HCl salt (0.047 g, 0.425 mmol, 1.25 eq), BOP reagent (0.188 g, 0.425 mmol, 1.25 eq), and triethylamine (0.116 mL, 0.833 mmol, 2.45 eq) in DMF (1 mL) was stirred at 50° C. for 2 h. The reaction mixture was partitioned between ethyl acetate (10 mL) and water (5 mL). The ethyl acetate layer was washed with brine, dried (MgSO₄), and concentrated under reduced pressure. Then, the resulting concentrate was purified on a silica column to get 100 mg (purity=99%) yellowish solid compound with a yield of 64%.

TLC R_{f}=0.7 (DCM:MeOH=10:1)

LCMS (m/z)=461.30 (M+H)

The above carboxamide (100 mg, 0.21 mmol) was stirred with POCl₃ (3 mL) at 120° C. for 4 h. The excess POCl₃ was removed in vacuo and the residue was partitioned between dichloromethane (10 mL) and saturated aqueous NaHCO₃ solution (10 mL). The organic layer was dried (MgSO₄) and concentrated under reduced pressure. Then, the resulting concentrate was purified on a silica column to get 20 mg (purity=95%) yellowish solid compound with a yield of 21%.

TLC R_{f}=0.5 (PE:EA=2:1)

LCMS (m/z)=441.10 (M+H)

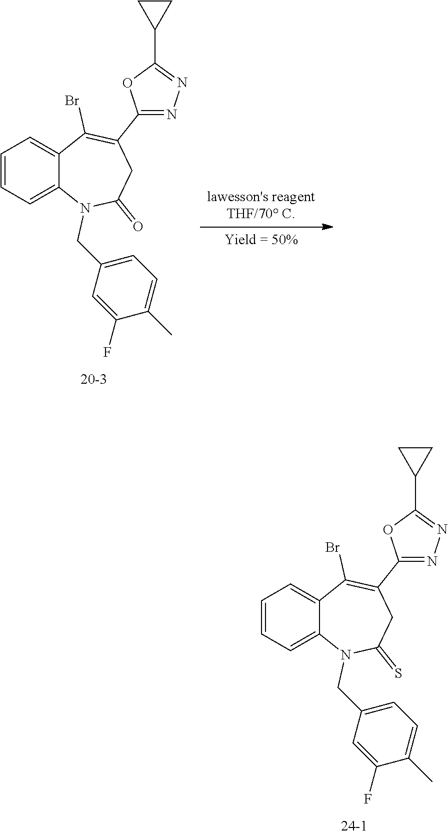 [SMILES: Cc1ccc(CN2C(=O)CC(c3nnc(C4CC4)o3)=C(Br)c3ccccc32)cc1F ; Cc1ccc(CN2C(=S)CC(c3nnc(C4CC4)o3)=C(Br)c3ccccc32)cc1F]

### 5-bromo-4-(5-cyclopropyl-1,3,4-oxadiazol-2-yl)-1-(3-fluoro-4-methylbenzyl)-1,3-dihydro-2H-benzo[b]azepine-2-thione (24-1)

Into a round-bottomed flask was added 20-3 (0.1 g, 0.21 mmol, 1 equiv), 2,4-Bis(4-methoxyphenyl)-2,4-dithioxo-1,3,2,4-dithiadiphosphetane (0.13 g, 0.32 mmol, 1.5 equiv) and THF (1.5 mL). The mixture was stirred at 60° C. for 1 hour. The mixture was then partitioned between DCM (50 mL) and sat. NaHCO₃(aq.) (20 mL). The organic phase was washed with water and brine. The solution was then dried over Na₂SO₄, filtered and concentrated. The residue was purified by silica gel column chromatography to get the product (50 mg) as a yellow oil. Yield: 50%; Purity: 96%.

TLC R_{f}=0.6 (PE:EA=2:1)

LCMS (m/z)=485.90 (M+H)

### Example 15. Representative Synthesis of Compounds of Formula 20

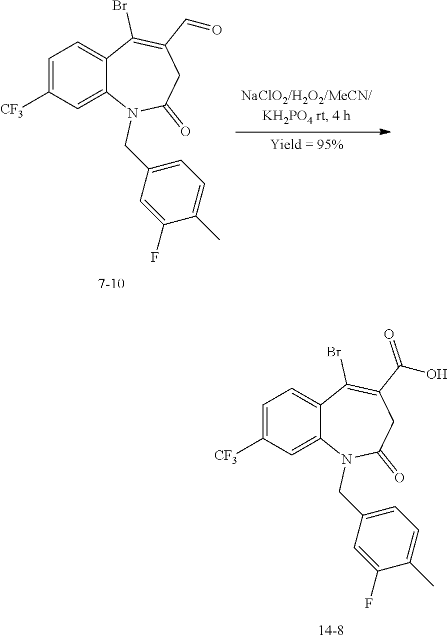 [SMILES: Cc1ccc(CN2C(=O)CC(C=O)=C(Br)c3ccc(C(F)(F)F)cc32)cc1F ; Cc1ccc(CN2C(=O)CC(C(=O)O)=C(Br)c3ccc(C(F)(F)F)cc32)cc1F]

### 5-bromo-1-(3-fluoro-4-methylbenzyl)-2-oxo-8-(trifluoromethyl)-2,3-dihydro-1H-benzo[b]azepine-4-carboxylic acid (14-8)

Reactions were carried out by addition of aqueous NaClO₂ (1.003 g, 11.14 mmol, 1.76 equiv) to a solution of 5-bromo-1-(3-fluoro-4-methylbenzyl)-2-oxo-8-(trifluoromethyl)-2,3-dihydro-1H-benzo[b]azepine-4-carbaldehyde (2.889 g, 6.33 mmol, 1 equiv) and (666 μL, 6.52 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜0.5 g) was added to destroy the unreacted HOCl and H₂O₂. Acidification with 10% HCl, the mixture was diluted with H₂O (10-20 ml). The compound was extracted with EA (10 mL). The organic layer was separated and extracted with water and brine and then dried over anhydrous Na₂SO₄, filtered and concentrated by rotary evaporation to get the crude compound. Then, the resulting concentrate was purified on a silica column to get 2.865 g (purity=98%) yellow solid compound with a yield of 95%.

TLC R_{f}=0.2 (DCM:MeOH=10:1);

LCMS (m/z)=474.70 (M+H)

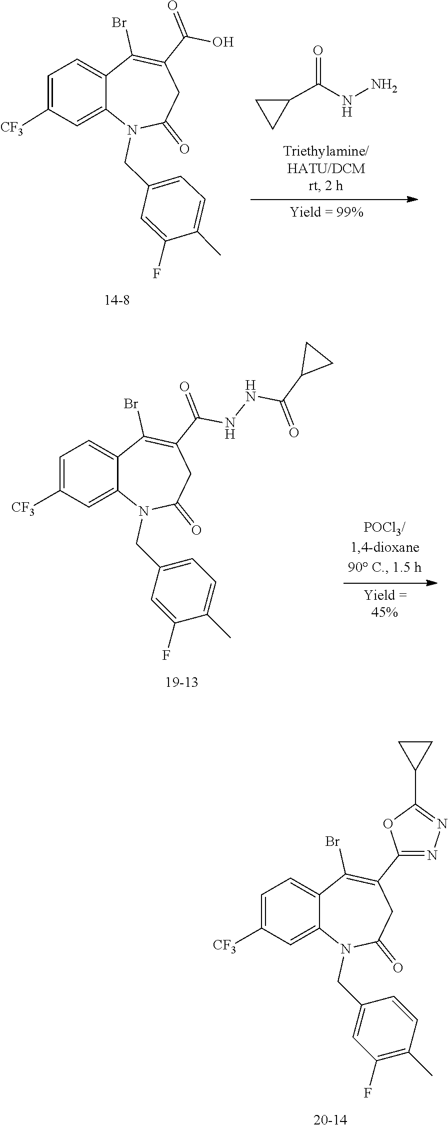 [SMILES: Cc1ccc(CN2C(=O)CC(C(=O)O)=C(Br)c3ccc(C(F)(F)F)cc32)cc1F ; NNC(=O)C1CC1 ; Cc1ccc(CN2C(=O)CC(C(=O)NNC(=O)C3CC3)=C(Br)c3ccc(C(F)(F)F)cc32)cc1F ; Cc1ccc(CN2C(=O)CC(c3nnc(C4CC4)o3)=C(Br)c3ccc(C(F)(F)F)cc32)cc1F]

### 5-bromo-4-(5-cyclopropyl-1,3,4-oxadiazol-2-yl)-1-(3-fluoro-4-methylbenzyl)-8-(trifluoromethyl)-1,3-dihydro-2H-benzo[b]azepin-2-one (20-14)

5-bromo-1-(3-fluoro-4-methylbenzyl)-2-oxo-8-(trifluoromethyl)-2,3-dihydro-1H-benzo[b]azepine-4-carboxylic acid (1 g, 2.12 mmol, 1 equiv) in dichloromethane (28.5 mL) was added Cyclopropanecarboxylic acid hydrazide (318 mg, 3.18 mmol, 1.5 equiv), triethylamine (442 μL, 2.18 mmol, 1.5 equiv), and HATU (1611.2 mg, 4.24 mmol, 2 equiv). The mixture was stirred at room temperature for 1.5 h. Then it was concentrated to give a crude, which was purified by silica gel column chromatography (DCM/MeOH, 10:1) to get the product 1.227 g (purity=97%) as a white solid. Yield: 99%.

TLC R_{f}=0.45 (DCM:MeOH=10:1);

LCMS (m/z)=556.90 (M+H)

5-bromo-N′-(cyclopropanecarbonyl)-1-(3-fluoro-4-methylbenzyl)-2-oxo-8-(trifluromethyl)-2,3-dihydro-1H-benzo[b]azepine-4-carbohydrazide (1.227 g, 2.21 mmol, 1 equiv) was dissolved in 1,4-dioxane (32.26 mL) and phosphoryl chloride (1355 μL, 14.54 mmol, 6.57 equiv) was added and the mixture was heated at 90° C. for 1.5 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (543 mg) as a yellow solid. Yield: 45%; Purity: 94%.

TLC R_{f}=0.9 (PE:EA=1:1);

LCMS (m/z)=537.20 (M+H)

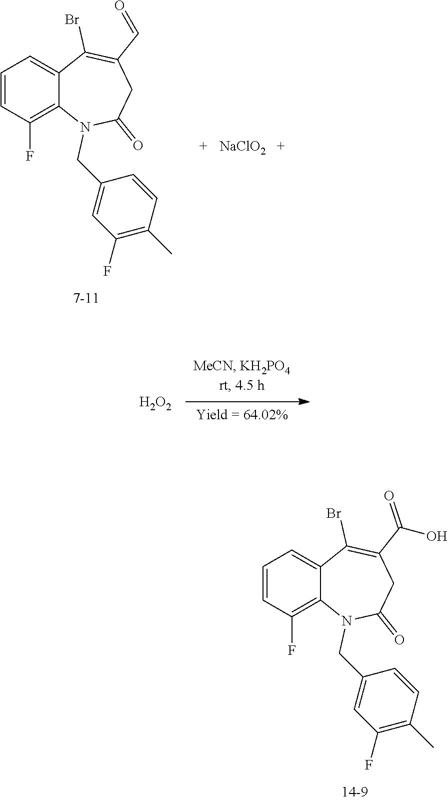

### 5-bromo-9-fluoro-1-(3-fluoro-4-methylbenzyl)-2-oxo-2,3-dihydro-1H-benzo[b]azepine-4-carboxylic acid (14-9)

Reactions were carried out by addition of aqueous NaClO₂ (506.9 mg, 5.63 mmol, 1.76 eq) to a solution of 5-bromo-9-fluoro-1-(0,3-fluoro-4-methylbenzyl)-2-oxo-2,3-dihydro-1H-benzo[b]azepine-4-carbaldehyde (1.3 g, 3.2 mmol, 1 eq) and (333.27 uL, 3.26 mmol, 1.02 eq) 30% H₂O₂ in aqueous acetonitrile, at 0° C., buffered with KH₂PO₄ at pH 4.3. The reaction mixture was stirred 4.5 h. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over anhydrous Na₂SO₄, filtered and concentrated by rotary evaporation to get the crude compound. Then, the resulting concentrate was purified on a silica column to get 865 mg (purity=90.73%) yellowish solid compound with a yield of 64.02%.

TLC R_{f}=0.3 (DCM:MeOH=10:1)

LCMS (m/z)=422.70 (M+H)

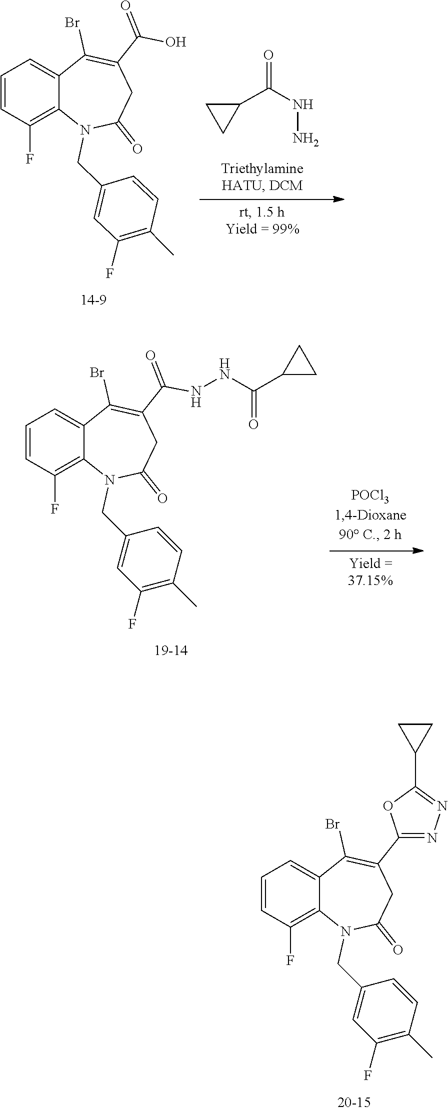 [SMILES: Cc1ccc(CN2C(=O)CC(C(=O)O)=C(Br)c3cccc(F)c32)cc1F ; NNC(=O)C1CC1 ; Cc1ccc(CN2C(=O)CC(C(=O)NNC(=O)C3CC3)=C(Br)c3cccc(F)c32)cc1F ; Cc1ccc(CN2C(=O)CC(c3nnc(C4CC4)o3)=C(Br)c3cccc(F)c32)cc1F]

### 5-bromo-4-(5-cyclopropyl-1,3,4-oxadiazol-2-yl)-9-fluoro-1-(3-fluoro-4-methylbenzyl)-1,3-dihydro-2H-benzo[b]azepin-2-one (20-15)

5-bromo-9-fluoro-1-(3-fluoro-4-methylbenzyl)-2-oxo-2,3-dihydro-1H-benzo[b]azepine-4-carboxylic acid (100 mg, 0.236 mmol, 1 equiv) in dichloromethane (3.4 mL) was added Cyclopropanecarboxylic acid hydrazide (35.56 mg, 0.355 mmol, 1.5 equiv), triethylamine (49.38 LL, 0.355 mmol, 1.5 equiv), and HATU (178 mg, 0.473 mmol, 2 equiv). The mixture was stirred at room temperature for 1.5 h. Then it was concentrated to give a crude, which was purified by silica gel column chromatography to get the product 117 mg (purity=98.86%) as a white solid. Yield: 99%.

TLC R_{f}=0.5 (DCM:MeOH=10:1)

LCMS (m/z)=504.80 (M+H)

5-bromo-N′-(cyclopropanecarbonyl)-9-fluoro-1-(3-fluoro-4-methylbenzyl)-2-oxo-2,3-dihydro-1H-benzo[b]azepine-4-carbohydrazide (117 mg, 0.234 mmol, 1 equiv) was dissolved in 1,4-dioxane (3.5 mL) and phosphoryl chloride (145.71 μL, 51.563 mmol, 6.57 equiv) was added and the mixture was heated at 90° C. for 2 h. then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (43 mg) as a yellow solid. Yield: 37.15%; Purity: 97.95%.

TLC R_{f}=0.7 (PE:EA=1:1);

LCMS (m/z)=486.80 (M+H)

### Example 16. Representative Synthesis of Compounds of Formula 20

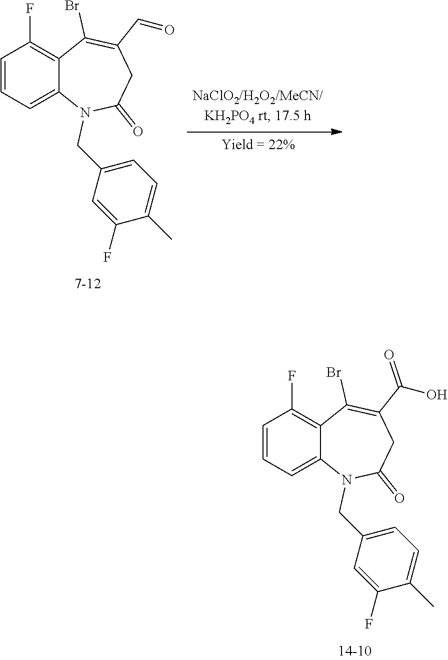 [SMILES: Cc1ccc(CN2C(=O)CC(C=O)=C(Br)c3c(F)cccc32)cc1F ; Cc1ccc(CN2C(=O)CC(C(=O)O)=C(Br)c3c(F)cccc32)cc1F]

### 5-bromo-6-fluoro-1-(3-fluoro-4-methylbenzyl)-2-oxo-2,3-dihydro-1H-benzo[b]azepine-4-carboxylic acid (14-10)

Reactions were carried out by addition of aqueous NaClO₂ (1549 mg, 17.12 mmol, 1.76 equiv) to a solution of 5-bromo-6-fluoro-1-(3-fluoro-4-methylbenzyl)-2-oxo-2,3-dihydro-1H-benzo[b]azepine-4-carbaldehyde (3.95 g, 9.73 mmol, 1 equiv) and (337 mg, 9.92 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜1.5 g) was added to destroy the unreacted HOCl and H₂O₂. Acidification with 10% HC, the mixture was diluted with H₂O (10-20 mL. The compound was extracted with EA (10 mL). The organic layer was separated and extracted with water and brine and then dried over anhydrous Na₂SO₄, filtered and concentrated by rotary evaporation to get the crude compound. Then, the resulting concentrate was purified on a silica column to get 900 mg white solid compound with a yield of 22%. Purity=92%

TLC R_{f}=0.1 (DCM:MeOH=10:1);

LCMS (m/z)=424.20 (M+H)

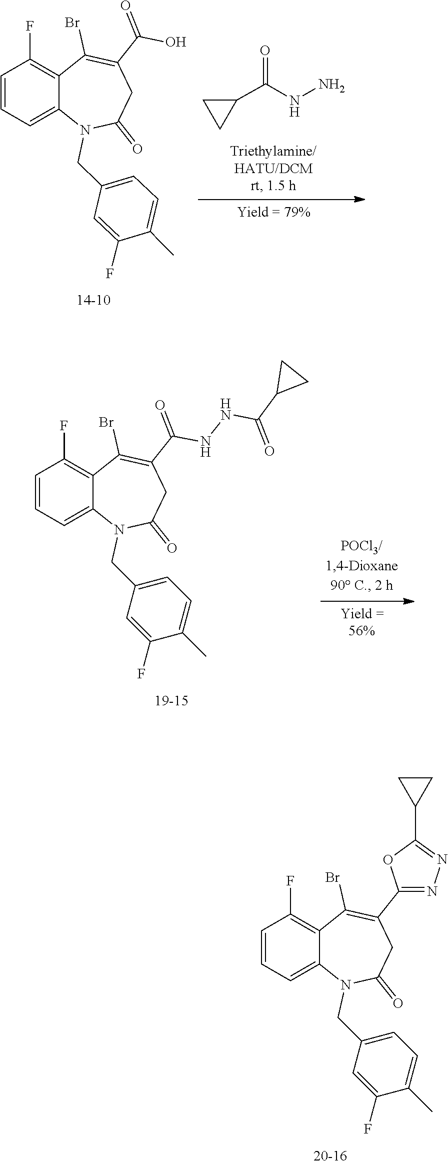 [SMILES: Cc1ccc(CN2C(=O)CC(C(=O)O)=C(Br)c3c(F)cccc32)cc1F ; NNC(=O)C1CC1 ; Cc1ccc(CN2C(=O)CC(C(=O)NNC(=O)C3CC3)=C(Br)c3c(F)cccc32)cc1F ; Cc1ccc(CN2C(=O)CC(c3nnc(C4CC4)o3)=C(Br)c3c(F)cccc32)cc1F]

### 5-bromo-4-(5-cyclopropyl-1,3,4-oxadiazol-2-yl)-6-fluoro-1-(3-fluoro-4-methylbenzyl)-1,3-dihydro-2H-benzo[b]azepin-2-one (20-16)

5-bromo-6-fluoro-1-(3-fluoro-4-methylbenzyl)-2-oxo-2,3-dihydro-11H-benzo[b]azepine-4-carboxylic acid (900 mg, 2.13 mmol, 1 equiv) in dichloromethane (21 mL) was added Cyclopropanecarboxylic acid hydrazide (320 mg, 3.20 mmol, 1.5 equiv), triethylamine (0.55 mL, 4.26 mmol, 2 equiv), and HATU (1.215 g 3.20 mmol, 1.5 equiv). The mixture was stirred at room temperature for 1.5 h. Then it was concentrated to give a crude, which was purified by silica gel column chromatography (DCM/MeOH, 10:1) to get the product 852 mg as a white solid. Yield: 79%. Purity=96%

TLC R_{f}=0.4 (DCM:MeOH=10:1);

LCMS (m/z)=504.10 (M+H)

5-bromo-N′-(cyclopropanecarbonyl)-6-fluoro-1-(3-fluoro-4-methylbenzyl)-2-oxo-2,3-dihydro-1H-benzo[b]azepine-4-carbohydrazide (852 mg, 1.79 mmol, 1 equiv) was dissolved in 1,4-dioxane (7.5 mL), followed by phosphorus oxychloride (1.1 mL), and then the mixture was stirred at 90° C. for 2 h. After the reaction mixture had cooled to room temperature, ethyl acetate was added to dilute it and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography (PE/EA, 2:3) to get the product (463 mg) as a yellow solid. Yield: 56%. Purity: 98%.

TLC R_{f}=0.3 (EA:PE=1:1);

LCMS (m/z)=488.10 (M+H)

### Example 17. Representative Synthesis of Compounds of Formula 22-17

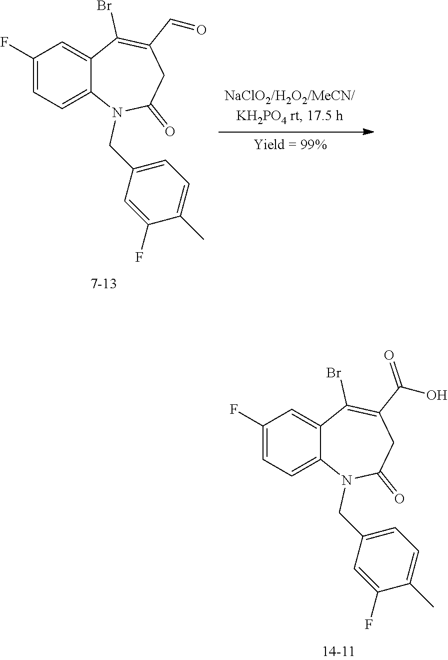 [SMILES: Cc1ccc(CN2C(=O)CC(C=O)=C(Br)c3cc(F)ccc32)cc1F ; Cc1ccc(CN2C(=O)CC(C(=O)O)=C(Br)c3cc(F)ccc32)cc1F]

### 5-bromo-7-fluoro-1-(3-fluoro-4-methylbenzyl)-2-oxo-2,3-dihydro-1H-benzo[b]azepine-4-carboxylic acid (14-11)

Reactions were carried out by addition of aqueous NaClO₂ (1.568 g, 17.34 mmol, 1.76 equiv) to a solution of 5-bromo-7-fluoro-1-(3-fluoro-4-methylbenzyl)-2-oxo-2,3-dihydro-1H-benzo[b]azepine-4-carbaldehyde (4 g, 9.85 mmol, 1 equiv) and (342 mg, 10.05 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜1.5 g) was added to destroy the unreacted HOCl and H₂O₂. Acidification with 10% HCl, the mixture was diluted with H₂O (10-20 mL. The compound was extracted with EA (10 mL). The organic layer was separated and extracted with water and brine and then dried over anhydrous Na₂SO₄, filtered and concentrated by rotary evaporation to get the crude compound. Then, the resulting concentrate was purified on a silica column to get 4.16 g white solid compound with a yield of 99%. Purity=97%

TLC R_{f}=0.2 (DCM:MeOH=10:1);

LCMS (m/z)=423.50 (M+H)

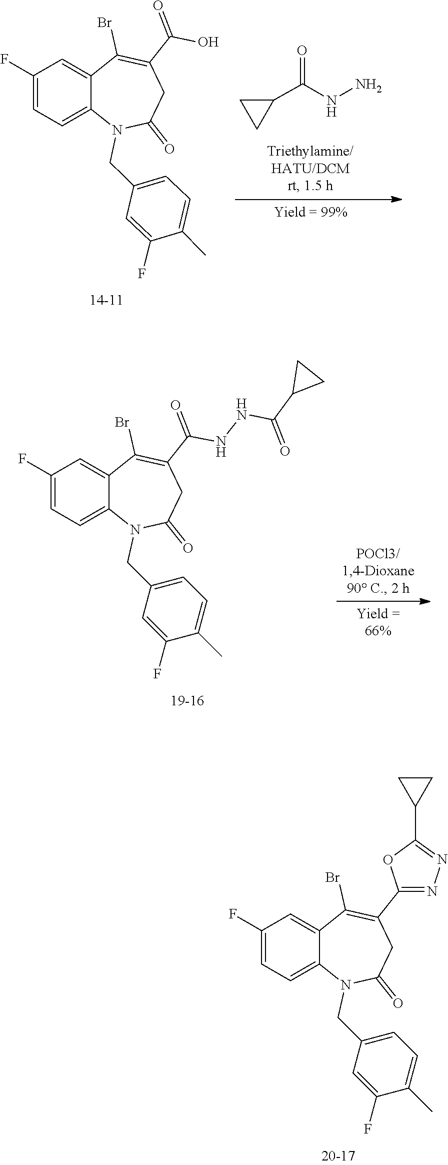 [SMILES: Cc1ccc(CN2C(=O)CC(C(=O)O)=C(Br)c3cc(F)ccc32)cc1F ; NNC(=O)C1CC1 ; Cc1ccc(CN2C(=O)CC(C(=O)NNC(=O)C3CC3)=C(Br)c3cc(F)ccc32)cc1F ; Cc1ccc(CN2C(=O)CC(c3nnc(C4CC4)o3)=C(Br)c3cc(F)ccc32)cc1F]

### 5-bromo-4-(5-cyclopropyl-1,3,4-oxadiazol-2-yl)-7-fluoro-1-(3-fluoro-4-methylbenzyl)-1,3-dihydro-2H-benzo[b]azepin-2-one (20-17)

5-bromo-7-fluoro-1-(3-fluoro-4-methylbenzyl)-2-oxo-2,3-dihydro-1H-benzo[b]azepine-4-carboxylic acid (4.16 g, 9.84 mmol, 1 equiv) in dichloromethane (100 mL) was added Cyclopropanecarboxylic acid hydrazide (1.476 g, 14.76 mmol, 1.5 equiv), triethylamine (2.54 mL, 19368 mmol, 2 equiv), and HATU (5.609 g, 14.76 mmol, 1.5 equiv). The mixture was stirred at room temperature for 1.5 h. Then it was concentrated to give a crude, which was purified by silica gel column chromatography (DCM/MeOH, 10:1) to get the product 5 g as a white solid compound with a yield of 99%. Purity=96%

TLC R_{f}=0.3 (DCM:MeOH=20:1);

LCMS (m/z)=506.00 (M+H)

5-bromo-N′-(cyclopropanecarbonyl)-7-fluoro-1-(3-fluoro-4-methylbenzyl)-2-oxo-2,3-dihydro-1H-benzo[b]azepine-4-carbohydrazide (5 g, 9.91 mmol, 1 equiv) was dissolved in 1,4-dioxane (39.6 mL), was added phosphorus oxychloride (6.07 mL), and the mixture was stirred at 90° C. for 2 h. After the reaction mixture had cooled to room temperature, ethyl acetate was added to dilute it and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography (PE/EA, 1:1) to get the product 3.16 g as a white solid compound with a yield of 66%. Purity: 97%.

TLC R_{f}=0.45 (EA:PE=1:1);

LCMS (m/z)=488.00 (M+H)

### Example 18. Representative Synthesis of Compounds of Formula 20-18

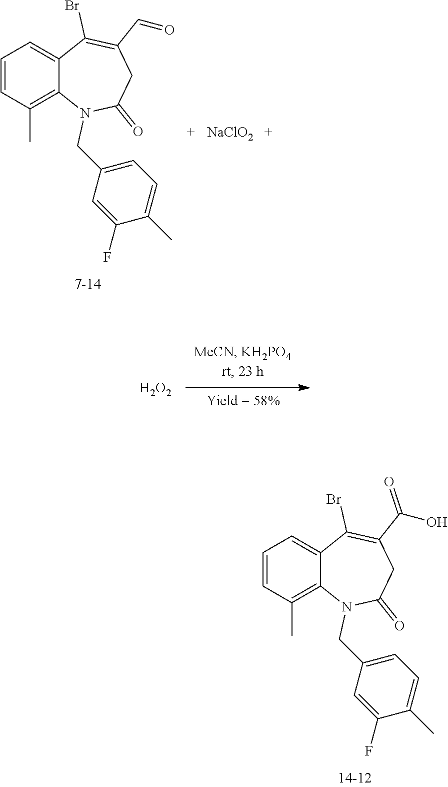

### 5-bromo-1-(3-fluoro-4-methylbenzyl)-9-methyl-2-oxo-2,3-dihydro-1H-benzo[b]azepine-4-carboxylic acid (14-12)

Reactions were carried out by addition of aqueous NaClO₂ (410.7 mg, 4.56 mmol, 3.6 equiv) to a solution of 5-bromo-1-(3-fluoro-4-methylbenzyl)-9-methyl-2-oxo-2,3-dihydro-1H-benzo[b]azepine-4-carbaldehyde (510 mg, 1.27 mmol, 1 equiv) and (130 μL, 1.29 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜300 mg) was added to destroy the unreacted HOCl and H₂O₂. Acidification with NH₄Cl, the mixture was diluted with H₂O (10-20 mL). The compound was extracted with EA (3×10-20 mL). The organic layer was separated and extracted with water and brine and then dried over anhydrous Na₂SO₄, filtered and concentrated by rotary evaporation to get the crude compound. Then, the resulting concentrate was purified on a silica column to get 310 mg (purity=83%) white solid compound with a yield of 58%.

TLC R_{f}=0.1 (PE:EA=10:1);

LCMS (m/z)=418.0 (M+H)

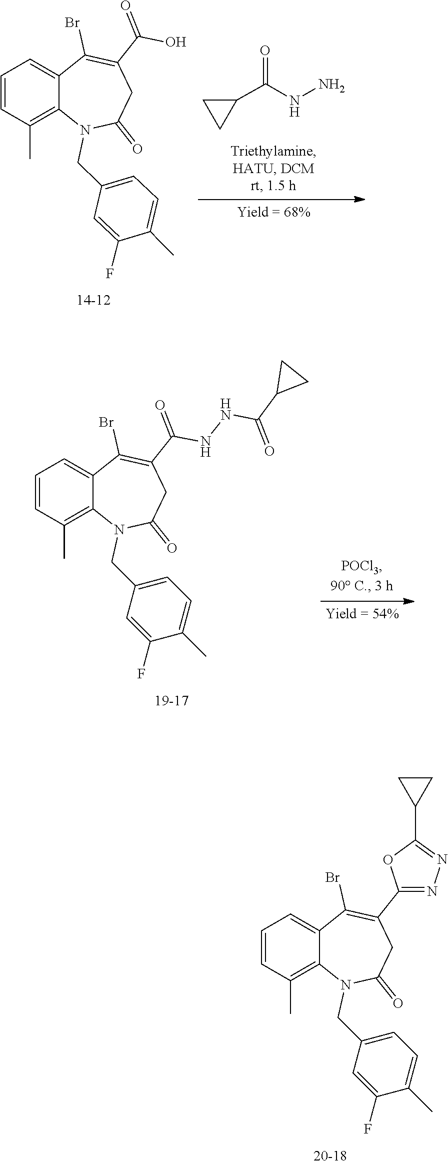 [SMILES: Cc1ccc(CN2C(=O)CC(C(=O)O)=C(Br)c3cccc(C)c32)cc1F ; NNC(=O)C1CC1 ; Cc1ccc(CN2C(=O)CC(C(=O)NNC(=O)C3CC3)=C(Br)c3cccc(C)c32)cc1F ; Cc1ccc(CN2C(=O)CC(c3nnc(C4CC4)o3)=C(Br)c3cccc(C)c32)cc1F]

### 5-bromo-4-(5-cyclopropyl-1,3,4-oxadiazol-2-yl)-1-(3-fluoro-4-methylbenzyl)-9-methyl-1,3-dihydro-2H-benzo[b]azepin-2-one (20-18)

5-bromo-1-(3-fluoro-4-methylbenzyl)-9-methyl-2-oxo-2,3-dihydro-1H-benzo[b]azepine-4-carboxylic acid (310 mg, 0.74 mmol, 1 equiv) in dichloromethane (7.3 mL) was added Cyclopropanecarboxylic acid hydrazide (111.3 mg, 1.11 mmol, 1.5 equiv), triethylamine (155 μL, 1.11 mmol, 1.5 equiv), and HATU (563.6 mg, 1.48 mmol, 2 equiv). The mixture was stirred at room temperature for 1.5 h. Then it was concentrated to give a crude, which was purified by silica gel column chromatography to get the product 250 mg (purity=91%) as a white solid. Yield: 68%.

TLC R_{f}=0.7 (DCM:MeOH=10:1)

LCMS (m/z)=502.40 (M+H)

5-bromo-N′-(cyclopropanecarbonyl)-1-(3-fluoro-4-methylbenzyl)-9-methyl-2-oxo-2,3-dihydro-1H-benzo[b]azepine-4-carbohydrazide (250 mg, 0.5 mmol, 1 equiv) in 1,4-Dioxane (5.1 mL) was added phosphorus oxychloride (306 μL, 3.28 mmol, 6.57 equiv), and the mixture was stirred at 90° C. for 3 h. After the reaction mixture had cooled to room temperature, ethyl acetate was added and the diluted solution was slowly poured into room temperature water under a water bath. The resulting solution was extracted with portions of ethyl acetate (3×20 mL). The combined organic extracts were dried over anhydrous sodium sulfate and concentrated to give a crude, which was purified by silica gel column chromatography to get the product 130 mg as a yellow solid. Yield: 54%. Purity: 92%.

TLC R_{f}=0.5 (PE:EA=1:1);

LCMS (m/z)=484.0 (M+H)

### Example 19. Representative Synthesis of Compounds of Formula 20-20

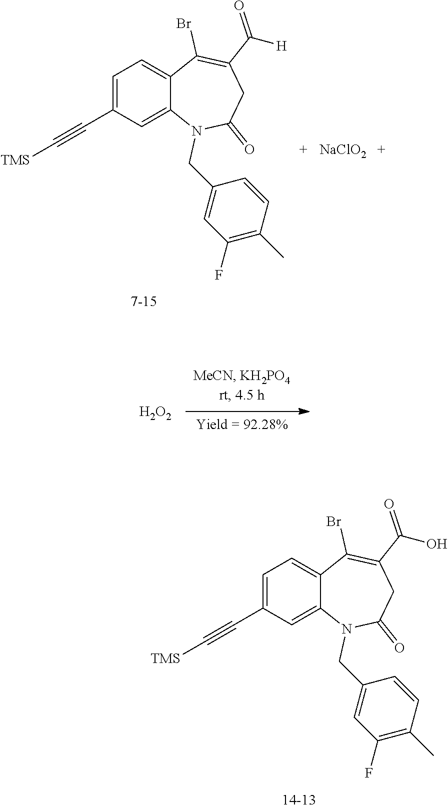

### 5-bromo-1-(3-fluoro-4-methylbenzyl)-2-oxo-8-((trimethylsilyl)ethynyl)-2,3-dihydro-1H-benzo[b]azepine-4-carboxylic acid (14-13)

Reactions were carried out by addition of aqueous NaClO₂ (330.245 mg, 3.669 mmol, 1.76 eq) to a solution of 5-bromo-1-(3-fluoro-4-methylbenzyl)-2-oxo-8-((trimethylsilyl)ethynyl)-2,3-dihydro-1H-benzo[b]azepine-4-carbaldehyde (1.01 g, 2.084 mmol, 1 eq) and (72.303 mg, 217 μL, 2.126 mmol, 1.02 eq) 30% H₂O₂ in aqueous acetonitrile, at 0° C. buffered with KH₂PO₄ at pH 4˜3. The reaction mixture was stirred 4.5 h. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 1.025 g (purity=87.79%) yellow solid compound with a yield of 98.28%.

TLC R_{f}=0.3 (DCM:MeOH=10:1)

LCMS (m/z)=501.80 (M+H)

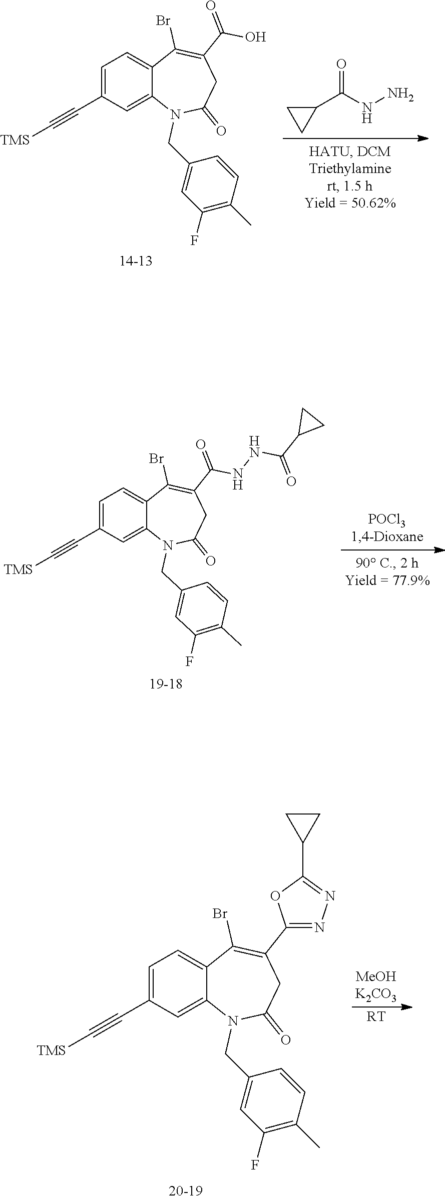 [SMILES: Cc1ccc(CN2C(=O)CC(C(=O)O)=C(Br)c3ccc(C#C[Si](C)(C)C)cc32)cc1F ; NNC(=O)C1CC1 ; Cc1ccc(CN2C(=O)CC(C(=O)NNC(=O)C3CC3)=C(Br)c3ccc(C#C[Si](C)(C)C)cc32)cc1F ; Cc1ccc(CN2C(=O)CC(c3nnc(C4CC4)o3)=C(Br)c3ccc(C#C[Si](C)(C)C)cc32)cc1F ; "RT"]

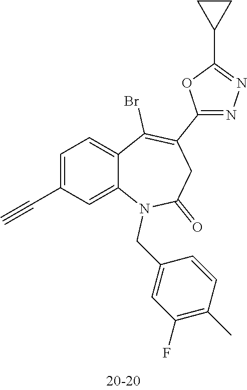 [SMILES: C#Cc1ccc2c(c1)N(Cc1ccc(C)c(F)c1)C(=O)CC(c1nnc(C3CC3)o1)=C2Br]

### 5-bromo-4-(5-cyclopropyl-1,3,4-oxadiazol-2-yl)-8-ethynyl-1-(3-fluoro-4-methylbenzyl)-1,3-dihydro-2H-benzo[b]azepin-2-one (20-20)

5-bromo-1-(3-fluoro-4-methylbenzyl)-2-oxo-8-((trimethylsilyl)ethynyl)-2,3-dihydro-1H-benzo[b]azepine-4-carboxylic acid (1.025 g, 2.048 mmol, 1 equiv) in dichloromethane (18.22 mL) was added Cyclopropanecarboxylic acid hydrazide (307.59 mg, 3.072 mmol, 1.5 equiv), triethylamine (427.04 μL, 3.072 mmol, 1.5 equiv), and HATU (1.557 g, 4.096 mmol, 2 equiv). The mixture was stirred at room temperature for 1.5 h. Then it was concentrated to give a crude, which was purified by silica gel column chromatography to get the product 604 mg (purity=97.70%) as a yellow solid. Yield: 50.62%.

TLC R_{f}=0.55 (DCM:MeOH=10:1)

LCMS (m/z)=582.10 (M+H)

5-bromo-N′-(cyclopropanecarbonyl)-1-(3-fluoro-4-methylbenzyl)-2-oxo-8-((trimethylsilyl)ethynyl)-2,3-dihydro-1H-benzo[b]azepine-4-carbohydrazide (604 mg, 1.036 mmol, 1 equiv) was dissolved in 1,4-dioxane (113.917 mL) and phosphoryl chloride (634.93 μL, 6.812 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (206 mg) as a yellow solid. Yield: 35.22%; Purity: 95.44%.

TLC R_{f}=0.7 (PE:EA=1:1);

LCMS (m/z)=564.30 (M+H)

5-bromo-4-(5-cyclopropyl-1,3,4-oxadiazol-2-yl)-1-(3-fluoro-4-methylbenzyl)-8-(trimethylsilyl)ethynyl)-1,3-dihydro-2H-benzo[b]azepin-2-one (206 mg, 0.365 mmol, 1 equiv) was dissolved in MeOH (0.2 M for substrate) and K₂CO₃ (5.04 mg, 0.036 mmol, 0.1 equiv) was added. Desilylation was conducted at room temperature for 2 hours and concentrated. The final product was purified by chromatography to get the product 140 mg (purity=87.22%) as a yellow oil. Yield: 77.90%

TLC R_{f}=0.6 (PE:EA=1:1)

LCMS (m/z)=492.35 (M+H)

### Example 20. Representative Synthesis of Compounds of Formula 20-21

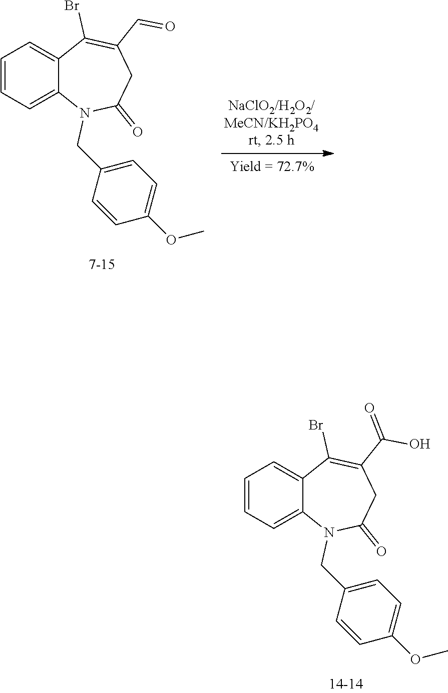 [SMILES: COc1ccc(CN2C(=O)CC(C=O)=C(Br)c3ccccc32)cc1 ; COc1ccc(CN2C(=O)CC(C(=O)O)=C(Br)c3ccccc32)cc1]

### 5-bromo-1-(4-methoxybenzyl)-2-oxo-2,3-dihydro-1H-benzo[b]azepine-4-carboxylic acid (14-14)

Reactions were carried out by addition of aqueous NaClO₂ (877.5 mg, 9.75 mmol, 1.76 equiv) to a solution of 5-bromo-1-(4-methoxybenzyl)-2-oxo-2,3-dihydro-1H-benzo[b]azepine-4-carbaldehyde (2.14 g, 5.54 mmol, 1 equiv) and (577 μL, 5.65 mmol, 1.02 equiv) of 30% H₂O₂ in aqueous acetonitrile, at 0° C. buffered with KH₂PO₄ at pH 4˜3. The reaction mixture was stirred for 2.5 h. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with EA (10 mL). The organic layer was separated and extracted with water and brine and then dried over anhydrous Na₂SO₄, filtered and concentrated by rotary evaporation to get the crude compound. Then, the resulting concentrate was purified on a silica column to get 1.62 g (purity=98%) yellow solid compound with a yield of 72.7%.

TLC R_{f}=0.2 (DCM:MeOH=10:1);

LCMS (m/z)=403.60 (M+H)

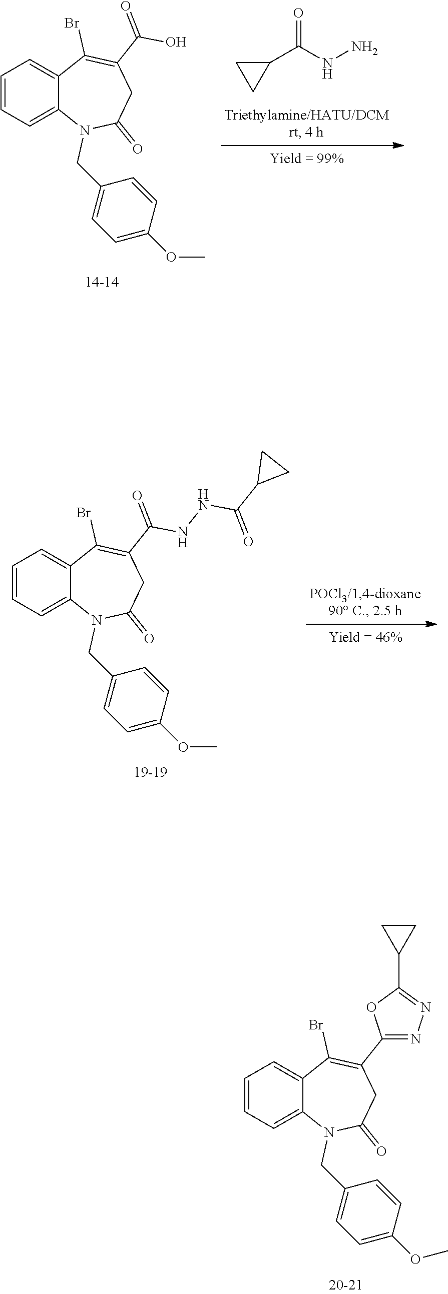 [SMILES: COc1ccc(CN2C(=O)CC(C(=O)O)=C(Br)c3ccccc32)cc1 ; NNC(=O)C1CC1 ; COc1ccc(CN2C(=O)CC(C(=O)NNC(=O)C3CC3)=C(Br)c3ccccc32)cc1 ; COc1ccc(CN2C(=O)CC(c3nnc(C4CC4)o3)=C(Br)c3ccccc32)cc1]

### 5-bromo-4-(5-cyclopropyl-1,3,4-oxadiazol-2-yl)-1-(4-methoxybenzyl)-1,3-dihydro-2H-benzo[b]azepin-2-one (20-21)

5-bromo-1-(4-methoxybenzyl)-2-oxo-2,3-dihydro-1H-benzo[b]azepine-4-carboxylic acid (1.62 g, 4.027 mmol, 1 equiv) in dichloromethane (53 mL) was added Cyclopropanecarboxylic acid hydrazide (604.8 mg, 6.04 mmol, 1.5 equiv), triethylamine (839.7 μL, 6.02 mmol, 1.5 equiv), and HATU (3.06 g, 8.05 mmol, 2 equiv). The mixture was stirred at room temperature for 4 h. Then it was concentrated to give a crude, which was purified by silica gel column chromatography (PE/EA, 1:4) to get the product 1.931 g (purity=93%) as a white solid. Yield: 99%.

TLC R_{f}=0.5 (DCM:MeOH=10:1);

LCMS (m/z)=484.20 (M+H)

5-bromo-N′-(cyclopropanecarbonyl)-1-(4-methoxybenzyl)-2-oxo-2,3-dihydro-1H-benzo[b]azepine-4-carbohydrazide (1.931 g, 4 mmol, 1 equiv) was dissolved in 1,4-dioxane (58.8 mL) and phosphoryl chloride(2.466 mL, 26.46 mmol, 6.57 equiv) was added and the mixture was heated at 90° C. for 2.5 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (857 mg) as a yellow solid. Yield: 46%; Purity: 91%.

TLC R_{f}=0.3 (PE:EA=1:1);

LCMS (m/z)=468.00 (M+H)

### Example 21. Representative Synthesis of Compounds of Formula 20-22

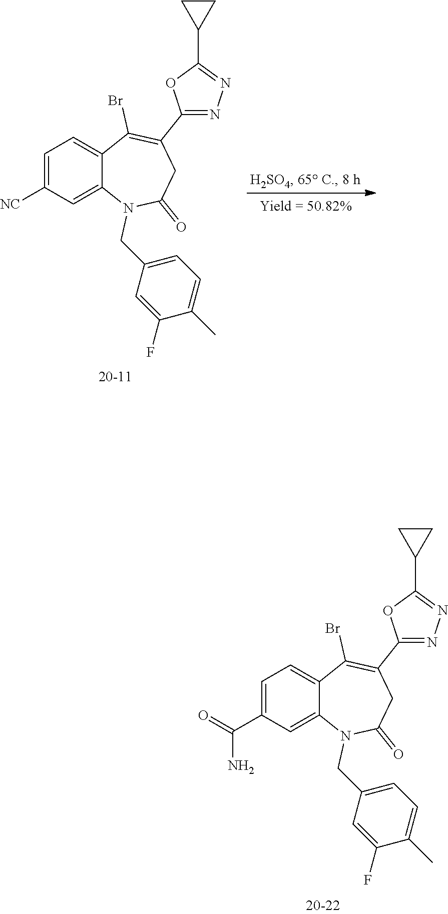 [SMILES: Cc1ccc(CN2C(=O)CC(c3nnc(C4CC4)o3)=C(Br)c3ccc(C#N)cc32)cc1F ; Cc1ccc(CN2C(=O)CC(c3nnc(C4CC4)o3)=C(Br)c3ccc(C(N)=O)cc32)cc1F]

### 5-bromo-4-(5-cyclopropyl-1,3,4-oxadiazol-2-yl)-1-(3-fluoro-4-methylbenzyl)-2-oxo-2,3-dihydro-1H-benzo[b]azepine-8-carboxamide (20-22)

A mixture of 5-bromo-4-(5-cyclopropyl-1,3,4-oxadiazol-2-yl)-1-(3-fluoro-4-methylbenzyl)-2-oxo-2,3-dihydro-11H-benzo[b]azepine-8-carbonitrile (150 mg, 0.304 mmol, 1 eq) and concentrated H₂SO₄ (290.7 μL) were heated to 65° C. for 8 hours. The reaction mixture was gradually poured into ice and the pH was brought to 8-9 by 6 M aq. NaOH solution or aq. NH₄OH. (If the product precipitated, it was filtered off and washed with ice cold H₂O). Otherwise, it was extracted with ethyl acetate and combined organic layer was dried over Na₂SO₄ and concentrated under vacuum. Then, the resulting concentrate was purified on a silica column to get 79 mg (purity=97.05%) white solid compound with a yield of 50.82%.

TLC R_{f}=0.3 (DCM:EA=1:1)

LCMS (m/z)=513.30 (M+H)

### Example 22. Representative Synthesis of Compounds of Formula 20-23

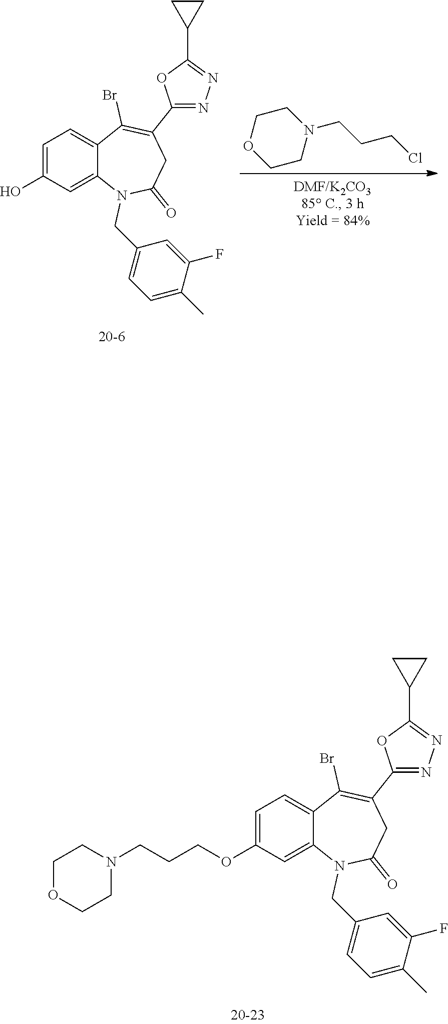 [SMILES: Cc1ccc(CN2C(=O)CC(c3nnc(C4CC4)o3)=C(Br)c3ccc(O)cc32)cc1F ; ClCCCN1CCOCC1 ; Cc1ccc(CN2C(=O)CC(c3nnc(C4CC4)o3)=C(Br)c3ccc(OCCCN4CCOCC4)cc32)cc1F]

### 5-bromo-4-(5-cyclopropyl-1,3,4-oxadiazol-2-yl)-1-(3-fluoro-4-methylbenzyl)-8-(3-morpholinopropoxy)-1,3-dihydro-2H-benzo[b]azepin-2-one (20-23)

5-bromo-4-(5-cyclopropyl-1,3,4-oxadiazol-2-yl)-8-hydroxy-1-(4-methylbenzyl)-1,3-dihydro-2H-benzo[b]azepin-2-one (59 mg, 0.122 mmol, 1 equiv) in DMF (1 mL) was added 4-(3-chloropropyl))morpholine (23 μL, 0.146 mmol, 1.2 equiv) and K₂CO₃ (28 mg, 0.20 mmol, 1.61 equiv), and the mixture was stirred at 85° C. for 3 h. After the reaction mixture had cooled to room temperature, ethyl acetate was added and the diluted solution was slowly poured into room temperature water under a water bath. The resulting solution was extracted with portions of ethyl acetate (3×20 mL). The combined organic extracts were dried over anhydrous Na₂SO₄, filtered and concentrated by rotary evaporation to get the crude compound. Then, the resulting concentrate was purified on a silica column to get the product 40 mg as a yellow solid. Yield: 54%. Purity: 84%.

TLC R_{f}=0.5 (CH₂Cl₂:MeOH=1:1);

LCMS (m/z)=613.10 (M+H)

### Example 23. Representative Synthesis of Compounds of Formula 20-24

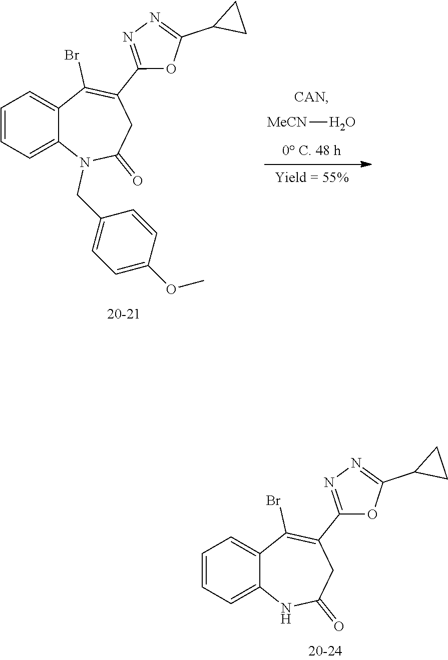

### 5-bromo-4-(5-cyclopropyl-1,3,4-oxadiazol-2-yl)-1,3-dihydro-2H-benzo[b]azepin-2-one(20-24)

To a suspension of 5-bromo-4-(5-cyclopropyl-1,3,4-oxadiazol-2-yl)-1-(4-methoxybenzyl)-1,3-dihydro-2H-benzo[b]azepin-2-one (500 mg, 1.07 mmol, 1 equiv) in 9:1 acetonitrile/water (7 mL) was added CAN (1.74 g, 0.837 mmol, 3 equiv) at 0° C. After stirring at 0° C. for 48 h, the reaction mixture was quenched with water (10 mL) and adjusted pH to 7-9 by using saturated Na₂CO₃. The resulting mixture was extracted with EA. The extract was washed with brine, dried over anhydrous Na₂SO₄, filtered and concentrated by rotary evaporation to get the crude compound. Then, the resulting concentrate was purified on a silica column to get 223 mg (purity=85%) white solid compound with a yield of 55%.

TLC R_{f}=0.25 (PE:EA=1:1);

LCMS (m/z)=347.60 (M+H)

### Example 24. Representative Synthesis of Compounds of Formula 20-25

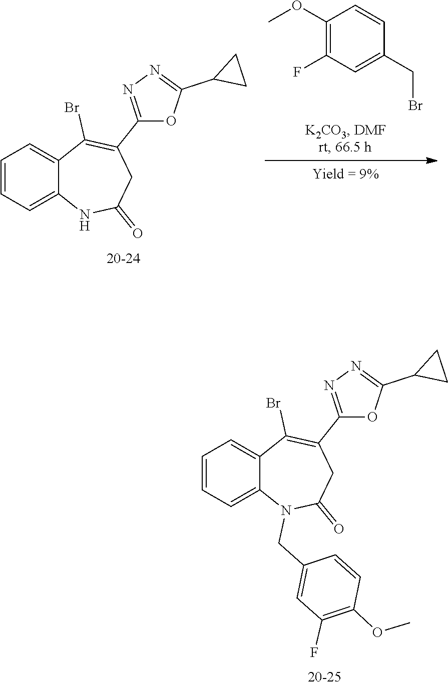

### 5-bromo-4-(5-cyclopropyl-1,3,4-oxadiazol-2-yl)-1-(3-fluoro-4-methoxybenzyl)-1,3-dihydro-2H-benzo[b]azepin-2-one(20-25)

To a stirred solution of 5-bromo-4-(5-cyclopropyl-1,3,4-oxadiazol-2-yl)-1,3-dihydro-2H-benzo[b]azepin-2-one (69 mg, 0.2 mmol, 1 equiv) and K₂CO₃ (83 mg, 0.6 mmol, 3 equiv) in DMF (0.8 mL), was added 4-(bromomethyl)-2-fluoro-1-methoxybenzene (52.5 mg, 0.24 mmol, 1.2 equiv) at ambient temperature. The reaction mixture was then stirred at rt for 66.5 h. The solution was quenched with saturated ammonium chloride and extracted with EA (3×20 mL). The organic solutions were combined, washed with H₂O (2×20 mL) and brine (20 mL), dried over anhydrous Na₂SO₄, filtered and concentrated by rotary evaporation to get the crude compound. Then, the resulting concentrate was purified on a silica column to get 8.5 mg (purity=91%) white solid compound with a yield of 9%.

TLC R_{f}=0.2 (PE:EA=1:1);

LCMS (m/z)=484.30 (M+H)

### Example 25. Representative Synthesis of Compounds of Formula 20-26

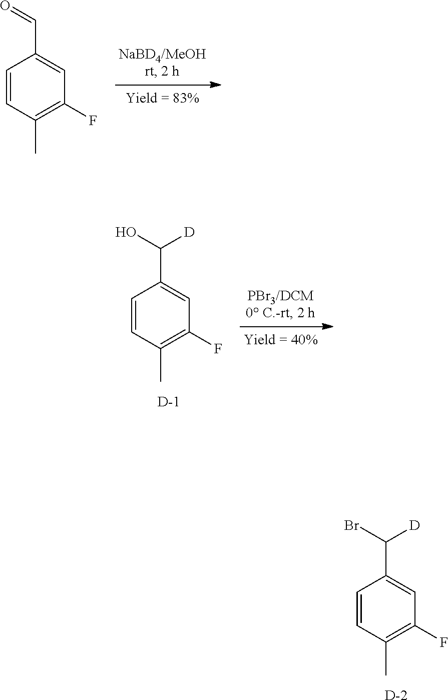

### 4-(bromomethyl-d)-2-fluoro-1-methylbenzene (D-2)

To a stirred suspension of 3-fluoro-4-methylbenzaldehyde (122 μL, 1 mmol, 1 equiv) in MeOH (5 mL) under N₂ at 0° C. was added sodium borodeuteride (84 mg, 2 mmol, 2 equiv) portion-wise. The reaction was warmed to rt and stirred for 2 h. The reaction mixture was then concentrated in vacuo, and subsequently diluted with CH₂Cl₂ (50 mL). The organic phase was washed with H₂O (50 mL), dried (Na₂SO₄), filtered, and concentrated in vacuo to afford title compound D-1 (117 mg, 83% yield, 87% purity) as a colorless solid.

TLC R_{f}=0.35 (PE:EA=4:1);

To a stirred suspension of D-1 (117 mg, 0.83 mmol, 1 equiv) in CH₂Cl₂ (2.5 mL) under N₂ at 0° C. was added phosphorous tribromide (135 mg, 0.5 mmol, 0.5 equiv) dropwise. After 15 min. the mixture was warmed to rt and allowed to stir for 2 h, at which point the reaction mixture was quenched with H₂O (20 mL). The aqueous phase was extracted with CH₂Cl₂ (3×20 mL), the combined organic layers were washed with saturated aqueous sodium bicarbonate (20 mL) and brine (20 mL), dried (Na₂SO₄), filtered and concentrated in vacuo to afford the title compound D-2 (67 mg, 40% yield, 98% purity) as a colorless oil.

TLC R_{f}=0.8 (PE:EA=4:1);

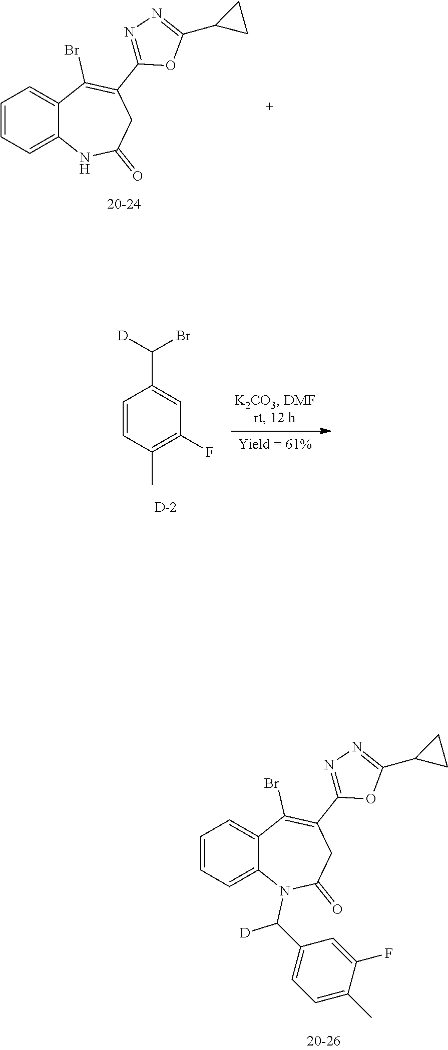

### 5-bromo-4-(5-cyclopropyl-1,3,4-oxadiazol-2-yl)-1-((3-fluoro-4-methylphenyl)methyl-d)-1,3-dihydro-2H-benzo[b]azepin-2-one (20-26)

To a stirred solution of 5-bromo-4-(5-cyclopropyl-1,3,4-oxadiazol-2-yl)-1,3-dihydro-2H-benzo[b]azepin-2-one (50 mg, 0.15 mmol, 1 equiv) and K₂CO₃ (62 mg, 0.45 mmol, 3 equiv) in DMF (0.5 mL), was added 4-(bromomethyl-d)-2-fluoro-1-methylbenzene D-2 (36 mg, 0.18 mmol, 1.2 equiv) at ambient temperature. The reaction mixture was then stirred at rt for 12 h. The solution was quenched with saturated ammonium chloride and extracted with EA (3×20 mL). The organic solutions were combined, washed with H₂O (2×20 mL) and brine (20 mL), dried over anhydrous Na₂SO₄, filtered and concentrated by rotary evaporation to get the crude compound. Then, the resulting concentrate was purified on a silica column to get 43 mg (purity=98%) white solid compound with a yield of 61%.

TLC R_{f}=0.5 (PE:EA=1:1);

LCMS (m/z)=469.20 (M+H)

### Example 26. Representative Synthesis of Compounds of Formula 20-27

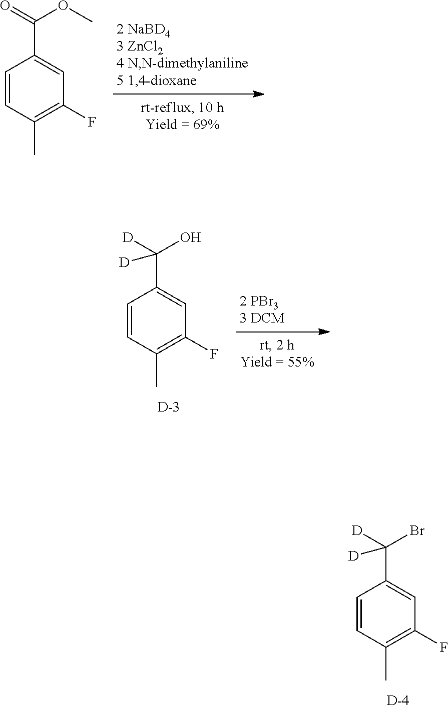

### 4-(bromomethyl-d2)-2-fluoro-1-methylbenzene (D-4)

To a 25 mL round-bottom flask was added sodium borodeuteride (168 mg, 4 mmol, 2.00 equiv) and ZnCl₂ (272 mg, 2 mmol, 1.00 equiv). Subsequently, anhydrous 1,4-dioxane (10 mL) and N,N-dimethylaniline (0.254 mL, 2 mmol, 1.0 equiv) were added and the solution allowed to stir at rt. After 3 h, methyl 3-fluoro-4-methylbenzoate (336 mg, 2 mmol, 1.0 equiv) was added and the solution was stirred at rt for 10 min. The reaction mixture was heated to reflux for 10 h, at which point the reaction mixture was cooled to 0° C. and 1.0 MI aqueous HCl (15 mL) and CH₂Cl₂ (15 mL) were added. The aqueous phase was extracted and the organic phase washed with water (15 mL) and brine (15 mL), dried (Na₂SO₄), filtered, and concentrated in vacuo. Then, the resulting concentrate was purified on a silica column to get 196 mg (purity=98%) white solid compound with a yield of 69%.

TLC R_{f}=0.2 (PE:EA=4:1);

To a stirred suspension of (3-fluoro-4-methylphenyl)methan-d₂-ol D-3 (196 mg, 1.38 mmol, 1.00 equiv) in —CH₂Cl₂ (5 mL.) under N₂ at 0° C. was added phosphorous tribromide (0.065 mL, 0.69 mmol, 0.500 equiv) dropwise. After 15 min, the mixture was warmed to rt and allowed to stir for 2 h, at which point the reaction mixture was quenched with H₂O (0.10 mL). The aqueous phase was extracted with CH₂Cl₂ (3×10 mL), the combined organic layers were washed with saturated aqueous sodium bicarbonate (10 mL) and brine (10 mL), dried (Na₂SO₄), filtered and concentrated in vacuo to afford title compound 157 mg (purity=99%) yellow oil compound with a yield of 55%.

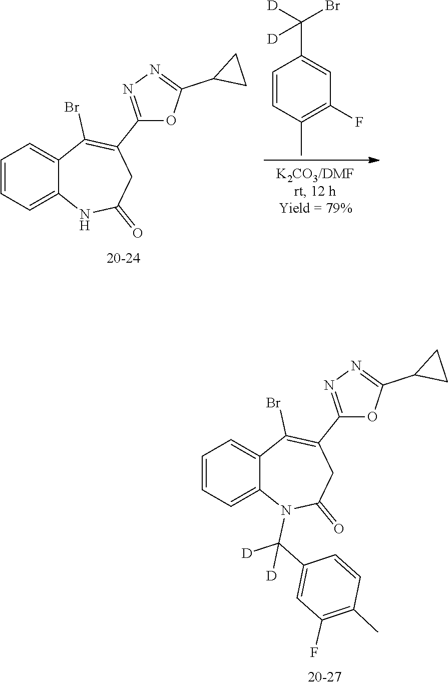

### 5-bromo-4-(5-cyclopropyl-1,3,4-oxadiazol-2-yl)-1-((3-fluoro-4 methylphenyl)methyl-d2)-1,3-dihydro-2H-benzo[b]azepin-2-one (20-27)

To a stirred solution of 5-bromo-4-(5-cyclopropyl-1,3,4-oxadiazol-2-yl)-1,3-dihydro-2H-benzo[b]azepin-2-one (69 mg, 0.2 mmol, 1 equiv) and K₂CO₃ (83 mg, 0.6 mmol, 3 equiv) in DMF (0.8 mL), was added 4-(bromomethyl-d₂)-2-fluoro-1-methylbenzene (49 mg, 0.24 mmol, 1.2 equiv) at ambient temperature. The reaction mixture was then stirred at rt for 12 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 75 mg (purity=99%) white solid compound with a yield of 79%.

TLC R_{f}=0.5 (PE:EA=1:1);

LCMS (m/z)=472.20 (M+H)

### Example 27. Representative Synthesis of Compounds of Formula 20-28

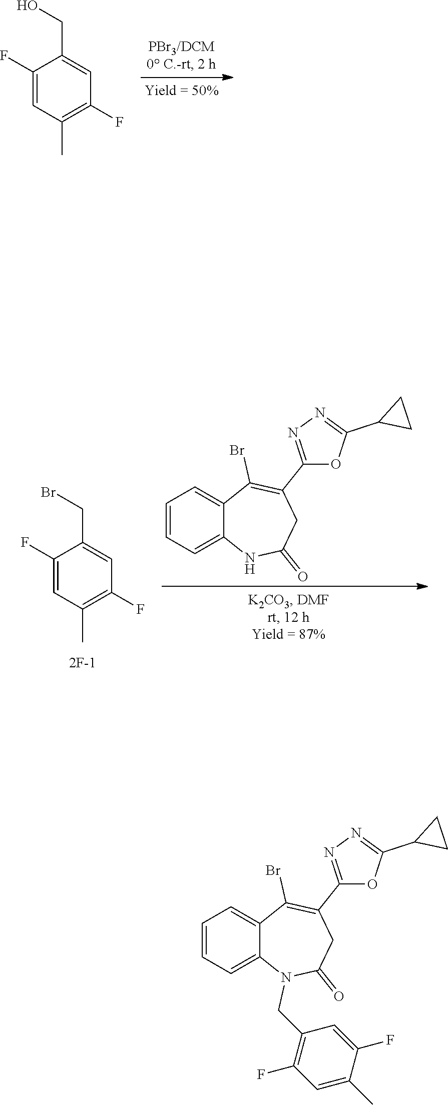

### 5-bromo-4-(5-cyclopropyl-1,3,4-oxadiazol-2-yl)-1-(2,5-difluoro-4-methylbenzyl)-1,3-dihydro-2H-benzo[b]azepin-2-one (20-28)

To a stirred suspension of (2,5-difluoro-4-methylphenyl)methanol (100 ng, 0.63 mmol, 1 equiv) in CH₂Cl₂ (2 ml) under N₂ at 0° C. was added phosphorous tribromide (26 mg, 0.32 mmol, 0.5 equiv) dropwise. After 15 min, the mixture was warmed to rt and allowed to stir for 2 h, at which point the reaction mixture was quenched with 1-120 (20 mL). The aqueous phase was extracted with CH₂Cl₂ (3×20 mL), the combined organic layers were washed with saturated aqueous sodium bicarbonate (20 mL) and brine (20 mL), dried (Na₂SO₄), filtered and concentrated in vacuo to afford the title compound (70 mg, 50% yield) as a colorless oil.

TLC R_{f}=0.9 (PE:EA=1:1);

To a stirred solution of 5-bromo-4-(5-cyclopropyl-1,3,4-oxadiazol-2-yl)-1,3-dihydro-2H-benzo[b]azepin-2-one (91 mg, 0.26 mmol, 1 equiv) and K₂CO₃ (109 mg, 0.79 mmol, 3 equiv) in DMF (0.9 mL), was added 1-(bromomethyl)-2,5-difluoro-4-methylbenzene 2F-1 (70 mg, 0.32 mmol, 1.2 equiv) at ambient temperature. The reaction mixture was then stirred at rt for 12 h. The solution was quenched with saturated ammonium chloride and extracted with EA (3×20 mL). The organic solutions were combined, washed with H₂O (2×20 mL) and brine (20 mL), dried over anhydrous Na₂SO₄, filtered and concentrated by rotary evaporation to get the crude compound. Then, the resulting concentrate was purified on a silica column to get 109 mg (purity=97%) white solid compound with a yield of 87%.

TLC R_{f}=0.6 (PE:EA=2:1);

LCMS (m/z)=486.20 (M+H)

### Example 28. Representative Synthesis of Compounds of Formula 20-29

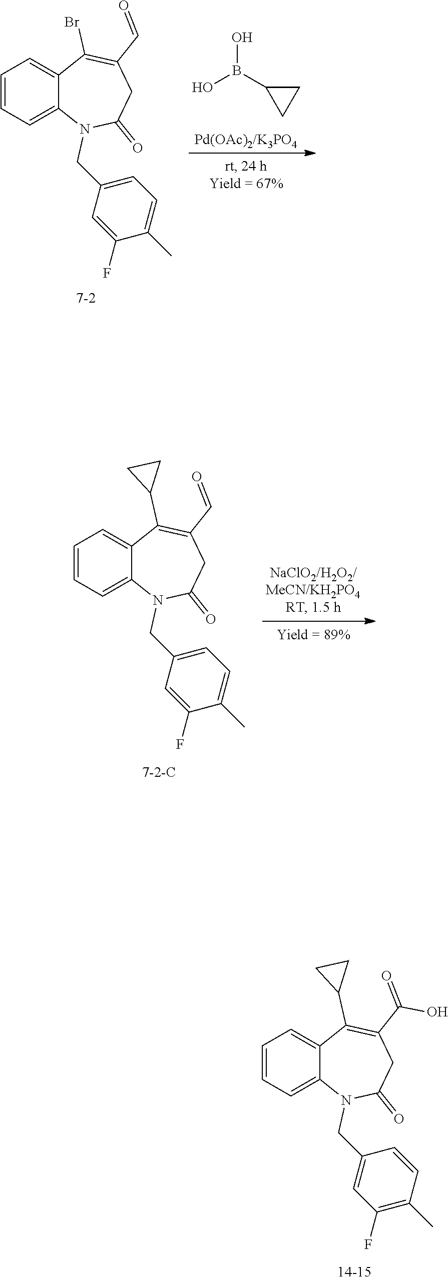 [SMILES: Cc1ccc(CN2C(=O)CC(C=O)=C(Br)c3ccccc32)cc1F ; OB(O)C1CC1 ; Cc1ccc(CN2C(=O)CC(C=O)=C(C3CC3)c3ccccc32)cc1F ; Cc1ccc(CN2C(=O)CC(C(=O)O)=C(C3CC3)c3ccccc32)cc1F]

### 9-cyclopropyl-5-(3-fluoro-4-methylbenzyl)-6-oxo-6,7-dihydro-5H-benzo[7]annulene-8-carboxylic acid (14-15)

K₃PO₄ (2 M in H₂O, 1.5 mL) and Pd(OAc)₂ (15 mg, 5 mol %) were successively added to a solution of 5-bromo-1-(3-fluoro-4-methylbenzyl)-2-oxo-2,3-dihydro-1H-benzo[b]azepine-4-carbaldehyde (582 mg, 1.5 mmol, 1 equiv) and cyclopropylboronic acid (155 mg, 1.8 mmol, 1.2 equiv) in THF (3 mL) under N₂. The reaction mixture was vigorously stirred at room temperature for 24 h. The reaction mixture was diluted with H₂O (10 mL) and extracted with EA (3×10 ml). The combined organic fraction was dried and concentrated. The residue was purified by chromatography to afford the product (155 mg) as a yellow solid. Yield: 67%; Purity: 92%.

TLC R_{f}=0.4 (PE:EA=2:1);

LCMS (m/z)=350.60 (M+H)

Reactions were carried out by addition of aqueous NaClO₂ (160 mg, 1.78 mmol, 1.76 equiv) to a solution of 9-cyclopropyl-5-(3-fluoro-4-methylbenzyl)-6-oxo-6,7-dihydro-5H-benzo[7]annulene-8-carbaldehyde (353 mg, 1.01 mmol, 1 equiv) and (105 μL, 1.03 mmol, 1.02 equiv) of 30% H₂O₂ in aqueous acetonitrile, at 0° C., buffered with KH₂PO₄ at pH 4˜3. The reaction mixture was stirred for 1.5 h. After complete reaction, a small amount of the Na₂SO₃ (˜0.5 g) was added to destroy the unreacted NaClO₂ and H₂O₂. Acidification with 10% HCl, the mixture was diluted with H₂O (10-20 mL). The compound was extracted with EA (10 ml). The organic layer was separated and extracted with water and brine and then dried over anhydrous Na₂SO₄, filtered and concentrated by rotary evaporation to get the crude compound. Then, the resulting concentrate was purified on a silica column to get 329 mg (purity=84%) white solid compound with a yield of 89%.

TLC R_{f}=0.2 (DCM:MeOH=10:1);

LCMS (m/z)=366.70 (M+H)

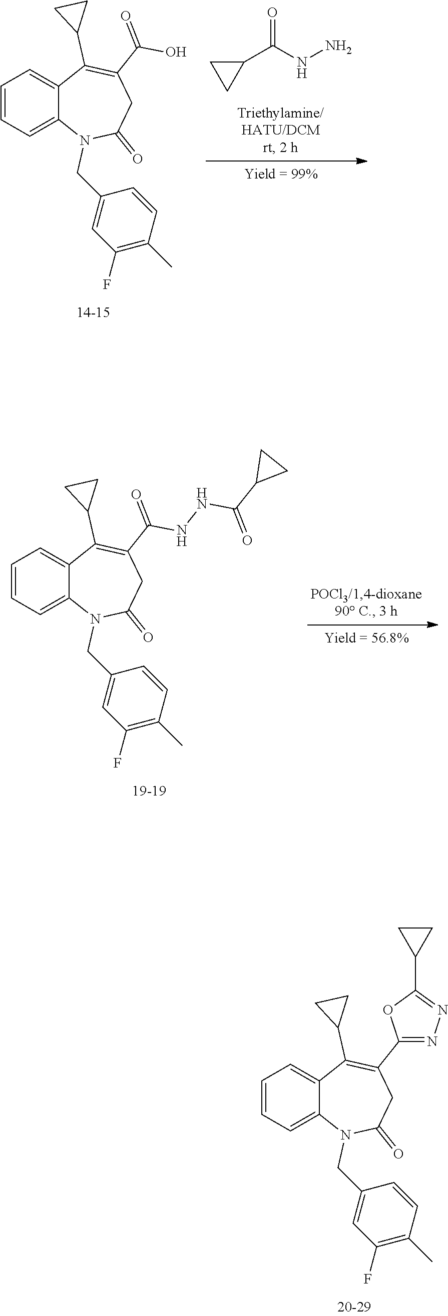

### 9-cyclopropyl-8-(5-cyclopropyl-1,3,4-oxadiazol-2-yl)-5-(3-fluoro-4-methylbenzyl)-5,7-dihydro-6H-benzo[7]annulen-6-one (20-29)

5-cyclopropyl-1-(3-fluoro-4-methylbenzyl)-2-oxo-2,3-dihydro-1H-benzo)[b]azepine-4-carboxylic acid (329 mg, 0.9 mmol, 1 equiv) in dichloromethane (8 mL) was added Cyclopropanecarboxylic acid hydrazide (135 mg, 1.35 mmol, 1.5 equiv), triethylamine (186.8 μL, 1.35 mmol, 1.5 equiv), and HATU (684 mg, 1.8 mmol, 2 equiv). The mixture was stirred at room temperature for 2 h. Then it was concentrated to give a crude, which was purified by silica gel column chromatography (PE/EA, 1:4) to get the product 398 mg (purity=87%) as a white solid. Yield: 99%.

TLC R_{f}=0.35 (DCM:MeOH=10:1)

LCMS (m/z)=448.90 (M+H)

N′-(cyclopropanecarbonyl)-9-cyclopropyl-5-(3-fluoro-4-methylbenzyl)-6-oxo-6,7-dihydro-5H-benzo[7]annulene-8-carbohydrazide (398 mg, 0.75 mmol, 1 equiv) was dissolved in 1,4-dioxane (9 mL) and phosphoryl chloride (545.5 μL, 5.85 mmol, 6.57 equiv) was added and the mixture was heated at 90° C. for 3 h, then cooled to room temperature and diluted with ethyl acetate. The mixture was washed with NaHCO₃ and brine, dried over sodium sulfate, filtered and concentrated in vacuo. The residue was purified by silica gel column chromatography to get the product (217 mg) as a white solid. Yield: 56.8%; Purity: 83%.

TLC R_{f}=0.7 (DCM:MeOH=10:1);

LCMS (m/z)=430.90 (M+H)

### Example 29. Representative Synthesis of Compounds of Formula 20-30

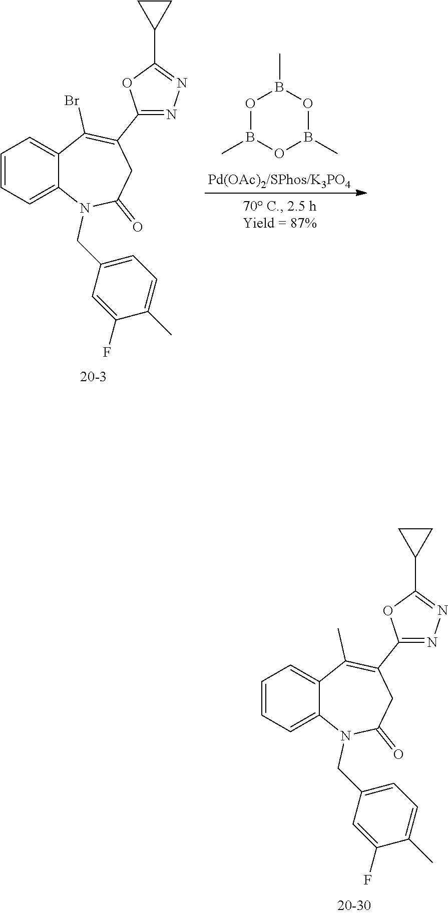

### (5-cyclopropyl-1,3,4-oxadiazol-2-yl)-1-(3-fluoro-4-methylbenzyl)-5-methyl-1,3-dihydro-2H-benzo[b]azepin-2-one(20-30)

A round-bottom flask was charged with 5-bromo-4-(5-cyclopropyl-1,3,4-oxadiazol-2-yl)-1-(3-fluoro-4-methylbenzyl)-1,3-dihydro-2H-benzo[b]azepin-2-one (100 mg, 0.21 mmol, 1 equiv). Pd(OAc)₂ (2.4 mg, 0.011 mmol, 0.05 equiv), SPhos (8.8 mg, 0.021 mmol, 0.1 equiv), K₃PO₄ (226 mg, 1.07 mmol, 5 equiv) and dioxane:H₂O (1.5:0.15 mL). Then the trimethylboroxine 3.5M in THF (183 μL, 0.64 mmol, 3 equiv) was added to the mixture under N₂ at room temperature, and heated to 70° C. for 2.5 h. The reaction mixture was filtered with Celite, followed by extraction with ethyl acetate. The combined organic fraction was dried and concentrated. The residue was purified by chromatography to afford the product (74 mg) as a yellow solid. Yield: 87%; Purity: 90%.

TLC R_{f}=0.3 (PE:EA=2:1);

LCMS (m/z)=404.60 (M+H)

### Example 30. Representative Synthesis of Compounds of Formula 20-32

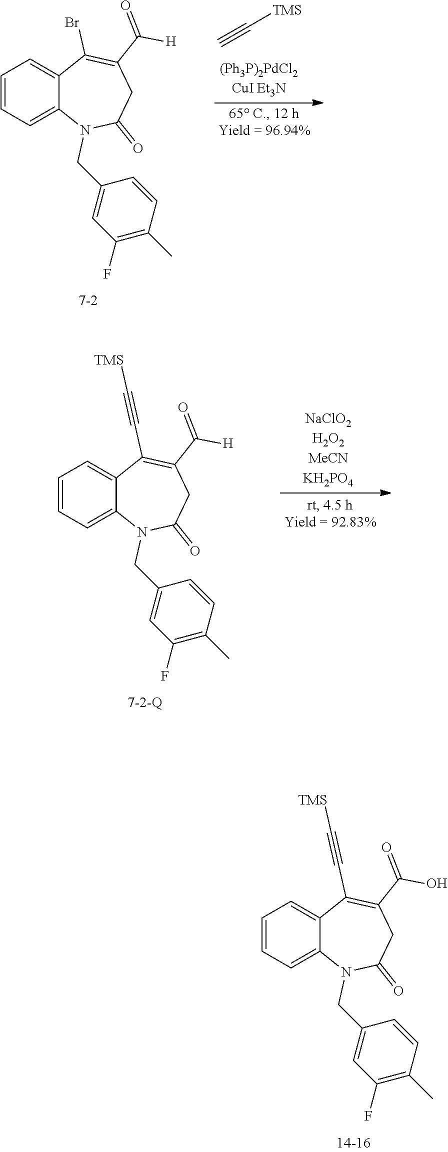

### 1-(3-fluoro-4-methylbenzyl)-2-oxo-5-((trimethylsilyl)ethynyl)-2,3-dihydro-1H-benzo[b]azepine-4-carboxylic acid (14-16)

A mixture of 5-bromo-1-(3-fluoro-4-methylbenzyl)-2-oxo-2,3-dihydro-1H-benzo[b]azepine-4-carbaldehyde (500 ng, 1.287 mmol, 1.0 equiv), (Ph₃P)₂PdCl₂ (11.323 mg, 0.02575 mmol, 0.02 equiv) and CuI (2.453 mg, 0.01287 mmol, 0.01 equiv) in a flask was degassed for 10 min, then Et₃N (0.2 M for substrate) was added under argon atmosphere. To this solution ethynyl trimethylsilane (910 μL, 6.440 mmol, 5 equiv) was added by syringe. The mixture was warmed to 65° C. for 12 hours. After cooling to room temperature, the reaction mixture was filtrated through celite. The filtrate was concentrated and purified by chromatography to get the product 506 mg (purity=63.78%) as an orange solid. Yield: 96.94%.

TLC R_{f}=0.7 (PE:EA=2:1)

LCMS (m/z)=406.40 (M+H)

Reactions were carried out by addition of aqueous NaClO₂ (197.64 mg, 2.196 mmol, 1.76 eq) to a solution of 1-(3-fluoro-4-methylbenzyl)-2-oxo-5-((trimethylsilyl)ethynyl)-2,3-dihydro-1H-benzo[b]azepine-4-carbaldehyde (506 mg, 1.247 mmol, 1 eq) and (43.27 mg, 1.27 mmol, 1.02 eq) 30% H₂O₂ in aqueous acetonitrile, at 0° C., buffered with KH₂PO₄ at pH 4.3. The reaction mixture was stirred 4.5 h. After complete reaction, a small amount of the Na₂SO₃ (˜0.5 g) was added to destroy the unreacted HOCl and H₂O₂. Acidification with 10% HCl, the mixture was diluted with H₂O (10-20 mL). The compound was extracted with Et₂O (10 mL). The organic layer was separated and extracted with water and brine and then dried over anhydrous Na₂SO₄, filtered and concentrated by rotary evaporation to get the crude compound. Then, the resulting concentrate was purified on a silica column to get 488 mg (purity=79.22%) yellow solid compound with a yield of 92.83%.

TLC R_{f}=0.3 (DCM:MeOH=10:1)

LCMS (m/z)=422.10 (M+H)

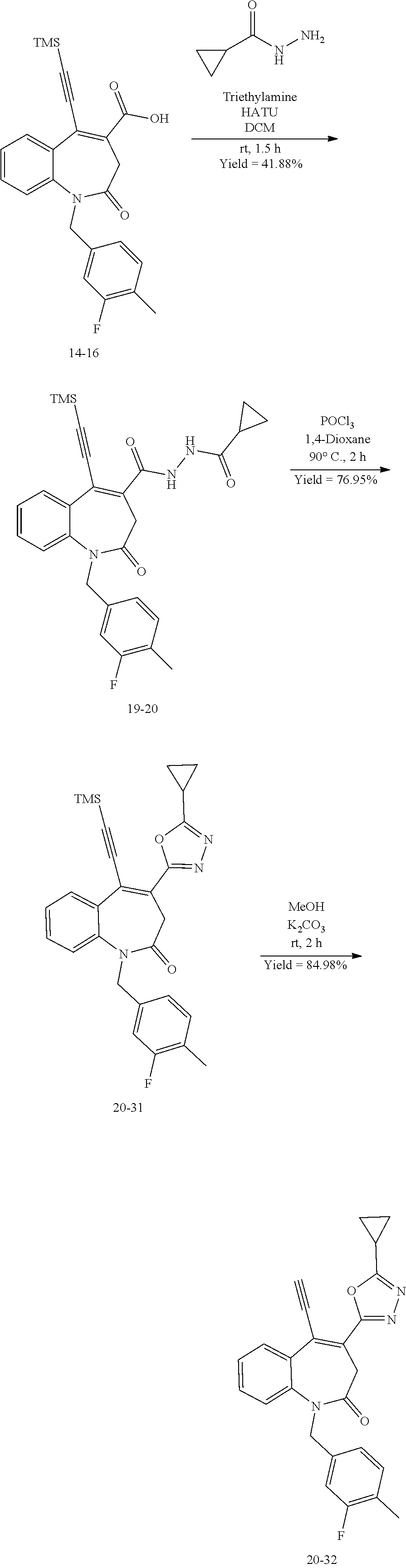 [SMILES: Cc1ccc(CN2C(=O)CC(C(=O)O)=C(C#C[Si](C)(C)C)c3ccccc32)cc1F ; NNC(=O)C1CC1 ; Cc1ccc(CN2C(=O)CC(C(=O)NNC(=O)C3CC3)=C(C#C[Si](C)(C)C)c3ccccc32)cc1F ; Cc1ccc(CN2C(=O)CC(c3nnc(C4CC4)o3)=C(C#C[Si](C)(C)C)c3ccccc32)cc1F ; C#CC1=C(c2nnc(C3CC3)o2)CC(=O)N(Cc2ccc(C)c(F)c2)c2ccccc21]

### 4-(5-cyclopropyl-1,3,4-oxadiazol-2-yl)-5-ethynyl-1-(3-fluoro-4-methylbenzyl)-1,3-dihydro-2H-benzo[b]azepin-2-one (20-32)

1-(3-fluoro-4-methylbenzyl)-2-oxo-5-((trimethylsilyl)ethynyl)-2,3-dihydro-1-benzo[b]azepine-4-carboxylic acid (100 mg, 0.237 mmol, 1 equiv) in dichloromethane (2.1 mL) was added Cyclopropanecarboxylic acid hydrazide (35.625 mg, 0.355 mmol, 1.5 equiv), triethylamine (49.46 μL, 0.355 mmol, 1.5 equiv), and HATU (180.405 mg, 0.474 mmol, 2 equiv). The mixture was stirred at room temperature for 1.5 h. Then it was concentrated to give a crude, which was purified by silica gel column chromatography to get the product 50 mg (purity=95.28%) as a yellow solid. Yield: 41.88%.

TLC R_{f}=0.45 (DCM:MeOH=10:1)

LCMS (m/z)=504.30 (M+H)

N′-(cyclopropanecarbonyl)-1-(3-fluoro-4-methylbenzyl)-2-oxo-5-((trimethylsilyl)ethynyl)-2,3-dihydro-1H-benzo[b]azepine-4-carbohydrazide (50 mg, 0.099 mmol, 1 equiv) was dissolved in 1,4-dioxane (1.33 mL) and phosphoryl chloride (60.79 μL, 0.652 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (37 mg) as a yellow solid. Yield: 76.95%; Purity: 92.86%.

TLC R_{f}=0.7 (DCM:MeOH=10:1);

LCMS (m/z)=486 (M+H)

4-(5-cyclopropyl-1,3,4-oxadiazol-2-yl)-1-(3-fluoro-4-methylbenzyl)-5-((trimethylsilyl)ethynyl)-1,3-dihydro-2H-benzo[b]azepin-2-one (72 mg, 0.148 mmol, 1 eq) was dissolved in MeOH (0.2 M for substrate) and K₂CO₃ (2.049 mg, 0.0148 mmol, 0.1 equiv) was added. Desilylation was conducted at room temperature for 2 hours and concentrated. The final product was purified by chromatography to get the product 52 mg (purity=94.84%) as an orange solid. Yield: 84.98%

TLC R_{f}=0.6 (PE:EA=1:1)

LCMS (m/z)=414.10 (M+H)

### Example 31. Representative Synthesis of Compounds of Formula 22

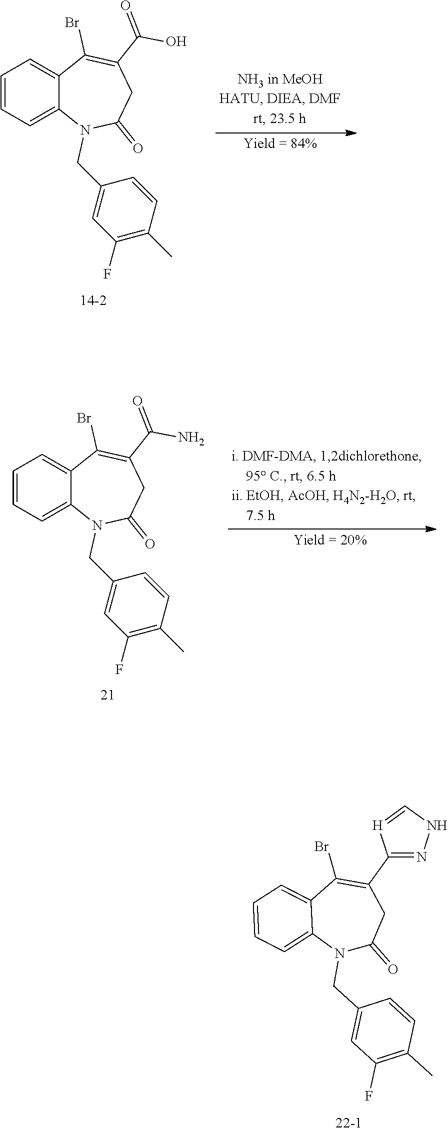

### 5-bromo-1-(3-fluoro-4-methylbenzyl)-4-(1H-1,2,4-triazol-3-yl)-1,3-dihydro-2H-benzo[b]azepin-2-one (22-1)

HATU (127 mg, 0.335 mmol, 2.7 equiv) and DIPEA (64.6 μL, 0.372 mmol, 3 equiv) were added to a solution of 5-bromo-1-(3-fluoro-4-methylbenzyl)-2-oxo-2,3-dihydro-1H-benzo[b]azepine-4-carboxylic acid (14-2) (536 ng, 3.04 mmol) in DMF (0.6 mL) at 0° C. The mixture was stirred for 30 min followed by the addition of ammonia (7 M in MeOH) (135.5 μL, 0.95 mmol, 7.6 equiv). The reaction was then stirred at room temperature overnight. The reaction mixture was concentrated and purified by flash column chromatography to afford the title compound (quantity: 42 mg, yield: 84%, purity: 96%) as a white solid.

TLC R_{f}=0.5 (DCM:MeOH=10:1)

LCMS (m/z)=404.90 (M+H)

5-bromo-1-(3-fluoro-4-methylbenzyl)-2-oxo-2,3-dihydro-1H-benzo[b]azepine-4-carboxamide (21) (100 mg, 0.248 mmol) in dimethylformamide dimethyl acetal (DMF-DMA, 0.33 mL, 2.48 mmol) was heated to 95° C. After heating for −30 min at this temperature, the reaction was cooled and concentrated in vacuo. Then it was concentrated to give a crude, which was azeotroped twice with water (40 mL) to ensure complete removal of any residual DMF-DMA. The resulting intermediate containing the crude DMF-DMA adduct was dissolved in 0.25 mL of ethanol and was immediately used in the following step. In a separate flask was prepared a mixture of ethanol (0.75 mL) and acetic acid (0.25 mL), and the resulting solution was cooled in an ice bath. Once cooled, hydrazine hydrate (150.5 μL, 2.48 mmol) was added dropwise, followed by a dropwise addition of the previously prepared ethanol solution of the crude DMF-DMA adduct via cannula over ˜15 min with stirring. After the addition was complete, the resulting mixture was allowed to warm to room temperature and stir for ˜4 h. The reaction mixture was concentrated in vacuo to remove the ethanol, which was purified by silica gel column chromatography to get the product 21 mg as a white solid. Yield: 20%. Purity: 90%.

TLC R_{f}=0.75 (DCM:MeOH=10:1);

LCMS (m/z)=429.40 (M+H)

### Example 32. Representative Synthesis of Compounds of Formula 24

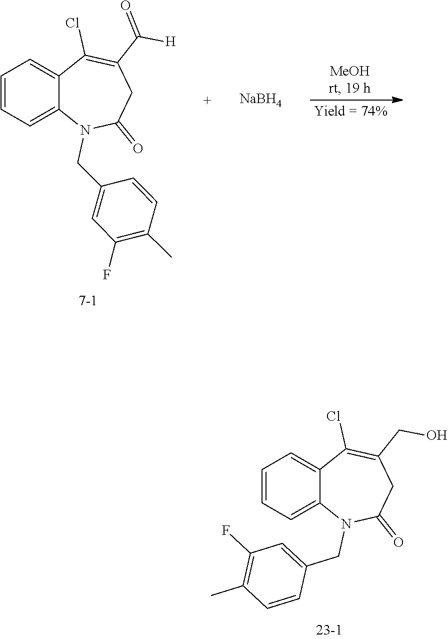 [SMILES: Cc1ccc(CN2C(=O)CC(C=O)=C(Cl)c3ccccc32)cc1F ; "NaBH4" ; Cc1ccc(CN2C(=O)CC(CO)=C(Cl)c3ccccc32)cc1F]

### 5-chloro-1-(3-fluoro-4-methylbenzyl)-4-(hydroxymethyl)-1,3-dihydro-2H-benzo[b]azepin-2-one (23-1)

To a solution of methyl 5-chloro-1-(3-fluoro-4-methylbenzyl)-2-oxo-2,3-dihydro-1H-benzo[b]azepine-4-carbaldehyde 7-1 (50 mg, 0.14 mmol, 1 equiv) in MeOH (0.5 mL), was added NaBH₄ (0.43 mL, 3.11 mmol, 1.2 equiv) at 0° C. The reaction mixture was then stirred at r.t. for 19 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column get 36 mg (purity=99%) of the light yellow oil compound with a yield of 74%.

TLC R_{f}=0.25 (EA:PE=1:2)

LCMS (m/z)=346.00 (M+H)

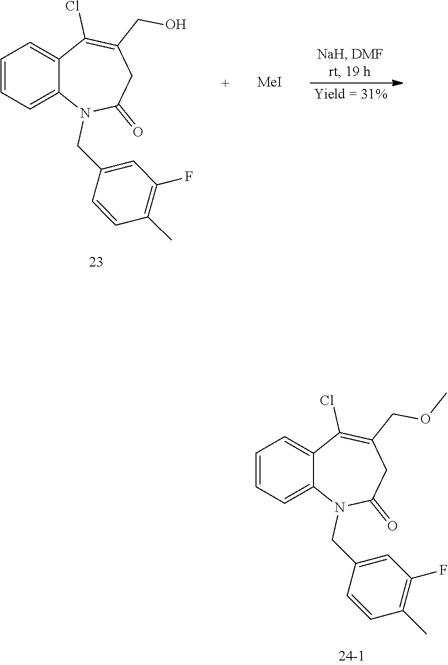 [SMILES: "MeI" ; Cc1ccc(CN2C(=O)CC(CO)=C(Cl)c3ccccc32)cc1F ; COCC1=C(Cl)c2ccccc2N(Cc2ccc(C)c(F)c2)C(=O)C1]

### 5-chloro-1-(3-fluoro-4-methylbenzyl)-4-(methoxymethyl)-1,3-dihydro-2H-benzo[b]azepin-2-one (24-1)

To a stirred solution of methyl (1-(5-chloro-1-(3-fluoro-4-methylbenzyl)-4-(hydroxymethyl)-1,3-dihydro-2H-benzo[b]azepin-2-one) 23 (200 mg, 0.58 mmol, 1 equiv) and Mel (72 μL, 1.16 mmol, 2 equiv) in DMF (2 mL) was added NaH 60% dispersion in mineral oil (28 mg, 1.16 mmol, 2 equiv) at ambient temperature under N₂. The reaction mixture was then stirred at room temperature r.t for 19 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 65 mg (purity=98%) white solid compound with a yield of 31%.

TLC R_{f}=0.3 (PE:EA=2:1)

LCMS (m/z)=360.00 (M+H)

¹H NMR (400 MHz, DMSO) δ 7.76-7.62 (m, 1H), 7.52 (d, J=8.1 Hz, 1H), 7.49-7.38 (m, 1H), 7.27 (t, J=7.3 Hz, 1H), 7.11 (t, J=8.0 Hz, 1H), 6.73 (t, J=8.0 Hz, 2H), 5.33 (d, J=16.1 Hz, 1H), 4.91 (d, J=16.1 Hz, 1H), 4.37 (d, J=12.9 Hz, 1H), 4.23 (d, J=13.0 Hz, 1H), 3.33-3.21 (m, 4H), 2.70 (d, J=12.8 Hz, 1H), 2.12 (s, 3H).

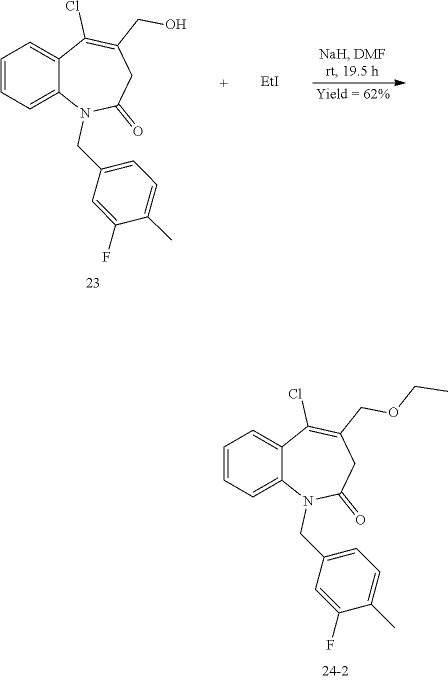 [SMILES: "EtI" ; Cc1ccc(CN2C(=O)CC(CO)=C(Cl)c3ccccc32)cc1F ; CCOCC1=C(Cl)c2ccccc2N(Cc2ccc(C)c(F)c2)C(=O)C1]

### 5-chloro-4-(ethoxymethyl)-1-(3-fluoro-4-methylbenzyl)-1,3-dihydro-2H-benzo[b]azepin-2-one (24-2)

To a stirred solution of methyl (5-chloro-1-(3-fluoro-4-methylbenzyl)-4-(hydroxymethyl)-1,3-dihydro-2H-benzo[b]azepin-2-one) 23 (100 mg, 0.29 mmol, 1 equiv) and EtI (47 μL, 0.58 mmol, 2 equiv) in DMF (1 mL), was added NaH 60% dispersion in mineral oil (14 mg, 0.58 mmol, 2 equiv) at ambient temperature under N₂ atmosphere. The reaction mixture was then stirred at room temperature r.t. for 19 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 67 mg (purity=99%) yellow solid compound with a yield of 62%.

TLC R_{f}=0.8 (PE:EA=2:1)

LCMS (m/z)=374.60 (M+H)

### Example 33. Representative Synthesis of Compounds of Formula 26-1

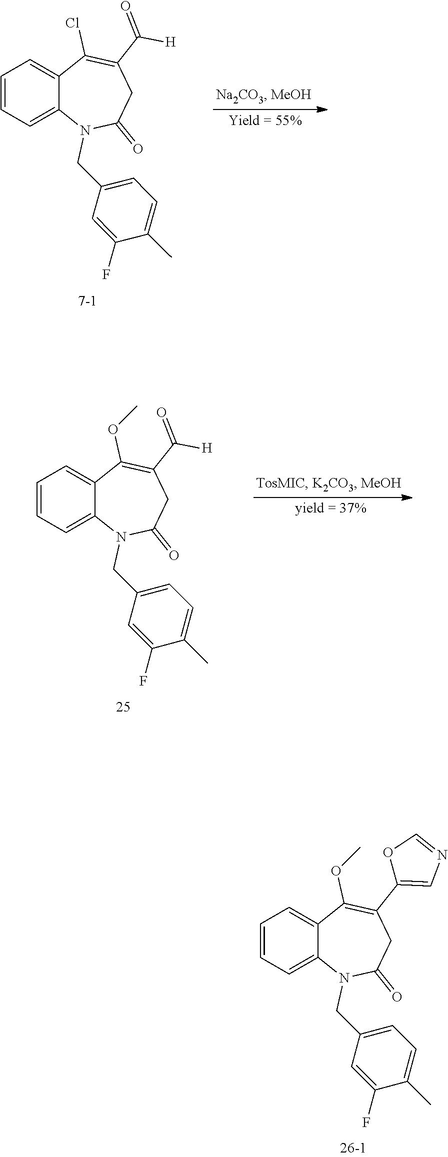 [SMILES: Cc1ccc(CN2C(=O)CC(C=O)=C(Cl)c3ccccc32)cc1F ; COC1=C(C=O)CC(=O)N(Cc2ccc(C)c(F)c2)c2ccccc21 ; COC1=C(c2cnco2)CC(=O)N(Cc2ccc(C)c(F)c2)c2ccccc21]

### 1-(3-fluoro-4-methylbenzyl)-5-methoxy-4-(oxazol-5-yl)-1,3-dihydro-2H-benzo[b]azepin-2-one (26-1)

To a stirred solution of 7-1 (0.29 mmol) in MeOH (2.5 mL), sodium carbonate (307.4 mg, 2 mol %) was added and the reaction mixture was refluxed (MeOH=65° C.) for 5 h. On completion, the reaction mixture was filtered, washed with methanol and dried in vacuo. The residue was taken in ethyl acetate (20 mL) and washed with water, dried over anhydrous Na₂SO₄ and evaporated in vacuo. The residue was purified on a silica column to get 54 mg (purity=94%) yellow solid compound with a yield of 55%.

TLC R_{f}=0.4 (PE:EA=2:1)

LCMS (m/z)=340.60 (M+H)

The toluenesulfonylmethyl isocyanide (TosMIC, 234 mg, 0.35 mmol) was placed in a dry round-bottom flask and dry MeOH (9 mL) added under an argon atmosphere. At it, solid K₂CO₃ (92.2 g, 0.87 mmol) and the 1-(3-fluoro-4-methylbenzyl)-5-methoxy-4-(oxazol-5-yl)-1,3-dihydro-2H-benzo[b]azepin-2-one 25 (100 mg, 0.29 mmol) were added to the mixture, and the mixture was heated to reflux for 1.5 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄. The solvent was then removed under reduced pressure and the residue purified by flash chromatography (silica gel) to give the pure yellow solid (purity=99%, 41 mg, yield=37%).

TLC R_{f}=0.3 (EA:PE=1:2)

LCMS (m/z)=379.60 (M+H)

### Example 34. Representative Synthesis of Compounds of Formula 28

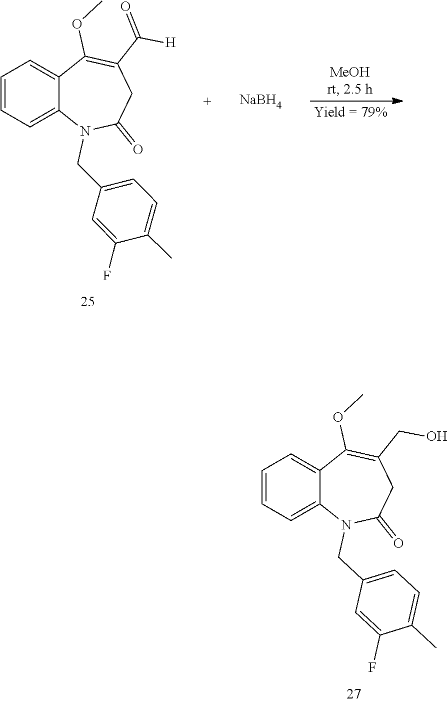 [SMILES: COC1=C(C=O)CC(=O)N(Cc2ccc(C)c(F)c2)c2ccccc21 ; "NaBH4" ; COC1=C(CO)CC(=O)N(Cc2ccc(C)c(F)c2)c2ccccc21]

### 1-(3-fluoro-4-methylbenzyl)-4-(hydroxymethyl)-5-methoxy-1,3-dihydro-2H-benzo[b]azepin-2-one (27)

To a solution of methyl 1-(3-fluoro-4-methylbenzyl)-5-methoxy-2-oxo-2,3-dihydro-1H-benzo[b]azepine-4-carbaldehyde 25 (300 ng, 0.88 mmol, 1 equiv) in MeOH (3 mL), was added NaBH₄ (166 mg, 4.4 mmol, 5 equiv) at 0° C. The reaction mixture was then stirred at r.t for 2.5 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 237 mg (purity=99%) of the white solid compound with a yield of 79%.

TLC R_{f}=0.2 (EA:PE=1:2)

LCMS (m/z)=324.50 (M+H)

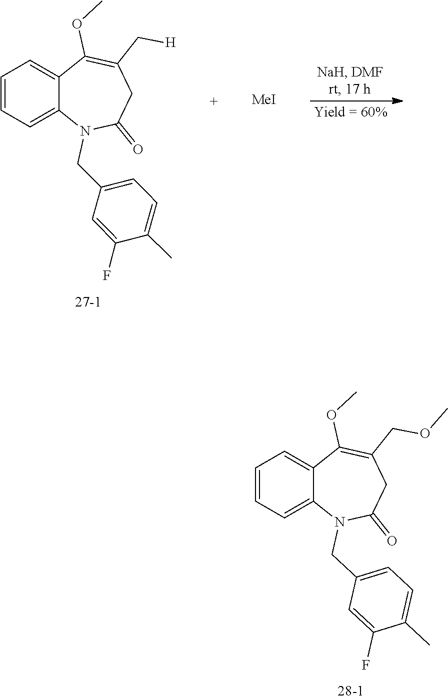 [SMILES: "MeI" ; COC1=C(C)CC(=O)N(Cc2ccc(C)c(F)c2)c2ccccc21 ; COCC1=C(OC)c2ccccc2N(Cc2ccc(C)c(F)c2)C(=O)C1]

### 1-(3-fluoro-4-methylbenzyl)-5-methoxy-4-(methoxymethyl)-1,3-dihydro-2H-benzo[b]azepin-2-one (28-1)

To a stirred solution of 1-(3-fluoro-4-methylbenzyl)-4-(hydroxymethyl)-5-methoxy-1,3-dihydro-2H-benzo[b]azepin-2-one 27-1 (100 mg, 0.29 mmol, 1 equiv) and Mel (90 μL, 1.45 mmol, 5 equiv) in DMF (1 mL), was added NaH 60% dispersion in mineral oil (14 mg, 0.58 mmol, 2 equiv) at ambient temperature under N₂ atmosphere. The reaction mixture was then stirred at room temperature r.t for 17 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 62 mg (purity=99%) white solid compound with a yield of 60%.

TLC R_{f}=0.8 (PE:EA=2:1)

LCMS (m/z)=378.80 (M+Na)

### Example 35. Representative Synthesis of Compounds of Formula 34-1

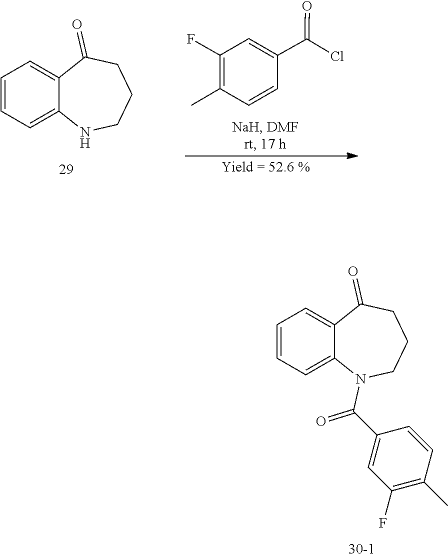 [SMILES: O=C1CCCNc2ccccc21 ; Cc1ccc(C(=O)Cl)cc1F ; Cc1ccc(C(=O)N2CCCC(=O)c3ccccc32)cc1F]

### 1-(3-fluoro-4-methylbenzoyl)-1, 2, 3, 4-tetrahydro-5H-benzo[b]azepin-5-one (30-1)

Sodium hydride (60% in oil) (163.2 mg, 4.08 mmol, 2 equiv) was added to a stirred solution of 1,2,3,4-tetrahydro-5H-benzo[b]azepin-5-one 29 (329 mg, 2.04 mmol, 1 equiv) in THF (11.6 mL). The mixture was stirred at room temperature for 1 h, and treated with 3-fluoro-4-methylbenzoyl chloride (1.056 g, 6.12 mmol, 3 equiv). After being stirred at mom temperature for 4.5 h, then mixture was treated with H₂O and then extracted with ethyl acetate. The extract was washed with brine, dried over anhydrous Na₂SO₄, filtered and concentrated by rotary evaporation to get the crude compound. Then, the resulting concentrate was purified on a silica column to get 319 mg (purity=96%) white solid compound with a yield of 52.6%.

TLC R_{f}=0.2 (PE:EA=4:1);

LCMS (m/z)=297.90 (M+H)

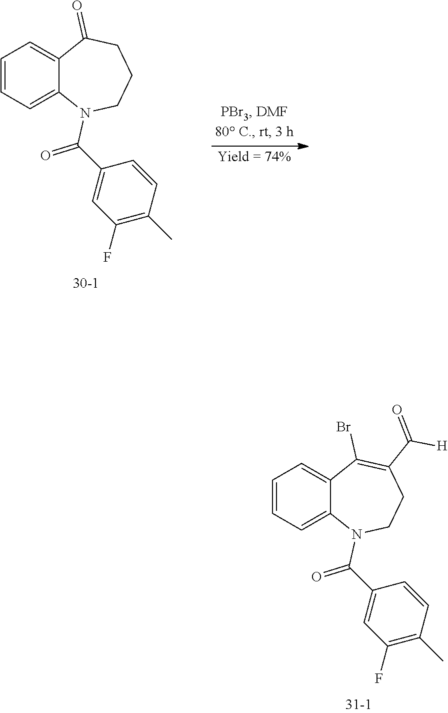 [SMILES: Cc1ccc(C(=O)N2CCCC(=O)c3ccccc32)cc1F ; Cc1ccc(C(=O)N2CCC(C=O)=C(Br)c3ccccc32)cc1F]

### 5-bromo-1-(3-fluoro-4-methylbenzoyl)-2,3-dihydro-1H-benzo[b]azepine-4-carbaldehyde (31-1)

Phosphorus tribromide (119.8 μL, 1.26 mmol, 1.3 equiv) was added to a flask containing N,N-dimethylformamide (9.7 mL) in an ice bath and stirred for 10 min. The ice bath was replaced with an ambient temperature water bath and stirred for an additional 10-15 min. 1-(3-fluoro-4-methylbenzoyl)-1,2,3,4-tetrahydro-5H-benzo[b]azepin-5-one 30-1 (289 mg, 0.97 mmol, 1.0 equiv) was added and stirred for 15 min. The reaction mixture was heated to 80° C. and stirred for an additional 1 h. The orange solution was diluted with ice water and neutralised with 20% sodium acetate solution and with diethyl ether (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 279 mg (purity=90%) yellow solid compound with a yield of 74%.

TLC R_{f}=0.25 (PE:EA=4:1);

LCMS (m/z)=389.60 (M+H)

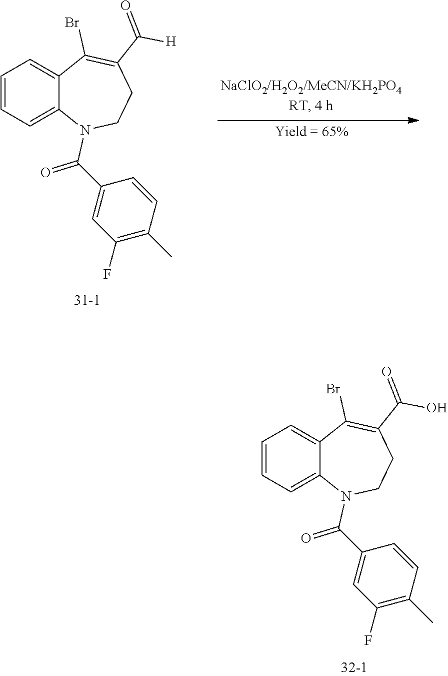 [SMILES: Cc1ccc(C(=O)N2CCC(C=O)=C(Br)c3ccccc32)cc1F ; Cc1ccc(C(=O)N2CCC(C(=O)O)=C(Br)c3ccccc32)cc1F]

### 5-bromo-1-(3-fluoro-4-methylbenzoyl)-2,3-dihydro-1H-benzo[b]azepine-4-carboxylic acid (32-1)

Reactions were carried out by addition of aqueous NaClO₂ (114 mg, 1.27 mmol, 1.76 equiv) to a solution of 5-bromo-1-(3-fluoro-4-methylbenzoyl)-2,3-dihydro-1H-benzo[b]azepine-4-carbaldehyde 31-1 (279 mg, 0.72 mmol, 1 equiv) and (74.8 μL, 0.73 mmol, 1.02 equiv) of 30% H₂O₂ in aqueous acetonitrile, at 0° C., buffered with KH₂PO₄ at pH 4˜3. The reaction mixture was stirred for 4 h. After complete reaction, a small amount of the Na₂SO₃ (˜0.5 g) was added to destroy the unreacted NaClO₂ and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 190 mg (purity=95%) white solid compound with a yield of 65%.

TLC R_{f}=0.25 (DCM:MeOH=10:1);

LCMS (m/z)=405.60 (M+H)

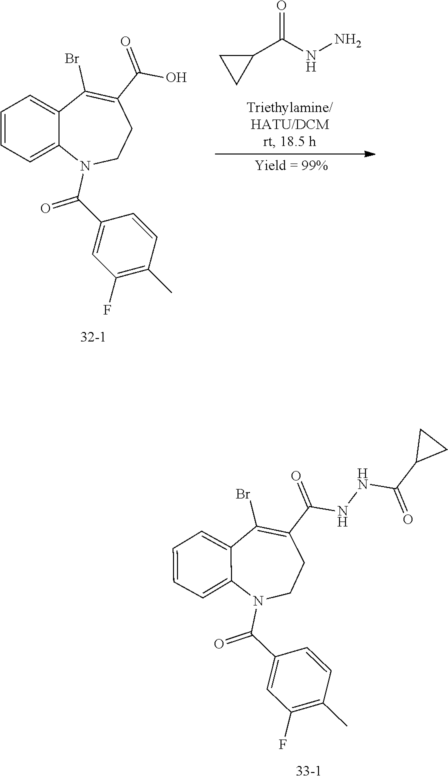 [SMILES: Cc1ccc(C(=O)N2CCC(C(=O)O)=C(Br)c3ccccc32)cc1F ; NNC(=O)C1CC1 ; Cc1ccc(C(=O)N2CCC(C(=O)NNC(=O)C3CC3)=C(Br)c3ccccc32)cc1F]

### 5-bromo-N′-(cyclopropanecarbonyl)-1-(3-fluoro-4-methylbenzoyl)-2,3-dihydro-1H-benzo[b]azepine-4-carbohydrazide(33-1)

5-bromo-1-(3-fluoro-4-methylbenzoyl)-2,3-dihydro-1H-benzol[b]azepine-4-carboxylic acid 32-1 (190 mg, 0.47 mmol, 1 equiv) in dichloromethane (6.2 mL) was added cyclopropanecarbohydrazide (70.6 mg, 0.705 mmol, 1.5 equiv), triethylamine (98 μL, 0.705 mmol, 1.5 equiv), and HATU (357.2 mg, 0.94 mmol, 2 equiv). The mixture was stirred at room temperature for 4 h. Then it was concentrated to give a crude, which was purified by silica gel column chromatography (PE/EA, 1:4) to get the product 226 mg (purity=97%) as a white solid. Yield: 99%.

TLC R_{f}=0.5 (DCM:MeOH=10:1);

LCMS (m/z)=488.10 (M+H)

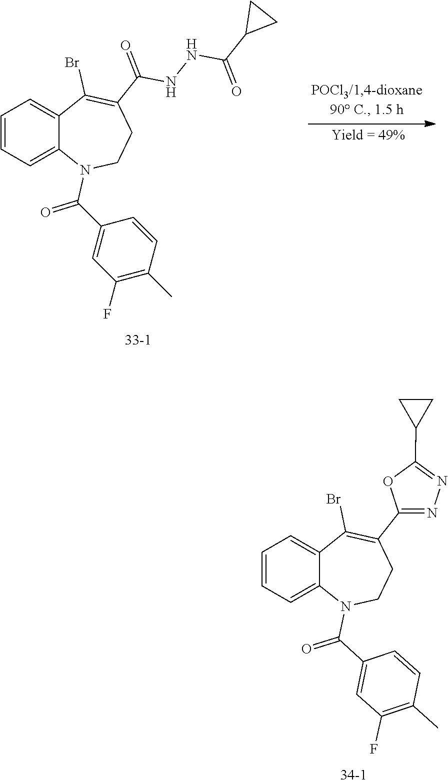 [SMILES: Cc1ccc(C(=O)N2CCC(C(=O)NNC(=O)C3CC3)=C(Br)c3ccccc32)cc1F ; Cc1ccc(C(=O)N2CCC(c3nnc(C4CC4)o3)=C(Br)c3ccccc32)cc1F]

### (5-bromo-4-(5-cyclopropyl-1,3,4-oxadiazol-2-yl)-2,3-dihydro-1H-benzo[b]azepin-1-yl)(3-fluoro-4-methylphenyl)methanone (34-1)

5-bromo-N′-(cyclopropanecarbonyl)-1-(3-fluoro-4-methylbenzoyl)-2-oxo-2,3-dihydro-1H-benzo[b]azepine-4-carbohydrazide 33-1 (228 mg, 0.47 mmol, 1 equiv) was dissolved in 1,4-dioxane (6.9 mL) and phosphoryl chloride (287.8 μL, 3.09 mmol, 6.57 equiv) was added and the mixture was heated at 90° C. for 1.5 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (108 mg) as a yellow solid. Yield: 49%: Purity: 90%.

TLC R_{f}=0.3 (PE:EA=1:1);

LCMS (m/z)=468.20 (M+H)

### Example 36. Representative Synthesis of Compounds of Formula 34-2

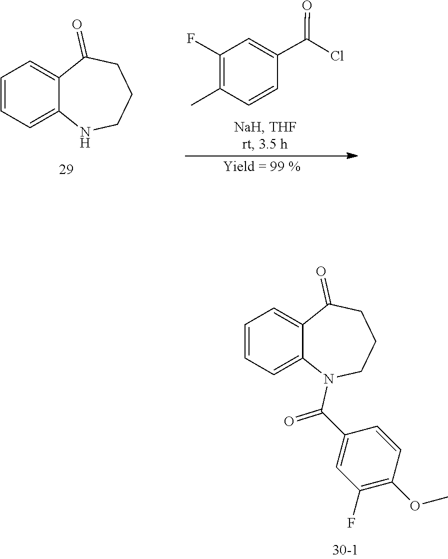 [SMILES: O=C1CCCNc2ccccc21 ; Cc1ccc(C(=O)Cl)cc1F ; COc1ccc(C(=O)N2CCCC(=O)c3ccccc32)cc1F]

### 1-(3-fluoro-4-methoxybenzoyl)-1, 2, 3, 4-tetrahydro-5H-benzo[b]azepin-5-one (30-2)

Sodium hydride (60% in oil) (149 mg, 3.72 mmol, 2 equiv) was added to a stirred solution of 1,2,3,4-tetrahydro-5H-benzo[b]azepin-5-one 29 (300 mg, 1.86 mmol, 1 equiv) in THF (10.5 mL). The mixture was stirred at room temperature for 1 h, and treated with 3-fluoro-4-methylbenzoyl chloride (1 g, 5.58 mmol, 3 equiv). After being stirred at room temperature for 3.5 h, then mixture was treated with H₂O and then extracted with ethyl acetate. The extract was washed with brine, dried over anhydrous Na₂SO₄, filtered and concentrated by rotary evaporation to get the crude compound. Then, the resulting concentrate was purified on a silica column to get 577 mg (purity=95%) white solid compound with a yield of 99%.

TLC R_{f}=0.3 (PE:EA=4:1);

LCMS (m/z)=297.90 (M+H)

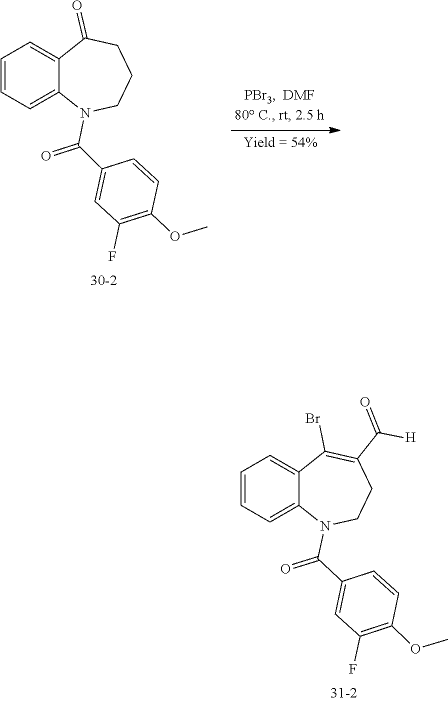 [SMILES: COc1ccc(C(=O)N2CCCC(=O)c3ccccc32)cc1F ; COc1ccc(C(=O)N2CCC(C=O)=C(Br)c3ccccc32)cc1F]

### 5-bromo-1-(3-fluoro-4-methoxybenzoyl)-2,3-dihydro-1H-benzo[b]azepine-4-carbaldehyde (31-2)

Phosphorus tribromide (230 μL, 2.42 mmol, 1.3 equiv) was added to a flask containing N,N-dimethylformamide (18.6 mL) in an ice bath and stirred for 10 min. The ice bath was replaced with an ambient temperature water bath and stirred for an additional 10-15 min. 1-(3-fluoro-4-methoxybenzoyl)-1,2,3,4-tetrahydro-5H-benzo[b]azepin-5-one 30-2 (577 mg, 1.84 mmol, 1.0 equiv) was added and stirred for 15 min. The reaction mixture was heated to 80° C. and stirred for an additional 2.5 h. The orange solution was diluted with ice water and neutralised with 20% sodium acetate solution and with EA (3×10-20 mL). The combined organic extracts were washed with saturated aqueous sodium bicarbonate solution (50 mL), brine 50 mL, and water (3×50 mL), dried over anhydrous Na₂SO₄, filtered and concentrated by rotary evaporation to get the crude compound. Then, the resulting concentrate was purified on a silica column to get 406 mg (purity=65%) yellow oil compound with a yield of 54%.

TLC R_{f}=0.35 (PE:EA=1:1);

LCMS (m/z)=406.90 (M+H)

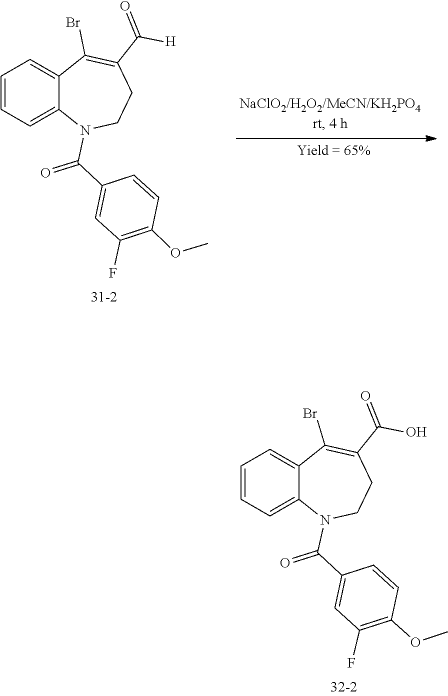 [SMILES: COc1ccc(C(=O)N2CCC(C=O)=C(Br)c3ccccc32)cc1F ; COc1ccc(C(=O)N2CCC(C(=O)O)=C(Br)c3ccccc32)cc1F]

### 5-bromo-1-(3-fluoro-4-methoxybenzoyl)-2,3-dihydro-1H-benzo[b]azepine-4-carboxylic acid (32-2)

Reactions were carried out by addition of aqueous NaClO₂ (158.4 mg, 1.76 mmol, 1.76 equiv) to a solution of 5-bromo-1-(3-fluoro-4-methoxybenzoyl)-2,3-dihydro-1H-benzo[b]azepine-4-carbaldehyde 31-2 (406 mg, 1 mmol, 1 equiv) and (104 μL, 1.02 mmol, 1.02 equiv) of 30% H₂O₂ in aqueous acetonitrile, at 0° C. buffered with KH₂PO₄ at pH 4˜3. The reaction mixture was stirred for 4 h. After complete reaction, a small amount of the Na₂SO₃ (˜0.5 g) was added to destroy the unreacted NaClO₂ and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 259 mg (purity=84%) white solid compound with a yield of 62%.

TLC R_{f}=0.1 (DCM:MeOH=10:1);

LCMS (m/z)=422.80 (M+H)

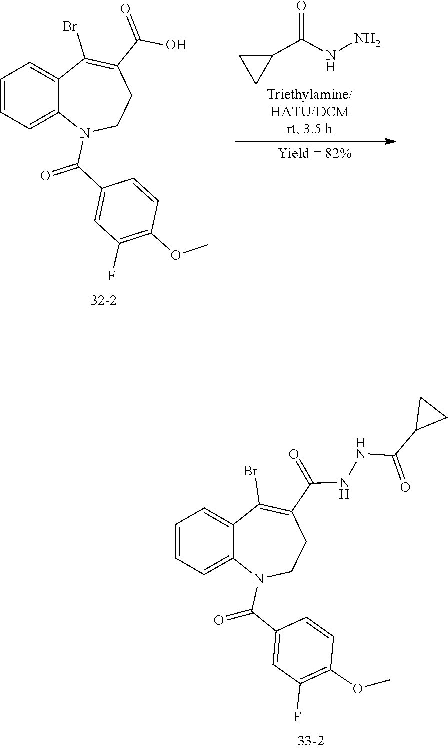 [SMILES: COc1ccc(C(=O)N2CCC(C(=O)O)=C(Br)c3ccccc32)cc1F ; NNC(=O)C1CC1 ; COc1ccc(C(=O)N2CCC(C(=O)NNC(=O)C3CC3)=C(Br)c3ccccc32)cc1F]

### 5-bromo-N′-(cyclopropanecarbonyl)-1-(3-fluoro-4-methoxybenzoyl)-2,3-dihydro-1H-benzo[b]azepine-4-carbohydrazide (33-2)

5-bromo-1-(3-fluoro-4-methoxybenzoyl)-2,3-dihydro-1H-benzo[b]azepine-4-carboxylic acid 32-2 (259 mg, 0.62 mmol, 1 equiv) in dichloromethane (8.1 mL) was added cyclopropanecarbohydrazide (93 mg, 0.93 mmol, 1.5 equiv), triethylamine (130 μL, 0.93 mmol, 1.5 equiv), and HATU (471.2 mg, 1.24 mmol, 2 equiv). The mixture was stirred at room temperature for 3.5 h. Then it was concentrated to give a crude, which was purified by silica gel column chromatography (PE/EA, 1:4) to get the product 255 mg (purity=92%) as a white solid. Yield: 82%.

TLC R_{f}=0.3 (DCM:MeOH=10:1)

LCMS (m/z)=505.10 (M+H)

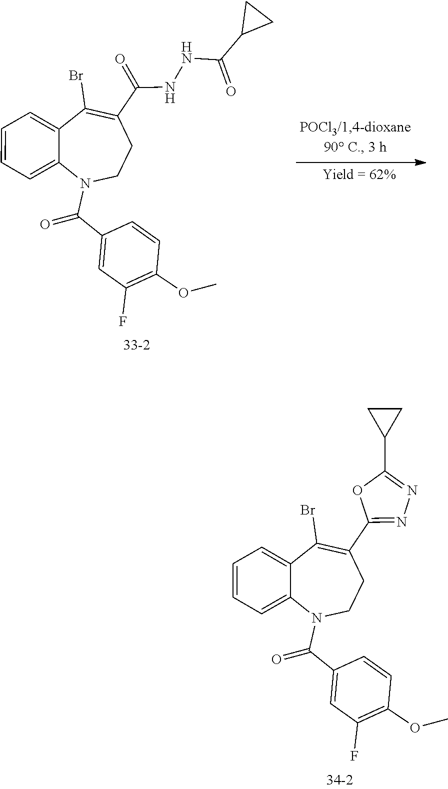 [SMILES: COc1ccc(C(=O)N2CCC(C(=O)NNC(=O)C3CC3)=C(Br)c3ccccc32)cc1F ; COc1ccc(C(=O)N2CCC(c3nnc(C4CC4)o3)=C(Br)c3ccccc32)cc1F]

### (5-bromo-4-(5-cyclopropyl-1,3,4-oxadiazol-2-yl)-2,3-dihydro-1H-benzo[b]azepin-1-yl)(3-fluoro-4-methoxyphenyl)methanone (34-2)

5-bromo-N′-(cyclopropanecarbonyl)-1-(3-fluoro-4-methoxybenzoyl)-2,3-dihydro-II-benzo[b]azepine-4-carbohydrazide 33-2 (255 mg, 0.51 mmol, 1 equiv) was dissolved in 1,4-dioxane (5.2 mL) and phosphoryl chloride (312 μL, 3.35 mmol, 6.57 equiv) was added and the mixture was heated at 90° C. for 3 h. then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (155 mg) as a yellow solid. Yield: 62%; Purity: 88.9%.

TLC R_{f}=0.45 (DCM:MeOH=10:1);

LCMS (m/z)=486.60 (M+H)

### Example 37. Representative Synthesis of Compounds of Formula ZLS43

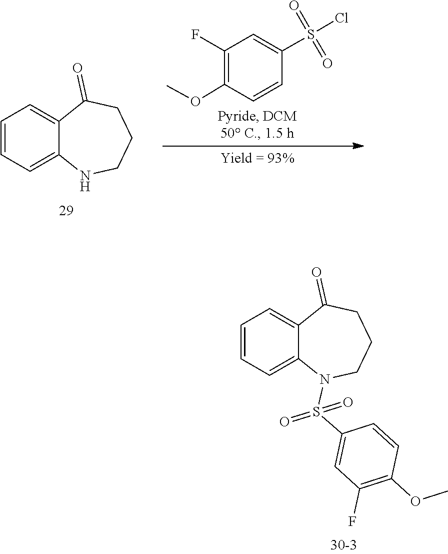 [SMILES: O=C1CCCNc2ccccc21 ; COc1ccc(S(=O)(=O)Cl)cc1F ; COc1ccc(S(=O)(=O)N2CCCC(=O)c3ccccc32)cc1F]

### 1-((3-fluoro-4-methoxyphenyl)sulfonyl)-1, 2, 3, 4-tetrahydro-5H-benzo[b]azepin-5-one(30-3)

To the solution of compound 29 (200 mg, 1.24 mmol, 1 equiv) in anhydrous DCM (7.8 mL) was added pyridine (145 μL, 1.8 mmol, 1.45 equiv). After the solution was cooled to 0° C. using an ice-bath, 3-fluoro-4-methoxybenzenesulfonyl chloride (557.4 mg, 2.48 mmol, 2 equiv) was added dropwise through a syringe. After addition, the resulting mixture was refluxed at 50° C. for 1.5 h. After completion, the mixture was adjusted to neutral pH with 0.5 M HCl, and extracted with DCM three times (3×30 mL). The combined organic layer was rinsed with saline, dried over anhydrous Na₂SO₄, and concentrated under reduced pressure. The crude product was purified by silica gel chromatography, eluting with petroleum ether/EtOAc to afford the desirable compound as yellow oil (405 mg, 93%). Purity=99%.

TLC R_{f}=0.25 (PE:EA=2:1);

LCMS (m/z)=350.58 (M+H)

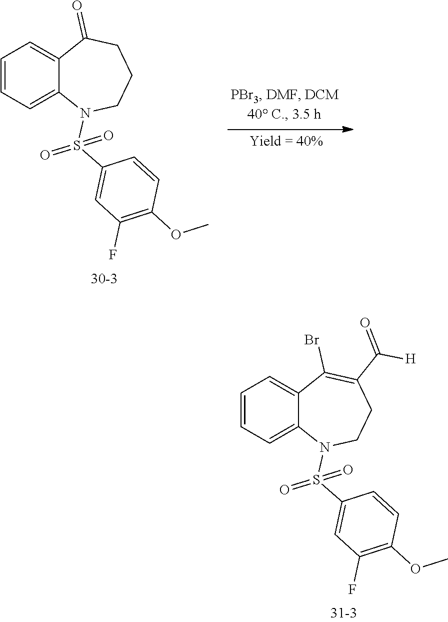 [SMILES: COc1ccc(S(=O)(=O)N2CCCC(=O)c3ccccc32)cc1F ; COc1ccc(S(=O)(=O)N2CCC(C=O)=C(Br)c3ccccc32)cc1F]

### 5-bromo-1-((3-fluoro-4-methoxyphenyl)sulfonyl)-2,3-dihydro-1H-benzo[b]azepine-4-carbaldehyde (MBICR-ZLS-499)

A solution of PBr₃ (620 μL, 6.53 mmol, 4 equiv) was added dropwise to an ice-cooled solution of DMF (652 μL, 8.42 mmol, 5.16 equiv) in DCM (3.2 mL) in argon atmosphere. Subsequently, the solution of 1-((3-fluoro-4-methoxyphenyl)sulfonyl)-1, 2, 3, 4-tetrahydro-5H-benzo[b]azepin-5-one 30-3 (570 mg, 1.63 mmol, 1 equiv) was added into the prepared solution via a syringe at 0° C. After addition, the resulting mixture was refluxed at 40° C. for 3.5 h. After completion, the mixture was cooled using an ice-bath, and the reaction was quenched by addition of ice-water (20 mL). Then mixture was extracted with DCM three times (3×40 mL). The combined organic layer was washed with water (20 mL) and brine (20 mL), dried over anhydrous Na₂SO₄, and concentrated under reduced pressure to afford the desirable compound as white solid (289 mg, yield=40%, purity=99%).

TLC R_{f}=0.5 (PE:EA=2:1);

LCMS (m/z)=442.63 (M+H)

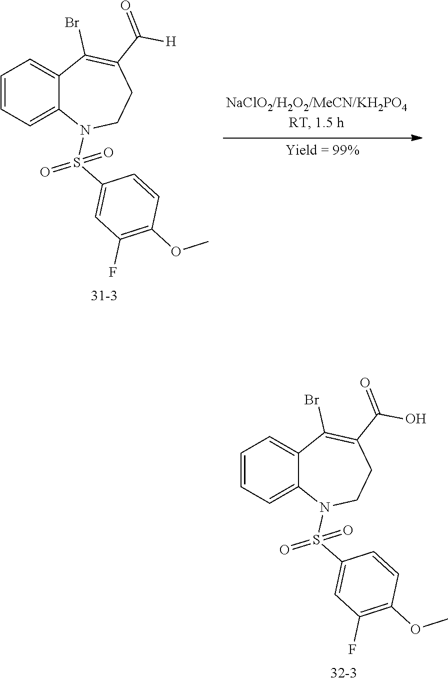 [SMILES: COc1ccc(S(=O)(=O)N2CCC(C=O)=C(Br)c3ccccc32)cc1F ; COc1ccc(S(=O)(=O)N2CCC(C(=O)O)=C(Br)c3ccccc32)cc1F]

### 5-bromo-1-((3-fluoro-4-methoxyphenyl)sulfonyl)-2,3-dihydro-1H-benzo[b]azepine-4-carboxylic acid (32-3)

Reactions were carried out by addition of aqueous NaClO₂ (104 mg, 1.16 mmol, 1.76 equiv) to a solution of 5-bromo-1-((3-fluoro-4-methoxyphenyl)sulfonyl)-2,3-dihydro-1H-benzo[b]azepine-4-carbaldehyde 31-3 (289 mg, 0.66 mmol, 1 equiv) and (68 μL, 0.67 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₄ (˜0.5 g) was added to destroy the unreacted NaClO₂ and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 296 mg (purity=42%) white solid compound with a yield of 99%.

TLC R_{f}=0.1 (DCM:MeOH=10:1);

LCMS (m/z)=422.80 (M+H)

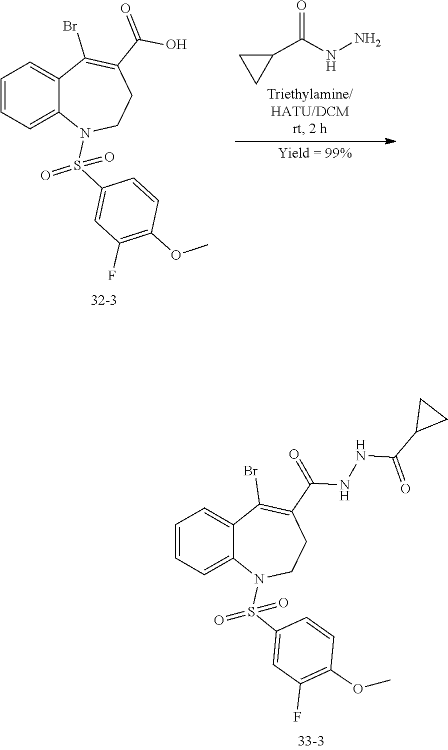 [SMILES: COc1ccc(S(=O)(=O)N2CCC(C(=O)O)=C(Br)c3ccccc32)cc1F ; NNC(=O)C1CC1 ; COc1ccc(S(=O)(=O)N2CCC(C(=O)NNC(=O)C3CC3)=C(Br)c3ccccc32)cc1F]

### 5-bromo-N′-(cyclopropanecarbonyl)-1-((3-fluoro-4-methoxyphenyl)sulfonyl)-2,3-dihydro-1H-benzo[b]azepine-4-carbohydrazide (MBICR-ZLS-513)

5-bromo-1-((3-fluoro-4-methoxyphenyl)sulfonyl)-2,3-dihydro-1H-benzo[b]azepine-4-carboxylic acid 32-3 (298 mg, 0.65 mmol, 1 equiv) in dichloromethane (8.5 mL) was added cyclopropanecarbohydrazide (98 mg, 0.98 mmol, 1.5 equiv), triethylamine (136 μL, 0.98 mmol, 1.5 equiv), and HATU (496 mg, 1.31 mmol, 2 equiv). The mixture was stirred at room temperature for 2 h. Then it was concentrated to give a crude, which was purified by silica gel column chromatography (PE/EA, 1:4) to get the product 346 mg (purity=99%) as a white solid. Yield: 99%.

TLC R_{f}=0.2 (DCM:MeOH=10:1)

LCMS (m/z)=540.30 (M+H)

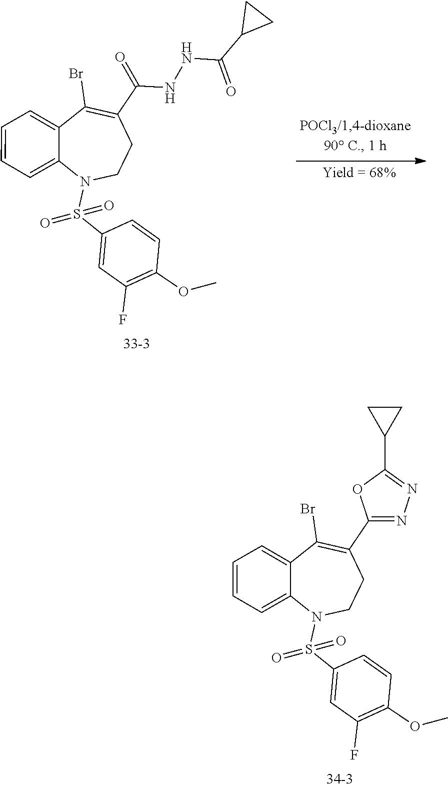 [SMILES: COc1ccc(S(=O)(=O)N2CCC(C(=O)NNC(=O)C3CC3)=C(Br)c3ccccc32)cc1F ; COc1ccc(S(=O)(=O)N2CCC(c3nnc(C4CC4)o3)=C(Br)c3ccccc32)cc1F]

### 2-(5-bromo-1-((3-fluoro-4-methoxyphenyl)sulfonyl)-2,3-dihydro-1H-benzo[b]azepin-4-yl)-5-cyclopropyl-1,3,4-oxadiazole (34-3)

5-bromo-N′-(cyclopropanecarbonyl)-1-((3-fluoro-4-methoxyphenyl)sulfonyl)-2,3-dihydro-1H-benzo[b]azepine-4-carbohydrazide 33-3 (350 mg, 0.65 mmol, 1 equiv) was dissolved in 1,4-dioxane (6.6 mL) and phosphoryl chloride (398 μL, 4.27 mmol, 6.57 equiv) was added and the mixture was heated at 90° C. for 1 h, then cooled to room temperature and diluted with ethyl acetate. The mixture was washed with NaHCO₃ and brine, dried over sodium sulfate, filtered and concentrated in vacuo. The residue was purified by silica gel column chromatography to get the product (155 mg) as a white solid. Yield: 68%; Purity: 99%.

TLC R_{f}=0.8 (DCM:MeOH=10:1);

LCMS (m/z)=522.20 (M+H)

### Example 38. Representative Synthesis of Compounds of Formula 34-4

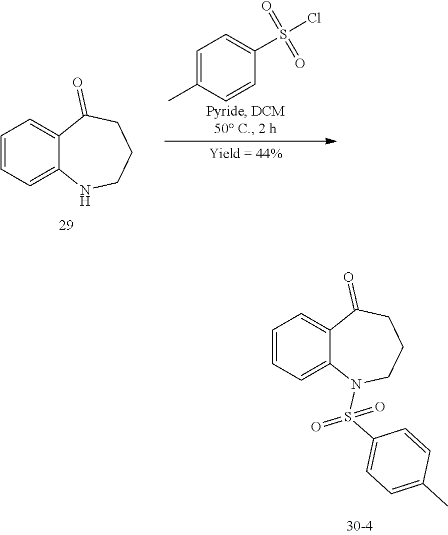 [SMILES: O=C1CCCNc2ccccc21 ; Cc1ccc(S(=O)(=O)Cl)cc1 ; Cc1ccc(S(=O)(=O)N2CCCC(=O)c3ccccc32)cc1]

1-tosyl-1, 2, 3, 4-tetrahydro-5H-benzo[b]azepin-5-one (30-4)

To the solution of compound 29 (5 g, 31 mmol, 1 equiv) in anhydrous DCM (194 mL) was added pyridine (3.637 mL, 45 mmol, 1.45 equiv). After the solution was cooled to 0° C. using an ice-bath, 3-fluoro-4-methoxybenzenesulfonyl chloride (557.4 mg, 2.48 mmol, 1.2 equiv) was added dropwise through a syringe. After addition, the resulting mixture was refluxed at 50° C. for 2 h. After completion, the mixture was adjusted to neutral pH with 0.5 M HCL, and extracted with DCM three times (3×30 mL). The combined organic layer was rinsed with saline, dried over anhydrous Na₂SO₄, and concentrated under reduced pressure. The crude product was purified by silica gel chromatography, eluting with petroleum ether/EtOAc to afford the desirable compound as white solid (4.32 g, 44%). Purity=97%.

TLC R_{f}=0.55 (PE:EA=2:1);

LCMS (m/z)=316.60 (M+H)

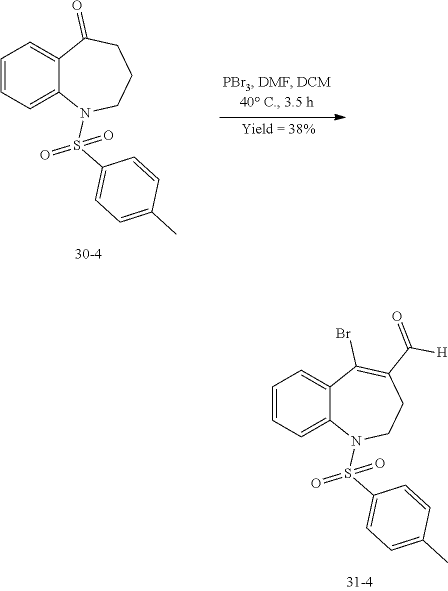 [SMILES: Cc1ccc(S(=O)(=O)N2CCCC(=O)c3ccccc32)cc1 ; Cc1ccc(S(=O)(=O)N2CCC(C=O)=C(Br)c3ccccc32)cc1]

### 5-bromo-1-tosyl-2,3-dihydro-1H-benzo[b]azepine-4-carbaldehyde (31-4)

A solution of PBr₃ (5.08 mL, 53.49 mmol, 4 equiv) was added dropwise to an ice-cooled solution of DMF (5.342 mL, 69 mmol, 5.16 equiv) in DCM (20.4 mL) in argon atmosphere. Subsequently, the solution of 1-tosyl-1,2,3,4-tetrahydro-5H-benzo[b]azepin-5-one 30-4 (4.22 g, 13.37 mmol, 1 equiv) was added into the prepared solution via a syringe at 0° C. After addition, the resulting mixture was refluxed at 80° C. for 3.5 h. After completion, the mixture was cooled using an ice-bath, and the reaction was quenched by addition of ice-water (20 mL). Then mixture was extracted with DCM three times (3×40 mL). The combined organic layer was washed with water (20 mL) and brine (20 mL), dried over anhydrous Na₂SO₄, and concentrated under reduced pressure to afford the desirable compound as white solid (2.078 g, yield=38%, purity=92%).

TLC R_{f}=0.7 (PE:EA=2:1);

LCMS (m/z)=408.50 (M+H)

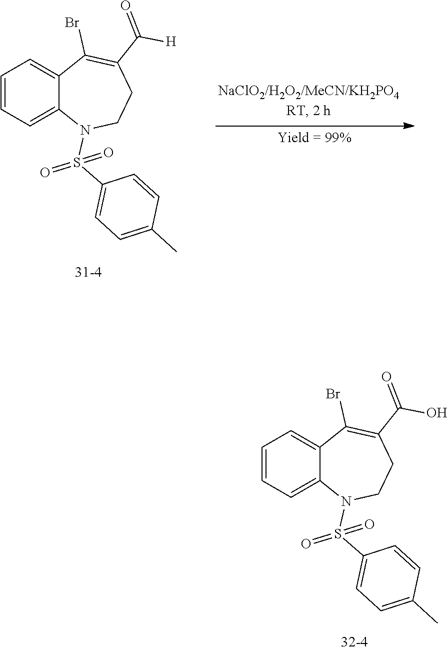 [SMILES: Cc1ccc(S(=O)(=O)N2CCC(C=O)=C(Br)c3ccccc32)cc1 ; Cc1ccc(S(=O)(=O)N2CCC(C(=O)O)=C(Br)c3ccccc32)cc1]

### 5-bromo-1-tosyl-2,3-dihydro-1H-benzo[b]azepine-4-carboxylic acid (32-4)

Reactions were carried out by addition of aqueous NaClO₂ (584.8 mg, 6.5 mmol, 1.76 equiv) to a solution of 5-bromo-1-tosyl-2,3-dihydro-1H-benzo[b]azepine-4-carbaldehyde 31-4 (1.5 g, 3.69 mmol, 1 equiv) and (3.845 mL, 3.77 mmol, 1.02 equiv) of 30% H₂O₂ in aqueous acetonitrile, at 0° C., buffered with KH₂PO₄ at pH 4˜3. The reaction mixture was stirred overnight. After complete reaction, a small amount of the Na₂SO₃ (˜0.5 g) was added to destroy the unreacted NaClO₂ and H₂O₂. Acidification with 10% HCl, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 1.54 g (purity=94%) white solid compound with a yield of 99%.

TLC R_{f}=0.2 (DCM:MeOH=10:1);

LCMS (m/z)=424.42 (M+H)

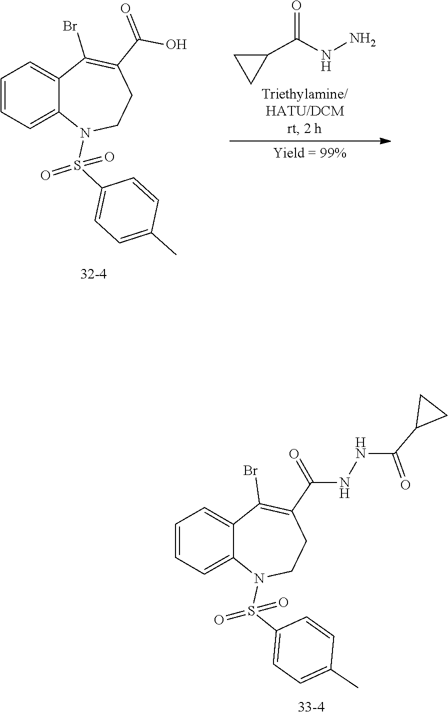 [SMILES: Cc1ccc(S(=O)(=O)N2CCC(C(=O)O)=C(Br)c3ccccc32)cc1 ; NNC(=O)C1CC1 ; Cc1ccc(S(=O)(=O)N2CCC(C(=O)NNC(=O)C3CC3)=C(Br)c3ccccc32)cc1]

### 5-bromo-N′-(cyclopropanecarbonyl)-1-tosyl-2,3-dihydro-1H-benzo[b]azepine-4-carbohydrazide (33-4)

5-bromo-1-tosyl-2,3-dihydro-11H-benzo[b]azepine-4-carboxylic acid 32-4 (1.59 g, 3.69 mmol, 1 equiv) in dichloromethane (48 mL) was added cyclopropanecarbohydrazide (554 mg, 5.54 mmol, 1.5 equiv), triethylamine (769 μL, 5.54 mmol, 1.5 equiv), and HATU (2.804 g, 7.38 mmol, 2 equiv). The mixture was stirred at room temperature for 2 h. Then it was concentrated to give a crude, which was purified by silica gel column chromatography (PE/EA, 1:4) to get the product 1.84 g (purity=94%) as a white solid. Yield: 99%.

TLC R_{f}=0.6 (DCM:MeOH=10:1)

LCMS (m/z)=506.50 (M+H)

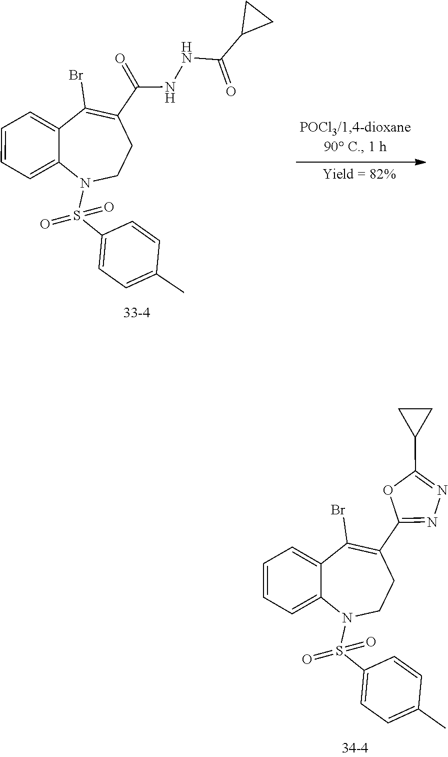 [SMILES: Cc1ccc(S(=O)(=O)N2CCC(C(=O)NNC(=O)C3CC3)=C(Br)c3ccccc32)cc1 ; Cc1ccc(S(=O)(=O)N2CCC(c3nnc(C4CC4)o3)=C(Br)c3ccccc32)cc1]

### 2-(5-bromo-1-tosyl-2,3-dihydro-1H-benzo[b]azepin-4-yl)-5-cyclopropyl-1,3,4-oxadiazole (34-4)

5-bromo-N′-(cyclopropanecarbonyl)-1-tosyl-2,3-dihydro-1H-benzo[b]azepine-4-carbohydrazide 33-4 (1.85 g, 3.69 mmol, 1 equiv) was dissolved in 1,4-dioxane (37.5 mL) and phosphoryl chloride (2.259 mL, 24.24 mmol, 6.57 equiv) was added and the mixture was heated at 90° C. for 1 h. then cooled to room temperature and diluted with ethyl acetate. The mixture was washed with NaHCO₃ and brine, dried over sodium sulfate, filtered and concentrated in vacuo. The residue was purified by silica gel column chromatography to get the product (1.476 g) as a white solid. Yield: 82%; Purity: 89.78%.

TLC R_{f}=0.3 (PE:EA=2:1);

LCMS (m/z)=488.50 (M+H)

### Example 39. Representative Synthesis of Compounds of Formula 40

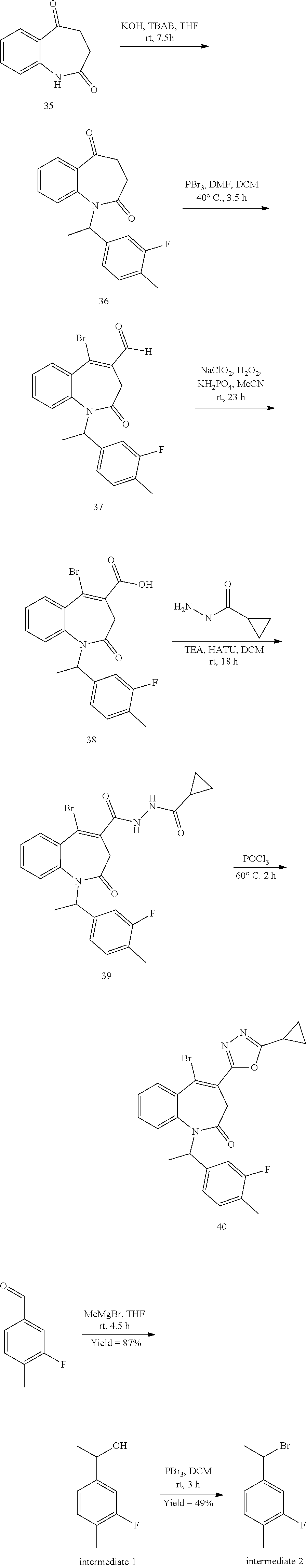

### 4-(1-bromoethyl)-2-fluoro-1-methylbenzene (intermediate 2)

fluoro-4-methylbenzaldehyde (1 g, 7.4 mmol, 1 equiv) in THF (10 mL) was added dropwise a 3 M solution (2896 μL, 8.69 mmol, 1.2 equiv) of methylmagnesium bromide in THF. The temperature of the reaction mixture was allowed to rise back to room temperature, at which the reaction mixture was stirred for 4.5 h. Then, 1 mol/L hydrochloric acid was added, and the mixture was extracted with ethyl acetate. The extract was washed with brine and dried over anhydrous sodium sulfate. The solvent was distilled of under reduced pressure. Then, the resulting concentrate was purified on a silica column to get 967 mg (purity=82%) oil compound with a yield of 87%.

TLC R_{f}=0.6 (PE:EA=1:1)

To a stirred suspension of intermediate 1 (964 mg, 6.25 mmol, 1 equiv) in CH₂Cl₂ (18.8 mL) under N₂ at 0° C. was added phosphorous tribromide (293 μL, 3.13 mmol, 0.5 equiv) dropwise After 15 min, the mixture was warmed to rt and allowed to stir for 3 h, at which point the reaction mixture was quenched with ˜120 (20 mL). The aqueous phase was extracted with CH₂C₂ (3×20 mL), the combined organic layers were washed with saturated aqueous sodium bicarbonate (20 mL) and brine (20 mL), dried (Na₂SO₄), filtered and concentrated in vacuo to afford the title compound (658 mg, 49% yield, 80% purity) as a colorless oil.

TLC R_{f}=0.85 (PE:EA=1:1)

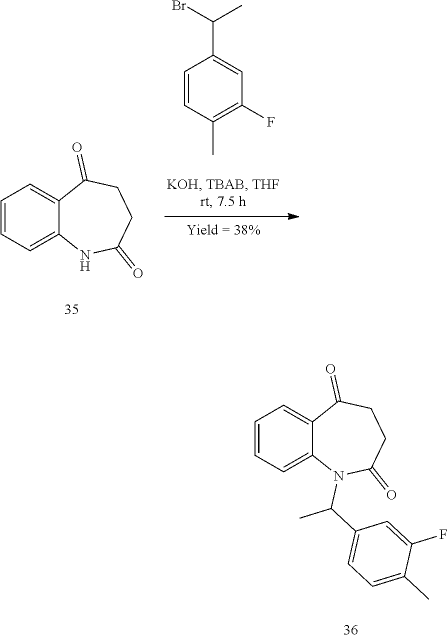 [SMILES: O=C1CCC(=O)c2ccccc2N1 ; Cc1ccc(C(C)Br)cc1F ; Cc1ccc(C(C)N2C(=O)CCC(=O)c3ccccc32)cc1F]

### 1-(3-fluoro-4-methylphenyl)ethyl)-3,4-dihydro-1H-benzo[b]azepine-2,5-dione (36)

To a stirred solution of ketone 35 (360 mg, 2.07 mmol, 1 equiv), Tetrabutylammonium bromide (67 mg, 0.21 mmol, 0.1 equiv) and KOH (162.5 mg, 2.9 mmol, 1.4 equiv) in THF (7.8 mL), was added 3,4-dihydro-1H-benzo[b]azepine-2,5-dione 35 (540 mg, 2.49 mmol, 1.2 equiv) at ambient temperature. The reaction mixture was then stirred at rt for 7.5 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 248 mg (purity=92%) solid yellow solid compound with a yield of 38%.

TLC R_{f}=0.55 (PE:EA=1:1);

LCMS (m/z)=312.20 (M+H)

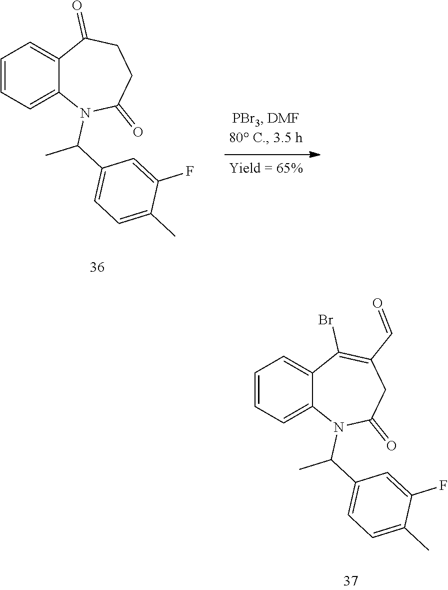 [SMILES: Cc1ccc(C(C)N2C(=O)CCC(=O)c3ccccc32)cc1F ; Cc1ccc(C(C)N2C(=O)CC(C=O)=C(Br)c3ccccc32)cc1F]

### 5-bromo-1-(1-(3-fluoro-4-methylphenyl)ethyl)-2-oxo-2,3-dihydro-1H-benzo[b]azepine-4-carbaldehyde (37)

tribromophosphane (98 μL, 270.68 mmol, 1.3 equiv) was added to a flask containing N, N-dimethylformamide (8 mL) in an ice bath and stirred for 10 min. The ice bath was replaced with an ambient temperature water bath and stirred for an additional 10-15 min. 36 (248 mg, 0.8 mmol, 1 equiv) was added and stirred for 15 min. The reaction mixture was heated to 80° C. and stirred for an additional 3.5 h. The orange solution was diluted with ice water and neutralised with 20% sodium acetate solution and extracted with EA (3×10-20 mL). The combined organic extracts were washed with saturated aqueous sodium bicarbonate solution 50 mL and brine 50 mL, dried over anhydrous Na₂SO₄, filtered and concentrated by rotary evaporation to get the crude compound. Then, the resulting concentrate was purified on a silica column to get 210 mg (purity=94%) orange solid compound with a yield of 65%.

TLC R_{f}=0.7 (PE:EA=1:1)

LCMS (m/z)=404.10 (M+H)

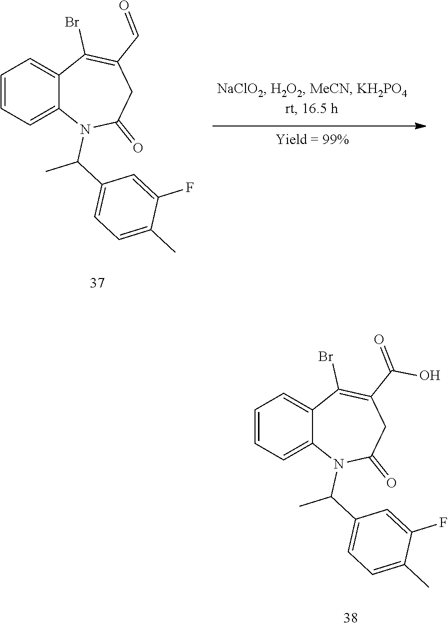 [SMILES: Cc1ccc(C(C)N2C(=O)CC(C=O)=C(Br)c3ccccc32)cc1F ; Cc1ccc(C(C)N2C(=O)CC(C(=O)O)=C(Br)c3ccccc32)cc1F]

### 5-bromo-1-(1-(3-fluoro-4-methylphenyl)ethyl)-2-oxo-2,3-dihydro-1H-benzo[b]azepine-4-carboxylic acid (38)

Reactions were carried out by addition of aqueous NaClO₂ (83 mg, 0.92 mmol, 1.76 equiv) to a solution of 37 (210 mg, 0.52 mmol, 1 equiv) and (54.4 uL, 0.53 mmol, 1.02 equiv) 30% H₂O₂ in aqueous acetonitrile, at 0° C., buffered with KH₂PO₄ at pH 4.3. The reaction mixture was stirred 16.5 h. After complete reaction, a small amount of the Na₂SO₃ (˜0.5 g) was added to destroy the unreacted HOCl and 1-H₂O₂. Acidification with 10% ICI, the mixture was diluted with H₂O (10-20 mL). The compound was extracted with EA (10 mL). The organic layer was separated and extracted with water and brine and then dried over sodium sulphate and concentrated. Then, the resulting concentrate was purified on a silica column to get 215 mg (purity=96%) yellow solid compound with a yield of 99%.

TLC R_{f}=0.1 (DCM:MeOH=1:1)

LCMS (m/z)=418.10 (M+H)

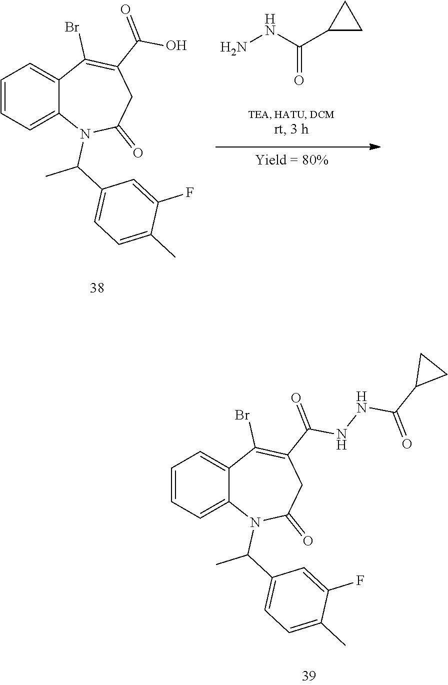 [SMILES: Cc1ccc(C(C)N2C(=O)CC(C(=O)O)=C(Br)c3ccccc32)cc1F ; NNC(=O)C1CC1 ; Cc1ccc(C(C)N2C(=O)CC(C(=O)NNC(=O)C3CC3)=C(Br)c3ccccc32)cc1F]

### 5-bromo-N′-(cyclopropanecarbonyl)-1-(1-(3-fluoro-4-methylphenyl)ethyl)-2-oxo-2,3-dihydro-1H-benzo[b]azepine-4-carbohydrazide (39)

5-bromo-1-(1-(3-fluoro-4-methylphenyl)ethyl)-2-oxo-2,3-dihydro-1H-benzo[b]azepine-4-carboxylic acid 38 (221 ng, 0.51 mmol, 1 equiv) in dichloromethane (6.7 mL) was added cyclopropanecarbohydrazide (76.6 mg, 0.77 mmol, 1.5 equiv), triethylamine (106 μL, 0.77 mmol, 1.5 equiv), and HATU (387.6 mg, 1.02 mmol, 2 equiv). The mixture was stirred at room temperature for 2 h. Then it was concentrated to give a crude, which was purified by silica gel column chromatography (DCM/MeOH, 10:1) to get the product 206 mg (purity=94%) as a yellow oil. Yield: 80%.

TLC R_{f}=0.5 (DCM:MeOH=10:1);

LCMS (m/z)=500.30 (M+H)

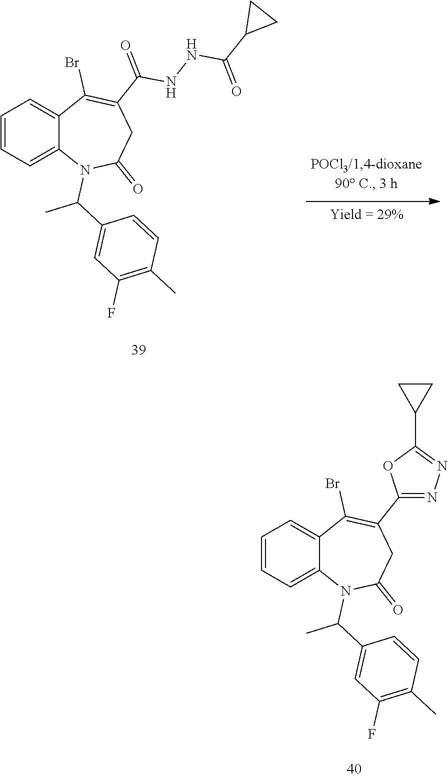 [SMILES: Cc1ccc(C(C)N2C(=O)CC(C(=O)NNC(=O)C3CC3)=C(Br)c3ccccc32)cc1F ; Cc1ccc(C(C)N2C(=O)CC(c3nnc(C4CC4)o3)=C(Br)c3ccccc32)cc1F]

### 5-bromo-4-(5-cyclopropyl-1,3,4-oxadiazol-2-yl)-1-(3-fluoro-4-methylbenzoyl)-1,3-dihydro-2H-benzo[b]azepin-2-one (40)

5-bromo-N′-(cyclopropanecarbonyl)-1-(1-(3-fluoro-4-methylphenyl)ethyl)-2-oxo-2,3-dihydro-1H-benzo[b]azepine-4-carbohydrazide 39 (206 mg, 0.41 mmol, 1 equiv) was dissolved in 1,4-dioxane (4.2 mL) and phosphoryl chloride (252 μL, 2.7 mmol, 6.57 equiv) was added and the mixture was heated at 90° C. for 3 h, then cooled to room temperature and diluted with ethyl acetate. The mixture was washed with sated, NaHCO₃ and brine, dried over sodium sulfate, filtered and concentrated in vacuum. The residue was purified by silica gel column chromatography to get the product (58 mg) as a white solid. Yield: 29%; Purity: 98%.

TLC R_{f}=0.4 (PE:EA=2:1);

LCMS (m/z)=484.40 (M+H)

### Example 40. Representative Synthesis of Compound of Formula 42

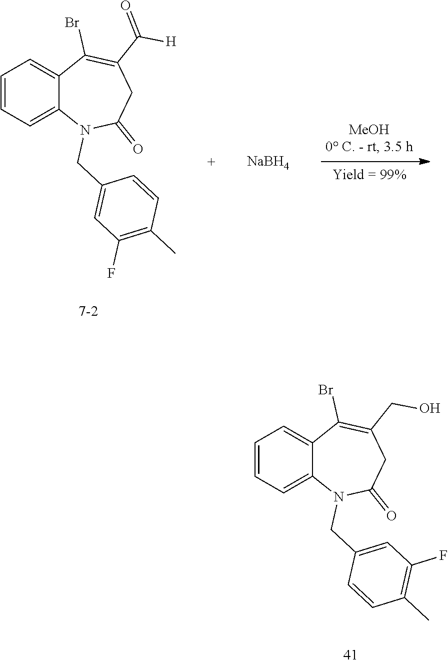 [SMILES: Cc1ccc(CN2C(=O)CC(C=O)=C(Br)c3ccccc32)cc1F ; "NaBH4" ; Cc1ccc(CN2C(=O)CC(CO)=C(Br)c3ccccc32)cc1F]

### 5-bromo-1-(3-fluoro-4-methylbenzyl)-4-(hydroxymethyl)-1,3-dihydro-2H-benzo[b]azepin-2-one (41)

To a solution of 5-bromo-1-(3-fluoro-4-methylbenzyl)-2-oxo-2,3-dihydro-1H-benzo[b]azepine-4-carbaldehyde 7-2 (970 mg, 2.5 mmol, 1 equiv) in MeOH (8 mL) was added NaBH₄ (463 mg, 12.5 mmol, 5 equiv) at 0° C. The reaction mixture was then stirred at rt for 3.5 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 965 mg (purity=98%) of the light-yellow oil compound with a yield of 99%.

TLC R_{f}=0.2 (PE:EA=2:1)

LCMS (m/z)=372.10 (M-17)

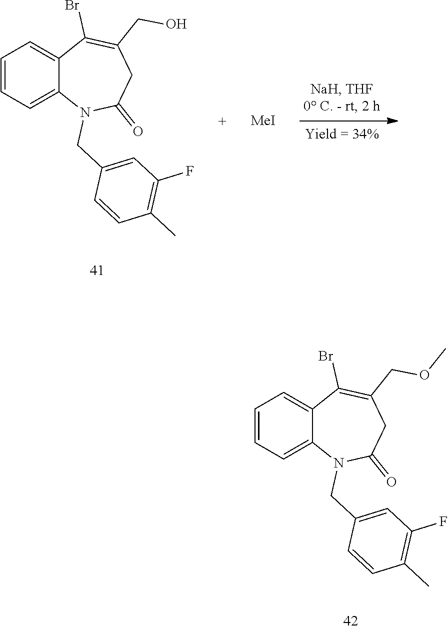 [SMILES: "MeI" ; Cc1ccc(CN2C(=O)CC(CO)=C(Br)c3ccccc32)cc1F ; COCC1=C(Br)c2ccccc2N(Cc2ccc(C)c(F)c2)C(=O)C1]

### 5-bromo-1-(3-fluoro-4-methylbenzyl)-4-(methoxymethyl)-1,3-dihydro-2H-benzo[b]azepin-2-one (42)

The NaH (30 mg, 0.75 mmol, 2.5 equiv) was added to the dry THF (0.5 mL) in the round bottom, 5-bromo-1-(3-fluoro-4-methylbenzyl)-4-(hydroxymethyl)-1,3-dihydro-2H-benzo[b]azepin-2-one 41 (117 mg, 0.3 mmol, 1 equiv) was dissolved in THF (0.5 mL) and slowly added into the NaH solution at 0° C., continue to stir for 20 min. Mel (37 μL, 0.6 mmol, 2 equiv) was added, and the reaction was stirred for 2 h at rt. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 42 mg (purity=98%) yellow oil compound with a yield of 34%.

TLC R_{f}=0.6 (PE:EA=2:1)

LCMS (m/z)=404.60 (M+1)

### Example 41. Representative Synthesis of Compound of Formula 44-1

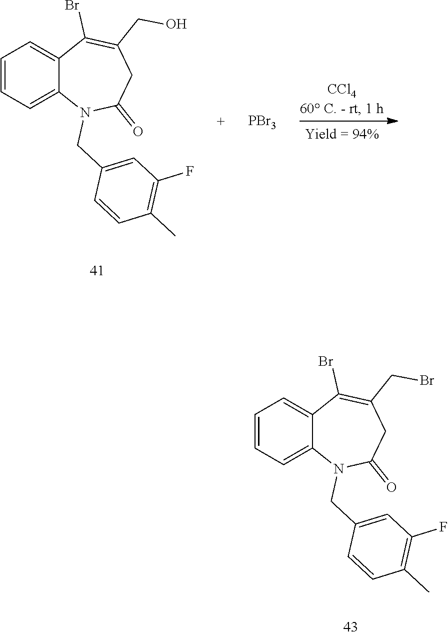 [SMILES: Cc1ccc(CN2C(=O)CC(CO)=C(Br)c3ccccc32)cc1F ; "PBr3" ; Cc1ccc(CN2C(=O)CC(CBr)=C(Br)c3ccccc32)cc1F]

### 5-bromo-4-(bromomethyl)-1-(3-fluoro-4-methylbenzyl)-1,3-dihydro-2H-benzo[b]azepin-2-one (43)

The 5-bromo-1-(3-fluoro-4-methylbenzyl)-4-(hydroxymethyl)-1,3-dihydro-2H-benzo[b]azepin-2-one 41 (1.1 g, 2.8 mmol, 1 equiv) and carbon tetrachloride solvent (9 mL) were placed in round bottom. Then PBr₃ (134 μL, 1.4 mmol, 0.5 equiv) was added and heating at 60° C. for 30 min. After that stirring was continued at room temperature for 0.5 h. The reaction mixture was washed with sodium bicarbonate solution and combine organics was extracted by dichloromethane solvent. The solution was dried over anhydrous Na₂SO₄ and after evaporation of solvent we got crude product. Then, the resulting concentrate was purified on a silica column to get 1.2 g (purity=99%) yellow oil compound with a yield of 94%.

TLC R_{f}=0.6 (PE:EA=2:1)

LCMS (m/z)=454.50 (M+1)

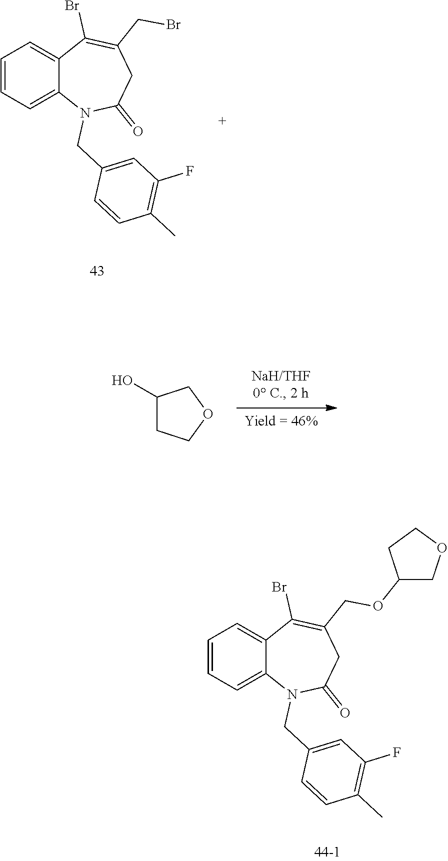 [SMILES: Cc1ccc(CN2C(=O)CC(CBr)=C(Br)c3ccccc32)cc1F ; OC1CCOC1 ; Cc1ccc(CN2C(=O)CC(COC3CCOC3)=C(Br)c3ccccc32)cc1F]

### 5-bromo-1-(3-fluoro-4-methylbenzyl)-4-(((tetrahydrofuran-3-yl)oxy)methyl)-1,3-dihydro-2H-benzo[b]azepin-2-one (44-1)

A round-bottom flask was charged with NaH (30 mg, 0.75 mmol, 2.5 equiv) and dry THF (1 mL), tetrahydrofuran-3-ol (53 μL, 0.6 mmol, 2 equiv) was added into the NaH solution in ice-cold condition. The resulting mixture was stirred continuously for 30 minute at 0° C., then 5-bromo-4-(bromomethyl)-1-(3-fluoro-4-methylbenzyl)-1,3-dihydro-2H-benzo[b]azepin-2-one 43 (135 mg, 0.3 mmol, 1 equiv) in THF (1 mL) was added in it. After completion of the reaction (2 h), the solution was quenched with saturated ammonium chloride and extracted with EA (3×10 mL). The organic solutions were combined, washed with H₂O (2×10 mL) and brine (1×20 mL) dried over anhydrous Na₂SO₄ filtered and concentrated by rotary evaporation to get the crude compound. Then, the resulting concentrate was purified on a silica column to get 64 mg (purity=91%) yellow oil compound with a yield of 46%.

TLC R_{f}=0.2 (PE:EA=2:1)

LCMS (m/z)=460.60 (M+1)

### Example 42. Representative Synthesis of Compound of Formula 44-2

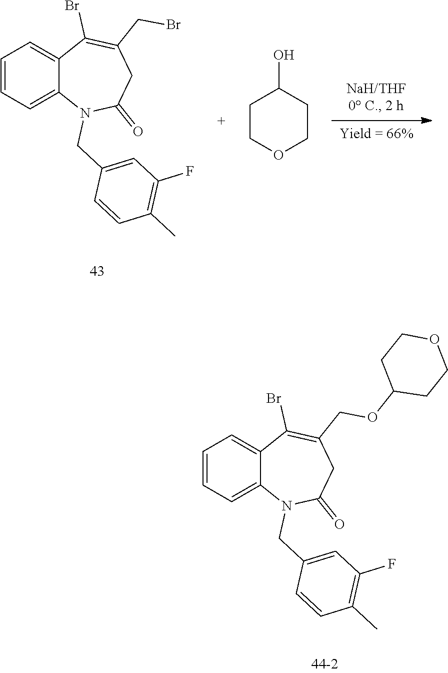 [SMILES: Cc1ccc(CN2C(=O)CC(CBr)=C(Br)c3ccccc32)cc1F ; OC1CCOCC1 ; Cc1ccc(CN2C(=O)CC(COC3CCOCC3)=C(Br)c3ccccc32)cc1F]

### 5-bromo-1-(3-fluoro-4-methylbenzyl)-4-(((tetrahydro-2H-pyran-4-yl)oxy)methyl)-1,3-dihydro-2H-benzo[b]azepin-2-one (44-2)

A round-bottom flask was charged with NaH (22 mg, 0.55 mmol, 2.5 equiv) and dry THF (1 mL), tetrahydro-2H-pyran-4-ol (41.75 μL, 0.44 mmol, 2 equiv) was added into the NaH solution in ice-cold condition. The resulting mixture was stirred continuously for 30 minute at 0° C., then 5-bromo-4-(bromomethyl)-1-(3-fluoro-4-methylbenzyl)-1,3-dihydro-2H-benzo[b]azepin-2-one 43 (100 mg, 0.22 mmol, 1 equiv) in THF (1 mL) was added in it. After completion of the reaction (2 h), the solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 69 mg (purity=80.69%) yellow oil compound with a yield of 66%.

TLC R_{f}=0.2 (PE:EA=2:1)

LCMS (m/z)=474.60 (M+1)

### Example 43. Representative Synthesis of Compound of Formula 44-3

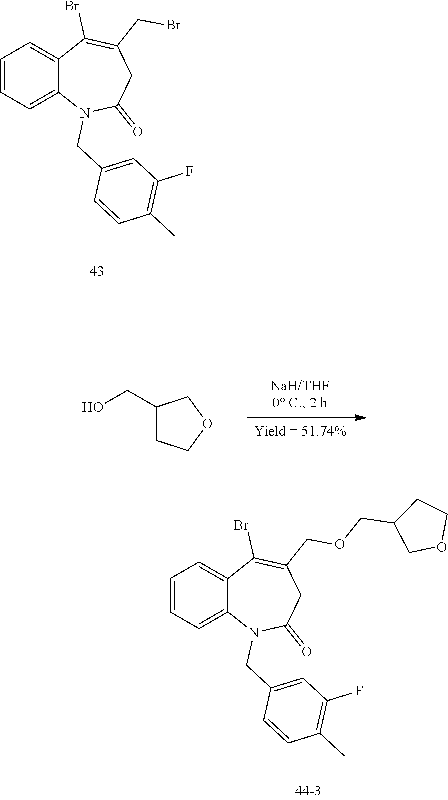 [SMILES: Cc1ccc(CN2C(=O)CC(CBr)=C(Br)c3ccccc32)cc1F ; OCC1CCOC1 ; Cc1ccc(CN2C(=O)CC(COCC3CCOC3)=C(Br)c3ccccc32)cc1F]

### bromo-1-(3-fluoro-4-methylbenzyl)-4-(((tetrahydrofuran-3 yl)methoxy)methyl)-1,3-dihydro-2H-benzo[b]azepin-2-one (44-3)

A round-bottom flask was charged with NaH (22 mg, 0.55 mmol, 2.5 equiv) and dry THF (1 mL). (tetrahydrofuran-3-yl)methanol (43.44 μL, 0.44 mmol, 2 equiv) was added into the NaH solution in ice-cold condition. The resulting mixture was stirred continuously for 30 minute at 0° C., then 5-bromo-4-(bromomethyl)-1-(3-fluoro-4-methylbenzyl)-1,3-dihydro-2H-benzo[b]azepin-2-one 43 (100 mg, 0.22 mmol, 1 equiv) in THF (1 mL) was added in it. After completion of the reaction (2 h), the solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 54 mg (purity=96.44%) yellow oil compound with a yield of 51.74%.

TLC R_{f}=0.2 (PE:EA=2:1)

LCMS (m/z)=476.60 (M+1)

### Example 44. Representative Synthesis of Compound of Formula 44-4

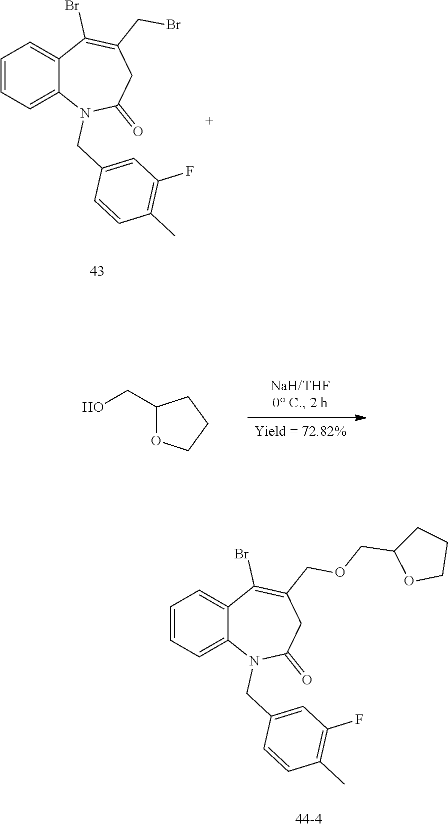 [SMILES: Cc1ccc(CN2C(=O)CC(CBr)=C(Br)c3ccccc32)cc1F ; OCC1CCCO1 ; Cc1ccc(CN2C(=O)CC(COCC3CCCO3)=C(Br)c3ccccc32)cc1F]

### 5-bromo-1-(3-fluoro-4-methylbenzyl)-4-(((tetrahydrofuran-2-yl)methoxy)methyl)-1,3-dihydro-2H-benzo[b]azepin-2-one (44-4)

A round-bottom flask was charged with NaH (22 mg, 0.55 mmol, 2.5 equiv) and dry THF (1 mL). (tetrahydrofuran-2-yl)methanol (43.44 μL, 0.44 mmol, 2 equiv) was added into the NaH solution in ice-cold condition. The resulting mixture was stirred continuously for 30 minute at 0° C., then 5-bromo-4-(bromomethyl)-1-(3-fluoro-4-methylbenzyl)-1,3-dihydro-2H-benzo[b]azepin-2-one 43 (100 mg, 0.22 mmol, 1 equiv) in THF (1 mL) was added in it. After completion of the reaction (2 h), the solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 76 mg (purity=93.03%) yellow oil compound with a yield of 72.82%.

TLC R_{f}=0.2 (PE:EA=2:1)

LCMS (m/z)=474.60 (M+1)

### Example 45. Representative Synthesis of Compound of Formula 44-5

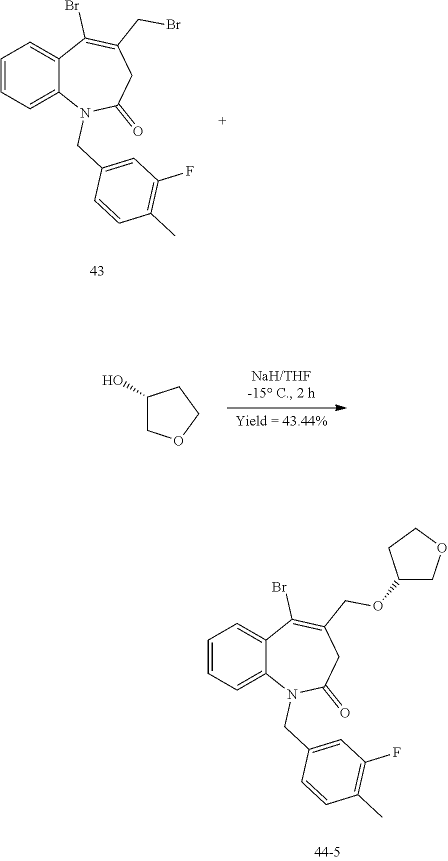

### (R)-5-bromo-1-(3-fluoro-4-methylbenzyl)-4-(((tetrahydrofuran-3-yl)oxy)methyl)-1,3-dihydro-2H-benzo[b]azepin-2-one (44-5)

A round-bottom flask was charged with NaH (28 mg, 0.71 mmol, 2.5 equiv) and dry THF (1 mL). (R)-tetrahydrofuran-3-ol (46.09 μL, 0.57 mmol, 2 equiv) was added into the NaH solution in ice-cold condition. The resulting mixture was stirred continuously for 30 minute at −15° C., then 5-bromo-4-(bromomethyl)-1-(3-fluoro-4-methylbenzyl)-1,3-dihydro-2H-benzo[b]azepin-2-one 43 (100 mg, 0.22 mmol, 1 equiv) in T-1F (1 mL) was added in it. After completion of the reaction (2 h), the solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 56 mg (purity=88.62%) yellow oil compound with a yield of 43.44%.

TLC R_{f}=0.25 (PE:EA=2:1)

LCMS (m/z)=460.60 (M+1)

### Example 46. Representative Synthesis of Compound of Formula 44-6

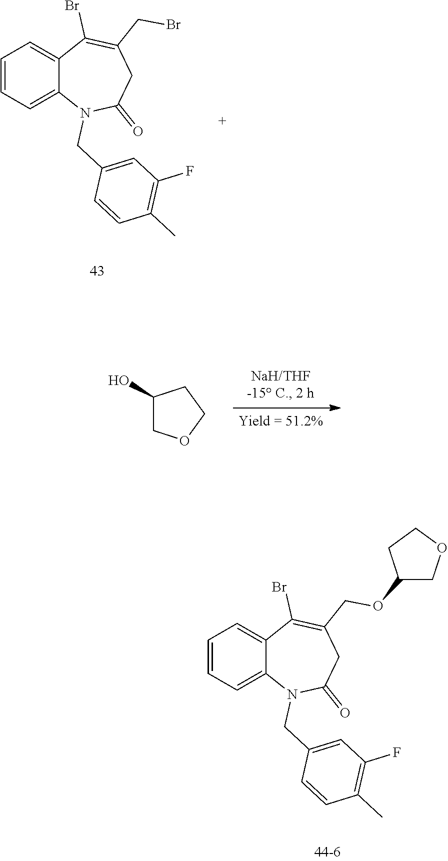

### (S)-5-bromo-1-(3-fluoro-4-methylbenzyl)-4-(((tetrahydrofuran-3-yl)oxy)methyl)-1,3-dihydro-2H-benzo[b]azepin-2-one (44-6)

A round-bottom flask was charged with NaH (28 mg, 0.71 mmol, 2.5 equiv) and dry THF (1 mL). (S)-tetrahydrofuran-3-ol (46.09 μL, 0.57 mmol, 2 equiv) was added into the NaH solution in ice-cold condition. The resulting mixture was stirred continuously for 30 minute at −15° C. then 5-bromo-4-(bromomethyl)-1-(3-fluoro-4-methylbenzyl)-1,3-dihydro-2H-benzo[b]azepin-2-one 43 (100 mg, 0.22 mmol, 1 equiv) in THF (1 mL) was added in it. After completion of the reaction (21 h), the solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 66 mg (purity=84.08%) yellow oil compound with a yield of 51.20%.

TLC R_{f}=0.25 (PE:EA=2:1)

LCMS (m/z)=460.60 (M+1)

### Example 47. Representative Synthesis of Compound of Formula 45

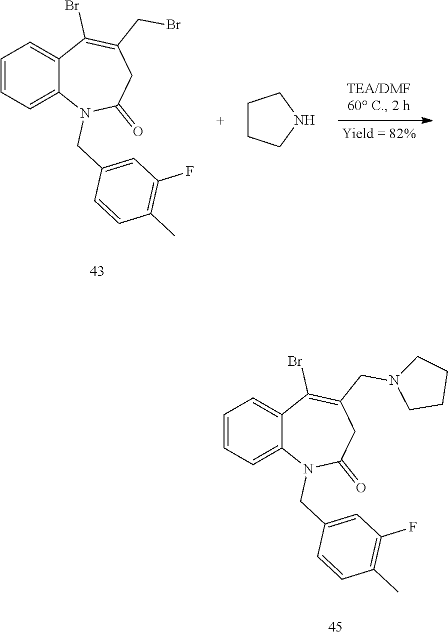 [SMILES: Cc1ccc(CN2C(=O)CC(CBr)=C(Br)c3ccccc32)cc1F ; C1CCNC1 ; Cc1ccc(CN2C(=O)CC(CN3CCCC3)=C(Br)c3ccccc32)cc1F]

### 5-bromo-1-(3-fluoro-4-methylbenzyl)-4-(pyrrolidin-1-ylmethyl)-1,3-dihydro-2H-benzo[b]azepin-2-one (45)

To a solution of 5-bromo-4-(bromomethyl)-1-(3-fluoro-4-methylbenzyl)-1,3-dihydro-2H-benzo[b]azepin-2-one 43 (135 mg, 0.3 mmol, 1 equiv) and TEA (42 μL, 0.3 mmol, 1.2 equiv) in DMF (1 mL) was added pyrrolidine (21 μL, 0.25 mmol, 1 equiv) at rt. The reaction mixture was then stirred at 60° C. for 2 h. The solution was quenched with saturated ammonium chloride and extracted with EA (3×20 mL). The organic solutions were combined, washed with H₂O (2×20 mL) and brine (1×20 ml), dried over anhydrous Na₂SO₄, filtered and concentrated by rotary evaporation to get the crude compound. Then, the resulting concentrate was purified on a silica column to get 110 mg (purity=96%) of the light yellow oil compound with a yield of 82%.

TLC R_{f}=0.2 (PE:EA=2:1)

LCMS (m/z)=445.20 (M+1)

### Example 48. In Vitro Assays

### Cell Culture and Treatment

Human cancer cell lines were obtained from ATCC and cultured in DMEM (Gibco, Cleveland. TN, USA) supplemented with 5% fetal bovine serum (Gibco), penicillin (100 U/mL)-streptomycin (100 μg/mL) (Gibco, Cat. No. 15140-122), 2 mM L-glutamine (Gibco, 200 mM solution, Cat. No. 25030081), and 1 mM sodium pyruvate (Gibco, 100 mM solution, Cat. No. 11360070) at 37° C. in a humidified incubator that was maintained at 5% CO₂.

### Phenotypic Screening Assays and MTT Assays

Screening was conducted as described before (Li et al. 2019). Briefly, RPE-Neo or RPE-MYC or RPE-MYC expressing an EGFP-Histone 2B fusion protein were passaged as batches of 96-well plates, 18-24 hours before exposure to the chemical compounds of the present disclosure at concentrations from 20 nM to 20 μM. At 24, 48 or 72 hours after initiation of treatment, cells were analyzed for either an arrest in mitosis or a change in DNA content by GE 1N-Cell Analyzer 2000. MTT assays of cellular proliferation were performed as previously described (Li et al. 2019).

### Immunofluorescent Staining

The immunofluorescence staining procedure has been previously described (Yang et al. 2010). Briefly, cells were cultured on coverslips in a 6-well plate, fixed with 4% paraformaldehyde, and then permeabilized with 0.3% Triton X-100 in PBS. After incubation with a primary antibody, cells were detected with TRITC-conjugated or FITC-conjugated secondary antibodies that were purchased from Jackson ImmunoResearch. After immunostaining, cells were mounted on microscope slides with DAPI-containing Vectashield mounting solution (Vector Laboratories, USA) and fluorescence was detected with an EVOS Auto FL fluorescence microscope (ThermoFisher, USA).

Antibodies Used in this Study

### Primary Antibodies:

Histone H3 (phospho-Ser10), Mouse Monoclonal Antibody. Upstate, Cat #05-806, 1:1000 Phospho-Aurora A (Thr288)(C39D8), Rabbit mAb, Cell Signalling, #3079, 1:1000 Aurora B Antibody, Rabbit, Absin, abs1460-100 ug, lot #AH22, 1:50 dilution CREST antibody, Thermofisher, Cat. #PA5-30500, 1:200 dilution Mouse Anti-β-tubulin, Sigma, Cat. #F 2043, clone TUB 2.1 1:50 dilution Secondary antibodies:
Rhodamine (TRITC) AffiniPure Goat Anti-mouse IgG (H+L), Proteintech, SA00007-1, 1:100
Rhodamine (TRITC) AffiniPure Goat Anti-Rabbit IgG (H+L), Jackson, 111-025-003, 1:100
Fluorescein (FITC)-conjugated Affinipure Goat Anti-Rabbit IgG(H+L), Jackson, 111-095-144, 1:100
Rhodamine RedTM—X-conjugated AffiniPure Donkey Anti-Human IgG (H+L), Jackson, 709-295-149, 1:100

### Crystal Violet Assay

Cells were fixed in 4% PFA, stained with 0.1% crystal violet (Aladdin, 548-62-9). Bound crystal violet was eluted with 95% ethanol and the absorbance of eluate was measured at 570 nm using a Microplate reader (BioTek ELX808iu).

### Soft Agar Assay

The assay was performed in 6-well plates with two layers of agar. For the first, 0.75% agar in DMEM medium was melted in a microwave oven and poured to form a bottom layer. Once solidified, 10-100K cells in 1 ml of DMEM containing 0.35% agar was added to form the top layer, which was later covered with 0.5 ml DMEM. Cell culture medium was changed once every two days until colonies were ready to photograph.

The results of these assays are summarized in Table 2. As set forth in table 2 below, an values of greater than or equal to 1 nM and less than or equal to 1.0 μM is marked “A”; a value greater than 1.00 μM and less than or equal to 10.0 μM is marked “B”; a value greater than 10.0 μM and less than or equal to 30.0 μM is marked “C”; and a value greater than 30.0 μM and less than 1000 μM is marked “D.”

TABLE 2

| | | Min. Effective | | |
|---|---|---|---|---|
| | | conc. for | | |
| | Mitotic Arrest | Polyploidy | Along with | Proliferative |
| Code | (>5%) | (≥5%) | Cell Death | arrest (>50%) |
| 7-1 | A | A | A | A |
| 8-1 | A | A | A | A |
| 8-2 | A | A | A | A |
| 8-2 | A | A | A | A |
| 8-3 | A | A | A | A |
| 8-4 | A | A | A | A |
| 8-5 | A | A | A | A |
| 8-6 | A | A | A | A |
| 8-7 | A | A | A | A |
| 8-8 | A | A | A | A |
| 8-9 | A | A | A | A |
| 8-10 | A | A | A | A |
| 8-11 | A | A | A | A |
| 8-12 | A | A | A | A |
| 8-13 | A | A | A | A |
| 10-1 | A | A | A | A |
| 13-1 | A | A | A | A |
| 13-2 | A | A | A | A |
| 15-1 | A | A | A | A |
| 15-2 | A | A | A | A |
| 16-1 | A | A | A | A |
| 18-1 | A | A | A | A |
| 20-1 | A | A | A | A |
| 20-2 | A | A | A | A |
| 20-3 | A | A | A | A |
| 20-3 | A | A | A | A |
| 20-4 | A | A | A | A |
| 20-5 | A | A | A | A |
| 20-6 | A | A | A | A |
| 20-7 | A | A | A | A |
| 20-8 | A | A | A | A |
| 20-9 | A | A | A | A |
| 20-10 | A | A | A | A |
| 20-11 | A | A | A | A |
| 20-12 | A | A | A | A |
| 20-13 | A | A | A | A |
| 20-14 | A | A | A | A |
| 20-15 | A | A | A | A |
| 20-16 | A | A | A | A |
| 20-17 | A | A | A | A |
| 20-22 | A | A | A | A |
| 20-23 | A | A | A | A |
| 20-20 | A | A | A | A |
| 20-18 | A | A | A | A |
| 20-21 | A | A | A | A |
| 20-26 | A | A | A | A |
| 20-27 | A | A | A | A |
| 20-25 | A | A | A | A |
| 20-28 | A | A | A | A |
| 20-30 | A | A | A | A |
| 20-29 | B | B | B | B |
| 20-32 | A | A | A | A |
| 22-1 | B | B | B | B |
| 23-1 | A | A | A | A |
| 23-1 | A | A | A | A |
| 24-1 | A | A | A | A |
| 24-2 | A | A | A | A |
| 26-1 | B | B | B | B |
| 27-1 | D | D | D | D |
| 28-1 | B | B | B | B |
| 42 | A | A | A | A |
| 44-1 | A | A | A | A |
| 45 | B | B | B | B |
| 44-2 | B | B | B | B |
| 44-3 | B | A | A | A |
| 44-4 | B | B | B | B |
| 44-5 | A | A | A | A |
| 44-6 | A | A | A | A |
| 40 | B | B | B | B |
| 34-1 | A | A | A | A |
| 34-2 | A | A | A | A |
| 34-3 | B | B | B | B |
| 34-4 | B | B | B | B |

### Example 49. Xenograft Studies

Xenografts were initiated in immunocompromised (Nu/Nu) mice with the human lung adenocarcinoma cell line NCI-H23 (FIG. 1) and the human mammary tumor derived cell line MDA-MB-231 (FIG. 2). Five million cells were injected subcutaneously into mice and treatment was initiated when the average tumor volumes reached 150-200 mm³ (n=8/groups). The indicated compounds were administered through oral gavage twice a day for five days followed by a resting period of 2 days. Day 0 on the X-axis indicates the day that treatment was initiated. For these experiments, 55 and 58 were first dissolved in DMSO and then diluted 1:10 into a mixture containing 70% PEG300 and 30% PBS, pH7.4. 100 ml of drug solution was administered with each dose.

### REFERENCES

Li et al. 2019
Purification, identification, and characterization of two benzophenanthridine alkaloids from Corydalis longicalcarata rhizomes with anti-mitotic and polyploidy-inducing activities Jinhua Li, Ziqi Yan, Hongmei Li, Qiong Shi, Linfang Huang, Thaddeus D. Allen, Dun Yang, Jing Zhang, bioRxiv 821124; doi: https://doi.org/10.1101/821124
Yang et al. 2010
Therapeutic potential of a synthetic lethal interaction between the MYC proto-oncogene and inhibition of aurora-B kinase
Dun Yang, Hong Liu, Andrei Goga, Suwon Kim, Mariia Yuneva, and J. Michael Bishop PNAS Aug. 3, 2010 107 (31) 13836-13841; https://doi.org/10.1073/pnas.1008366107

1. A compound of formula (I): 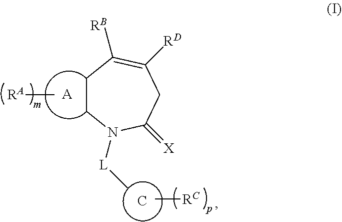
or a pharmaceutically acceptable salt thereof
wherein
X is O═ or S═;
Ring A is selected from the group consisting of optionally substituted phenyl, optionally substituted 5-membered heteroaryl, and optionally substituted 6-membered heteroaryl;
L is selected from the group consisting of a bond, optionally substituted C₁-C₃ alkylene, —CH(D)-, —C(D)₂-, —C(O)—, —C(O)O—, —C(O)NH—, and —S(O)₂—;
Group C is selected from the group consisting of C₁-C₆ aliphatic, —C≡CR₃, optionally substituted phenyl, optionally substituted 5-membered heteroaryl, optionally substituted 6-membered heteroaryl, optionally substituted 6-membered carbocyclyl, and optionally substituted 6-membered heterocyclyl;
each R^{A} is independently selected from the group consisting of halogen, —CN, —C≡CR³, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂; or
R^{B} is independently selected from the group consisting of halogen, —C≡CR³, optionally substituted 5-6-membered heteroaryl, optionally substituted C₁-C₃ aliphatic, and optionally substituted C₁-C₃ alkoxy, and optionally substituted 3-6-membered carbocyclyl;
each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —N(R³)₂, —C(O)OR³, —C(O)N(R³)₂, —N(H)C(O)R³, and —N(H)C(O)N(R³)₂;
wherein optionally two instances of R^{C} are taken together with the atoms on which they are attached to form an optionally substituted 6-membered aryl or optionally substituted 6-membered heteroaryl;
R^{D} is selected from the group consisting of optionally substituted 5-membered heteroaryl, —C(O)OR³, C(O)N(R³)₂, —(CH₂)₁₋₃N(R²)₂, —(CH₂)₁₋₃OR², —(CH₂)₁₋₃O(CH₂)₁₋₃R², and —CHO;
each R¹ is independently selected from the group consisting of hydrogen, —C(O)R³, —(CH₂)₁₋₃OR³, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein optionally two instances of R² are taken together to form an optionally substituted 3-7 membered heterocyclyl ring;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
m is 0, 1, 2, 3, or 4;
p is 0, 1, 2, 3, 4, or 5.

2. The compound of claim 1, wherein the compound is of Formula (I-a), (I-b), or (I-c): 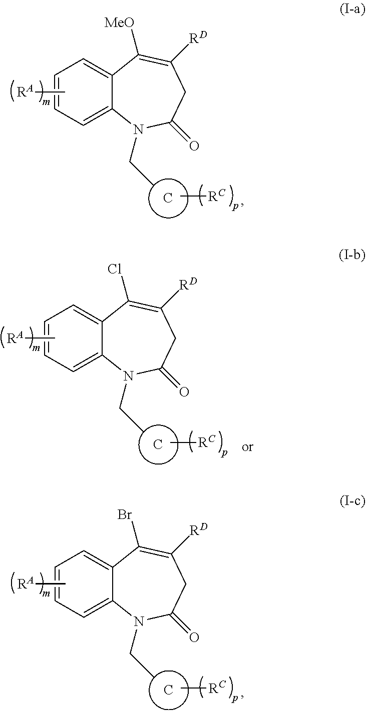
or a pharmaceutically acceptable salt thereof.

3. The compound of claim 2, wherein Group C is optionally substituted phenyl.

4. The compound of claim 3, wherein Group C is an optionally substituted pyridinyl.

5. The compound of any of claims 1-4, wherein R^{C} is selected from halogen, optionally substituted C₁-C₆ aliphatic, —COOR³, —CN, and —OR³.

6. The compound of any of claim 1-5, wherein p is 2.

7. The compound of claim 6, wherein one instance of R^{C} is halogen and the other is optionally substituted C₁-C₆ aliphatic or —OR³.

8. The compound of any of claim 1-7, wherein R^{C} is selected from —F, -Me, and —OMe.

9. The compound of any of claims 1-8, wherein p is 2 and one instance of R^{C} is fluoro and the other is -Me or —OMe.

10. The compound of any of claims 1-9, wherein L and Group C are taken together to form

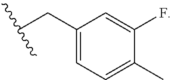 [SMILES: *Cc1ccc(C)c(F)c1]

11. The compound of claim 1, wherein the compound is of Formula (I-a1), (I-b1) or (I-c1): 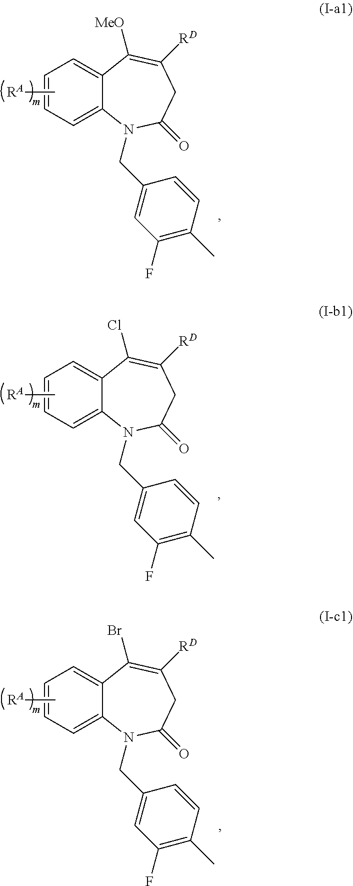
or a pharmaceutically acceptable salt thereof.

12. The compound of any of claims 1-11, wherein R^{D} is an optionally substituted 5-membered heteroaryl.

13. The compound of any of claims 1-12, wherein R^{D} is —(CH₂)₁₋₃OR².

14. The compound of any of claims 1-12, wherein R^{D} is —CHO.

15. The compound of any of claims 1-12, wherein R^{D} is selected from the group consisting of

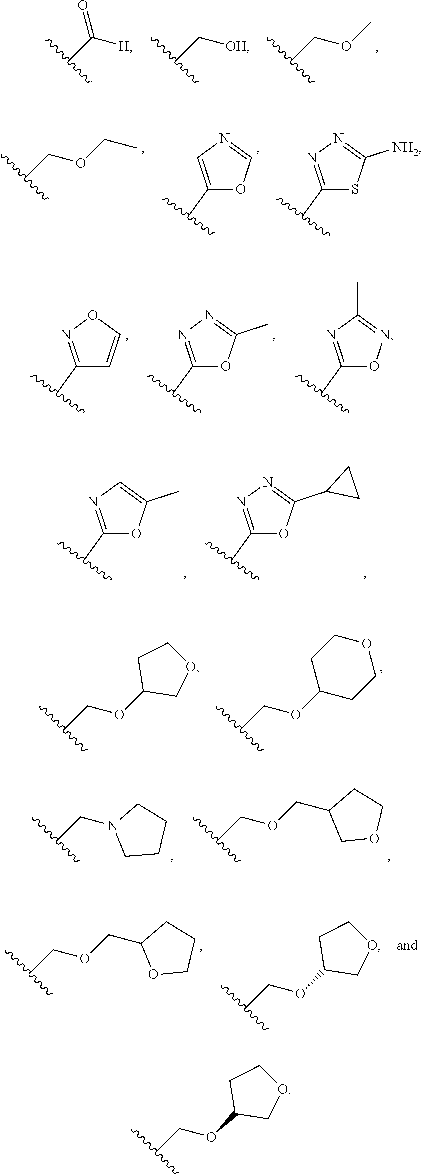

16. The compound of claim 2, wherein the compound is of formula (I-b2): 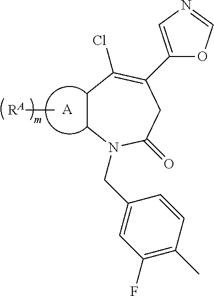
or a pharmaceutically acceptable salt thereof.

17. The compound of claim 2, wherein the compound is of formula (I-c2), (I-c3) or (I-c4) 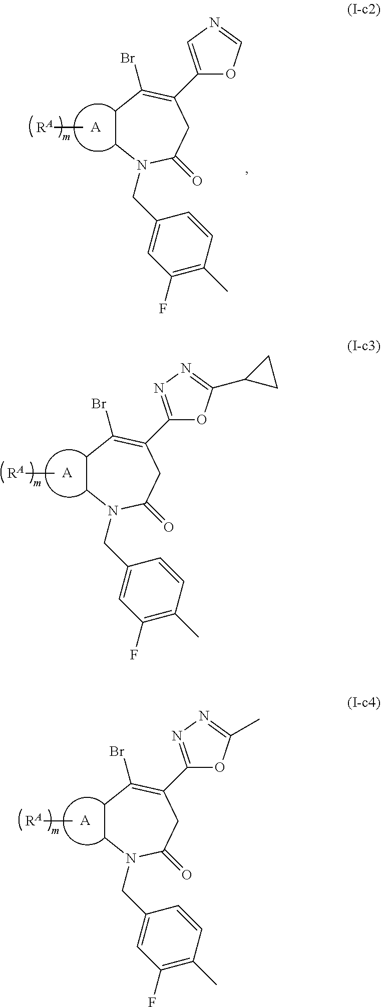
or a pharmaceutically acceptable salt thereof.

18. The compound of any of claims 1-17, wherein R^{A} is optionally substituted phenyl.

19. The compound of any of claims 1-18, wherein R^{A} is selected from the group consisting of

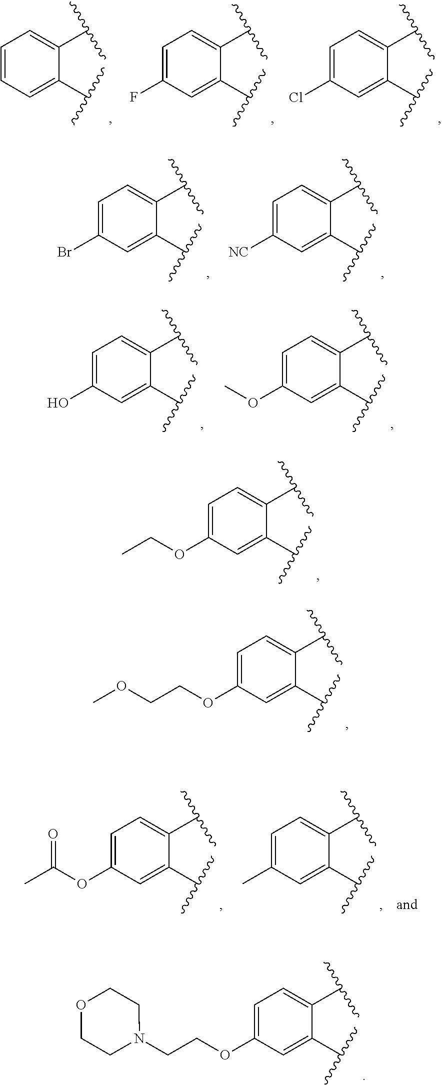

20. The compound of any of the previous claims, wherein the compound is described in Table 1.

21. A pharmaceutical composition comprising a compound of any of the previous claims and a pharmaceutically acceptable excipient.

22. A method of treat cancer comprising administering to a patient in need thereof the compound of any of claims 1-20 or the pharmaceutical composition of claim 21.
